SEQUENCE LISTING (113 sequences)

SEQ ID NO: 1 - DNA, 6459 bp, Propionibacterium acnes
atgaattattttccagacaatcttgtaaacgctgtgctaaaacctgtacatggggttctagaacaaacgaataatggtttccagggacatcaataatcttaatttcttgagccgccattacagaaaataattctacccaaactaaggtcgggtcaggagccataatatgtttttccctggctcgaaatacagtgacttttcctggatatggttgtcttatataggaataagttgcttttaaagttcccaccaaaacatcaagaatacggcgattattttgacgttctacaccaggcgggaatattctagcgctccgtgctttatcaatgatgtaattaattttttcttctacagttaaattttctatttcttcaggtgtgactagattatcttgaccaaacataccgccaaaaactctggagagaacaccaactaaataaacgtcatcaatgggtttttgtttatccagcagaatcggtacgtaagaatctaatattgctagtaaagatacttcttgtccttgtctatgtaactgctgtgctacttcataagctacgactccaccaaatgaccaccccccgacacgataaggcccttggggttgaaattctctaatagttttgacgtagagactagccatatcttcaactcgcgtcaagggtgcttcatctccataaaatccttgagcttgtaagccataaaatggttggtcagttcctatatattgtgcgagtttaaaatagcataaaacatgaccaccagcaggatgtatacaaaagaaaggctgctgcttaccttgtggttgaattggaactaggggtgaattatggatttggttgtttgcttgaataaccttggctaaatctgcaattactggatttgttaaaagtgttgctaaggatatctctttagcaaataactcttcaatttgagaaattaaatgtagagctttgagggaattaccaccaatagcgaaaaagttttctgtcacacctactttaggtaaatgcagaatattcgaccagatttgtactaatttttcttcaacttcattccgaggcgctacataggaattatgttcactataattaaataaatcaggcttaggtaatgccttacggtctactttaccactgggagttaaaggaagatgctccagcatcacaaaagcggctggaatcataaaatcaggtagccttgcttttaggaaatcacgcagattatcaagctgaggtttgatggagttataggtaatataagcgatgatttgtttttcttgagcgttatcatcccgcgctatgactacagcttctctgacttgtgggtgtgaagataaaacattttcaatctcgccaatttcaattctataaccccgaatttttacttgataatctgttctaccaagatattcaatatttccatcgggtaaataacgagctaaatcacctgttttataaagtcgcttaaactcagaattgggaaagggattaataataaatttttctttggtcaattcttctttattcaaataaccacgagcaacccctacaccaccaatataaatttcaccagtgacaccaatatttactggttgtaaatcggcatcaagaatataaatttgagtattagcaatgggacgaccaataggtacactctttaaattactatcttttctacattgccaaaatgtgacatcaattgctgcttctgtcgggccatagaggttatgtaattcacattgcaaatgctggaaaaatctattttgtaaatctatagataaagcttcaccgctacaaataactctttttagagagctgcatttgcttacatggcgattttgtaaaaacacttgcagcattgaggggacaaaatgcaacgtagtgatttgttcttgagtaattaaatcgatgaggtaagcactatctttatgtccgcctggtttggctattaccaaacgtgcgccagttaataaagtccaaaagaactcccaaacggaaacatcaaaactaaagggggttttttgtaaaatgctatctgtggaatcgatttgataagcttcctgcatccacaataagcgattacagatacctttgtgggtgttcattgcaccttttggtttaccagtggaaccagaggtgtaaattacataagcaagattatccgtttttatattacttttgggattggtattagcttgtgtggaaattttctcccattccctatctacacagatagtttgtgcttgatggtggggaatttgattgagtaatttttcttgagttagtagtaccttcacctgagaatcttctagcatataagctatgcgttcttgaggatattcagggtcaataggaacataagcacccccagctttgaggattcctaaaagacagataaccatttctaaggaacgttctaaacaaacgcctaccagggtttctggctggactcctaatgtttgtaaataatgtcctagctggtttgctttatgatttagttcttgataagttagttgttgcttgtcaaaggtgacagcgatcgcctcaggtgttcgttctacttgagctacaattagttcatgtaaactctgggaaagatcataatctctgtgggtcgcgttccactctacaagtaacttacgaatattaaaatctatagtctgcatatcttctaactttgctcaataataaaaaatttctcacgcagagacgcagagaaaacacactccgcgtccctctctcttgaaaagtttcctacggagggaaaccctcctccagaacttttcgctgcgctaacctcagcgtccctctgcgtttaaaaactaatctccccccaattccaccaacttcccaatattgaaatctatccgcgtccaacctttaagacgacgcaaattattcaataacagtaggggaatttgccaatgatgtaccatcaaaaacggtaaggggtcatctggctgataaatcgcatcagtcccgcgccaaatattccccagccatctttgcaactgggtgaaagaacggataccagttaaacgccaaacttcgtgataagtccaataggtaggcttgcttgtcgttagtggttgtatggtttcagtcgtcggtgcttgactcaagtacgcttctgctacctgggggtggtcgtaaaatgtggtaattgctgaatgtgtgcgggggttacactcgatggcgtaaactgttccgtcttcggcttggataaagtcaaaggaaatctgtcctgtcagtttcagttccttgacaaaatgctgtacccattcggtaatttgcgggttatttacattctcataattaacttggaaggctgaagattcgcaacagcaatgcagtctgagttccccattccgaacggtgctatgggtgcagaattccttaccggggataaattcctgcataatccacggtttttcgggagtaattggcaaacttctgacgaatgctgctgtttcctctggagtagcacaggggagtttggttaagtccaaccgccgcactgagtcgtagggaatgcttttgaggatgtatttacgtgtctctccagaaaaatcgaagttgatgacttgttctggtgaggtaattttaaaggatttgggtactgataaaccaagcgatcgcgctttttgtgtcaacgcaaatttatcatccaacatttgggtaatatctgcgtcaaagtgaaacacttcgcaataatgggataactctggtttggctaatgagtcgtagtagctacccactggactggtgacgggaatataaacatcgatgttttcttgtttgacgatatctaccaaagcctgaatgtaagcttggggattgtcctggggtgcggggactgtgtaaaacttatccactgcttgggaaaaacgatgaccagtcaaccagtatttatgggtttccaccaagacaactctatgtccagccgcgtggaatgaccttgctagttgtaaagctttggtcatcttaccgccactgataagaatggtttgggggtttgctgctttgaccttttgcggtcggaagactaacaaggatataaaaacaatggtggcattaatgggcaatgctagtaataacaaagccaaagtgcagatattttggataattgcggctattttcgtctgggaaggaagagacggtgtagcaggtgcggaagaaaggggaagggattgtgccatagtcgattggacaattaaggttgtattctgcggataattgttaaaccatctcgcaacggcaacaaaacctgttctacacgggggtctatagctactgtatgattaaattgcgcgatcgcttcaccattgacgctacgttcctctgctggtagataaacttccccttgtaataaggtgttatctacacaaataaagccatctggtgctaacaaactgctacctagcaacttgtgaaaataggctacatactcttttttatctgcgtcgataaataccaagtcaaaagactccccagcttctgctaacttatcaagagttgctaaggctgcatccaattccacacgaatctttccaccgtggggagattgttgaaaggctttctgtccaatttccgccgcgtaagggtcaacttcacaagccacaagcagtccatcctctggtaatgcttccgccatcgccagcgccgaataaccggtaaacatcccaatttctaagacttttttagctttggtcatgtgaacaaacatctttaaggtttgtccttcgatatgaccagaaagcatctcttgttctagaggacggacggttgtacctccgtggaagtgttctccccaggcttcggtggctgtggtttttgccaatgcagcgagttcaggagattctggagtggtgcattcttccaaataagggtctatacctgcggctaaacgccaagcctgatggatgtttgctatcaattccccaggtaaatctggatgttgcttaacctcttggactatggcttctaactgcttggttaaaattcccaatggtgtaacaggtctagctgttggttggacaatcacatttgtcaagtcgcttcgctccaattcaaaattcaaaattcaaaattcaaaattaaagacaattagtgtccgattatttgcgtagccttctctttccctacgggacgctccgcgaacagaaatgctaccgcgctcgcgcagtgtatccgtggagtattttgcattttgaattcaaaaaagtcattatttaacactcccgattaattctttttgataaacgggatacacatccacaccttcaccaccacggggataactggtacaaagttctttgtggtcagctaatgcggctgctaattcttctcttgtcaggtcattgacgaagacacaatcaccaatgggttttggcatagcagctcttaacaaaccatcacgagttaatgtgatagattcagtaccacgccacaaaatatctatatccaacatgggatggtcgagggatagaccaacgcgactcattaatcctaaaatacgatcgcgttctgcaattgtaatatatcctctacgggcggcgatcgttgccgagaaagccatatctacattaacggcgtgtccgtggaacatgggtagacgaggcgcaagttccaaggtgggactccaagtgtgaccgtaagcaatcaccctatctaggtctaactcatgcaggttgggaacttccaattccaacatcttatggatagctttgtaagtcaaacgatgggctatttctttaatctctggagttgcatctatattgccaaaatgagtacgtagtaattcttcgccgtacttctccaacaattcaaaaacttcttgatgcgctactacagcgatttttaccaattccgccatcccgttacgtacttggtctgtagggagagtacgcaacaaggagaaatctaaaaatactttgcgagaagcatgataagcacccaaacggtttttcagtttgcgatgattaactgctaccttaattgctacactggcatcaattaatccaatcaatgtagtaggaatgcggatgtaattgctgctgcgacgatatgtagaacaagcaaagccgacaacatctgtaattaaaccgccacccacgactaatactggttctttgcggactaatttgaaatctgcaaagacatctataactctctcgaaagtttgaatagtcttatctggttcagtaatggtaataggaaatagcctcagttctataccataatactggaaatatgcctgaatttgattaccatacaaccgactgacgttagcatctacaatcgccaagcatcgtccaaaaccttgatatacatctgctagtgcagaattctggatttcaaaaataccatctacatacaccaaatcatactcaatcttttcgtaaccttctacatgaaaagatgtttccttagcttcaaactttgcttggacgatactcat

SEQ ID NO: 2 - DNA, 7210 bp, Propionibacterium acnes
cttttaaacattctgctaaaacctgtacatgaggttctaaaacaaacgaataatgatttcctggcacatcaataatattaatttcctctgctgccattacagaaaataactccacccaaactaaagtgggatcaggagccataatatgtttctccctagctctaaaaattgttacttttccaggatatggctgtcttttataggaataagttgcttttaaagttcccaccaatacatctaaaatgcggcgattattttgacgttccacaccaggaggaaaaattttcgcttttctggctttgtcaatgatataattaattttttcttctaaacttaaatgctggatttcttctggtgtaactaaattatcttgaccaaacattcccccgaagactctagaaagtacacccacgagataaacatcatcaatttgtttttgtttatctaataaaatgggtacgtaagaatcaagtatggctaacaaagatacttcttgtccttgctttattagctgctgtgctacctcataagcaaccactcccccaaacgaccaaccccctaattgataaggcccttggggttgaaattctctgatagttttcacatagaggctggccatatcttcaactttagttaaaggttcctctttcccataaaaaccctgagcttgcaaaccataaaacggttgttcattgcccatgttatgggctaatttgaagtaacataaaacatgaccaccggcaggatgtatacaaaagaaaggttgcttcttaccttgtggctgaatgggaactaaaggagaattctgaatcagattactggaatcttgaataactgctgctaaatctgtaattactgggttctttaatagtgttgctagagggatttcttttccaaaatcttgttcaattttagagattaaatgtagtgctttaagtgaatttccacctaacgcgaaaaagttatcttttactcctatttgtggtaaatttaggatgtctgaccaaatttttactaattgtgcttctacttgattacgaggagcgacaaaatcattaatttcaatgaaatgagaaatatcaggttgtggtaaagctttacggtctacttttccactaggagttaaaggtagtgcttccagcatgacaaaagcggctggaatcataaaatcaggtagctttgctttcaaaaaatcacgtaggctgttaagtgttggtttttctgaatcgtaggtaatgtaagcaacaagttgtttttctaaattttgatgattacgcgcaataattacagcttctcgcacttgcgaatgtaagcagagagtattttcaatttcgccaatttcaatccgataacctcggatttttacttgatagtctactctgccaatatattcaagattgccatctggtaaataacgagctaaatcaccagttttgtagagacgttccgatactaattttgattttttaaaaggattgggaataaatttttcattagttaattctggacgattccagtaaccacgtgctacgccgacaccaccaatatacatttcaccgatgacaccgacatctacaggctgcaagtgttcattaaggatgtaaatttgggtgttagcaatgggacgaccaattggtacagtttttaaattactgtgcttttgacactgccaaaaagtgacatcaatagctgcttctgtagggccgtacaggttatataattcgcagtccaaacgctcaaaaaatctattttgtaaatctacaggtaaagcttcaccactacaaattactctttgaagagaagtacatttttctataccacgactagctaaaaacatttgcagcatggaggggacaaaatgcacagtagtgatttgctcttgaataatcagattaattaaataattactatctctatgtccacccggttgagcaattactaggcgcgcgcctgttaataaagtccaaaagaattcccagacagagacatcaaagctaaaaggtgttttttgtaaaatgctatctgtagaatttatttgataagtttcttgcatccacagcaagcgattacatatacctttgtgggtgttcattgcacccttgggtttaccagtagaaccagaagtgtagatgacataagcaagattatctgcttttacttcactttggggattagtctttggttgtgtagaaattttgttccattctgtatctacacaaatagtatgtgcttgatggtggggaatttgatttagtaatttttcttgggttagtaatacctgaatttgggaatcttctagcatataagctatgcgttcttggggatattctgggtcaatgggtacataagcaccaccagctttgagaattcccaataaacacacaaccatttctaaggaacgttctagacaaacaccaactaatgtttcttgttgaactcctagtgtgtgtaaatgatgtgctagttgattggctctattatttaattcttgataggttatttgttgctgttcaaactttacagctatggcgttgggggtgcgttctacttgcgttgtaaataactcatgtaaaccttgagaaaggtcgtaatctctgtgtgtagcgtcgggatttatttgtgtggtttgcatttttaaatcagggaaaaatgtcggttaatggctttgtggaatgggggtgatttactcacccagagatgcaaagacgcaaaaaaacctaatcttttcttctttgcgcctttgcgcctttgcgtgagataaaaaaaatccttaatctcccccgaattcgactaatttaccgatgttgaaatctatccgcgtccagcctttgagttgacggagattatttaacaacaacagaggaatttgccagtgatgtaccatcaaaaatggtaggggatcatgtagctgtaaaattgcatctgttccgcgccaaatgtttttcagccatgtcttcaactgggtgaaggaacgaataccagtcaagcgccaaatttcgtgataagtccaataggttggtttgctggttgctaatggttgtaaggtttccgccattggttgtttaccaatgtaagcttctgcaacttgggggtggttgtagaaggtggtaatagctgagtgtgtgcgggggttacactcgatcgcgtatacttgtccgtcttcagtttggatgaagtcgaaggatatctgtcctgtgagtttgagttctttgacaaagtgtctcacccattccaagatttgcgggttttctatgttctcatagttgacttggaaggctgatgattcgcaacaacagtgtagtcgaatttctccgtccctaactgtgctgtgggtgcagaattcttttcctgggatgaattcttgcataatccacggtttttcgggactgatgggtagttttctgacgaaggctgctgtttcctctggggtggcgcaggggagtttggttaagtccaaacgccgcactgagtcgtaagcaatgcttttgaggatgtatttgcgagtctcacgggaaaagtcgaagttgatgacttgttcgccagaggtgattttaaaggatttgggtactgataaaccaagcgatcgcgctttctctgccattgcaaatttatcatccaacatttgggtgatctctgcatcaaaatgaaacacttcgcaatggtgagataactctggtttggctaaggagtcgtaataactcccgacaggactggtaacgggaatgtatacatcaatgttttcccgtttgactatatctactaaagctttgatatagtcttctggttttttttgcggtgcgggagttgtgtaaaatttatcgaccgcttgggaaaatcgatgtcccgttaaccaatatttatgtgtttccagcaataccacccgatgtccatctgcgtggaatgaccttgctagttgcaaagctttggtcatttttccgccactgatcaggatatttttggggttgctggtttttgtagtttggggacggaagatagtacccaaaaccaaagctatgcaaacaataatggcgttgatgggcaatgctaatagtagcaaagccaaagtcagaatattttggataattgcggctatttttgtctctaaacctagagacggtgtagcaggtgaggagtcaaaggaaatagattgtgccatagtttacactcggcggataatcgtcaaaccgtcgcgcaggggaagtaaaacctgttctacacggatatcaagggctacggtacgattgaagtcagcgatcgcttgaccgttagcactgcgtttttggggaggtaaatatacctctccttgtaggagtgtgttatcaacacaaataaagccttgcggtgctaacaaattagtatctagcagcatttgcaagtaagctgtgtactcttttttatcggcatcaataaataccaagtcaaaagtttctccagccgttgctaatttctccaaagttgctaaagctgcacccaattccacacgtatctttccaccgtggggagattggttaaaagccttctgtgcaacttccgccgcataagggtctacttcacaagccaccaataccccatcttctggtaaagcttccgccatcgctaaggctgaataacccgtgaacatcccaatttccagaactcttttcgctttagtcatgtgaacaaacatctttaaagtttgtccttcgatatgaccagaaagcatttcctgttctagaggacgcacagttgcaccggctgtaaaatgctcaccccaagcctcttttacagttatcttagcgagtgctgctaaagcctctgattctggggtagtacattcctctaaataagggtcaatacctgctgctaactcccaagcttgggtaatctcagctaccaaatcagcaggtaaatcttggcgttgtttaacctctcgaacaacagcttccaacttcttagttaaaattcccaagggtgtgacaggtctagctgtcggtatgtctatcaaattcgtcatgatttaaaggtgtagccacataaattagaaaattgctgattgctcaaagcctgaaagcaactgaattaagactcagcactcatcactttcctacagcactccctacaagttccttctgactgctgagaggatacatatctacaccctcaccaccacgaggatattgactacaaagttccttatgttcagctaaagctgcggctgattcggcttttgtcaggtcattgacgaagaaacattctccaataggtcttggcatagcagctctgagtaaaccatctctagttagggtgatagactcagtagcacgccataataactcctcatccaacagaggatggtcgagggctagacctatacgactcatcaatcctaaaatgcgatcgcgctcttgagtagtaatgtagcctctccgtgctgcaatagtcgcagacaaagccatatcaatattgactgcgtgaccgtggaacataggtatatgtggcgcaagttccaaggtcggactccaagtatgaccgtaagcaattaccctatctaggtctaattcgtggaggttgggtacttccaactccaacatctttttaatcgctttgtaggtcacttcatgggctacatctttaatttctggcatagcgtcaatattgccaaaatgcgtatgcagtaagtcttccccgtacttctccaacaactcaaatacttctttgtgagcaactacggcgattttgactaattccgccataccgttacgtacttggtcagttggtagagtccgtaaaaaggagaaatctaagaaaacttgacgagaagcatgatatgcacccaaacggtttttcagcttcttgtggttaactgcgaccttgatggcaacactagcatcaattaagccaattaatgtagtaggaatacggatataattactgctgcgacggtatgcagagcaagcaaaaccgacaacatctgtaatcaagccaccaccaactactaatactggttctttgcgaactaatttgaaatctgcaaagacatctaccactttctcaaatgtttgaatggtcttgtttggctcagtaatagtaatggggaaaagagtcaagtcaatgccgtgatactggaaatatttttgaatttgtgtactgtagaactgactcacattagcatctacaacagccaagcaacgtccaaaatttttgtagatatctgctaattggtgatttttaatctcaaaaactccatctacataaactaagtcgtactcaattttttcgtagccttcaatgtgaaaagctgctgcttgcgcttcaaactttgcttggacgatgctcatattctttgacctttagtgcagtaaatgactgtatgtgttgactggaatatttgactcaagccaaaattgaataattctagtcctagaattaaaccgattgattggcataccaaataactctaatttctcatcaagaaatagaggtaatcgctggcagaatttgaggtaatcaaaacttgattgtaaaaagcagaacgttgattatatataacgtacagctttttaaatatagtgaatgacttgtgagtcttggttgaactgtaagactctgcaaatacgagcaaactacattaagtttctactgttttgcagtgatggtactaaatgaagtccatcatgattgatctgattgtagacagatatagtatgccagaagttagtagaaatttgctcaaaaatcttaagattttcttcacgattttaaagataacattatttgcgaaatatttgtacataattatgagatttttctaagaaatctcataactataagctgcaacttagtttatataagcatcataatttttgatctgaactgcaaaccaaagaaattagaggagagtttgatattaatttttatcataagtatcagcactactaaaaaccatgaattttaatcaacacacagcaaatgtttcccctaattctgaatcaagaacaggagtatggaaggaaaatttacagcagattgttgttaattagaaatacaaaaatggagtgctaaagataaagcacatctacttttatgagcgcagcagaaatgtcattggcattggctaaaacttaaggcttctaccaatacttgtaacaaaacttaactaatttgctctcatttttaagttagtgacactaatgaaagtcctaagcaatagcggactttttgcagttgggca

SEQ ID NO: 3 - protein, 888 aa, Propionibacterium acnes
MQTIDFNIRKLLVEWNATHRDYDLSQSLHELIVAQVERTPEAIAVTFDKQQLTYQELNHKANQLGHYLQTLGVQPETLVGVCLERSLEMVICLLGILKAGGAYVPIDPEYPQERIAYMLEDSQVKVLLTQEKLLNQIPHHQAQTICVDREWEKISTQANTNPKSNIKTDNLAYVIYTSGSTGKPKGAMNTHKGICNRLLWMQEAYQIDSTDSILQKTPFSFDVSVWEFFWTLLTGARLVIAKPGGHKDSAYLIDLITQEQITTLHFVPSMLQVFLQNRHVSKCSSLKRVICSGEALSIDLQNRFFQHLQCELHNLYGPTEAAIDVTFWQCRKDSNLKSVPIGRPIANTQIYILDADLQPVNIGVTGEIYIGGVGVARGYLNKEELTKEKFIINPFPNSEFKRLYKTGDLARYLPDGNIEYLGRTDYQVKIRGYRIEIGEIENVLSSHPQVREAVVIARDDNAQEKQIIAYITYNSIKPQLDNLRDFLKARLPDFMIPAAFVMLEHLPLTPSGKVDRKALPKPDLFNYSEHNSYVAPRNEVEEKLVQIWSNILHLPKVGVTENFFAIGGNSLKALHLISQIEELFAKEISLATLLTNPVIADLAKVIQANNQIHNSPLVPIQPQGKQQPFFCIHPAGGHVLCYFKLAQYIGTDQPFYGLQAQGFYGDEAPLTRVEDMASLYVKTIREFQPQGPYRVGGWSFGGVVAYEVAQQLHRQGQEVSLLAILDSYVPILLDKQKPIDDVYLVGVLSRVFGGMFGQDNLVTPEEIENLTVEEKINYIIDKARSARIFPPGVERQNNRRILDVLVGTLKATYSYIRQPYPGKVTVFRAREKHIMAPDPTLVWVELFSVMAAQEIKIIDVPGNHYSFVLEPHVQVLAQRLQDCLENNS

SEQ ID NO: 4 - protein, 458 aa, Propionibacterium acnes
MAQSLPLSSAPATPSLPSQTKIAAIIQNICTLALLLLALPINATIVFISLLVFRPQKVKAANPQTILISGGKMTKALQLARSFHAAGHRVVLVETHKYWLTGHRFSQAVDKFYTVPAPQDNPQAYIQALVDIVKQENIDVYIPVTSPVGSYYDSLAKPELSHYCEVFHFDADITQMLDDKFALTQKARSLGLSVPKSFKITSPEQVINFDFSGETRKYILKSIPYDSVRRLDLTKLPCATPEETAAFVRSLPITPEKPWIMQEFIPGKEFCTHSTVRNGELRLHCCCESSAFQVNYENVNNPQITEWVQHFVKELKLTGQISFDFIQAEDGTVYAIECNPRTHSAITTFYDHPQVAEAYLSQAPTTETIQPLTTSKPTYWTYHEVWRLTGIRSFTQLQRWLGNIWRGTDAIYQPDDPLPFLMVHHWQIPLLLLNNLRRLKGWTRIDFNIGKLVELGGD

SEQ ID NO: 5 - protein, 279 aa, Propionibacterium acnes
MTNVIVQPTARPVTPLGILTKQLEAIVQEVKQHPDLPGELIANIHQAWRLAAGIDPYLEECTTPESPELAALAKTTATEAWGEHFHGGTTVRPLEQEMLSGHIEGQTLKMFVHMTKAKKVLEIGMFTGYSALAMAEALPEDGLLVACEVDPYAAEIGQKAFQQSPHGGKIRVELDAALATLDKLAEAGESFDLVFIDADKKEYVAYFHKLLGSSLLAPDGFICVDNTLLQGEVYLPAEERSVNGEAIAQFNHTVAIDPRVEQVLLPLRDGLTIIRRIQP

SEQ ID NO: 6 - protein, 410 aa, Propionibacterium acnes
MSIVQAKFEAKETSFHVEGYEKIEYDLVYVDGIFEIQNSALADVYQGFGRCLAIVDANVSRLYGNQIQAYFQYYGIELRLFPITITEPDKTIQTFERVIDVFADFKLVRKEPVLVVGGGLITDVVGFACSTYRRSSNYIRIPTTLIGLIDASVAIKVAVNHRKLKNRLGAYHASRKVFLDFSLLRTLPTDQVRNGMAELVKIAVVAHQEVFELLEKYGEELLRTHFGNIDATPEIKEIAHRLTYKAIHKMLELEVPNLHELDLDRVIAYGHTWSPTLELAPRLPMFHGHAVNVDMAFSATIAARRGYITIAERDRILGLMSRVGLSLDHPMLDIDILWRGTESITLTRDGLLRAAMPKPIGDCVFVNDLTREELAAALADHKELCTSYPRGGEGVDVYPVYQKELIGSVK

SEQ ID NO: 7 - DNA, 1233 bp, Propionibacterium acnes
atgagtatcgtccaagcaaagtttgaagctaaggaaacatcttttcatgtagaaggttacgaaaagattgagtatgatttggtgtatgtagatggtatttttgaaatccagaattctgcactagcagatgtatatcaaggttttggacgatgcttggcgattgtagatgctaacgtcagtcggttgtatggtaatcaaattcaggcatatttccagtattatggtatagaactgaggctatttcctattaccattactgaaccagataagactattcaaactttcgagagagttatagatgtctttgcagatttcaaattagtccgcaaagaaccagtattagtcgtgggtggcggtttaattacagatgttgtcggctttgcttgttctacatatcgtcgcagcagcaattacatccgcattcctactacattgattggattaattgatgccagtgtagcaattaaggtagcagttaatcatcgcaaactgaaaaaccgtttgggtgcttatcatgcttctcgcaaagtatttttagatttctccttgttgcgtactctccctacagaccaagtacgtaacgggatggcggaattggtaaaaatcgctgtagtagcgcatcaagaagtttttgaattgttggagaagtacggcgaagaattactacgtactcattttggcaatatagatgcaactccagagattaaagaaatagcccatcgtttgacttacaaagctatccataagatgttggaattggaagttcccaacctgcatgagttagacctagatagggtgattgcttacggtcacacttggagtcccaccttggaacttgcgcctcgtctacccatgttccacggacacgccgttaatgtagatatggctttctcggcaacgatcgccgcccgtagaggatatattacaattgcagaacgcgatcgtattttaggattaatgagtcgcgttggtctatccctcgaccatcccatgttggatatagatattttgtggcgtggtactgaatctatcacattaactcgtgatggtttgttaagagctgctatgccaaaacccattggtgattgtgtcttcgtcaatgacctgacaagagaagaattagcagccgcattagctgaccacaaagaactttgtaccagttatccccgtggtggtgaaggtgtggatgtgtatcccgtttatcaaaaagaattaatcgggagtgttaaataa

SEQ ID NO: 8 - DNA, 174 bp, Propionibacterium acnes
tgacttttttgaattcaaaatgcaaaatactccacggatacactgcgcgagcgcggtagcatttctgttcgcggagcgtcccgtagggaaagagaaggctacgcaaataatcggacactaattgtctttaattttgaattttgaattttgaattttgaattggagcgaagcgac

SEQ ID NO: 9 - DNA, 840 bp, Propionibacterium acnes
ttgacaaatgtgattgtccaaccaacagctagacctgttacaccattgggaattttaaccaagcagttagaagccatagtccaagaggttaagcaacatccagatttacctggggaattgatagcaaacatccatcaggcttggcgtttagccgcaggtatagacccttatttggaagaatgcaccactccagaatctcctgaactcgctgcattggcaaaaaccacagccaccgaagcctggggagaacacttccacggaggtacaaccgtccgtcctctagaacaagagatgctttctggtcatatcgaaggacaaaccttaaagatgtttgttcacatgaccaaagctaaaaaagtcttagaaattgggatgtttaccggttattcggcgctggcgatggcggaagcattaccagaggatggactgcttgtggcttgtgaagttgacccttacgcggcggaaattggacagaaagcctttcaacaatctccccacggtggaaagattcgtgtggaattggatgcagccttagcaactcttgataagttagcagaagctggggagtcttttgacttggtatttatcgacgcagataaaaaagagtatgtagcctattttcacaagttgctaggtagcagtttgttagcaccagatggctttatttgtgtagataacaccttattacaaggggaagtttatctaccagcagaggaacgtagcgtcaatggtgaagcgatcgcgcaatttaatcatacagtagctatagacccccgtgtagaacaggttttgttgccgttgcgagatggtttaacaattatccgcagaatacaaccttaa

SEQ ID NO: 10 - DNA, 14 bp, Propionibacterium acnes
ttgtccaatcgact

SEQ ID NO: 11 - DNA, 1377 bp, Propionibacterium acnes
atggcacaatcccttcccctttcttccgcacctgctacaccgtctcttccttcccagacgaaaatagccgcaattatccaaaatatctgcactttggctttgttattactagcattgcccattaatgccaccattgtttttatatccttgttagtcttccgaccgcaaaaggtcaaagcagcaaacccccaaaccattcttatcagtggcggtaagatgaccaaagctttacaactagcaaggtcattccacgcggctggacatagagttgtcttggtggaaacccataaatactggttgactggtcatcgtttttcccaagcagtggataagttttacacagtccccgcaccccaggacaatccccaagcttacattcaggctttggtagatatcgtcaaacaagaaaacatcgatgtttatattcccgtcaccagtccagtgggtagctactacgactcattagccaaaccagagttatcccattattgcgaagtgtttcactttgacgcagatattacccaaatgttggatgataaatttgcgttgacacaaaaagcgcgatcgcttggtttatcagtacccaaatcctttaaaattacctcaccagaacaagtcatcaacttcgatttttctggagagacacgtaaatacatcctcaaaagcattccctacgactcagtgcggcggttggacttaaccaaactcccctgtgctactccagaggaaacagcagcattcgtcagaagtttgccaattactcccgaaaaaccgtggattatgcaggaatttatccccggtaaggaattctgcacccatagcaccgttcggaatggggaactcagactgcattgctgttgcgaatcttcagccttccaagttaattatgagaatgtaaataacccgcaaattaccgaatgggtacagcattttgtcaaggaactgaaactgacaggacagatttcctttgactttatccaagccgaagacggaacagtttacgccatcgagtgtaacccccgcacacattcagcaattaccacattttacgaccacccccaggtagcagaagcgtacttgagtcaagcaccgacgactgaaaccatacaaccactaacgacaagcaagcctacctattggacttatcacgaagtttggcgtttaactggtatccgttctttcacccagttgcaaagatggctggggaatatttggcgcgggactgatgcgatttatcagccagatgaccccttaccgtttttgatggtacatcattggcaaattcccctactgttattgaataatttgcgtcgtcttaaaggttggacgcggatagatttcaatattgggaagttggtggaattggggggagattag

SEQ ID NO: 12 - DNA, 156 bp, Propionibacterium acnes
tttttaaacgcagagggacgctgaggttagcgcagcgaaaagttctggaggagggtttccctccgtaggaaacttttcaagagagagggacgcggagtgtgttttctctgcgtctctgcgtgagaaattttttattattgagcaaagttagaagat

SEQ ID NO: 13 - DNA, 2667 bp, Propionibacterium acnes
atgcagactatagattttaatattcgtaagttacttgtagagtggaacgcgacccacagagattatgatctttcccagagtttacatgaactaattgtagctcaagtagaacgaacacctgaggcgatcgctgtcacctttgacaagcaacaactaacttatcaagaactaaatcataaagcaaaccagctaggacattatttacaaacattaggagtccagccagaaaccctggtaggcgtttgtttagaacgttccttagaaatggttatctgtcttttaggaatcctcaaagctgggggtgcttatgttcctattgaccctgaatatcctcaagaacgcatagcttatatgctagaagattctcaggtgaaggtactactaactcaagaaaaattactcaatcaaattccccaccatcaagcacaaactatctgtgtagatagggaatgggagaaaatttccacacaagctaataccaatcccaaaagtaatataaaaacggataatcttgcttatgtaatttacacctctggttccactggtaaaccaaaaggtgcaatgaacacccacaaaggtatctgtaatcgcttattgtggatgcaggaagcttatcaaatcgattccacagatagcattttacaaaaaaccccctttagttttgatgtttccgtttgggagttcttttggactttattaactggcgcacgtttggtaatagccaaaccaggcggacataaagatagtgcttacctcatcgatttaattactcaagaacaaatcactacgttgcattttgtcccctcaatgctgcaagtgtttttacaaaatcgccatgtaagcaaatgcagctctctaaaaagagttatttgtagcggtgaagctttatctatagatttacaaaatagatttttccagcatttgcaatgtgaattacataacctctatggcccgacagaagcagcaattgatgtcacattttggcaatgtagaaaagatagtaatttaaagagtgtacctattggtcgtcccattgctaatactcaaatttatattcttgatgccgatttacaaccagtaaatattggtgtcactggtgaaatttatattggtggtgtaggggttgctcgtggttatttgaataaagaagaattgaccaaagaaaaatttattattaatccctttcccaattctgagtttaagcgactttataaaacaggtgatttagctcgttatttacccgatggaaatattgaatatcttggtagaacagattatcaagtaaaaattcggggttatagaattgaaattggcgagattgaaaatgttttatcttcacacccacaagtcagagaagctgtagtcatagcgcgggatgataacgctcaagaaaaacaaatcatcgcttatattacctataactccatcaaacctcagcttgataatctgcgtgatttcctaaaagcaaggctacctgattttatgattccagccgcttttgtgatgctggagcatcttcctttaactcccagtggtaaagtagaccgtaaggcattacctaagcctgatttatttaattatagtgaacataattcctatgtagcgcctcggaatgaagttgaagaaaaattagtacaaatctggtcgaatattctgcatttacctaaagtaggtgtgacagaaaactttttcgctattggtggtaattccctcaaagctctacatttaatttctcaaattgaagagttatttgctaaagagatatccttagcaacacttttaacaaatccagtaattgcagatttagccaaggttattcaagcaaacaaccaaatccataattcacccctagttccaattcaaccacaaggtaagcagcagcctttcttttgtatacatcctgctggtggtcatgttttatgctattttaaactcgcacaatatataggaactgaccaaccattttatggcttacaagctcaaggattttatggagatgaagcacccttgacgcgagttgaagatatggctagtctctacgtcaaaactattagagaatttcaaccccaagggccttatcgtgtcggggggtggtcatttggtggagtcgtagcttatgaagtagcacagcagttacatagacaaggacaagaagtatctttactagcaatattagattcttacgtaccgattctgctggataaacaaaaacccattgatgacgtttatttagttggtgttctctccagagtttttggcggtatgtttggtcaagataatctagtcacacctgaagaaatagaaaatttaactgtagaagaaaaaattaattacatcattgataaagcacggagcgctagaatattcccgcctggtgtagaacgtcaaaataatcgccgtattcttgatgttttggtgggaactttaaaagcaacttattcctatataagacaaccatatccaggaaaagtcactgtatttcgagccagggaaaaacatattatggctcctgacccgaccttagtttgggtagaattattttctgtaatggcggctcaagaaattaagattattgatgtccctggaaaccattattcgtttgttctagaaccccatgtacaggttttagcacagcgtttacaagattgtctggaaaataattcataa

SEQ ID NO: 14 - DNA, 6469 bp, Propionibacterium acnes
tcgagatgaattattttccagacaatcttgtaaacgctgtgctaaaacctgtacatggggttctagaacaaacgaataatggtttccagggacatcaataatcttaatttcttgagccgccattacagaaaataattctacccaaactaaggtcgggtcaggagccataatatgtttttccctggctcgaaatacagtgacttttcctggatatggttgtcttatataggaataagttgcttttaaagttcccaccaaaacatcaagaatacggcgattattttgacgttctacaccaggcgggaatattctagcgctccgtgctttatcaatgatgtaattaattttttcttctacagttaaattttctatttcttcaggtgtgactagattatcttgaccaaacataccgccaaaaactctggagagaacaccaactaaataaacgtcatcaatgggtttttgtttatccagcagaatcggtacgtaagaatctaatattgctagtaaagatacttcttgtccttgtctatgtaactgctgtgctacttcataagctacgactccaccaaatgaccaccccccgacacgataaggcccttggggttgaaattctctaatagttttgacgtagagactagccatatcttcaactcgcgtcaagggtgcttcatctccataaaatccttgagcttgtaagccataaaatggttggtcagttcctatatattgtgcgagtttaaaatagcataaaacatgaccaccagcaggatgtatacaaaagaaaggctgctgcttaccttgtggttgaattggaactaggggtgaattatggatttggttgtttgcttgaataaccttggctaaatctgcaattactggatttgttaaaagtgttgctaaggatatctctttagcaaataactcttcaatttgagaaattaaatgtagagctttgagggaattaccaccaatagcgaaaaagttttctgtcacacctactttaggtaaatgcagaatattcgaccagatttgtactaatttttcttcaacttcattccgaggcgctacataggaattatgttcactataattaaataaatcaggcttaggtaatgccttacggtctactttaccactgggagttaaaggaagatgctccagcatcacaaaagcggctggaatcataaaatcaggtagccttgcttttaggaaatcacgcagattatcaagctgaggtttgatggagttataggtaatataagcgatgatttgtttttcttgagcgttatcatcccgcgctatgactacagcttctctgacttgtgggtgtgaagataaaacattttcaatctcgccaatttcaattctataaccccgaatttttacttgataatctgttctaccaagatattcaatatttccatcgggtaaataacgagctaaatcacctgttttataaagtcgcttaaactcagaattgggaaagggattaataataaatttttctttggtcaattcttctttattcaaataaccacgagcaacccctacaccaccaatataaatttcaccagtgacaccaatatttactggttgtaaatcggcatcaagaatataaatttgagtattagcaatgggacgaccaataggtacactctttaaattactatcttttctacattgccaaaatgtgacatcaattgctgcttctgtcgggccatagaggttatgtaattcacattgcaaatgctggaaaaatctattttgtaaatctatagataaagcttcaccgctacaaataactctttttagagagctgcatttgcttacatggcgattttgtaaaaacacttgcagcattgaggggacaaaatgcaacgtagtgatttgttcttgagtaattaaatcgatgaggtaagcactatctttatgtccgcctggtttggctattaccaaacgtgcgccagttaataaagtccaaaagaactcccaaacggaaacatcaaaactaaagggggttttttgtaaaatgctatctgtggaatcgatttgataagcttcctgcatccacaataagcgattacagatacctttgtgggtgttcattgcaccttttggtttaccagtggaaccagaggtgtaaattacataagcaagattatccgtttttatattacttttgggattggtattagcttgtgtggaaattttctcccattccctatctacacagatagtttgtgcttgatggtggggaatttgattgagtaatttttcttgagttagtagtaccttcacctgagaatcttctagcatataagctatgcgttcttgaggatattcagggtcaataggaacataagcacccccagctttgaggattcctaaaagacagataaccatttctaaggaacgttctaaacaaacgcctaccagggtttctggctggactcctaatgtttgtaaataatgtcctagctggtttgctttatgatttagttcttgataagttagttgttgcttgtcaaaggtgacagcgatcgcctcaggtgttcgttctacttgagctacaattagttcatgtaaactctgggaaagatcataatctctgtgggtcgcgttccactctacaagtaacttacgaatattaaaatctatagtctgcatatcttctaactttgctcaataataaaaaatttctcacgcagagacgcagagaaaacacactccgcgtccctctctcttgaaaagtttcctacggagggaaaccctcctccagaacttttcgctgcgctaacctcagcgtccctctgcgtttaaaaactaatctccccccaattccaccaacttcccaatattgaaatctatccgcgtccaacctttaagacgacgcaaattattcaataacagtaggggaatttgccaatgatgtaccatcaaaaacggtaaggggtcatctggctgataaatcgcatcagtcccgcgccaaatattccccagccatctttgcaactgggtgaaagaacggataccagttaaacgccaaacttcgtgataagtccaataggtaggcttgcttgtcgttagtggttgtatggtttcagtcgtcggtgcttgactcaagtacgcttctgctacctgggggtggtcgtaaaatgtggtaattgctgaatgtgtgcgggggttacactcgatggcgtaaactgttccgtcttcggcttggataaagtcaaaggaaatctgtcctgtcagtttcagttccttgacaaaatgctgtacccattcggtaatttgcgggttatttacattctcataattaacttggaaggctgaagattcgcaacagcaatgcagtctgagttccccattccgaacggtgctatgggtgcagaattccttaccggggataaattcctgcataatccacggtttttcgggagtaattggcaaacttctgacgaatgctgctgtttcctctggagtagcacaggggagtttggttaagtccaaccgccgcactgagtcgtagggaatgcttttgaggatgtatttacgtgtctctccagaaaaatcgaagttgatgacttgttctggtgaggtaattttaaaggatttgggtactgataaaccaagcgatcgcgctttttgtgtcaacgcaaatttatcatccaacatttgggtaatatctgcgtcaaagtgaaacacttcgcaataatgggataactctggtttggctaatgagtcgtagtagctacccactggactggtgacgggaatataaacatcgatgttttcttgtttgacgatatctaccaaagcctgaatgtaagcttggggattgtcctggggtgcggggactgtgtaaaacttatccactgcttgggaaaaacgatgaccagtcaaccagtatttatgggtttccaccaagacaactctatgtccagccgcgtggaatgaccttgctagttgtaaagctttggtcatcttaccgccactgataagaatggtttgggggtttgctgctttgaccttttgcggtcggaagactaacaaggatataaaaacaatggtggcattaatgggcaatgctagtaataacaaagccaaagtgcagatattttggataattgcggctattttcgtctgggaaggaagagacggtgtagcaggtgcggaagaaaggggaagggattgtgccatagtcgattggacaattaaggttgtattctgcggataattgttaaaccatctcgcaacggcaacaaaacctgttctacacgggggtctatagctactgtatgattaaattgcgcgatcgcttcaccattgacgctacgttcctctgctggtagataaacttccccttgtaataaggtgttatctacacaaataaagccatctggtgctaacaaactgctacctagcaacttgtgaaaataggctacatactcttttttatctgcgtcgataaataccaagtcaaaagactccccagcttctgctaacttatcaagagttgctaaggctgcatccaattccacacgaatctttccaccgtggggagattgttgaaaggctttctgtccaatttccgccgcgtaagggtcaacttcacaagccacaagcagtccatcctctggtaatgcttccgccatcgccagcgccgaataaccggtaaacatcccaatttctaagacttttttagctttggtcatgtgaacaaacatctttaaggtttgtccttcgatatgaccagaaagcatctcttgttctagaggacggacggttgtacctccgtggaagtgttctccccaggcttcggtggctgtggtttttgccaatgcagcgagttcaggagattctggagtggtgcattcttccaaataagggtctatacctgcggctaaacgccaagcctgatggatgtttgctatcaattccccaggtaaatctggatgttgcttaacctcttggactatggcttctaactgcttggttaaaattcccaatggtgtaacaggtctagctgttggttggacaatcacatttgtcaagtcgcttcgctccaattcaaaattcaaaattcaaaattcaaaattaaagacaattagtgtccgattatttgcgtagccttctctttccctacgggacgctccgcgaacagaaatgctaccgcgctcgcgcagtgtatccgtggagtattttgcattttgaattcaaaaaagtcattatttaacactcccgattaattctttttgataaacgggatacacatccacaccttcaccaccacggggataactggtacaaagttctttgtggtcagctaatgcggctgctaattcttctcttgtcaggtcattgacgaagacacaatcaccaatgggttttggcatagcagctcttaacaaaccatcacgagttaatgtgatagattcagtaccacgccacaaaatatctatatccaacatgggatggtcgagggatagaccaacgcgactcattaatcctaaaatacgatcgcgttctgcaattgtaatatatcctctacgggcggcgatcgttgccgagaaagccatatctacattaacggcgtgtccgtggaacatgggtagacgaggcgcaagttccaaggtgggactccaagtgtgaccgtaagcaatcaccctatctaggtctaactcatgcaggttgggaacttccaattccaacatcttatggatagctttgtaagtcaaacgatgggctatttctttaatctctggagttgcatctatattgccaaaatgagtacgtagtaattcttcgccgtacttctccaacaattcaaaaacttcttgatgcgctactacagcgatttttaccaattccgccatcccgttacgtacttggtctgtagggagagtacgcaacaaggagaaatctaaaaatactttgcgagaagcatgataagcacccaaacggtttttcagtttgcgatgattaactgctaccttaattgctacactggcatcaattaatccaatcaatgtagtaggaatgcggatgtaattgctgctgcgacgatatgtagaacaagcaaagccgacaacatctgtaattaaaccgccacccacgactaatactggttctttgcggactaatttgaaatctgcaaagacatctataactctctcgaaagtttgaatagtcttatctggttcagtaatggtaataggaaatagcctcagttctataccataatactggaaatatgcctgaatttgattaccatacaaccgactgacgttagcatctacaatcgccaagcatcgtccaaaaccttgatatacatctgctagtgcagaattctggatttcaaaaataccatctacatacaccaaatcatactcaatcttttcgtaaccttctacatgaaaagatgtttccttagcttcaaactttgcttggacgatactcatagctc

SEQ ID NO: 15 - DNA, 11058 bp, Propionibacterium acnes
  modified_base (3323)..(3323): a, c, t, g, unknown or other
  modified_base (3325)..(3325): a, c, t, g, unknown or other
  modified_base (3327)..(3327): a, c, t, g, unknown or other
  modified_base (9863)..(9863): a, c, t, g, unknown or other
ccgctcatttttggggtccagctggttcagctggtcagtatggctgaaagccatggtcttaaaaagcagttcggcgatttttgctgatctgctttttgggggttgaaaccgtcgttttttcgacggtttcttcttatcttgatactattagaaacaacgtcattttaaaaaaccgggataaacccttgacacaactgggcttaggcgtattatgagtttataaaatgaataaagaaaaaacccacgtgagaattcctagtttggcgacccggaacacgtgagttaatcttgaatattcgtatttactagacatagtttaaagcttgagttagcaagcgtcaagcccttggctttagtaaatacataaaagattagctcttctcacgtggctgaatgaggggagctttttagtttggctgatagaaaagttttagttgatcgatcgcagtcgggcaaagtacgaccatggcgagaacataagttagaaaatttacagtatggtgattatttacaaatgttgcactacaagaaagcccatcgagttaaagagtgtggtgaagtattacgttttgtggaagataaaaatggtcacaaaaaactggctcagacttggttttgccattcccgtttgtgtccgttatgtaattggcggcggtcaatgaaacaatctaaccagttaactcaaattttgacagaagcagttaaacagcgaaaaacgggtcggttcttgtttttaacattgacggtagagaatactacaggggatttgttgaagagtgaattacggcagatgggacgagccattgcaaagatctttcagtataaaaaagtggctaaaaatttgttgggctatgtacgttcaactgaggttaccattaatcacgaagcggatcagccgatgtatcaccaccatatgcatgttttgctttttatgaaatctagttattttacaggaactgataattatatttcacaaacagaatggactagatattggcaacgagcgatgaaattagcttatgtgccggttgtgaatgttgaagcggttaaaccgaatgtgaaacgccagaaaaattccttactggctagtgcccaagaaacggctaagtatcaggtgaagtccaaagatattttaactaataatcaagaacaagatttacaagtaattgatgatttggaacaagctttggctggttcccggcaaattagctatgggggtttgttaaaagaaattcgtaagcaattgcaactagaagatgttgaaaatggtgatttaattaatacggatagtgatgatcaaaaagttgaccaagtggtacgcgagattgttgctaagtgggactatcagcgaaaaaattactttatttgaatgagtgctatattatatataaagacaggaaatcatttgtctagcggggggaactcttttatgatttatgctactgctgttaaatttgaagatgaaaattctgatagaacacctaaagcgattgattctatttatttagattctacttcggatgaaacttggcattttggagagggagaaaacactactcctattaagggttggtatgataaacatgatgtttatcgttggttatttttgaattttgataaaggccttgtgatgaaggttgttactggtgaaaaaccggatataaagcctgttggtaaagataaagatgacccggatggatatgttagatctgaaaagaatggtattgttgttgataatcttgaaatgcttccagattcgccttctcctttgtgatattaaagaatggagaacgtttattttgattttgaatttgttttttattaataatcattgggagcgaaagcgacctttgattatttttttgccaacggcaaaaatcgcctcgcagagcccaaactttacaaggtaaagtatattgggctataccttgcatggaggtttgccgaattctgtgctatgctctaaccaaatttagctgtttggaaatggagtggtgaaatgagttatttagtggctaatatgcagaaattaaaagctgataatttagttggcttgggtaatcatgatcaacgccgaacgcaacatcacaaaaatactgatattgacgttgaccgttctggcttaaattatgatttagttgctggtcggactaaccatttcaaaacggatattgcggcttatattaacgagcataaaaccagtcagcgagcggtcagaaaagatgccgttttagtcaatgaatggattatttcgagcgatagcaatttctttgctaatttaacggcggctgatacgcgcaaatattttgaaacagctaaagcttactttgctgaaaaatttggtgaagaaaatattcgctatgcaattgttcaccttgatgagagtacgccacatatgcatatgggaattgtgccctttgatgatgaatataagttgtctgctaaacgggtgtttaatcgtgcggctttgcaaaacgttcaagatcaattgccgacttatttgcaacagcatggttttaatattcaacgtggggttcaagaatcggaacgcaaaagtttaacggtgccagaatataaagctatgcgggaagatttgaaaaaggcgacgcttcaaaaacaagaaatacaagctgaacttgaagatgccagaaaacgccttgctgaacttaaacctcgtgatcagcaggaaattgagagcaaacctacttttttaagcaaggataaagtggttgttagaaaaagtgatcttcatgacttagaatctcgagcagctgtcagtgatatttataatcaacaacagaaccgtttaaaacttgataatcaaagcctaaattatcaactgcttgaagttaaagacaataattatgagttaagcaagaaaaatgagaagctccaaaaattagtggatacgttacaaggaattgttcggagcgttgaccggttcttacagcgcaaattaggtgttggcttaccaagtgagtggctagaacgagctggactaaaagaaccgtctaaaaacgcccctcagaggccgcaggaacgttcggagggacagcatgatgaattagatggtccaagtctttgaatttgtcctatggctttaaattacccgctgatgagcattgaagctggttaatggccgtcagtcaacggtaaatcgaattaaagggacttactgctttagcagttagtccctttttgaggctttaaggagttgactgactcactagaccaagacacttttgcgcatgcaaagaaaagcacacctgctttttttgcctgcctcacggcgagtgcggggtgagtttgagcgggagctcshnrnrnwthrmtragatctagcgctatagttgttgacagaatggacatactatgatatattttgctatagcgatgaattattttccagacaatcttgtaaacgctgtgctaaaacctgtacatggggttctagaacaaacgaataatggtttccagggacatcaataatcttaatttcttgagccgccattacagaaaataattctacccaaactaaggtcgggtcaggagccataatatgtttttccctggctcgaaatacagtgacttttcctggatatggttgtcttatataggaataagttgcttttaaagttcccaccaaaacatcaagaatacggcgattattttgacgttctacaccaggcgggaatattctagcgctccgtgctttatcaatgatgtaattaattttttcttctacagttaaattttctatttcttcaggtgtgactagattatcttgaccaaacataccgccaaaaactctggagagaacaccaactaaataaacgtcatcaatgggtttttgtttatccagcagaatcggtacgtaagaatctaatattgctagtaaagatacttcttgtccttgtctatgtaactgctgtgctacttcataagctacgactccaccaaatgaccaccccccgacacgataaggcccttggggttgaaattctctaatagttttgacgtagagactagccatatcttcaactcgcgtcaagggtgcttcatctccataaaatccttgagcttgtaagccataaaatggttggtcagttcctatatattgtgcgagtttaaaatagcataaaacatgaccaccagcaggatgtatacaaaagaaaggctgctgcttaccttgtggttgaattggaactaggggtgaattatggatttggttgtttgcttgaataaccttggctaaatctgcaattactggatttgttaaaagtgttgctaaggatatctctttagcaaataactcttcaatttgagaaattaaatgtagagctttgagggaattaccaccaatagcgaaaaagttttctgtcacacctactttaggtaaatgcagaatattcgaccagatttgtactaatttttcttcaacttcattccgaggcgctacataggaattatgttcactataattaaataaatcaggcttaggtaatgccttacggtctactttaccactgggagttaaaggaagatgctccagcatcacaaaagcggctggaatcataaaatcaggtagccttgcttttaggaaatcacgcagattatcaagctgaggtttgatggagttataggtaatataagcgatgatttgtttttcttgagcgttatcatcccgcgctatgactacagcttctctgacttgtgggtgtgaagataaaacattttcaatctcgccaatttcaattctataaccccgaatttttacttgataatctgttctaccaagatattcaatatttccatcgggtaaataacgagctaaatcacctgttttataaagtcgcttaaactcagaattgggaaagggattaataataaatttttctttggtcaattcttctttattcaaataaccacgagcaacccctacaccaccaatataaatttcaccagtgacaccaatatttactggttgtaaatcggcatcaagaatataaatttgagtattagcaatgggacgaccaataggtacactctttaaattactatcttttctacattgccaaaatgtgacatcaattgctgcttctgtcgggccatagaggttatgtaattcacattgcaaatgctggaaaaatctattttgtaaatctatagataaagcttcaccgctacaaataactctttttagagagctgcatttgcttacatggcgattttgtaaaaacacttgcagcattgaggggacaaaatgcaacgtagtgatttgttcttgagtaattaaatcgatgaggtaagcactatctttatgtccgcctggtttggctattaccaaacgtgcgccagttaataaagtccaaaagaactcccaaacggaaacatcaaaactaaagggggttttttgtaaaatgctatctgtggaatcgatttgataagcttcctgcatccacaataagcgattacagatacctttgtgggtgttcattgcaccttttggtttaccagtggaaccagaggtgtaaattacataagcaagattatccgtttttatattacttttgggattggtattagcttgtgtggaaattttctcccattccctatctacacagatagtttgtgcttgatggtggggaatttgattgagtaatttttcttgagttagtagtaccttcacctgagaatcttctagcatataagctatgcgttcttgaggatattcagggtcaataggaacataagcacccccagctttgaggattcctaaaagacagataaccatttctaaggaacgttctaaacaaacgcctaccagggtttctggctggactcctaatgtttgtaaataatgtcctagctggtttgctttatgatttagttcttgataagttagttgttgcttgtcaaaggtgacagcgatcgcctcaggtgttcgttctacttgagctacaattagttcatgtaaactctgggaaagatcataatctctgtgggtcgcgttccactctacaagtaacttacgaatattaaaatctatagtctgcatatcttctaactttgctcaataataaaaaatttctcacgcagagacgcagagaaaacacactccgcgtccctctctcttgaaaagtttcctacggagggaaaccctcctccagaacttttcgctgcgctaacctcagcgtccctctgcgtttaaaaactaatctccccccaattccaccaacttcccaatattgaaatctatccgcgtccaacctttaagacgacgcaaattattcaataacagtaggggaatttgccaatgatgtaccatcaaaaacggtaaggggtcatctggctgataaatcgcatcagtcccgcgccaaatattccccagccatctttgcaactgggtgaaagaacggataccagttaaacgccaaacttcgtgataagtccaataggtaggcttgcttgtcgttagtggttgtatggtttcagtcgtcggtgcttgactcaagtacgcttctgctacctgggggtggtcgtaaaatgtggtaattgctgaatgtgtgcgggggttacactcgatggcgtaaactgttccgtcttcggcttggataaagtcaaaggaaatctgtcctgtcagtttcagttccttgacaaaatgctgtacccattcggtaatttgcgggttatttacattctcataattaacttggaaggctgaagattcgcaacagcaatgcagtctgagttccccattccgaacggtgctatgggtgcagaattccttaccggggataaattcctgcataatccacggtttttcgggagtaattggcaaacttctgacgaatgctgctgtttcctctggagtagcacaggggagtttggttaagtccaaccgccgcactgagtcgtagggaatgcttttgaggatgtatttacgtgtctctccagaaaaatcgaagttgatgacttgttctggtgaggtaattttaaaggatttgggtactgataaaccaagcgatcgcgctttttgtgtcaacgcaaatttatcatccaacatttgggtaatatctgcgtcaaagtgaaacacttcgcaataatgggataactctggtttggctaatgagtcgtagtagctacccactggactggtgacgggaatataaacatcgatgttttcttgtttgacgatatctaccaaagcctgaatgtaagcttggggattgtcctggggtgcggggactgtgtaaaacttatccactgcttgggaaaaacgatgaccagtcaaccagtatttatgggtttccaccaagacaactctatgtccagccgcgtggaatgaccttgctagttgtaaagctttggtcatcttaccgccactgataagaatggtttgggggtttgctgctttgaccttttgcggtcggaagactaacaaggatataaaaacaatggtggcattaatgggcaatgctagtaataacaaagccaaagtgcagatattttggataattgcggctattttcgtctgggaaggaagagacggtgtagcaggtgcggaagaaaggggaagggattgtgccatagtcgattggacaattaaggttgtattctgcggataattgttaaaccatctcgcaacggcaacaaaacctgttctacacgggggtctatagctactgtatgattaaattgcgcgatcgcttcaccattgacgctacgttcctctgctggtagataaacttccccttgtaataaggtgttatctacacaaataaagccatctggtgctaacaaactgctacctagcaacttgtgaaaataggctacatactcttttttatctgcgtcgataaataccaagtcaaaagactccccagcttctgctaacttatcaagagttgctaaggctgcatccaattccacacgaatctttccaccgtggggagattgttgaaaggctttctgtccaatttccgccgcgtaagggtcaacttcacaagccacaagcagtccatcctctggtaatgcttccgccatcgccagcgccgaataaccggtaaacatcccaatttctaagacttttttagctttggtcatgtgaacaaacatctttaaggtttgtccttcgatatgaccagaaagcatctcttgttctagaggacggacggttgtacctccgtggaagtgttctccccaggcttcggtggctgtggtttttgccaatgcagcgagttcaggagattctggagtggtgcattcttccaaataagggtctatacctgcggctaaacgccaagcctgatggatgtttgctatcaattccccaggtaaatctggatgttgcttaacctcttggactatggcttctaactgcttggttaaaattcccaatggtgtaacaggtctagctgttggttggacaatcacatttgtcaagtcgcttcgctccaattcaaaattcaaaattcaaaattcaaaattaaagacaattagtgtccgattatttgcgtagccttctctttccctacgggacgctccgcgaacagaaatgctaccgcgctcgcgcagtgtatccgtggagtattttgcattttgaattcaaaaaagtcattatttaacactcccgattaattctttttgataaacgggatacacatccacaccttcaccaccacggggataactggtacaaagttctttgtggtcagctaatgcggctgctaattcttctcttgtcaggtcattgacgaagacacaatcaccaatgggttttggcatagcagctcttaacaaaccatcacgagttaatgtgatagattcagtaccacgccacaaaatatctatatccaacatgggatggtcgagggatagaccaacgcgactcattaatcctaaaatacgatcgcgttctgcaattgtaatatatcctctacgggcggcgatcgttgccgagaaagccatatctacattaacggcgtgtccgtggaacatgggtagacgaggcgcaagttccaaggtgggactccaagtgtgaccgtaagcaatcaccctatctaggtctaactcatgcaggttgggaacttccaattccaacatcttatggatagctttgtaagtcaaacgatgggctatttctttaatctctggagttgcatctatattgccaaaatgagtacgtagtaattcttcgccgtacttctccaacaattcaaaaacttcttgatgcgctactacagcgatttttaccaattccgccatcccgttacgtacttggtctgtagggagagtacgcaacaaggagaaatctaaaaatactttgcgagaagcatgataagcacccaaacggtttttcagtttgcgatgattaactgctaccttaattgctacactggcatcaattaatccaatcaatgtagtaggaatgcggatgtaattgctgctgcgacgatatgtagaacaagcaaagccgacaacatctgtaattaaaccgccacccacgactaatactggttctttgcggactaatttgaaatctgcaaagacatctataactctctcgaaagtttgaatagtcttatctggttcagtaatggtaataggaaatagcctcagttctataccataatactggaaatatgcctgaatttgattaccatacaaccgactgacgttagcatctacaatcgccaagcatcgtccaaaaccttgatatacatctgctagtgcagaattctggatttcaaaaataccatctacatacaccaaatcatactcaatcttttcgtaaccttctacatgaaaagatgtttccttagcttcaaactttgcttggacgatactcatgactbactsantartmwcsagatctagcgctatagttgttgacagaatggacatactatgatatattttgctatagcgatggttgtaattggggagcaccgccacacacaagtcacagtcgacttgcaggcaattaagacaaatattagtaatgaaatggcgcaaaaggatgagttgaccgagttatgggcagtcgttaaagcgaatggttatggacatggaattatccaagttgctcaggccgccaaagaagccggggcgaccggcttttgtgttgcaatcctggatgaggccttagcgttgcgggccgctggctttgcggaacccatcctagtacttggaattacggaaccggaatacgccccactggtagctgaaaaggatatttcactagctgttggaacgcaagattggctgactacggccgcagcaattttagcggctaatcaagtgacgacaccacttcacgttcatcttgcattagatacgggtatgggacgaatcgggtttcagacgcccgaagaattggcaacggcggttacgactttgcgtcaaccgcagtcaccatttgactttgaagggatttttacgcattttgcaacggctgaccaggcagatgatacgtattttactcatcaattaaataattggaaacacttgattgcagtggtggatgagctaccacgctatgtccacgtgtccaattcggccaccagtctctggcatcaagcttgcaatggcaacatggtgcgctttggggttgcactctatggtctaaatccttctggtcgcgaactcagcgcaccataccccttgcaacccgcgttgtcgctaacggcacgcttgacgtttgttaaacgcttggctcggggcaaatcggtcagctatggtgccacgtatacggccgcacaggatgaatggattggcacggtgccgattgggtatgcggacggctatgaacgccgattacaaggcttccatgtacttgttgatggtgagttttgcgaaatcgtcggacgggtctgcatggaccagctgatggttcgtctgccacatgaagtaccggttggagctaaggtaactttggttggcacggacggtgctcgtaccatttcgttgcaagatattgctgactattgtgggacaattcattatgagattgcttgtgggttagcaccacgagtgccgagagtttatatagattaa

SEQ ID NO: 16 - DNA, 31 bp, Propionibacterium acnes
gagatcccatatgagtatcgtccaagcaaag

SEQ ID NO: 17 - DNA, 37 bp, Propionibacterium acnes
gtacctcgagtcatgaattattttccagacaatcttg

SEQ ID NO: 18 - DNA, 982 bp, Propionibacterium acnes
ggtccccaccccgcgggccgccgcccgggcggcgtcgacgaactccagggcgcgcggctgctgccgttccccgccaactgacccgccccgcgctctcttggagcactcgcacatgaccgtccggaagaaccaggccaccctgaccgccgacgagaagcggcgcttcgtcgccgccgtcctggaactcaagcgcagcggccgctacgacgagttcgtcaccacccacaacgccttcatcatcggcgacaccgacgcgggtgagcgcaccggccaccgctcgccctcgttcctgccctggcaccgcagatacctgctggagttcgagcgggccctgcagagcgtggacgcctcggtcgccctcccctactgggactggtccgccgaccgcaccgcacgggcctcgctgtgggcgcccgacttcctcggcggcaccgggcgcagcctggacggccgggtcatggacggaccgttcgccgcctcggccggcaactggccgatcaacgtgcgcgtggacgggcgcgcgtacctgcggcggtcgctcggcaccgcggtgcgggaactgccgacgcgggcggaggtggagtcggtgctcggcatggccacgtacgacacggccccctggaacagcgcctcggacggcttccgcaaccacctggagggctggcgcggcgtcaacctgcacaaccgcgtccacgtctgggtgggcgggcagatggccaccgggatgtcgcccaacgacccggtgttctggctgcacaacgcctacgtcgacaagctgtgggccgagtggcagcgccgccacccgggatccggctacctccccgccgccgggacgcccgacgtggtggacctgaacgacaggatgaagccctggaacgacacctccccggccgaccttttggaccacaccgcccactacaccttcgacaccgactgacccggccggccgtcggcaggcatcctcccgcaggtcaggggtacc

SEQ ID NO: 19 - DNA, 15 bp, Propionibacterium acnes
  modified_base (4)..(12): a, c, t, g, unknown or other
ccannnnnnnnntgg

SEQ ID NO: 20 - DNA, 7 bp, Propionibacterium acnes
  modified_base (4)..(4): a, c, t, g, unknown or other
rggnccy

SEQ ID NO: 21 - DNA, 22 bp, Propionibacterium acnes
  modified_base (7)..(22): a, c, t, g, unknown or other
ctgaagnnnnnnnnnnnnnnnn

SEQ ID NO: 22 - DNA, 7 bp, Propionibacterium acnes
  modified_base (4)..(4): a, c, t, g, unknown or other
tccncga

SEQ ID NO: 23 - DNA, 6 bp, Propionibacterium acnes
tctaga

SEQ ID NO: 24 - DNA, 6 bp, Propionibacterium acnes
tgtaca

SEQ ID NO: 25 - DNA, 6 bp, Propionibacterium acnes
gagctc

SEQ ID NO: 26 - DNA, 6 bp, Propionibacterium acnes
grgcyc

SEQ ID NO: 27 - DNA, 6 bp, Propionibacterium acnes
gagctc

SEQ ID NO: 28 - DNA, 15 bp, Propionibacterium acnes
  modified_base (8)..(15): a, c, t, g, unknown or other
cacctgcnnnnnnnn

SEQ ID NO: 29 - DNA, 6 bp, Propionibacterium acnes
gcatgc

SEQ ID NO: 30 - DNA, 6 bp, Propionibacterium acnes
rcatgy

SEQ ID NO: 31 - DNA, 6 bp, Propionibacterium acnes
tgcgca

SEQ ID NO: 32 - DNA, 6 bp, Propionibacterium acnes
cycgrg

SEQ ID NO: 33 - DNA, 6 bp, Propionibacterium acnes
ctcgag

SEQ ID NO: 34 - DNA, 6 bp, Propionibacterium acnes
cycgrg

SEQ ID NO: 35 - DNA, 24 bp, Propionibacterium acnes
  modified_base (4)..(8): a, c, t, g, unknown or other
  modified_base (12)..(24): a, c, t, g, unknown or other
aagnnnnncttnnnnnnnnnnnnn

SEQ ID NO: 36 - DNA, 6 bp, Propionibacterium acnes
gkgcmc

SEQ ID NO: 37 - DNA, 6 bp, Propionibacterium acnes
atgcat

SEQ ID NO: 38 - DNA, 10 bp, Propionibacterium acnes
  modified_base (4)..(7): a, c, t, g, unknown or other
caynnnnrtg

SEQ ID NO: 39 - DNA, 6 bp, Propionibacterium acnes
grgcyc

SEQ ID NO: 40 - DNA, 6 bp, Propionibacterium acnes
yccggr

SEQ ID NO: 41 - DNA, 6 bp, Propionibacterium acnes
aggcct

SEQ ID NO: 42 - DNA, 8 bp, Propionibacterium acnes
ttaattaa

SEQ ID NO: 43 - DNA, 6 bp, Propionibacterium acnes
gkgcmc

SEQ ID NO: 44 - DNA, 12 bp, Propionibacterium acnes
  modified_base (5)..(9): a, c, t, g, unknown or other
rgcannnnntgc

SEQ ID NO: 45 - DNA, 6 bp, Propionibacterium acnes
rcatgy

SEQ ID NO: 46 - DNA, 6 bp, Propionibacterium acnes
atgcat

SEQ ID NO: 47 - DNA, 10 bp, Propionibacterium acnes
  modified_base (4)..(7): a, c, t, g, unknown or other
caynnnnrtg

SEQ ID NO: 48 - DNA, 10 bp, Propionibacterium acnes
  modified_base (6)..(10): a, c, t, g, unknown or other
ggatcnnnnn

SEQ ID NO: 49 - DNA, 7 bp, Propionibacterium acnes
  modified_base (4)..(4): a, c, t, g, unknown or other
ggtnacc

SEQ ID NO: 50 - DNA, 6 bp, Propionibacterium acnes
gttaac

SEQ ID NO: 51 - DNA, 6 bp, Propionibacterium acnes
ccatgg

SEQ ID NO: 52 - DNA, 11 bp, Propionibacterium acnes
  modified_base (8)..(11): a, c, t, g, unknown or other
gctcttcnnnn

SEQ ID NO: 53 - DNA, 6 bp, Propionibacterium acnes
yccggr

SEQ ID NO: 54 - DNA, 6 bp, Propionibacterium acnes
ccatgg

SEQ ID NO: 55 - DNA, 15 bp, Propionibacterium acnes
  modified_base (4)..(12): a, c, t, g, unknown or other
ccannnnnnnnntgg

SEQ ID NO: 56 - DNA, 7 bp, Propionibacterium acnes
  modified_base (4)..(4): a, c, t, g, unknown or other
rggnccy

SEQ ID NO: 57 - DNA, 22 bp, Propionibacterium acnes
  modified_base (7)..(22): a, c, t, g, unknown or other
ctgaagnnnnnnnnnnnnnnnn

SEQ ID NO: 58 - DNA, 7 bp, Propionibacterium acnes
  modified_base (4)..(4): a, c, t, g, unknown or other
tccncga

SEQ ID NO: 59 - DNA, 6 bp, Propionibacterium acnes
tctaga

SEQ ID NO: 60 - DNA, 6 bp, Propionibacterium acnes
tgtaca

SEQ ID NO: 61 - DNA, 6 bp, Propionibacterium acnes
gagctc

SEQ ID NO: 62 - DNA, 6 bp, Propionibacterium acnes
grgcyc

SEQ ID NO: 63 - DNA, 6 bp, Propionibacterium acnes
gagctc

SEQ ID NO: 64 - DNA, 15 bp, Propionibacterium acnes
  modified_base (8)..(15): a, c, t, g, unknown or other
cacctgcnnnnnnnn

SEQ ID NO: 65 - DNA, 6 bp, Propionibacterium acnes
gcatgc

SEQ ID NO: 66 - DNA, 6 bp, Propionibacterium acnes
rcatgy

SEQ ID NO: 67 - DNA, 6 bp, Propionibacterium acnes
tgcgca

SEQ ID NO: 68 - DNA, 6 bp, Propionibacterium acnes
cycgrg

SEQ ID NO: 69 - DNA, 6 bp, Propionibacterium acnes
ctcgag

SEQ ID NO: 70 - DNA, 6 bp, Propionibacterium acnes
cycgrg

SEQ ID NO: 71 - DNA, 24 bp, Propionibacterium acnes
  modified_base (4)..(8): a, c, t, g, unknown or other
  modified_base (12)..(24): a, c, t, g, unknown or other
aagnnnnncttnnnnnnnnnnnnn

SEQ ID NO: 72 - DNA, 6 bp, Propionibacterium acnes
gkgcmc

SEQ ID NO: 73 - DNA, 6 bp, Propionibacterium acnes
atgcat

SEQ ID NO: 74 - DNA, 10 bp, Propionibacterium acnes
  modified_base (4)..(7): a, c, t, g, unknown or other
caynnnnrtg

SEQ ID NO: 75 - DNA, 6 bp, Propionibacterium acnes
grgcyc

SEQ ID NO: 76 - DNA, 6 bp, Propionibacterium acnes
yccggr

SEQ ID NO: 77 - DNA, 6 bp, Propionibacterium acnes
aggcct

SEQ ID NO: 78 - DNA, 8 bp, Propionibacterium acnes
ttaattaa

SEQ ID NO: 79 - DNA, 6 bp, Propionibacterium acnes
gkgcmc

SEQ ID NO: 80 - DNA, 12 bp, Propionibacterium acnes
  modified_base (5)..(9): a, c, t, g, unknown or other
rgcannnnntgc

SEQ ID NO: 81 - DNA, 6 bp, Propionibacterium acnes
rcatgy

SEQ ID NO: 82 - DNA, 6 bp, Propionibacterium acnes
atgcat

SEQ ID NO: 83 - DNA, 10 bp, Propionibacterium acnes
  modified_base (4)..(7): a, c, t, g, unknown or other
caynnnnrtg

SEQ ID NO: 84 - DNA, 10 bp, Propionibacterium acnes
  modified_base (6)..(10): a, c, t, g, unknown or other
ggatcnnnnn

SEQ ID NO: 85 - DNA, 7 bp, Propionibacterium acnes
  modified_base (4)..(4): a, c, t, g, unknown or other
ggtnacc

SEQ ID NO: 86 - DNA, 6 bp, Propionibacterium acnes
gttaac

SEQ ID NO: 87 - DNA, 6 bp, Propionibacterium acnes
ccatgg

SEQ ID NO: 88 - DNA, 11 bp, Propionibacterium acnes
  modified_base (8)..(11): a, c, t, g, unknown or other
gctcttcnnnn

SEQ ID NO: 89 - DNA, 6 bp, Propionibacterium acnes
yccggr

SEQ ID NO: 90 - DNA, 6 bp, Propionibacterium acnes
ccatgg

SEQ ID NO: 91 - DNA, 15 bp, Propionibacterium acnes
  modified_base (4)..(12): a, c, t, g, unknown or other
ccannnnnnnnntgg

SEQ ID NO: 92 - DNA, 20 bp, Propionibacterium acnes
acgacaccagcacccacaac

SEQ ID NO: 93-99 - skipped (7 entries)

SEQ ID NO: 100 - DNA, 2299 bp, Propionibacterium acnes
ttggtcagcgcccaccgatgccttacaccgtgacacgctcgaagtggcgatggaacacctacgcttcgaacctgattcgatcgcttcggtggcctcctgctgacgctttcatggatcgtgtcagcctggaggatggtaatgcgcccaaaactgtatgtcttcggtagatctgcacaagcggatcatctcgcgcaagaaggaaccgtgaggtactcgatccggtacactctccacatcaaaaccaacaatacaagtcacagtcatagatagttgtgtcattaacaagaaatcccaacagcaccccaatgcaaaattgcacagaacgacctagccagatgattttcagcaagtctaccatgacagcaaaggccgcagatagaaactgtgacatatacattgcacgctgaacttactaccccaccactaaatttacatagattccataccgacccgaacgccaccagtcaaccatcagtaatcatcatcgagaagtcaaaaataaccaacaccattgataagatccatgccatggacgggcagacgtctgtgatcagcagagtcgagaaacgaggtaaagcaaactatcacgagccaccagataatacccctagaagcttgtccaatatgttagctctggttaacatgccatcgccagcacaagattaacacgcttgttcatgtcttcagaaagcaacgaagctcgcctagatacttggacatttatatccttgcaaacccccccctaaagcctccaaaaatccttgatttacctcactaatataccattaatttgccccacctcaagccaacaaaagaacggagcatcagagccataggaagcctccgccagacataggggactaaacattatctccttctacgtcaccaactagaattcataaagcatcaacctacttatttaattcgatctaaaatccaatatccttagtatcagggatcaataaaaccaaaattaggaaacatacttaaaatcatgacatttcacttagtagacatactttgggattgtatcgacgcgtgacattgacacccatatttctgcggcagaatgcaccccggcaatccattgccaacatgcatgacacaccagcccatcagacccgaggtgaggtgatacactgttggagttagttcctggtacgcaaaccgcacagcccggtaggaagtccgggcggcgcaatagacgtccgtcgctcccctcatggagaatccagtgtcgccaggtccgcttcggcgtactgggagatgatatgcctacgatgttcactgtctacttcgatggccagttctgggtcggagtcctagagaggcgcgacgagggtttggtgcgtgccgtaaaagtcacgtttggcgccgaaccgtctgacacggaattgtacgagtgggttagccgtcatggcaacgcacttatagagcgattggagtctaccgctgctgtccctaccacccgcagtccccgagccaagcgactgaaccccaagagggcgttacgagatgcagcgcgagctgcccaagcaccccgtgccagcacggccgcacaggccgcgcttaaggccgatcaggaagctcggggtcgcgccgccacagtgaagcgcaagcaggcccggatcgacaaagctgcggagcagtgggctaagaaacgagagcgcgcgaaggcaaagcaccgtggccactaatgcgtcgaccgtgcccttcccaactgtgattcttggttcgggacctatggcttcacggtcaaggcggtcaaatcgctcacacatgagtactattcacccgacatgccatcgtctgggcaatcacccaactgcaagctgatgacgttttcctcctaggcttgatgcactagtttagtgtggacaggaagactcatggcgaagggaacggcctgttctcaagatcgccaccgcgaacgagattcacttcccacggtgtacgcatactaaggggcggagcttgtctgacatcatctgggccagcacgcacatggcccagatgatgcccaccggatggtgagcctgacccgtaacaagaacgcatcagcgagcacctcgccgacgcggctgcgcacgcggtcgtaacctcgccactgcctgcgtgaacaacgcgcttcggcgcattcaacacgccgtgaccgcggcgcccacggcttgcgcaaccgctacaactaccacctactgtcgcctcctggtcgttggcggcataacccataaccatcttcaaatccgaagcgccgagtcctgccataattcgaaccttcagcctgcatgactggaacaag

SEQ ID NO: 101 - DNA, 2299 bp, Propionibacterium acnes; identical to SEQ ID NO: 100

SEQ ID NO: 102 - DNA, 2299 bp, Propionibacterium acnes; identical to SEQ ID NO: 100

SEQ ID NO: 103 - DNA, 2298 bp, Propionibacterium acnes
ttggtcagcgcccaccgatgccttacaccgtgacacgctcgaagtggcgatggaacacctacgcttcgaacctgattcgatcgcttcggtggcctcctgctgacgctttcatggatcgtgtcagcctggaggatggtaatgcgcccaaaactgtatgtcttcggtagatctgcacaagcggatcatctcgcgcaagaaggaaccgtgaggtactcgatccggtacactctccacatcaaaaccaacaatacaagtcacagtcatagatagttgtgtcattaacaagaaatcccaacagcaccccaatgcaaaattgcacagaacgacctagccagatgattttcagcaagtctaccatgacagcaaaggccgcagatagaaactgtgacatatacattgcacgctgaacttactaccccaccactaaatttacatagattccataccgacccgaacgccaccagtcaaccatcagtaatcatcatcgagaagtcaaaaataaccaacaccattgataagatccatgccatggacgggcagacgtctgtgatcagcagagtcgagaaacgaggtaaagcaaactatcacgagccaccagataatacccctagaagcttgtccaatatgttagctctggttaacatgccatcgccagcacaagattaacacgcttgttcatgtcttcagaaagcaacgaagctcgcctagatacttggacatttatatccttgcaaaccccccctaaagcctccaaaaatccttgatttacctcactaatataccattaatttgccccacctcaagccaacaaaagaacggagcatcagagccataggaagcctccgccagacataggggactaaacattatctccttctacgtcaccaactagaattcataaagcatcaacctacttatttaattcgatctaaaatccaatatccttagtatcagggatcaataaaaccaaaattaggaaacatacttaaaatcatgacatttcacttagtagacatactttgggattgtatcgacgcgtgacattgacacccatatttctgcggcagaatgcaccccggcaatccattgccaacatgcatgacacaccagcccatcagacccgaggtgaggtgatacactgttggagttagttcctggtacgcaaaccgcacagcccggtaggaagtccgggcggcgcaatagacgtccgtcgctcccctcatggagaatccagtgtcgccaggtccgcttcggcgtactgggagatgatatgcctacgatgttcactgtctacttcgatggccagttctgggtcggagtcctagagaggcgcgacgagggtttggtgcgtgccgtaaaagtcacgtttggcgccgaaccgtctgacacggaattgtacgagtgggttagccgtcatggcaacgcacttatagagcgattggagtctaccgctgctgtccctaccacccgcagtccccgagccaagcgactgaaccccaagagggcgttacgagatgcagcgcgagctgcccaagcaccccgtgccagcacggccgcacaggccgcgcttaaggccgatcaggaagctcggggtcgcgccgccacagtgaagcgcaagcaggcccggatcgacaaagctgcggagcagtgggctaagaaacgagagcgcgcgaaggcaaagcaccgtggccactaatgcgtcgaccgtgcccttcccaactgtgattcttggttcgggacctatggcttcacggtcaaggcggtcaaatcgctcacacatgagtactattcacccgacatgccatcgtctgggcaatcacccaactgcaagctgatgacgttttcctcctaggcttgatgcactagtttagtgtggacaggaagactcatggcgaagggaacggcctgttctcaagatcgccaccgcgaacgagattcacttcccacggtgtacgcatactaaggggcggagcttgtctgacatcatctgggccagcacgcacatggcccagatgatgcccaccggatggtgagcctgacccgtaacaagaacgcatcagcgagcacctcgccgacgcggctgcgcacgcggtcgtaacctcgccactgcctgcgtgaacaacgcgcttcggcgcattcaacacgccgtgaccgcggcgcccacggcttgcgcaaccgctacaactaccacctactgtcgcctcctggtcgttggcggcataacccataaccatcttcaaatccgaagcgccgagtcctgccataattcgaaccttcagcctgcatgactggaacaag

SEQ ID NO: 104 - DNA, 1019 bp, Propionibacterium acnes
gggacagcgaccggtgaagggtccgctgggagcaaggggatcgtcagtcggtgtctgcaggctgaaggggcgcttggatgacgtggtgtccgctccggcggaaggcaccgagcttctcgctcagctcactgttcgacgactggacggtgacgatccccgtccggggcggtatcgatgcccaatatggttcgctaccggtggtgaggatcttggtgggatcagcgaggatccacgacgtgcgagcccgcgtcatcgcttcgtgtttgaactcgacggactccaggagtgggcagttgagcccgtcggtcgcagagactgcgtcgcagccgatgaacgcgacatcaggggtgatgctgcgcaggtttgatgacgcccacggtcccagaaggcttccgctgtgctgccttaacgccccgccgagcaccatgacgttggcggtgccttcggccacggcctggagcacggacaggccgcctacgaggatggtgatgtcgttacgagtcgacagaaggcggcccagtgctgcggtggacgttccggaatcgagcagaactaggtcactctcgttcacgaggtgtttggcggcatatgtggccattgctgccttggagacgtgctcgcattgttccttctcgtgccaggtaggctcgtgtcgtcggatcatgacgtctcccccggaaatcttccagatcttggattcatccgagagggcatcgacatcgcggcgaatcgtcgatgtagagacaccgaacagttgggaaagatctccgatggactccgagtgccccaaagaacgcagatggtcgacaatgtgtcggcgtcgggcagctgtcgccgtgcgcggctgtttgggtgttgtgctcatggtgtcttgatcggtgacgtggtttggctgatccaatttgaggaccgcctctaaggataacccagcggcatcactgctggactcggtctgtttgggtggtcggtccaagaatttctcaggtcacaggctaataaggttacctcgtcggtgcact

SEQ ID NO: 105 - DNA, 55585 bp, Propionibacterium acnes
tgccgacgccttcggcgtcgggtgagcgcgtggataggcgccccttcggggcacctgtaagcctgtatcgctgcggtttacagccttggccacccaaaggggcagcaaggggcccggagggggcattctgcgccgctgatagcatctcacatgtgagttattctctcaactatccttcaagtcaagcgtattagattatttatctagtactcttgacagtccgtctatcgggctatatatttataagcagaacgagagatcgggaaaggaatcaacgatgaagtacaccgcacacgtcaacatggtgaccaagccctacatcggacttcccactacccgaaccccctacaccctcgtcgacgccgactgggccacaacatggatgatcgacgaaacctacgaagcccactaccacggcaccagcgtggagatcagcatctacggcgaaggccccaactgctccgtactcaccaaccgcatctacaccgaccagcccaccgatgtgaccgctgcactcgccaactacggcttcaccatcgacaccaccaacacatccaaaaccatcctcacgctcacccgcacagccaccatctcccacctactagccatcctcgatggcctaccccaagccgaggggactcactggctcaacactcaacccgacctccaccacatcaccctcgacgactccaccaccggctgggccaccaaccgcaccgacgacaccgacaccggcgtctgctacgacaccagcacccacaactggcagatctgctgacagcataacgggcctgcccttgaagggttggactctgcacccatttcagccgggtacgttgccggctgggcgaaggaagcagacggcgccgcgattgaagctgccgccgagcacgtcgcgaagacctcccacgcgattgccgaggcgttgcagctcaccgcacagtgagccccacggggcgaccggctcaccccggtcgcccctcaccccccgacaggggaaccgttcccgccgcctgaatctccatacccctgatacgtactacgcggggtcgaggagtggactcccccaccctcgtgtagtacactatactacacagtcattgaggatagaggtgacgtcatgagtttttcgatccgtttaaccgaccatgagcgccgcttagctgattcctacgcccgcctgcactcgcagtccctgagtgaggctttcaagcaagccctcttcgaccgcatcgccgacgagttcgatctgcaagtagcccgtgaggcactcaccgactacgcccgcgacaactaccagtctcgccccatcagcgagctatgggacgagtgtgacctatgagctggcagatcgaaaccacgagccggttcgataaagaattcaaaaagctagaccgctacacccaaaaactcattcacggctggatcacgaaaaacatcgacaacaccgacgatccccgccgccacggcaagcccctcaccggtgatctttccggactgtggcgctaccgcatcggcgactaccgcctcatctgcacactcaacgataaccgcctcaccatcctcgccctgtccatcggtcaccgccgcgacatctaccgataagccgttgcaagactgcggcgtcgacaccgccgggaccctggcccaggccttgggcctggaggaggacgagagcgaggagcacgccgcctgagcggtcgccggcacttcccataacagcaccggtcccggcggcccggccccgcgtctggtgctcggaccgatgccggcgcctggcctccgacgcccgccggggccagcggctgggctacctcgacgtcgacgtacaacgggccgaacgaaccgtgcgctgatttaccaccgcttccgcacagagaacatgcctctgactaggagaaatgacgttttccacagggcctctattactaatatccctattatctctatccacgcgcgcgagcctcagcgttcaagcttcaggtcttgctcgtctccctcgcgcctcgctgcgctcgttgctactgccgccccacccggcaagccgggtgggttgccatcggaataccgtggaccatcccggcgcatacacccagacggtccaagatccaatccacgctcacccgcgacgccgctaacgatcctcatacaccgtagatgccgttctgagagccttccagacccgcacgtgggtttattgcaccgagacccaaatcccctcagaagcgatcctgaaaccgtctacgccccatttccgacggacgctggacaggctgggcggcttgtctcccgccgcagacatggccctggcatatgcggttgccgactggtgggatcagggattggatcacacggcagacggctggtccaaggcgggtgtgcaggtgcggtgagcggcagacgtgaccggtggggtgcactgtcatggccgggcatctgccgagccaccatgcgggccctggaccaggaggcagcccgctgcgaggactgggggaagacggcggcacggatgggccgtcgggatgcggcggccgaattcaccgagactgcggtgcagcatcgtggacagatgcaccacctcatgtcggcacaaaaagtgtgggtgtcgaccgacatggtgtcggctacactggacgccgcaggagacgtgccgagtttcgaggccgtcgacgtccctgccgccgggctcatgggtttggccagccccctgccgccggttaccctgcagcatccgctgtatctgcgcagcgacgagggtgtgacaaccttcactgatccggttctcgtcgacgcgctgggatggtggatggattctgggcgtgtgcacgtggtgatgtgcacacgcacaccccggctgcccaacccggtttatgcggtggcctccccactgacggtggtggagaaaatcacggtgcccaccggcatcgagttcgagaccatggagtccaggatcctgacccccggcggcgtgacaggcgtctcacggcagggcacacgcatgatgatccggttggcgtcatggctgggtgccgcgtgggtgctcatggcgactccgacggtgtcggagccgcaccctatggatggccggtggggaggcccagcaacggggcagacccggccgcgtgatcgggtgaccgtggtcgacatgcggcccatgaggcaggtgcacaccaccaccgaccccaccggcaggcggttgacgacccggcatgtcgtgcgaggacactggacacaccaaccctacggcccggcccgcagtctccgacgcctgcagtgggtcgccccattcatccgcggccccgaaggggctccgttcgtcggcaccgacaccgtcaccgtatggcgccgctagatacgcgggcgcggggggttgacgaacgatgcccccctctttgtgttgtgatcagtcgtgatcggcgggttttaggtcggggtaggtgtcgagaatggccttgaggacgaggtcttggattgtggtgtcattctgggctgccttgaccttcagtccaacgccgatgtcccgggggattctggtctgcaacttgaacaggtctgcctccttggttttgaacagtttggctgcagggttggcaacgggctgttcttggcgctggcgtccaaggttgcttttctttcgggcggtcatgacatcatctccaacagttcgtcggcaacatcggcgaagaaggcgatagtttccggctcaggggtacagccgtaggcgcgcttgatgacttgacgcttggggatgacgtgatcgaccaactgcgtctgggagtcgttggctagaaggtcgcgaccttccttgaacagggaggtattcggttcagctttggaaagcagcaccacgcgcggggttcctgcggcggcgtcgtgcgtctcccaggtgcgcgccatgtcgagcccggacgtgtcggtggggatgataaccagatcggcggcgcgaatggcggcggtgatgatgtcaccgttgcccggcggggtgtcaatgatgacaacttcttcgggtccggcgacgaggtgctggagcgtcgccttgttggccggttccacggggaaccccaaggggtcggcgttactcagtgtgcactcggcccaggatgatgctgacccttgagggtcggcatctttgacgcggaccatacggcccgtctcatggagagctgcagccagcaggatcgctgccgtggtcttaccgacgccacccttggcgttgataatgctccagatcatggtgacacctttcagtgaggatgcatatcagtttacctgcaaggagtaaagacggcaagcagttatgccgtttctgcggcaagccgtcttgcatccttggatgtatagatagttgtctacttatggttgtggatcgtggtgtctcgtaccaggatttagtgtgctcgctgatatgcacgactttcgggcggcctgatctggcggtggtgccactaacccaggagtctgatccgcaaggtgtcctcgactttgaggaaggggtctctgctttcgtgttgacctaaagtcaacggccctgggcgatcactgaatcctaataactttgtatccttccctgattgcatcttagcatccttgaattcaaacagggatggagccatgctgttttgacgacatggatgcactggggtttggttgttgccgatgcggagcgtcaggcggcccgggacgccgccaccgccgacacgctgcgggagatcgtgggactgctgggccggggcggcgaggtcccgcccgccaagacggggcgtaagacgaccggcaggccggccccggcccgcaagtcgtcggccacgcagtaaaggcagggatccgtccctacgcagagagcggaggcagatcctgggggatccgactcttcggttccgtgatcttcccggagatcttcccggatcccccagggctcatcctgatacggggtccgccgacggaccccgtatcagtgggggcgctgccaccagtgatggcgggcgggtcggtcggcgtctaggccccgcccgatggtcgcctggtctacctcgcaggcccgtgctgttgccgaggccgagaacgtctgcccacttgttgccgattcagtcccctctccgtctgccatgtcgccaacctttcctatctggcgtgctcatggacatgcccgggcacgccctcgctgctcagaggcctgctcgttccacctctggccgcgaaccgtatttctgctgaccactcctcgtgcgtgaatatttgccagccttggccattcatatttggactactctgcaccagatatcggactgggaaacctcttgcacatctctaattgtcatctattcgttgggcgcgcgctgttagatcgtgatcataccaatcagaagacaaagcgacgcccttttgagcggcttttcgaacgattctagaccgcgctatatcagcatttctaccgcgcggccagagtgctacactaaattcctctctgcaatccttaagtccattctttacttcctcatccaatttctgcgacagcatttttatggaagcatcgcactctgactcatcatcaaatgcctcaattgcgtgcgttcgccattcacggcgcgtattatcgtagccgtaatgaagtattacccaccgatccacagaatcatcgttaggatctacaactgtcatgaatggcccaactctcgctctggtgtattctcaattgcagttcagtgactttacttgaccagtcccggatttagtagacgtagacggagagtaccccgccgggaacttgactgacatcactgctgtaccgtactgctttcccgggtggcaaccatcgtaagcagcatttgccgtgacgtacctagtgctctttctctgagtgccggtaatcgccttatatggtacaggtgggtgaatcgtagccgttacggctaccgatgacacgtttgcatcacatgaagctgtggcgacataattagttgtcccattgacatgatgagagtcgtggacatactgtcgaggatcgagagtgcaggttattgttgacgccgttgttctggtaccagctgctttcgagctctttgttgcagttacagtctgcggccctgaaggttttactgacgcgactgatgtgttcgcgtgttttgcggcagtgctttgatttggttgtgcttgtgtagcagttgctgatgtcgccagtgatgcccccattaccatagctgtgattatggcgtagtctttacgcttcattgcgcccccaaataagcatgttagtttgctattttgaataggaaacgagacatcgccatgacttgtcaagggcagaagtagaaagaatcggggggtatcaagaaagatgtgctttcatcaggacgaaagcggatgggtccgttcgacttggtgacgaatgatccggccttgcgtcgcggagaccaggccggcgagtttgtcgagcagggccgccgtggcctcatcgtcgggcacatcactaaagggacgaacaaccatgacggctagggccagaaacgaccggaactgaatcgcatgtggacccccatctggcaccaggcacacacctaccgaccggaaaccctgcaccctgtcgctcccagttcacctcctaatatgcgcctatagtctgtgctagtagacattattttgtgtgttgtttcccatctggcatgtctggccccggacccgccgcgacacgcggtggtggataacagaccaggacccccggaccatagagaccagggggacgagacagtcccccggtgaggatgtctcgatgtgcagatcaggaacagaatcgccgggcacgacccggcgccggtgccggtgcagtgaacccgtggcccatcgactccacgaacgaacgtctcgccttcctcaactggtgttggcggcaaggctactcagcagccatcgtggaaacgatggacgccgcgggagcctacgaccacctcgacgacgaccagaccagcaaccagcacgactaacgccgctcccagcacgggggaaggcggcgcaccaggtgcgccgccttcccccgtgctggtgtctcgtcggcttactggtgggggtcgtaggggaacccgtaggtgtgttccagggccttgcggagaaggtcgttcatagttgtgtgttcggcgacggccttcatcttcagcgccgtgtgggtggcggtgggtagcttgacgttgaacgcggtgatcggggtcttggccgactggaaggtttcagagacactggctccctggctggccgccatttggtcggcgtaacggctggctttgtcggcggcggtgggtttgcggttcttactcacttcagcatctccttgattcggtcggcaacctgggcgaactctcctagatctgttggccgggttccgatggcttgccgataggcgacccgtcgatgaacctccgcatcgagaacgtctaggccctcaacggcggcggccccctcgtgggcatcacggtattgcctggtccaatcggctttgttgaagagcagggcccggcgtgtgccgtgggccacctggtaggtcattagcgctcggtccatgtccaggtcccccgactcggtcgggatgatgaccacatcggcgatgtcgagggctgcggtgatcgtctgagagtcaccggggggagtgtcgatgatgacaaggtcggcgtcgatgtcacggcagcgcctgacgataggggcattagccgggcggacctcgaagcccagcgggtcaccagtctcgaaagcgtcggtagcccatgacgtcgccgatccttgcgggtcggcgtcaagcaagatgactcggtactgagctgctaaggccgttgccaggtagaccgccgtggtcgttttcccgaccccgcccttggtgttgacaatagaaatactacgcataaatacatcattacacatgtgcatcgatgtatctgtatatatgtgtatcggcgtgttgtgagggaaaacccgggccacatacaacgatcccccagccaccacagccgggggatctctcagtaaagccagcaactcccacagaagttgcacccccacgatacccaccacatccgtgtcaagcccccacaacacccaccagcgccctaggcttcccccatgaacattgacatggacacaccaaccgaaaccgaagccgacaccttcgatgccgtcctcgccgacattaaccacatcaaccgcctcaccgccgacgaaatcgagtgctactccatgggagcctgtctcctcctcctcaaccaccaccccttctccgaactccgccaacactgggccgacaaccccaccgacgaaaccacgtggaccctctacggggccatcatcgccgcctccaccctggacccccacctcagcatcgacgccctagcccgcctctaccagctccccctcgacctcgtgacccaagccctcaccgtctaagcgtcgatgagcgggggagaggtagcgcactatcggctaaggaaccccacaaacaccaaacccttgtcactgggaagggtttggtgttatgctggtctcacaactcaacagaggaggtgagacatgggaaatgagtaccagaagtcgctcaaagtcctgttcaagaaactggaatctgagcaaggagcccgcatcgagacccggcgcaaaggatggatgatctacccgccggatacgtcacgatcagcggtcatgatccacaagactccatcagatcgcagagcatgggccaacatgctttccgaactgagacgctcaggattcaccgtctaactcagtgaagggggctggagctccagccccctttcaaccacctcccccggacaccacacaagaaaaggagaacatgatgtggcacgttcggttgtcgcttcgcgcaatcagcgaggacatgctgttcgacgccctggacaccctcgaccccttatccccggtggccacactgggccccgatgagcacaccggatcattggcggtgttcgtcgaggctgactcactgacagacgccatcgagactgcccgcacagccatcaccaacgcatgcaacaccgccaccataaccggcgtagaagcccgccccgaagacgaatgcttcgcagacgttgaccggcccctgttcccgccagtggtcggatacaccgaaatcgctgaaaccgcaggactctcacgtcagcgcatccgccagctcgcaggcaccgccgggttccccgcacctgtcatcaagaccgcggccggcccgctgtttcccaaagccgccgccgagcagtgggcgcggacacgccagcctaaggcaggccgcccaaaactccagaccaccacctcataacacatcccgtgtgctgaacctgttggccggcccagcacacacccccaggggccgggcccagcccggccccgccccacacccaaccgccccctgttgccgccgtggcgaaacgatcaggcgagccacgaccatcgccaagctgaatggagagcgagcatgagcaccacccctgccgggttcgacttcgacgctctggccgagtgggccgagagcgatgaggcgacccacaccccgcaaaccagccccgtgttccggggcaaggacgccgcgcgcgccagccgcgcattcctcggccgaggacggcccaccctcggctcagaccacgccaccggcgagggccggtcgccccgccgtcaggtacggctcgacgcacgcaccaacgcgcgactcgacgcctacgcagccgcaaccggcaccagcgcctcccagatcatccgcgacgccctcgctgactacctgcccgcctgatcccaccccaatccactcgggcgcctgtcagtgccccctcttctgggtggtggtccggggcgtcaccgccccggaccagttgggtagtcatctcagttgaaataagcgattagtgatacgcccctcatgctttccggtgctgtcatgagcgtctgacaacagcgacacctcggggcagccgacggacagtcctctggctgccgctctgatcccatgcccacggcacgaaggtgtcggacagctcggtctcgtcatgtgcccaaatagcgtcgtgggcctgtcgccaagcttctcgggagccccacacgggcacgaccgggccacaccggtgtcctggtctccagtgtcgcgacactgtcgatttcggtacgtgcaatgcggcggcggtttgacggatcgacatcccagactcccgcgcgtcccagatcgccatgtggagagtatttgcctccagtgcggccgccacacttgcacgctctcgcgcacgctcgaatgcccgcctagtgctctcaaatgtcgtcacgacgcctccccctgaaaatactcagggtgtcccacggtgggacaccctgagtatttcataccccaactcgcctcaccacacgtcgacctcgacaccgatcaagcgacccgtatcagcgacattgccgtaccgctgccccgcctggtcctccagccacgacaacgcatcccgaaacagcccgacgtcttccaaagctaaaccacagtcgtagccgcccttgtcccccaaccgcacaatctggcgctcccggtggcctccctcgtcgacctcgatccacagtttcgcgtgcatccgctggcctcggccctctgacaggatctcggtgagcaggtcggacgcctgccctcgcgcatcctcgttactgcgggggaattggagtatgtgggaggaacggtggtggggcaggtttgtgacctcgtgacgcagcaggttgcgcttgtgcatctgccgttcgagacgctcgggctgacctgttgacggggttgagccgtggcgccgcttcctcggcggggtctcgatccgttgctcttccggcggttcggcgtcaggttgtggtgcgcgatctcccctaggacgggggggctcgccacctatatccgagggacgatgagacggccttccacgcttgctgcgcactttcgcagcccgccccttgcggtcacatcgacgcggcggcggagtgacgtcctcaccgcgcgctaactgcgtcaacgccgccaccaggttggtgcccggtttcagccctttcagcacgaaccgcaccaggcgggggtctcggccgattcgtcgggcaatctcggcttggctccacccttcggctaacagggtgcgcaacaagtcttgggcccggttatcacgctcagtcactggtcgcctcctcggggagtcctgcctgctcggcctgctctgatagagcagcccgttccgatccggtctggggcgctttcaggggaccgggtaggacacctttgacacgcagtcgcccgtccttgccgtcgtcgccgcccccctgggccacagggagcgcccaggggggcacgttgctggtgtagatggcgtcgccgtggatagccagcagggtgcgggagttcatgcgtagtgctcctcccttggttgtggcgttttttccggtggtggggtcggcgatgggtgtgttgagggtgcgtacgcgggacagggcccagatacgggcggcgatttcggggtgccaggtgtcggggtcggggcggcggcgggcggtttggtattggtaggcgatggtgtcaccggttttgttgcggatgacgtcgaagccttcgatgtcggaggggatgtcgtcggcgtcgtagacgacggtggtggtgacggggttggaggccgacatttggccgatggtcacgcgcagcatgtttttcaacgcggaggtgaggattgtcgtggcggtggggttcttgtctgcccagcgtttctcggcgatgtcgatcatgtcttggatggttttgacccatcctcggatgggatcgactttggtcagtttgatccctccaaggaactcgatgggcttagtaacgggcccgtcaggaccgaattcggtgtcccagccttcgtcgatggctagtttcagttcggcggcgtcaacccatgtttcgtgggtggtgccgggcacgttgggccaaaaccatcctgctcgtcctttggtgcgtttgaccggtagtaggccgatgtcgtcccaccaggatggcacggtgaagcggatgtggtagcgggccgggtgccaggggttgttggtgaacaggtcgtgagcttcggtggcgctgagcagagtggcgggtgcggcaccgatctctccggtcactgatccggcatacatgaatcggccgtcgatgtacacgaagttagggatagggccggcaggcacggtggggcgccaaccttgttcgcggtcctcggggttgccgtcgtcgacgcatagttcgtagcggtgctgaggggaggtgtgttggatgagtgcgcccagttcggggtccatcggctccatgcgataggactcgggggtccgtagcttccagaggttgattccttctgctccggggcggtccatgagggcccagtcgcggtcgattctggtggcgatgatgtgggttaattcgcgccatgcccagcgggcctggattggggtgagggtctggccagggaaccattcatcagcgaccagaatgtcgaggttgcggtcggtttctttgtgggtgaaccggcctcgggcgggccgcccggacagcatgaacaggccccgcacgtgccagtcctggttgaactcgcgggtgtgagcgggggtgtcaggcagcagccaggattgaggctggtcggggatgttaccgcagacgatcaggcgtcgggcgttgtggtgtagcgcggttgttagcaggtctgacagggtcgcgtgcttggtgcgccgcccccctagcactggcaggatctcgtttccggccgcgtcgactccccgggcggtgtcgatgtctagccacactgtttcggggtcggggggccgatgtggggcgggacggcgctgctcctggtcgtctctcatggtttcctcctttgggccagggagcggccggcgtcgagggcgttcatgcgctcgcgttgttcttcgcgtagttggtagacccggcctcgtgacactcctgctgcctcggcgatggtggtggccggcaggtggccgatggcggcagcgtcggcaaggacgtcacgccaggccaggtcaatggttcccagctgggctagagcttcgtcgcggcggcgggtggcatcagccactacggaggcccaccctgtggggtggatggtcttgacgacgattcggttgtggcggatggggccccatgcatcagcggggacttgccagccgtgattggcggcggactcggtgatcttgaggcgtcgctcaggtgcgcgggtgcgggcagtgacgtcggtgttccaggtctggactggtacccacgtcccctctcggatgcgttcgatgacgcacgtggtgacctcgtcggtgactgtgatgaccagacgataatctttcccgtctggggtggttcggtagctgggcatgtcaccctcccctatctgtcaaatgagctagacacagtgtctatttaatttgacgcaaagtcaagtgcattagacgcctttgtgtggatccgccccaccagacaaacactcgcgcctcatgacccgccgcgaagccttaacgaaaccaaaccgcttatagaaccgctccgcagcgctccccgagggctttagctccaaggtaagaccttgctcgtcggcccattggcagacttgtgtgaggatctgtgagccgtagccctgatttcggggggcggcatgcacgcacatcagcatgtgaccatgacggcgggtcggcgggctgacataagccccaccacgccagtccggtgtgatctgccaacttcgccatgccgcagcgcgccagcggcgggcctcggtagccaccatgatcaccatgatgatggacagggttacgacgccgactatggtcagctcactccagccaggccagttcccggagatgatccagcctcccagcgcccagatgatcatggcttccacgcccgccgcaatcgggacgagtggggcagtacggatgcggcctgccatgatctcggtgatctcgctgcgatgccatgacaaggagaattcccacgactcttgaatcatgctgatgtgtgaacggcttcctcggcttcgccctgccatagcttctctagacggcgctcggctagctcgtcggggcacggcgacagcctgccgcactcggagcagaccccgaagtggcgcgagagagtgatgaccggctcatcgggagcatgaaccgattcaacgaccgggtgctgctcatcggtgagtcctgaggcgtgaccaatctcggctagacggaggtgaaccataccgtcgtagtagtcgaggccttcaacgtggaacgggtaggagccgtccgcggtgtggcctgcaaccaggtccccgacttccagctgactggcggtcatggttcgcaccggcggcagcggggtccgggtcgcgttgatcctcatggtctctccttgcttctgcgccttcatagatgaaggtgccacagccggggttgatgtgcggaaccacagccactttttcaataaatccatcaccacggtctcaccccttcccaaatgtgtaaaccgtggtttacgctttaaggcatgatcgaacacacaccgacacccgccgcgcctcgcgtggccatcatttcccctagcgggccaccacggttcttggccgagcctcccagcgccgcgctgcgacgccatggtcggacccgcatgaggtcccttcccctatcccctcacattcgcgcctgggtctccaccagcggccgtaaacccaatcccatagccaccgccctgatctggatgcataccggagccctgctgccggtgtgggacaccgccgttttgactgggcctgccctcggcagacgtgtgcgccctctcagcagtgacgccgaactgacctctcggcggtggctcgaccaggttgccgaccatcccggctttctcgacacccttatcgaggccgcccgcatcaccgatggatggcatcacgcgacgcctccgcacattcaggccccttaccggacccttcatcagtgaacggaggtacaacgacatgaccagcgacattgacattgaccggacccacaaacccgacgaccgcgccgaggctcctctttaccggtcctgggccaggagactcgtgcacgtctccgggatgccctggcgtatcgtcgccgccttggccggggtctcccccacctccatgcaccggcttctattcggccgcaacggtcgacccgtggaatggatcggcatcaatgatgctcgcgccctgatggatatcggcatcgatgatctcgcctcagcgtccaccgaccgtatcccggcccgcgaatcccgcgagctgttggtagccctgcacaccctcggctggaccgatgagcatctaagccgctggctgaccagttccgacctcgacctgaccaccgcccccaaagccctctacgtcactcgactatccgccgcccgcatccaggccacctacgacatgctcatcagccagcccgtccgccgctgcggccacccccgcaccccgcccgtctcgtccccgacccctgctgtcctaccccagcccagacccaacaacaccgaaccgttccaacccgccctgttcgagctggccgactgcgcatgaggaccctgcacagacgtgattaaatcgaagggctcaaccagtcatcaggagtcccccgtgcgtttgtaccgcgctgttctcgccgacaccgacatccatatcaccatccgtatctggaacactaccgatcgtgactggacctgggccccccttgacacttgggcccccgacccggccccgaccacaccagcccagttagccgatgaacttcaccgtcacggatggatcacccctgaggtccctactaccctcaccgaggtggctgtcatccccgagaactggcaagctttcgtcgagcatgccctcgctgtccggaaccagcaggctgaccaattgcgcgtcgccgagaacatcctcaccgacatcctcggcgacgccgccgacgccggcctgtctgtcaccgctctcgcccgcacgaccggactatcccgagtcgccgtctacaaacgcagcgccaaaaccatcgactccatgagacacgccacccaggccggcggaatcctcaccccctcctgtctcacccacgctgagagaacagcgctcggcctgcccgacgagtaacccatcgtttacatcctcgactggtccctgcgaccagtgaaccgtgcccccactgcgcccacaatgatcagcacacccacggcgatcatcaggtgggcagcactaactgatcccaacgaaccaagatcaaacaagccctgcccgggagcgtcgagcagtccgtgggcgcgaagccatgctccgagattgccaatgatcttggggatcatcttgttcagtagggctatccctccgatgccgacaacgaggaagacgaggatttcttcgcctgcgttgtttcttcctgtgtttgcggtcatgctgccttctcggggtggatggccagccagctgatgaggttgtcggtgatctgccctgagatttggatcagttcgacttgcccggcctgccatgcccgcgatgtgccggccagcaatgatgctgggacccctactgcgcggccacgggatgcaacctcagccaaggcggcgcgtaactcctcgacggtgctttcgatgtaccaactgccgtcagtactagtctcagcggcatcggtggtagcgataagagcatcggtgaggtcgtcgacggcgcggtccagatcagcgatgtccggtagggctacaggagtcatggtgtgtcttccttctagggtgccttcactggtgaagggtccctagccccggccgatgtgcggtgctgtgggaaaaatgcttcccattggtccgtcttagttaggaaacttggcctaagtgctgtcgagggtcgcgtgttcgtgaccggttcgaagacactgccgccttgtcttcttgcccgtggcgcttccgagatggtcagtgggcgatggctaggccgatgagggcgccgctgcacaggctgggcgcccagggaaggaatcgggtggtgtggtgtcggaaccggtcgattagggcatatagggcgatcagggtgaggccggctatgacagtgacctggatcgtgtcgatgccggttgctgcggccggggctgcccacagtggtgtgatgcgcacatcagagaaaccgaatccgcctcgggagatgagccaggcgatcatccacgctgcgaagatgatggcggtagcgatgacagcacgtatcgccgcagtatggtccgagagggcggtaacgactacagccccgatccatccggcccatccggtccacgctaggactgccggtatccatgtggtgatggcgtcaatgacggcaacccatgccccgatggtggctagcagagcccaggccggccaggtgggtgctggggcccacgctacggcggcgatcatggttgtcgcagatgcagcacagacagcgatcaagcttcggaggctgtaggtgtcgcggtagacgatcttgtcgggattggcggtagctggttctggcaggacgcgggtgagggcaggccatcctgcgaccgcaatagccgcgacggcgaccacccacacaatctgttgcatgggtctcatcatggcccatcatcgatgagtatgggtggccgacatggggtcactcgtagaggggatcgaggggtgtgcgggcgagcatggcttcctgtgtgccgagcatgagggtgccagaatcctcgccagccgattcccagtcgacgatgacctggtcgccgtcaacgctggattcggcgatggtcacccattctccgtcgatattgatgagcaccccagcggccagctccgtggcagcaaccgggtgaggtgggttgtagaagcggtcgatgtcgcaggcagggtcgtcgggttcctcgactaccggaggtggggtcgatctggctgtctcggtgtcgtcggccaggtcggctcccaggatgcgctcccattcggggctggttttcttgcccgctgaggccatcgtctcgtcgatctgttcgtcagtgggccacacaaatttctttttggtccaggtcgtggtgtcgggtcgcagctgatccagcagttccaggtcttgcggggagtgggccttgcgggggtcgggggtgtagaagaattggacttccttgggggctgattcgccggataggtaggtgccgcggccgcgtttgccgaacgggatgcccacgccgacgtgttcggagccgaacatcatgcgggctccgtcagcggacaggggtccggtggcggcacgcccggagaagttgtctcggatttcgccgcccaggaactcggcgtcggggcgttgggtccccagatcgacgtggatacggcaggcggctgccatgcgcagcagggagccgatccagccgaaaacggggcattctccgggcatcccagagaccttgatcgttgaccaccagtttttcgcgttgccgtagaactggcggtactcgtcgatgaggaccagcacgcgggtgaagtcggtctcgcgggccccttcttcttcgatgcggcggtagcggtcctccatgagagaccacagccagtggatgagggcgacctggtcggggacggtagtggcgaccatctcgatgttgggccactcccgcaggcccaggtactcgatgcgtttagggtcgatgacgaagacgcgccagttgcggcgggcaccctcgacggcgtcgccgatcatggacaccgtttttccggtacgggtgcggccggccttgagctggtgggccatgacaccggagatgtcccagaaacaggtgttgccgtcctcgtcgacggcctgggggattttctcccagtctggggagtctgggcccggaagggccacgggtcggggcaccatcgagggcagggggtggcggcgggtgacggtgatggtgtcgtgttccaggtcgaagtcgctgcgccactggccgagcatcatcgccgaggtcaccgcggccaggcgcaattggatgttcgggctggacaaatgggcgccacgttggtggtgaatggtgaagccgtccaggttcccccgatcggttttccaggaatcgacggtgaccgccgacccatagatctgagtggctacttctttagcccgttccttgtctcggtccagatcggttttgtcagggtcaggggtcggggcgcggtattgcaggacgaaccggccggccgggatcgttgcggatgtcaccgtgaactctgccggcgaccaggcagccgcggccgcctgggtgagggccattggcatggtctcgacggcagcctcctcgggggggatggtgagggtcaggcgtcgcggatgcgtcttcttgcgattccatgtgactttctgcctcaccggcaccttcgccgccgactgaatggctaggcgagctgattcggcagtttcggtacgccggccagcgcgtagcgcccgtcgtgcgtgaaggctgcccaccacagcacagcccatacacggcagccacgagcgcggatcagcggctgctgcatagcccccggtagctagtaccgcacctgaccagccggccgcgatacttttccacgccggtttcgcgccaccaggaccagcgttatggacggtctgcttcgaggctgcggagggacgagacagcggagtgctggtcatgccgcggcctccttgtggcaggtcatttcacaggcatgccggccagcgatgatgccgtggcgcaccgccccggtagccgcatagaccgcgcacaactcaaacacggggcacccatcgcaggccgccgggttcatgctttctttgtcccacgatggtgggtcatcggtgcacggcacggccaggccatcctgttgagcggcgctgatagcggtgaataggcgcagctgggcgcgaacttggtctggggtcaggttgacggtgcgggctacacgcatggggttccttcctcttcaccagtgaaggttccctagacccggccgatgtgcggagggatttcagtgttttatcgaggtgaccagccagccggtctgcgctgcgggttcaggtaccagggtcagatgcagcatggtgtggtcggctaggggcacggcgaccaggggcccgtcggcgctcgccccggtgagggtgggagttccggccacggtggtggcgggaatctggttgggtttcgcggccatcagggagcccatgagggtcggggcggcgatctggacgagggcgactttgcgggcggcgggatcgcgggtcaaccagccgcgggtgaaggtgagcgcagccgacgtgatatcggggctcagcggggctggtgtggcggcggggtgggccgttgaggagggccatacgcgcggtagcgctggcgacatcgagcgggtcggagtggggtcaggtgtggggcttagggtccgatgtggcgatggggctaccgccccggtggtgggagacgagactggttgggggtggtgttgagggcgtgggatcagggtgaaggcagccaaactggccgggatgaggatgacggccgcgccgacacttcggagcgaggtcatgtgacgatccttcggtagctcgacgggttgccgtagatcttttccacgttgatgccactatgtgggttgtcggcgctaaccatttggcctccaccgatgtagacggccacgtggtgggcagggcttccccagaggatgaggtcgccgactttgagctggtcacgggccaccgcggtgccgactttctgctggtcggcggcggtgcggggaaggctaatgccgatacgccggtacagcgccgaggtcaggcctgagcagtcgtagccgccatcggctttgccgttgccaccccacacgtaggcgtaggagttgttgtcggcgatcgatttggcccatgccacggcttcggccaggttcttcgacacaccggtctcgttagcggccccggtggcacagttgtttgacaggtgccctgtagtagtggatcccactatttgtttggccatggtttcccatttggcgtaggcatcgggcagggctgagccttgcactgcctgagctgcctgcgtcagcggcatcttctgccagtcagcgatgtcgactaaaccgggaatgtggaatccagggccgccggccgatccggggccagcgtaggtgatggtgtggcctttcaagaacgcctcggtggcgtaggcatcattgttgacctgatccagagtcccgtaccagcccggcaggatgcgctgctgaaaggggccggcgtcaccgtcaccgttagccttcgctgaggtagggtcggccccaagatctgattcttgtttggcggtcataatagcgatgaccgctgcctgggtaggcagtttgtcacgctggaccacagtgacgatggtggtcacgagctgcttctgcctggccgtgaggcctccggcacccacagtgcctccggtgacggtacccacgcaagcatccccgccgacactgccaccgccgacgccagagttgtcgtcgagggccccagagcctactgacagcagcggcacaatgatcacggtcgcgggcaagaccgccaggcccgccagcatcagcactgctgcactcaccacggcgatcacgtgagatgctttcaacgctgcccacctttcgctggtttgagggggcgacgcctgctggtacgcacaggcttggttggtccagtccgccgttggttggcaggcctgggtttgatcggcttgctgggacccggtttgggctccggagtcttaggctccggcttcacatgcctaggcacttcggctgcggtgggaggagtcagcacgtggcgtaccggcttggtgaccttctgggcacccgaaacaaccggcttagacaccaccttggcacctttgaccaccggtgcggctgccttcgcgccggccttcttgactgcacgatgcgctttgatagccccggcctcggcccggccaaccctgctagcagtagcatcgagggctctgtggcccgtcgttgccctcaccggtgcatccttgccctggcgggtctgcttagcgatcgccgacatgccgcctcgcaatgccgaaacatgattgtgggcgtgggcgatcgccgacatgccggccgtcgcgatttttgactggccctgcggggcccctgccccagcacgaatctgctggtaagccgccgacgtgccacgagcacccgccgccattcgcgccaccttcgcagccctagtcactcccgtcgacacgccacccgtggctaccgcagcagcacccgagaccacagtcgaggccatccgccttcgacccaatccggtgagcttgcgaccaccgctgcgcaccatccggcgagcaccccgcccagcccactggccgatcaccgagcccctttcggaacgcttgctgcgcatccaatcgttgatcttgtccctcagccccttctcgcccttgtgacgacgccacatggacatcaagaacccgataatccccacgagctcgagcatgttgacaaccatgaccttgatggcgacatcaccgtcagaggcgaaaaccgacttgatgatgagcatgaccacacccaacatcaccatcgacccgacaatgccgatcatcgagtacaagatcgaacacagatcccggatgagcggatctgtgcggccagggatagcccgcaacacgttgagcatcgacatgaacgccgaccacaccaacttgcacacgtagaaccagcacatgcccgcgaacaccagcaggaccacagccaaacccgccaccgtcgacgagtaaaagaagaaccccaccaaccacgaaccatccgcaatatcgccggcgtaatccgacaacccctcatcacagtcaccaaggtcgtcgcgagcatccgagacgtcatcgtaagggccacccttgagcacctcgatgtacttgtcgtggcacttcgcatcaataatgcggccatagttgagctgctgatgaaccggcgccacgaacgagtccacaatgatcgccgacagcttcggatccttcgagtctcccgtcgaggtcatgtcatcaggaatctcgacctcaccagcggtcgccgtcgcaggatcaaccgaactggtaatcatatgagacagcgccacaccgaactgctgggtgcgagccagcggagtagccaacgtcgtcgtatcgcctgcgaacttcgccaccggggccgccaagaaacccaacgccagcgccgcagcaatcgccgcacgcgcacccgtagcccacgcccctgccgtacgtcccctcatcatcttgacgaccacgcccacaccggcgcacgtcgtcagcaacgtcaacaccgttccaccccagctgtccgctcccagcggggccaaaaccttgtcgtggatgtcgttctgtaaccccaccagcggtccggtaatccacccgacccagctcatcgccacggtccagtccaacaaccggcacaacagggcaatgaccatcaggtacgtgttccaacacatgccaataatggaggcccacatagccttcagcggggccgtaacgccaccatcgtccatcgacatctggtactgaaacaagtgaataccgttggagtcagtcacattgaaaccgtcacccatccagcccagccccgggcccgatgccatggcaggcacactgctcagcgccaccaccagaaccagcgtggtgacgatgatacggaaccgccgctccccggccccggggcggcgtggatgacggctcacgctgtctcctggtcgtgctcgggcgggttggagctggcggcttcgcggtggggagcgtaggcaggtctcagtagtcgaatttcaccgacgttgccgtgttggtcggcgtacaggccacaaccggggcctccggggttgagggcaatcttgaccaggtcagcgaactcttcaggggacgttttagggtccacgcccaggaactcgacacctttctgggcgaggttgatgttgtcgcacagcaacacgatgcgatgctgaatgaggttgcggacggtctcgttggggtaatcggcctcggggtcgtgggagccggtgataagccaggcattggctcggcgggattcgcgcacgtactcgtcaatggcgtgggaggcttccggcgaggacgacatgtgatgaaactcgtcaacaatgaaagcggcgtcgcgggcccggtcgctgaagcacaccaggcgagccagacgggcaatgagcgcgtagagggctcggccaaacaccttgtgcgggggcagctgccgaaacagatgttctgacagcatctcctcggcgttaggaagagcgatgccacaagtgccaatgatgaggatgtcggcactcaggtcggcgggaggaaggtccgggtcaaagatggccgaagctagggaggccgagttctcgatgtcagcgaacacggcaatcctgtcagcgatcgcggtggcctcgggaaggtcgcactcctcggccaggtggcgggccaaacgcccacacgaggtgatctggtggtcacgcatgtaggccggtttgaggaccttggccagcgtggtcccggccacaccgatggtctccaagttaagcaaggtcagcaagaacgactgggcgacctgcccggccatctccgggggaaggatgcgcagcgggtccagcgacagcttggggtgggccgtgtcgacgatctggcgtctaaccccgtcgatagagttgacaaaggttacccactcccgctcatcggagttgtcggtggcaataatgcgcccgccccgggccgcgatggctgcgcacagccgcttctcggccatcgtcttgcccgcgcccatcttgccgatgaacgccaccgtgttgcctttctgatcctgagtgtcggcatgcaaatcgaggtaggccagcgactgcaacggggagcctgtgaccacagccaacgggatgccggtggtacggcccaaccggtactcaaccaccgggatagatgcggcgaacgcggaggcgtgagtcagctgacggtaggcgttaagcgaggaggagaaccggccaccgggccgggcagcccacagcaagtcctcctgacggcccacgggacgcacccacgtgaagtcaccgaacaccggatcattgacgaacgctcctgccagctcctcggtggtctcaaagtcaggagacgagacagtgacggtgataagagcctggatctcgacctcacgctcatcggcggccagctcggcgttgtagtcgacgagcacctgagcggaatgctcgatagacatgagggtcgaagcctgggtcatctcaccgccctcgacgttgtcgagctgatcgaccgtttgacggatagcccgctgatttttctgcatcgcagcgtgacgggtccgggccactccccggatcgccacgtcgaccggcacgccagtgtcgtcgatacggcccaaaaactcgtaggcaggccacgtcatcgtgcggggcaccttacccagcacgatatgggactggtagacggtctcgccggtgtcggcaccgattttgaggacgcgacgattccacgcctgagccgtccgggacatgtgggagactcgcttgtcatcctcgctggccagcaggtcggaggtcgcggcctcatcgatgaccaactcccccagcgcggcacgtccaacctcatccatgagggcgattccctcgccaggggcatcatcacgcaaccgggtcgggtggcctgacaggtgctgccgaagccagatctgttcatcaacggtgaccgggcggggttggaacgctgccggcagtttcgcaccgaattccttagcctgacggagggcccactcccgtgcgacacggccaggagcggtggtcatgagtccggcggcttcggtgattttgttgagggcggcggtccaggcggtgtgggtgcgtattttctggtcttggttgagtttgatcgataggaaccacacgcgccggcctagggattcggtttcaggctggtcgagggtggcgtcgatttcgtcggcccacacggggtgttcgctcaggtcaaggccggcggccatggcgtcgacgatttctgtggggtcggtccaggtgagggctccgttgaggcagaactcgtggcgggcgaggtggcggaataggtcgcggtgagctgcggcgacggtgagggaggcgttctcggtgacgggtcgctggaggggagtgagtcgccaggtggcccagatgtcgccggtggaggtccagacgaggttgtcgaagaggaaccgttcggggatggacatcatgactccttttcggcggcggcgaggaaggcttccaggccgctgcggcgtacagcgttcccggtagggtgcaggggttttctgggggtgggttcagatttcgaatgtgggaggggtttgtggatggtatcggtgtcctcggcgagggcttggtcgagcaggcaggtggccggggtgtcggtagtctcgtccagctcgtcgatcatctcgtcgaggaggttgggggtggtggatgcggcccacacaggtgggatagccggtgcgggtcgaggcggtgtggggaggcggagtttggtttggccccagaggggtgtgtgggtcatgccggcgggagtggtggcggcgtgagtggtcacacgccaccagcccgccagccatgccagaggtgaccaggtccagtcgatgcgaccaacaacgaactgggcgagccatccggcagccagcgcgaaaatgaaaccgcggatggtcatgcccccgtcgataattttgaagcccatcatcgcggcaggaactacgccgccgacagcgagcacctgatagatcgtgtagggcccgccaggaatccttcgacctgaggaatccttaccgataatgcggggctgctggcgggcctcggtgtgccactgaatatccacgatcagctcttgcccaatccttgcatcatctgtccgaagaattggatcgcgttaccttcgataagccagtgcgccataccacacacaaacagcatggagacgatcttggccaggctcataccgtgtttgaagatgttgaacgcggcaatgcccacgactacgaccatggcgacggctcgcaccgcggactgaccttcacctgccttgatcgtcgtccattcgaagaggttctgcgattccagtggcagcaggaggaattgatcgagcatgagaggttttcctttcaggaagccgaccgggatggtcccggtactgacgggctggacggcacgggtgtgccggccaggggcagaaggcgtgacggctggtcgagggaggcaacctcccagcggcctgcacggctgacaagagtcaggccgtactggctggtccgggtagtggattcgtcgccggtgggctggcatgtcacggtcaccgtgacggtaatcttctgccggtctgagggggtggggacctcgtcaggtcccaccacatgagtggttttcactgatacgcatggggctgggtcaaccgcggtgaaagcggtccctgggctgctccatctcgacaggtcggtattcccggtcagcatggcggttaggaagcctgaagaggcttgggcggccgggtcggcggtgcgaagctccgaggtgtctagggtgggggctgaggccggtgcaggcatggggatctgtccaggtagacgcaaggcgatggccttgccgttggaatcgacgtggaccgggactgaccaggcctgtccgctcccggattggccgcccatggtgaccacggtgacctgccacaggctcggatcagacggggagacggtagcgtcataggcggtgacggtggtgggcgctggaccaggtttgcggggcatgcgcgcatcggtgatcttggccgccaccaggtgttccagattgtccaagtgcccggagtcagaggtcagcagaagagtagtcacctcgaccgcggtggctgaggcgatctgagctgtgcggcggtcgtgatgggtggtgtcagtgctgacagctgtggtgggagcggatgccggggccgccgaacggaccagggcaacggggccgcatgcgactccgcacagcacgagggcaaggaagcctttggtggccatcgatccggtgtggggccagggacccacgttttcctgtgcctgttttttcttggccgaccaacggcgggacttagggttagcggatgcaccttctggggtgcatgctcgtctcaatcgcatcggtcacctccttcactggatgaagggaccaaggaggtgaccgatgtgcggtcgaacccgaaaaagttcaaacttttttctgggtctgcctcaagtacgacagaaggcctgcatcatcacggcgttgtggtagcagatcttgcgtcagcgcaaatgtatgggcctcgtaggagtagctcttaggtgggtcccggtggttgcggccagggcgagggtgtcgggtatgtgtagcgctagtcccgggttcgtggtgtgagggcttttgggtgctgacatgcactacccattgctagacgctgtgctgtggcatggctggacgtctagcagacgggttgacatcacctttaggctggcagcgcccgaggccgcaggccagaggaagcggacagcctaacgattgacgtccccctctcctttccccaacctcctggagtcgatgacccggagatagatcgatgtctatgcatctgtgcatcgatatacctatgtatctgtctgtggtttttagaggccactcccgtcgccccgggccgctcagcgataccgcaaggctgtgtgggatcagctgtgccgctctcccgacatggcagacagcaccctgcggactgactgacactgtggcgccgcggaggtctcccgcggcgtccgctggggcgagtgcatgcctgtcagctcatcttttatcgtcatgtttccccttgtcagagcggtctttgcaaagtgacaacgtcaggggtcttgacgacaacatcctcgacaacatcgtggcggtggtctgtgtctcgtcagttcaacgcaccggggaaagaggcaccgctgatgttcaccaccacaaacaccctcaacgatgagtgggaccagattcgcgccactgagatcgattggcatgactgccctgaactgatgcactgcatcgacgttgatgatgtgttggccgtcattcctgccgcccccgacgccatcttgggctacctcattgcccgggctcagggcggtgacgagttggccacccgcactatcatccaagcgttcacaggcaagcttatcttgatggccacggccaccaaggtgcgccgtactaatgacggcttcaatgatctgttggcgggtctgtgggagacgatcatcacgtacccgttggatcgtcgccctgacaagatcgctgcgaacctcatcctcgacacgttgcaccgggttacccggttctggcgtgctgattcccccgatgaggaagaggctcacggtctggtgcccttccctgacactctcatcgccccagagcccgatgaggacgtcacagctagccaggccatcgccttggcggtggaccgcaactggattaccgaggacctagcccgcctcatgagccatatttactgtgacggtatgactggagctgatgcgggtcgtctgcatggctgcgctccagctacggtgcgatctcgatgccgtgatggtcgggccgtgttgcagcgcaacgccgagacgatcctggccgtctgctgatccctctcttgctcatgcctgccgccaccctacccctacctgctagaaacatagccatgtctccagcaacgcatctagcgtatcttctagcaattgatctagctatgtatatagctgatgatctagctaccatatagctgtgtctccagcccaaaggacagcccaatggcatgcttgccacaggaaggtgcgtcagcctcccgaaatactggaaggagctttaccgtgcaggacatgacctcgaccagaccgattgaccgatccccgttcgaccgcactattgcggtgtggaaccacaaaggcggcacgttcaagacgtccgtcgtggcgaacctgggatacctgttcgccgccggtggcaacaaggtgctgctggtcgacatggacccccaggccaatctcgacattgacttcggtatacccgcgggagaacgtgaacggggcatgggattagccgaggcgctacgtgaggggacggcccttcccccaccgcagcatctcagcgaaaaccttcacctggtcagtggcggcgctgctctcaacgagttcaccgaccccgcatccttagcggccatcctcgaccgagtcaccaccgcacgctacgacctgctagctcaggcccttgccccgctggcctgggactatgacctcatcttcatcgactccgggccggcacaaactctgctgtcccagaccatccttggagtagcccgctggctggtagtgcccacccgcaccgataacgcctcaatcactggcctcgtcgacgttcaagacgccatcgacggggttgcctcctgcaaccctgaccttcaactactcggcgttgtcctagccggcgtgggggcccgggccacccggatcgccgcagataaacgccacgccatcgacacagtgctgggggcaggaaccgttttcgatgcggtcatccactactccgagaaggtgtccgtgctcgcacgccagcagggcaagaccgtcgccgagctagccaacgagtaccacaacacccagcccgcctacacctacctagctaaaggccagaacatccccaacgtcgccaaggcagccgtcagcatcgaaactgattatctgaggctggccaccgagatcagcgaccgcatgttcaccagcgacgagcaggagcctgatcatgactgacgatccgactccatccgagcagaacaaccccaccagcgacgacatgcgcttggccgaagaatgggccaacagcggccgcacagtgggagccctgccgcgccgtcaacggcctcagcgacccacaccccgcaccaccagccaaccccctgtctcgtcccctgacacaccggctccgaaaaaaccccagaagacccccaccagccagcctggccgtcaacggatcaacgtcaccgtctacgttgtgcccgaactccatgagcggctgcgctcccgatcggcggcgacgggggtgacggtctcggatctggtggtgcatgcgttagcgttcgtcgccgatcatgcgggggaggcgatcgccgatgatctgcgcgtcgagacgggccccggcatgggggccggactgttcgatgtgaccccatcgcggccagtaggggtcgccaaaacccagctgggggtgcgaatgacccgccacaacaaggatggtttggaccagctgacccggacgagtggagcccgtgaccgcagccatcttgtgtctgtcgccgtgcgcgactatctcgactccaaccctgtaaagaaaggccgacatgtccgctgatgaaactcctgccgcccggggtgggcgacgctttgatacgaccgtcaagcccaggcgccgtcccatgcttatcgccgccggtatcgccatcgtgatagtcggcgcgctcctcatcactcagttgattcgctccgcccaaaccgagcatcgcgtgctggaagtccacgccgatgtggctcgcggcgaggtcatccgtgacaccgatctggtatcggtgactgtcggccaggtcgacaacgtatcaacggttcccgcagaccagctcgactcactggtcggcaaacgcgccacggcagatcttcgagccggatcactcttgcctgctggggctatcggcccggccgatgtcgttccagctccgggcaagtcgctggtcgggctgaagctagccgccggacagatccctatcggagaccttgctgtcgggacgaagctgcgcctgatccagacgtcggcacccagcggctcatccaccaccactgatagcagcaccaataccgatggccagtcgtgggaggcaacaatggcgaccggcaccaaaaagaccgagcaggtcaccttgatcaacgccgaagtgaactcgcgtgatgccgcccggatcgcccaactgacgtcacagggccgtatcgccgtagtgaaggatccgatccgatgagcaccattgttttgaccagcgtctcgggagccccaggggtcaccacgacagcgatcgggctgggcagggtctggccacaatcgagcctggtcgtcgaggatgacacccaccacgccatgctggcgggctacctgcgagcctcccagcacgccgaaccgaacctggccgcagtggcgaacctgacgtcgactccaaccaacgcccaaacggtgtgggaatctatcgccagacccctacccaccgatgacccagtcggcgggctgcgacgcaaaggcatcctcggcccgcccacgccgtggtctcgcgcaggcatcgacccccgatggggattcatgctggccctgtggcggcaactggaggaggctggcatcgacaccatcatcgatttgggtcgcctagccacaccactgacctcaacgccacacctgatcgccgcacccatcgtcgaggacgccgacatgatcttggtgatgatcgaggcgacactacgcgacatcgccggcgcccgcacgatggtggagggactcgctgagcagatgaacttggccggcgcctaccgacgtctcggactgcttgtccaccgtggcggccaggcccgcggcgtcgccgagttcaatgacaaggagatcacctcggcgttacgactcccggtcatcggggccattacccacgacccggccggagccgcccaactctccgacggggtcgggcagcggttcgacaagtcacgtctggcccactcactatcaaaggtggcggccagcctcgtcgatgccataaacaagcgccacgctgatgacgaggaggatctgtgatggccagttttgctgataatccgttcgacaagctgcgctcccaggatgcggcaagagcttcggtagagcaggagcctgatgccggattagcgtcggagttgttttcgacgtcgtcggggtgggccagctctcagcaggtgtcccaagctcaaccggtgatgacccgttcggagaacgtggattggccggttgtcgcggagctagccagtacggccactgatgaggttgaggctgagatcagcaggtggagttccactcatgatggggtggcgacgctagacattcgccaggccatcgcggagcctgctattgcctcggcggtgtctacgtatgctgaccgacgccagatcgatgtaggggagacctggcctgatctggtacgccagcgataccgcaaggctgtgtgggatcagctgttcgggatggggcggttgcagccgctgttcgaaatcagcgacgccgaaaacatcatcgtggtgggtaaccatgaggtggttgtcgaccacaacgatggttcccggtcaacgctgccgccggtggccgactcggatgccgagctggaatcccagatcgcccggatggcgaggaatgcgaccccgcggcgagccttcgacgccgatcacaccgatgtgacgatcatgcaccaacagaagttccgtatccacgcgatctcttcggaggtgtcgttgcaaccgtcggtggtcatccgccaacatttgctcacccaaatcagtctgggggacctgtcccagcggggaatgatgccggtggaagtggcccggttcctcgatgaggcggtacaagctggcaagtccatcgtggtcgccggcgagcagggggcaggcaagacgacgtttttacgagcgttgatccacgccattccgatgcgggagcggttcgccacgttggagaccgatcaggagttgttcgctcatctgatgcctggccgggaaaacacgctggtgctgttcgcccgtgatggtaacggcgaggtggatccggcgactggcacccagcacggcgctatcgagatcgcccagctgattcctcctagcctgcgccaggcgttaactagggtcattgtcggtgaggtgcgcggcaatgaggcttcggccatgttccaggctatgcagtcggggactggcactatgagcagtatccactccccgcgagcctctgaggtgccgtcccggctagcccaaatgatttcgatgggcccggtctacgatctggatcaggccatgctgcagatcggtcattcgatcgactacatcgtctttgttcgtaagcgtgacctgcctgatgggagccggctgcgatttgttgagcagatccgttcagtctcgcctggtgattcgacaactcccagtcttggcgaggtgtacacggctgactcgtggacaggccagccgcttacgccactcatgcctggttcggccgccgacgagctgtctcacttcgcccgagacctggattactgcgaggcccactgatgaacaccccagcgctgatagcagccctcatcgtgaccggcctgggactgatcatggccatcaacggcctgatcccggccacacccaaacccggcaccacgctgacgctgtcacagcggtgggcccgggccacccaccgcccagccggggcagccgggcgccgacgcgacctgcgctgggcgctggccggtctcgtcggcatcgtgctgttcgtcctcacaggttgggtcgctgccctggccctgggccccatcaccgtgctgctagcacccaccctgctgggggcggccccaccgaccgatattccgctgatggaggcgttggatcggtgggtgcgtcaggtcgctgcggtgctgccacaggggcgagacatcatcacagcggtgcggatctcacggccgcgggccccgaagctgatcgccgggtcggtggatcaactggtgtcccggttggatggccgtatggagccgagggaggcgttccagcggatggccgacgagctggactcggctgaggctgacgcggtgctggcctcactagcgttggcggccactcatccgcggggagcctcgacaacgttgaaggctattgctggcaacatccaacagcggttgaaagtgctgcggcaggtggaggctgagcggtctaagccgcgtaactcggtgcgccagatcacactggtcacgatcgccatgttcggggggctgctcgtgatcggacggtcgtttctggccccgctggcgacccctcccggtcaggtgctggtggccgtggcggtggtcatgtatgtcatcgcactagtgcagatgtatcggatggcgcggccccgtcgacgcacccggatcatggtcaggaggacccgatgaccattactgctttgtccctgctggccggggccatcgtcactgtcggactgctggtcatcgtggcagcgttccgcccagccccaggccctgacctggttgccgctctggaggtggtgtcgggtcgcacctcaagcgtcgccgagaacgtcgaccagacccggatcgggcggataggtcgcagcgtgacccgcacattccatgtgtcggtgtccccggccatgcgggccgcactgcgactccaaggcacgacccccgaagcgttctacggtcgacgcctgatctgcgcactgatcggggcgatcctgccgtggctgctcaacgcggtagccatcgcggtaggggcgaattccccgaacctactcatcccttcggcgctgtgtgtggcgttagctggcgctggatggatgctgccggcagcacggttgaaggctgcggccgggcccaccaacgatgactcgttcgaagcgctgctggtattcatcgatttggtcgtgctggaacggctcgccaacgaaacctccgtcgacgcgctcaccaatgcggcaaacatgtcggactcgccgctattcgtccagatccgtcaagtcctcaaccgtgcctccctggaaaacgtcgacccgtggaacgggctagaccggctagccgaggacatcaaactgcccgaactcaccgatgtggtctccatcgcccgcctccaaaacgaaggcgcctccctcgtcgacagtttccgggcccgagtcgccgagttgcgcgacgcctacctgctgcgcctgcaacaagaatccaccgccatcacccagcggcttggtttgtggacgatcctgcaagccggctcggtcatgctcatcttgctgggggcggccgccctcacactcatcacagccggataaaccgacatcgtgtcgagcccccatatgggccgcgaacctttcataatgatgcctgtaagcaccacaatattgatcacaacctaacaatgaaggagaaatcatgatgaccgatctcgacctggctaaaaaacagatcacctggctggcggcgcacctgcgttcccggatcgttgacgagcgtggcggtggtagctctaccgtggagacactgatttggattggtgtcatcgtggcgctggtgattggtgtgggcgtcggggtgacggcctatatcaggtcgaagatgcctcactgacgacgatgcgtagcaggttcacaactgatcagcgtggcggtggtagcgcgtcggtggggatgctcctgctgatgcctgcgatcatgctgctggcattcggtgggatcgaggtcgggatgtggtgtcacgcccatcagtcgaccatcgcggcagcccaaagcgccgcggaggctcagcgtgtcgtccatccggtcccggggtcggcacaggaggcagcctcgcagatcactagtcatggcggggtgcgtgatacccggatatcggtcagtgatgacggggccactgtcacggtgaccgtctcaggtcgggccccatcgatgctggggctgcatcttccggctgtgtcgtcgacagcgtcgatgcccaaggagcgcctgtcgtgaggcgaagtgagcgcggtggagcaagtctaagcgtcgaggtgctcatgtgggcgccgatcgcgctggtgatcatcgggtttgttgtgggtatcggccggatgtcgatggcccaggacgcggtgaatggcgcggcgggtgctgcggcccgtgcggcgtcgttggagcgtgatggacagtcggctcagtctgcggcgcagcaggcggcgtcggcgaacctgtcgtccggtgggctggcatgtgcgccgaatgtgagcgtggacacgtcagtgtttgcccgtcctgccgggcaggcgggcacggttcacgccacggtcacgtgtgtcacctcgctgggtcttgggttcgggtcccggacggtgcacgcaacggggtctgccccggtagacacctatcgggaaaggagaggctgagatggcccgtgatgagcgtggtggcggctcggtgtcggtgtggatgctgctcatggtgccggtgattctggtcatggccggcctggtttttgacgggtcccgtcagatatcggcaacccaggcggcccaggacgcggcggttgcggcatcccgcgccggaactgatgcggcagcgacaccgcagcttgctggccacgacggggcggccgtagcagtccaagcggcccgccaggcactatctgctgccggggtggacgggtcggtgcaggaggacgggtcgaccatcacggtgaccacgtctcaaagtcggccgacagtgttcctgtcagcgatcggaatcagccaggtaagggggcatgggcaggcccatgctcagcttgtgggaccgggagaacgcccatgacagtgatacgtcgtatcggtgccctggtggcactgctggtggtgatagccggcttaccggccgggctcgtggccgcaggcgccccgctagtaccggcgggcctgtcatgggcccatgtgcggcatctgttggttaccccagacatgaccgggtcggtgctggtgtggctggtcagcctcatcggctggatcgggtgggcatggttcgccctggccgtcgcagccgaagcggtgacgatgctctcagggcagcggctgcactggcacatgcccggccctcgcctagtgcgccgagtggcagccggtcttctcattgccgcgttcgcggccgcaccggcagccacaagcacggctcacgccgccgaggccacccatgtggcggtcgccgcccaggctggaccggcacacgcggcccccgcccaggacagccccgccaccacctctcaggccccggacgcctccaccactccgaaaatctggaagacctacacggtgcgggccaacgactacctgtggaagatcgccgagcactactacggtgacggcgcccaattccgtcgtatcgccgaagcctccggtatcgacccgcactcagagctgaaagtcggccagaaactgatcctgccggttcctaagaacacggccgcagttcattcggttaaagcgggggagtatctgtgggagatcgccgagcactactacggtgacggcgcccagtaccataaaatcgctgaagcttccggtatcgatgcccattccgatctagccgtgggccaaaaactcgtcatccccgggcccctccgccacgacgccacgccgccacaccactcggcgtcgacccacgcgaaggccgccaccccagcccacacgcccacgcgacggcctgcgcctgcccacaaagccacgcccgccccgacgccggccaccccaactactccgacccacacggtgaccccagctccggcctctgccggccactcgaccagcgacgagcacccactccatacggataccgccgcagcggacaaggccagcaacgaggacgccctgtccccaatccaggtgggtttgaccgccagcgtcggactggtgctggtcgccggactgatcaccaccctcaaccgccgctaccggacccggttcacccgccagccccgcggcaaggccatgaccctgcccagcccggacgcccaaactgccgagatagcgctgcgcagcaccggggcgactgacactctgacgatcacctgcctcgaccaggcgctccgcgcgatcggcgcatggtgccaccactgcgggcacccgctgccacccctgctggccgcccgcgtcgatgacgaccggatcgacctgctgctatcccaggcggcccccgaccatcccgaggcggtggagctggccgccgacggatcggtgtggaccctcaccgccgaccggatcgacgacctgctagcccacaccgatgacaaccaagcggctccgtggccgtctctggtcacactgggacgcgacgacgacggcgcccacatcctcatcgacttggaagccgcaggaaccttgcacctgatcgccgatgacgaccaggtcgacgcggccctcgccgcgatcgccgtggaactggccacctgcgactggtccgacgaagtcaacgtcaccctcgtcggccaggtgtgcccgggcctagaagacgcactagagtctccgaccctgacccgcgccaccgacgtggacaccctcctcaccaccctagaagctcgcgccgacgaccagcgacacatcctcaccgaaggaaaccccctcgccgctcaccgagccgaccccgccatctccgacgggttcgacgccgaagtgatcctcctcgacaccgaactcaccgaagaccaccgcaaccgcctcgccagcctcgtcgaggctcttccccgcgtgtcagtcgccgcggtaacaaccagccccaccagctcagacgaatggtccctcaccctgacaggtgacccactagccgccgacctggctcccctcggctggcacatccacccccaaaccctctcccccgacctgtacaaccgcatggttgaactactcgccaactccgccgccgcagactacgaacccgccagctggtggaaccacgacgccgacgacgagcccaccaccgaccccaccaacgaggaagaatctaccccgagcaggcgagcccgcccgaatatccgcctgaccaccctgaccggcggcatcgacctatccagcgtcgacatcgacgacatcatcgacgaagccaaccaggccctcgacaacaccgaccagaccaccaaccaggttcgcgtcgacaccaaccaacccggcaccgacacccccttccccatcgacgacatcgacccgctgagctccgaccatccggtcctatccatcatcggccaccccgacatcacaggtgccactggaaccgttgggcgctccccgtggcgctgccaacagttggctctctacatcgctgagcatccgggagcatcgggggcgactatcgccgatgatttggggctatcagcgtcgacggtgcgctcgattgctacacacctgcgacactggttgggagccgacgatgccggggtggcttacatgccggcggcgacgcgcggctaccggctcgatgagcggatcgtcacagatgtggacctgatcgacgctgctgtggccggggcaggcattaacaccgctgagacggccacactggtggccattttgaagctgggccgcggacgggtcttcgctggggttcctgactcggaacttcgccagttcagggcgtcgatgtaccacgtggaggcccgcatcgtcgatgctgccctccaagtgacagaccgggccctggaagccggggacctggggctggcccggtgggcgttgacccaagggctactggtgtctcccgatcatgaggacctggtcaccggttgtctgcgcaccgagtaccaggccggcaacatggacaaggtttccgagctggtcgatcacctgtcggctaccgcacgacgcctgggcgtcgatttgagcgccgacaccacccggatcatcgactccgttattacccatacacgcaggagagcatcatgatctcccgttgcactcatctccgtcaggaaggttcctatgcaccattatcgtcttggtatcggcactgccctcatcgcggtagcagccgtggccggctgtcgctcaacagactcgacacccacaccacctccatcccgtgtctccgccacgacatcagcgagctctaccctcagcccgaccccgtcagccggagcgatcagtgccgccgacgccgaaaacatctaccgcaccgtccaagctaacaagatcgcactgtacaaaaagggtggcctggcacccggggaacccgcacccgcgacactgtcgaactacgccaccggccaggctctcaccgactacatgacctacatgcaccaaatctcggggcagggcatcaaaatcacatcggggaactcctcggttaccgcggtgcgggagaaaaagggtgataccacctaccccgaagccgctatcgccttggaatcgtgcgaagacgacagttcgattcgtaccgtggaccgccacggaaaggctgatcatggacgcattttccatgtcgacagttggtatgcccgcgataagaagggcaccgtcaagatgatcgcattcaactcaacggaggtccccacatgcgacgtcaagtaatcaccgcggccgtactgggcctggccctggccaccacctccctgcccgccctcgcaggcggcggcgacttgaacggcagtgtggacacggatggccctggttacgatgtgcgtactgaggtggattaccatcacaccaccggtggtgcgggtgggggtgccacgacagggggccatcatgggggcaaaacccacggtgatggtgaaggtgaggctattgatgggcgttctcgcgccgatgccgaagccgaactagacgaagccgaccgtaaccaggacttgacttgtgatgtcttgctcccaccggcgccgaataccccagccggccaggaatgggcgaaagaatgcaatcccccagcgaaaaaggggcagaaggccaccccgaagcccgatccggtgttagtgggccgtcacgcggccttgcagctcacccttcccggcgcggcccctcgtatcgatccgtcccccgaccttaaccggtggcactcggcagcagtgggccaggctttgtggttgtcggttgatgaccccaccgccactacgcagaaatcgatcacgtttatgggccaggttgtctcgttgagcgccaaacgtaacggtttgtcgtttgatatgggtgatggtcatgtcgtgcactgtgatgccacgacgacatggacggagtcggtggaaccggggaccccctcacccacgtgcggttacacctaccaacaggcagccccggccgggggctataaggtcaccgcaatcacgtcgtgggatgtgacctggtcggtactaggacacacgggcacagtccacatccagaaggccggtgggcagatgcttccggtcggcgagttggagtctgttgttacacggcgatgactcctgatagtccgacgctgtatgaggtgttggaggtctgccctgatgccactgatgaccagatcaagacggcgtggcgtcgtgccgcgaaagtgactcaccctgatgccggtggcactagtgaggcgttcgcggccgcccggcacgcctgggaggtgttgtctgatcctgcccggcgcaccgcctacgatgccgacctggcgggggaggatgatcccgacgacgaagccgccgacgccttcgacgttgatccgttcacggcatggccctggtgcgccccgtacatcgacgccacccccaccctgcgccacacctgccccggcacgatcaaggccatcgtggtcgccatcgccgggatcatggccggaatcggctggcaggaagcgctggaggccgtggtgtcgccgccggggtttctgggggcgtgctggatagtctcagtgccggttgtcgtcgcagtgacatggctggcagcggtgacgggtctggcatttttcaccatggcggccatctggtgtggatggtcgttgataggtggtgcgttgctgtggggccagacgtcggtggtgtggctgattgctgcggggtggctggcatggctggtgtgtgcgtgggtgtggtggaaccgtcatctggcgtggagtagcgagctgatgcaggaaggcaatttgtacggccttcccgaggaagacccgttgtggggcgaggccgtggagctggtagcgggacttatcccctctgtgcgggcgatgtggtcgcacgatgggcagaccgtgactgtgtcggctggccggcgggtggcgacactggggatgccgatgcggggatggcgtggtatcgagatgcgcggctggaatccagtgggcgccgatacgtggtggatcgttgaccaggtgggtagttggctggtcgatcaggatgagccgatggtcgttgacgggagggtgctccatgaggcgtgggaacggtcgcagatggtccgttcaggccgctgagaaaaaatttaggtccgccctgcatatcggggcctcttcataccttcaaaagtgtgaagggaatacagactcttcgactggaattaaaatggtatatggcgtgtccggtccgcacccagctccacgatgcgccataggggagccataacttcaccaatcgtatacgatatgagtgtaggtgagcatctaccggtggccgacagtcagatacaagcatgggcggatgaagcagaagctgattatgacctcagcatgcttccgccgtctcgacgtggtcgcccaccggtggggaggggcccgggcactgtggtccctgtgcgcttcgacgcggacactctcaaggcgttgtcacaacgagcccctgacgaaggattgaccacgcgttctgacgcgatccgtgctgcggttaaccagtggctcggcctcggctcgtaaacattatcggcgcgacaaccttactgatgacgctgttggttacgccgtagagcacatcttgtactcgtgggcgctcgacgatgctgatgatcctcgttgctggatgatgattcggtgttgacccagctggcctcctcatggagctagttatcctcatctacgacgatggctatgaactcctgatccacgccatgaaagcccgccctcagtatctgagctacttcgctatctagtgttgttgggcgagttgaactcaacttttctccgacgcaagcgccaataaaaggctgatccatccgaataccaccaaacacggctgacgtgtggaacagattcagcaagacagggtgcggtgctcgggtgcgttggggagatgaggatattcaggcttctggcgggatgtggcttgatgacgtccaggatcaccggttcggttttgaaggtgccttgcattaatgtcgacagggtcgtacgactcgaggactggatcgaccagcccgttgcgtctgaagaacttcatccaccccggtggagctccggccgcgtactggtgcaacggatcattagcatgggtagcgtgagtatgccttcgatcgtggtcagtgctgtcatcatccaaggttctgacgggcggcttctcactgtccgtaaacgaggcaccgaggcattcatgctgccaggcggcaagccggagcccggcgaggactcgcgtcaggcagtagtccgggaagtgcacgaagagctcggcgtcgcattgtcttccgacgacctgcgtcgggtgggagtgttcaccacgcgagcagcgaatgaggccggccatcaggtggtggcgacgattttcacccacaccccggttgcggtgagtgagccagctgctgagatcgagcagattcgttggctcgattggagtgtcgacgccctgcctgatgacctggccccgctgctggtcgaggcagtcatcccgtggctgcggcgccgtatccggtcggtcgctgtattcacgggtgcgaaggatggaaccgaccctcattatcgtgtcgaagcaaccgccttgggccggggtctcgcacacgccgggatcaccctggtttatggcggcgggaaggtcgggatgatgggtgctgttgctgatgcggccctcgctgctggcggcgctgtgatcggagtgatgccgcagcacttggtggacggggagatcgcccatcctagtctgactcacctcgaggtagtgcggactatgcatgagcgcaagcagcggatgagtgacctagctgacgcgttcgtcgcgctacccggcggcggcggaacccttgacgagctattcgaagcatggacatggcagcagctcggtgtacacagcaaacccgtggccttgtacgactcgaccttctgggcaccgctgaccgcgctactcaaccacatgaccatcgaaggcttcatccgccctgaggaccgcgcctcgctcgtgatcgccgataccatacatcagctgatggccgatcttgagggatggaccccaccaccaccgaagtggcgctcgtgacatagaacaaatgattctgactatggctcattgacatctgcgcagcggctactagctccattgacttcaaatcgggccttggccgaggctcggttcaggtggcccggaatggatccccacaaattgaatgctcatgatcgaggtgatgaacgcccactctattgtcctacgcccggttgtatccagcgagcaattgaaggtgctgcgcctgttggtcgaccaggccgagccactcatcatcgacgggcgcatcttgtacgaggcatgggacaggctgcaaacagcccgtccggcccgctagaaaaaatttttagggcccgctccgcacatcccggtctgtacccacaccttcaagggtgtgaagggaataaaaacacacctaacaacaactttgtttcatgatttggcgcgtcggaccctttctgaccgatgtcgtgcgcaacaatggacccatgagttcgcctaccaagatgcgccggaaggggacacgcggcccgaaacctcgcagcgacgaggagctaacggctgtgatgttccgcgtcccagagtcagtggccgaacgtgtcagcgatatggcttgggagctgaggctctcccgatccgatcttgtgggctggtcggccctctatgcactcaactcgatgctcagacagcgcggagaggacacgattccggttccggagtatctggacaaggccgtgctcgctgcgttgtatcccgatgggttgcccttcgacgaggacaccgaggagccgggcgagaatgccgggcaggaggagcttgcgatgactggatgagcccgaacatggttgaaggccgccccgccaggcgaccttcaatcaatggtttcggttgtcagctgcagccacccttgccaggtgctgtggttcttctccagaggaggagggaccgccatgaagacccagaagacctagaagatccaaaagaaccggtttcggctgttgcttcggtcttcatggtagctgatagcccgcgtcgttggaagtgcgacgacgccgatcctcatcggtatcgcaccctgttccagggtcgagaatgtgaaggattaacaaccatgacaagtcatgagacgccctcccggagggcgtcatgaccgctttaattcaagctctgccgacagctgaactgtctgctggacctggtgctagcagtccagctagaccctctgctgagggcttcatcaagattcaccacgacctcattagtgcgggcgtgtcgggtaacgcgatggcgttgttcgtcgcgttacgtaaccagcccggctgtgatcagtggacccgtcactcgtacctgcggctagcccaatggtgcggctgggatgggctgtcggaggcggcgggctgtaagcgggtgcagcgggccgctgctgagctcgctagcggtggttggctggaatcgcgggtcggccatgatcgtcgcaccgctaagacgctggtgtggcatcggttgaccagccccgacaccgaccgctgggagcaattgccgcggattgtgtgggcgaggatctgccagatcgccggggagacgtcgggagagtgggtgaggcattggctggtgtggcggatgctggccggtcgtactggtgtggctcaggcccctacgtccatcgtggcccggttcctgggctgttcttcgcgccaggtctctcatatccgtggggctctcgttgatgccggtctgctcggctgtgacgaggtctcgggggcggcgagccgggtgtggttcccctcgatggccgaggagtgtgcagcggatcgtgaggtggtcgatggggccgtcgtcgatgctgaggtgggctctggtgaaggtgtcgaggagggcggggaacccctgtcgattttgtccacccacccctgtcgattttgtccacccacccctgtcgattttgtccgccaagtattagacaaaccattagacagtgaattagaccctgggtcgcgtcagcgtgtgaactcaccaacgcgcgcgcgcgcggcggccgagccgccgaagccaaagaaacccataccaccaacgcctgaaccagcagcccccgcttcagacagtcctgtgtcagcgcacgctgatgaggctgccgagttggcgtggcagttcgtggggtggtgtcctcagctggtaggggcaccgaagaaggtgcgtggccagctccgccagatggtcgctgccgagatccgccggaatccgggctggctggatgcgccagcgttggaggttgttgcccagcgggtacgtgctgaaggtgcgctgggggtccagcactgtgagattgtgcgagccgagctgcacggggtgatcgccgaccagaaggccgcccccgcccgccccgacgccctgcacgcgcgggagcccaagtggtgggacgacgtgtactacgaaggaaacacaaccgtgtggtacgactctgacgaaaccagtgacgacgacgaggcgtggaaccggtcgcaggtatgtcatgttgctgagcgtgacgacatggctggcgggattgctaccaacctgctggcctcccacgctgatgatccgatggcctggctgactcgccgtcaaaccatgctgatggctcgtttcccccacgcccagaacgaggtcatcgcgatgtgccacatcgtgcgcaccgccctggaggccgacatggccgccggaacccggtgggaggactgatgagggtctcccggcgcgccctcatggccgtggcccgtgccgacgctgccgcagcagaccatgccaccggccgatgggtggccacagccaacgccaccgtcgcccggctgaccggactgtgcgaacgcaccgtccaatacgcccgcgccaccctggtccacctgggcctgtgccgcgtcgtggccaccggccgctacctcaccgccgacgagcggcgcacggctgctcgcacccatggcggccgccagatgcgcgccgcctccacccgcgtcctcaccatgtcctgcgcaatcgtttgcgccctaccccgaaggggtcaccactacaggaaagttaagttcctagatggtcaccaacgcacgcgcaggcgcgtgcgaaggccagcaacaccagagaagaaacccctgtggctgcaaaaactcgcagccctcctcgaccagcacctgccctggctggtgagaaaccatcacatcggccgactgtgccacgccctcaccgccctgggcatcgacgaacactggcaacccgcagacctcgacgccctcctcgacgccatgaaccgcaggaaccgtcgcctcggcctcgacgtgccatgcgccgacgaccagcacaaccccttgggactgttcatccatcaggcccgagacaccctcacccacgacaacccccatgaacgccgcctagcccgtgacgctgaacgcgcccggctcatcgacgaccaactccgccgccgcgcagaaaccgccacctgcgcagcccgcctcgcagccgagcacgccgaccccgactaccagacccgccgtcgtgaacgcagggaaaccctgcgagccacactcagggccgcccagcagcatgtcaggacacactgatacccgctcatacgcacaccatgaaacacgacaagccacctgacagtatcagctgcaagacgcatgacaacatagaaggacatgatgaaatacacatagacacaaccccgcacagcagtttcgcaacaaatcatgatagtctcatggcagtatcgcttgatacacacatagcatcactcccacataggcagttatgctgatatactcctactaggttatccgtccaacatgctgagtgggttcctgtacagcatgaaaggagcatggcatgatatgggccgttctcaactgcaagggtggtgtcggcaagacgaccagtgccctcctgctggcggcagccgccgccaagcagggccacaccaccctggtggccgacgctgacccccagggcaccgcatcccagtggtcagccctggcaaccaaaaacgacgaacccctgcccttcccggtgcaagcggtcaacatcgccaccatggaggcactgcccaccaccaccgacgcccatgatctcatcctcgttgacacacccccctcggcaggcgatctgatgttccaggcatgccgcatcgccgacctcatcatcatccccaccgccacctccggacccgacatctcccgaacatgggtcaccatggacgccacacaaggcacaccacgcgcgatcctcctcacccaaaccgaacacaaccgtgtcgtctaccgccaagctcgcactgccctcgccgccgacgacaccgtcgttctcctggaccacgacatcccccgccgtgaatccatccgcacagcatggggcaccaaccccccagacgacgtcatcacctactacctgcccgtcctcaacgaactcatggaggccctgtcatgaccaccaagaaaaccgacccattcgcccgccagaaagcagcagaagaccgcgcccaccaactccaagacgaagcccaacgccacgcccaccccaaacgcgtcatcggccccaccgcacgattccagttcgtcctgccagccgacgtgctcaccggcctcaaacacctcgccctcacccgcggcaccaccgcccgccaactcaccctcgacgccctcgacaccgcctacggcctcgacaccctcgccaacagcgggaagaaaacccacccatgacccgtgggtccccctgccaccaccccctcgacggcaactgtgtgcgagccacaggcccgcacacaggcggcagcccagtgccgtccccgccaggaaaggaactgaacttttctggagtgctggctccctatcaccccttatggtgctatcagatccagtagcgccagcccgagaggaaacccatgagctacatcgtccgcaccggaaatctagccggcacccccgagctgcgcgaggggtagccgacacggcggaccgtgcccctgacagtaagcggcactactggagacatcaccatgaccgctcagatcctcgccccgtgcaaccagaaggctgccgtcatcgaaggagccctgccatgaaccaacctaccccccacaaggccgacgagtcacagacgagcaacaccacgaggaagtggcggcacaaggtgtcgttctaccaagacccggccgacaccgaccgagtccgcggagcgatcctccacaccatgacaaccgaaggcaaccggaacctgagccagttcgtcaacgatgccgtcatggccaaggtgaccgaactggaagcaaaatacaaccacggcgagccattccccgccgtgggagcccgaggactcccccagggcggcgcagccgccaacagataagagggcggggaccaccaatagcagtcgatttcggatccagatcgggtagacaggacccatgatgaatttcatatcctccctgctctcggccatcctgaccctctttgcgatggtagccgtggcgataggagtggtgaacattgtgccgggaatatgcgccatcatcggtgcacggaaatacggcgaccaggaccgtgtgcgggcaggacaacgaaagatccttcttggtgctatcgccttggtggtagcagtaggcattcacccgtgacccacggttaccggaagccccgccgcaatcgataccactgtcagtaccatcaggggcgataggggtcaagctctccttgtcacaatcccttccccggcagcccagtaaccgtcgcttacagccatacatggatatgcgcccattcgtcggcaccgacgccgtgcgggttggcgacgctgacccgtctgctcacaaacgacccttccagcccagcaccacccactacaccgacacgatccacgaagctgaaaccgaacctcccatccccgtccgaaacatcaacgaactcatcaaccccaccaaccatgcatgaacgcacgatcgcctaccggccagccttcctgcgcgacgtgaagcggctcaaaaacaagcactacaacatgaacacacactcaaatgggcggtaaggtcacgaagtcgacgtggctgcctcacggatggcttcggcgagctcttggggtcgactccacatgggccagtggccggttggcagatccatcgcggtgaagttggtcaggtgcgcgacggcgctgaacatcggattgcctgctgcggccagctcgaggacctggacgctgggaatggagcagcagatcaacgtggttagtacatcatggcgagcgctattggacagcgcgacccgtgcccgggccacttgcgcgggttcaggaaccgcccgtgcccggaaccggtcaaggtgctcgttgctgaggccctctaaactggcctgcttgccgagagtatcgaagtcaggaagtggcagctcagctgtttcctcaggtagatctggtgcgaaggcacctccatcgctcatgggaccggagtccacccagacgatccgatggaccaactctgggtgcctgtctacaactaggctcaccggcccgttcgcgccactgtgtcccaccaggaccgcatcgccgcccatctcgtccaggatggctgagatggcgtcagcttggtcgtcgagcgtgcgcgtagtgcgctgggtgtcctccggatcgaggccaggcagcgtcagccccacggctcgactgccagcagtgttcaataactcgactacctcgtcccaagcccaggctccgagccagtaaccaggaacgaggacgataggccgatgctggtcaggtcgcgatgttgaagtcatggagacatagttgcacagtgcgcggacaagggagtggcactatttctggcatgaatttagatctgggtgtccggtgaggcgatcagaacgcctccatgtactgtccgagtgtctacgatgcagcggacacagtcgagcagctggctcacaagtttccaggtgtccttctgcccctatgagggaagtcggctgctctcgcaacctttgagggctgctctggggtcggaaccagttacagatcgttcaagttccgggaactaccgccggtcacgctgattgattcccgggcgatctcgcttctgacgacagtggtcggtaaccgagaggcgtcctactctgaccccgctgctgcggtgagtaaatcacgggcgtcccggatccggccacccgtccaagtgccagccagcttgatcggctaggcatcgtcaaggagcgacgatgaggtgatgatgcgatgtgagcgcaccgcgaagtgaggtataaagaagccttcccgggcgcatgcgtcgcccaggaaggccaggttggcccctggacggctgaatggttgggcggatcaggctgtagcggagacgtgcagtacctcgtaggcgatggcgttggttgggctctggtctgcatcgtggctactgagttcgacgaggatgacggtccaggtggtgatgtcgacggcggtgacgcgggcgatggtggaatcagtggatgctgcaaggaagtcggcggttatgtcttcgatgcagtgggacggagcagtgctggtgggcaggggcacggtcttgacggtcaggcttcgggcagcagagtaatcggcggttccgttaacaagctggtggacggtccagtcctggatcgggtgccggccgaagtcgcgtacgatagccgagtaaccaagttcgccccagcagaactggcgcatcccctctatgtcgcgccagacgtagaagggcgcgtaggagttctggggtgcgcctttgaccttttcttggatgaggtaggccttgaactccagtccatggtagccgtccatgaggtgtccagtctggatgacgcggtcgcggatgatctgcatgtcatagtcggtgggcagggtgatctgatattgcatggcgtacatcagcggttctccttggtttcgtgggagtaggtcgtggggatgacgggttcctgccaatcgatggttgacaagtcgtcggcggcttgttggtaggtaaggtggatcattggctgtttgagtgcagcgccatgccagtgtctctggccggcgtcgatccagatggattcgcccttggtcaggtgctggggtgcttgtccttcaatatggaccagggcaacgccgcccgtgacgtagaggccctgtcccttggggtgtgtgcctcagcggcaaacgtgacggcgtgggcggcacgcccggcgtcttccgtggcacgatcggccggatccatggcggcgcgcagctgctgttctgcagccaggcgtccaggtcacggcgggcagcatcgacacccagggcagcctcgacggcttcagggctacgacgctggccgggctggttgaccgcagaccactggtgccgggcgtcgtccagatcatggccggcggcgtcgatgagggcggtggtggcttcggcgcagcatcgcctgatccactccgaacgtgacaggccagctgtctcggcggcactgtcaatgcgcgccaggagagatttcatgggcgctcatctgtagcctccagataggcgttgatgtagcgcagagcctgatccgtcatgtcgatggcggctgcctcggcagacgacacaacgggctgtggatgataagaccacccaacatagacggcgaacatcaatttctcgacagctttcatgaggtccccggcggcaaccgcatccgcctcatcatccgaggagaaccacttcaggtctgaacggctgtacatagcccctaggtcctgatgtgggtcgaggtccttggaatgatggaagttcctacacacccatcgccaaagacctcaacatggacaacgctaccttcaccactccctcatctgtgaagagccagtttatgcacggtgttgaaagaggcgtcggtctacgacgaatgcgggttaatggtcttgcggtgtggggcgctgggcgatggcatcgagaacgcgtgagtgggtggcgatccattccgcgtcggggttgatgttgcgggcgacggtgtcgatcgctgttacgtagtgggcaagcctgcgaatctcagcttgttggtcagggtgatcgctgatgagaggctcttgatgggaggcgcggttacggacgtggcggatgagctccacgttttcctcgaagtcgcgtcggctacgccccgggtagtgaggcatacctccgttacgtaagtcagtgagagctttccatacggttgcttccaggcgcggggtgagcagaaagcgccagttgtccagtgacagactcgccacgatctggatcaggggtcgcatcgagtcctcgcgcgcccgcagtcgcttctcggccctggcgacgttacgcgggaaaccaccgagccgatagctcggggattcccaccaacgccgtatgccagtatcggcgctcaggcgcacgtcaatgaagtttctcaggagtacctcgacatggctgatgtcttccaagaacgctttcgagatgcgggtgttccacacgtaccacgcatcgactggttcgatccggtagcggctgagcctggcgggtgagaaccactcctgcaacgtctcacgactcaccgtgggagcgtggtaggattgatgacgataggcctcgggaaggtcaatgccgcgaaatatcggtatacccccgaggtcattgttttgcccaggcccctcctcgcatacttgcggggcgtcattggctgccgtcatctcgtctcttcccggtacgtggctgtccatgttcacgggcaggtacggcaatgaggttcccgcacacaaacctctaccaagcatacgctggcaagccccgacgagacgacgcctccgcccgccacaccaacggtcaccgccacggccacggtgaccgtgaccgtggcaccgaaacccaagccaaagcccaaggccaagccaaaggtaaaacccaaaccgaagcccaaggcccattcatgatcgcagtgcccgatacaaggtggagcggcccacaccaagctgggccgccaccttcgttttcgggacaccctcggcaacgaggcggcgagcgcgggcaagctggccctcgctcaatgccggtttccggcctcgatagcggccctgcttcttcgccaccgcgatcccctcggcctgtcgttcatggatcaacgcccgctcgaactcggccaaggcacccagcagatgcaacatgagctggctcaccggatcagcggaccccggcccatagacctgcccttcaagggcaggcccgttatgctgtcagcagatctgccagttgtgggtgctggtgtcgtagcagacgccggtgtcggtgtcgtcggtgcggttggtggcccagccggtggtggagtcgtcgagggtgatgtggtggaggtcgggttgagtgttgagccagtgagtcccctcggcttggggtaggccatcgaggatggctagtaggtgggagatggtgg

SEQ ID NO: 106 - DNA, 1010 bp, Propionibacterium acnes
atgaagaaaaactggttactcacaaccctccttgccacaatgatgatcgccatgggcacgacgaccaccgccttcgccagcccgcctaccgacatcactcccgaacatccaggcggggttacccgcctcacagccccgacggaatcccctcgaatattgaggggccaagtatgcccagctggacctctgcaatcaggttcgcaatgaagaaccccggcacgaaagtcccgggcaccaacgacttcacctgcaaaccgaggaaaggcacccatcccgtcgtgctcatcccgggcacatccgaggacgccttcatcacgtggtcgtactacggtccccgcctcaaggcagcaggattctgcgcctacacgttcaactacaacccggaaacacatccgcttgtggaagccgctgagaccagcggcaacatctactccacggcagctttcatggcccacttcgttgacagagtgctcaaggcaaccggtgctcagaaggtcaacctcgtcggccattctcagggcggcggccccctgccgcgcgcgtacatcaaatattacggggcgccaagaaagtcctcatctcgtcggtttggttccttccaacaggggaacacgcatgctcggcctggagaagttcctcaatgccagcggaaacccgctcagcactatcttcaatgctgcagcacagtttcgaaagctggaatccctgccccaacagttgcaagactccacatttctcagggaactcaacgcggatggaatgaccgtccccggcatcacatacaccgtcatcgccacccagttcgacaaccgagtatttccgtggactaataccttcatcaatgagcccggggtcaagaacatcgtcatccaagacgtctgtcccttggaccacagcgcccacacggatatccctaggacccgatgacccttcagattgtcatcaacgccttggaccccgagcgggccgccccggtcacctgcaccattcgcccattcaggcccagttag

SEQ ID NO: 107 - DNA, 2387 bp, Propionibacterium acnes
atggcgctcgccgcttctcccacagtgacggacgccattgccgcccccgggcccgacagctggtcggcgctgtgcgagcgatggatcgacatcatcaccggacgcagagccgcccggacctctgacccacgtgcccgagcgatcatcgccaagaccgaccggaaggtcgccgagatcctcaccgacctcgtatccggctcgagccgtcgaccgttctgatctcggcagacctccgcaaggagcagtcgcccttcatcaccaagacagcccgagccatcgagtcgatggcctgcgcctgggccacacccgggtccagctaccacaaggatcccgaaatcctctccgcgtgcatcgaggggctcagggacttctgccgacttcggtacaacccctcccaggacgagtatgggaactggtgggactgggaggacggcgcgtcaagagctgtcgccgatgtcatgtgcatcctgcacgacgtcctgccgcccgaggtcatgtccgcagcggcagccggcatcgaccacttcatccccgacccctggttccagcagccggcgtcggtcaagcccactgccaaccccgttcagcccgtggtctcgacaggcgcgaatcgcatggacctgacccgtgccgtcatgtgccgttccatcgcgaccggcgacgagaagaggctgcgtcatgccgttgacggattgcctgacgcctggcgcgtcaccaccgaaggtgacggtttccgtgccgacggcggattcatccagcactcccacatcccctacaccggcggctacggcgacgtcctgttcagcggactggcaatgctcttcccgctggtctccgggatgaggttcgacatcgtcgaatcggctcgtaaggctttccacgaccaggtcgaacgcggcttcatccccgtcatgtacaacggccagatcctcgacgacgtgcgcggccgatccatctcgcgcatcaacgagtctgccgccatgcacggcatctcgatcgcccgtgccatgctcatgatggctgatgccctgccgacacaccgcgccgaacagtggcgagggatcgtgcacggttggatggctcgaaacaccttcgatcacctgtccgagccgtccacccttgtcgacatctccctgttcgacgccgccgccaaggcgcgccccgtcccggagtcgtcgacgccgagctacttcgcgtccatggaccgtctcgtccaccgcaccgcggactggctaatcaccgtctccaactgttcggatcgcattgcctggtacgagtacggcaacggggagaacgaatgggcgtccaggaccagccagggaatgcgttacctcctgctgcccggagacatgggacagtacgaggacgggtactgggccaccgtcgactactcagcaccgacggggacgacggtggactccactccgctcaaacgcgccgtcggagcctcgtgggcggccaagaccccgaccaacgaatggtccgggggcctcgcatcggggtcgtggtctgccgccgcgtcccacatcacctcccaggactccgccctcaaggcacgccgcctatgggtgggtctgaaggacgccatggtagagctgacgaccgacgtgaccaccgacgcatcgcgggccataaccgtcgtcgagcaccgcaaggtggccagctcgtcgacgaaactcctcgtcgacggcaaccgggtctcatccgcgacctccttccagaacccccggtgggcccatctggacggagtcggcggttacgtcttcgccactgacaccgatctctccgcagatgtggcgacgagaaagggaacgtggatcgacgtcaatccctcccgcaaggtcaagggggctgacgaggtcatcgagcgcgcctacgcatccctgcacgtcacccaccacgatcgtccagtcgcgtgggcgctgcttcccactgccagccgttcccacacgatggccctggccacgcgcccaggagtcgagccgttcaccgtgctccggaatgacgcaaccgtccaggccgtccgctctgcgggtgccctcttgacgaaggaccccactgtcgtcaccaccttggctttttggaagccagctacctgcggcggcgtggcagttaaccgtcctgcgctggtgcagactcgggagagcgcaaaccaaatggaggtcgtcatcgtcgaacccacccagaagaggggatcacttaccgtaactattgagggaagctggaaggtcaaaaccgcagatagccacgttgatgtcagctgcgaaaacgcggccgggactctgcatgtcgacacggcggggctaggcggccagtccgtgcgagtaacgctggcacgccaggtaactcaaactccctccggcggcggccgccacgaccgagcctga

SEQ ID NO: 108 - DNA, 1113 bp, Propionibacterium acnes
atgcgtgttggtgttcctactgaggttaagaatagtgagtttcgtgtggctgtgacgccggcgggtgttcatgcgttggttggtcgtggtcatgaggtgttggttcaggctggtgctggtgtgggttcgggtattccggattcggattttgtgggtgctggtgcgcgggttgtgggtgatgtggagtcggtgtggggtgatgctgatttggtgttgaaggtgaaggagcctgttgcggaggagtatgggcggttgcatgagggtttggttctttttacgtatcttcatttggctgctgatgaggcgttgactcgtgagcttttggggcgtggggtgacgtcgattgcgtatgagacggtggagttggctgatcattcgttgccgttgttgtctccgatgtcggagattgcgggtcggttggctgctcaggtgggtgcgaattgtttgttgcagtctgctgggggtcgtggtgtgttgtttggtggtggttcgggtgtgcgtcgtggtcgggtgagtgtgcttggtggtggtgtggctgggttgtgtgcggctcgtgtggctgcgggtatgggtgctgatgtgacggtgtttgatgtggatgtggcgcggatgcgttatatcgatgaggtgtggggtgggcgtattggtacgcggttttcgagtccgttggcggttcgggaggcgtgtggtgagtctgatgtggtgattgggtcggtgttggtgcctggtgctcggactccgcatttggtggatcatgagatggtgttggggatggtgccggggtcggtgttggtggatgttgcggtggatcagggtgggtgttttgaggattctcatccgacgacgcatgcggatccgacgtttgtggtggggggttcggtgttttattgtgtggcgaatatgccgggtgcggtgccgcatacgtcgacgtatgcgttgacgaatgcgacgatgcggtatgtgttgttgttggctgatgagggttggaggggtgcgtgtgggtcgcgtgatgatttgcggcgtggtttggcgacttgtgatgggaagttggtgagtgcgccggtgggtgaggcgttgggtattgagtgtgtgcctgtgtctgaggtgttgtga

SEQ ID NO: 109 - DNA, 952 bp, Propionibacterium acnes
atggacaaaccagcgatagagatacgcgatctcgtcaagtcgttcccccaggccggcagccgcgaacgcctcattgccgtcgaccacttgtcaatgacgatcaaccgcggagaggtcgtggccttcctcgggcccaacggcgccggtaaatccacgaccgtcgacatgctccttggcatgaccagacccgacagtgggaaggtcactgtcctgggctccgatccgagaacagccgcccgtactggttgcatcagcgccgtctttcaaactggcggactgctacccgacttcaccgtcgcagagaccatgaaggccatcgctgccgcacacgggcagcgctcgagggtcaagcccctcaccgagaggtgggagttggccccattcgccggaaccaaggtcggcaaatgctcggggggggggtcagcggcagcgacttcgtttcgccctcgcgatgctgcctaaccccgatgtgcttattctcgacgagccgacaaccggtctggacgttgaagctcgtcgacgcttctggcaggtcatgggtgaggaggccgacgccggacgtacggtcattttcgccacccactacatcgaggaggccgattccttcgcccgtcgcgtcgtcctcgtcagtggtggacagctcgtcgcggacggtcccatcaatgaggttcgcgcctcggtgtccggatccactgtcagagcgactctcactgatccctccgtactggccgagggtctgcgcaccttccctgggattaacgacatcaccgtccagggccagcaactcatcgtgcacactagtcagcctgacgacctggcacgtcaccttctgacctacaccgacgctcacgggctgctcatctcgacgatgacccttgaggacgccttcgtccggctcacaggctccaatgattcccgtgacgatgttgacgactgggaggcagcagcatga

SEQ ID NO: 110 - DNA, 621 bp, Propionibacterium acnes
atgatcgataagaccatgatcaagctcgtgctggccgacgaccagaccctcgtacgcggcgctctggcagccctgcttagcatggagaacgacctggagatcgttggtacgtgtggtcggggagacgaggtgttcgcccttgtgcaggccacccatgccgacgtgtgtctgctcgacatcgagatgccaggtatcgacgggatcaccgtcgccgctgagctgagagaccaggcatcgtggtgtcgagttctcatcgtgacgaccttcgggcggccgggttatctgcggcgggccatggatgccggtgtggctgggttcctcgtcaaggacaccccagctgaggacctggcccgagtggtgcgcgaggtccatgccggaggtcgagttatcgacccagctttggccgccgaatccctcatcgagggacacaatcccttgtcggagcgggaacgggagattctgcggctggccgagtcgggggcctccatctccctcatcgcctcccagctctatctgtcggtgggcacggtgcgcaaccacgtgtcctcggccatcggcaagacgggtgctgcgaatcgcaccgaggctgccgtcacggctcgacagcgggggtggttgtga

SEQ ID NO: 111 - DNA, 707 bp, Propionibacterium acnes
gtgaccgtcctgctcttacgcctcgcggggcccttgcaatcctggggagattccagccgatttacgacccgggccacacgacgggaaccgacgaagtccggcgtcatcgggttgttggctgcggcgcagggacgacgtcgcaccgacagtctcgaagacctgctcaccctgcgattcggtgtgcgaaccgaccagccggggtcgatcgtgcgggacttccagacagccatggactgggcacatcccaaaaaggacggccgcgtcaaagccatgccgttgtcaaaccgctactacttggccgacgcagtcttcgtggcggcggtcgaaggagacccgtcctcctccaggcgttggacgaggcaatcagagacccggagttcccgctctacctcggccgaagatcctgtcccacggaaggacaggtgtccttaggcgtgcgagagagcgagctggtgaagaccttggaaaatgagccttggcacgccaagctgtggcaccaacgccgactgggacgatccgtccgacttcccatcgcctatgacgccgggccgggacagatcggcgacaccgtccgcgacataccgctgagcttcaaccctgagcggcgcgagtacggctggcgcgacgtcacgacgacaacgattgtcgtcgacaaccccaacggaagcgacgagcccgactggttcgcaggcctagaaggagcttga

SEQ ID NO: 112 - DNA, 588 bp, Propionibacterium acnes
gtgagtcgcatcgtcatcgtcaggcacgggcagtcgacgtggaatcgtcaagggcgcatccaagggcagacaatgggtattcccctgacgatgctcgggagacgccaggctcgccaggccgctcacacggtggcaggtctggtaccccacgacaccccgatcatcgcctccgaccagaagcgggcgcgtcagacagctcgtcctatcgcgcgggtgctgggcgtaccagcgacgaccgatccacggctgcgcgagcaagggttgggagccatggagggtcacaccgcggatgatctcgagccccttccccagccaacgggtgtacatccggccgacgtgcgatgggctggtggggagtcgctcgcggatgtggcggagaggtgccgcagcctgttggatgacgtggcagctcgcgacctaccggcgatcgtcctcgtcacccacggtgacaccatgcgggtcctgctggggattctcgacggtcgcagtcaccgcgacctcgactgggaccttccactgacgaacggaagtgtcatagcgcgagatgtgaacctcagcgagtcgcatcggcggctttcgctgtcgtaa

SEQ ID NO: 113 - DNA, 1010 bp, Propionibacterium acnes
atgaagaaaaactggttactcacaaccctccttgccacaatgatgatcgccatgggcacgacgaccaccgccttcgccagcccgcctaccgacatcactcccgaacatccaggcggggttacccgcctcacagccccgacggaatcccctcgaatattgaggggccaagtatgcccagctggacctctgcaatcaggttcgcaatgaagaaccccggcacgaaagtcccgggcaccaacgacttcacctgcaaaccgaggaaaggcacccatcccgtcgtgctcatcccgggcacatccgaggacgccttcatcacgtggtcgtactacggtccccgcctcaaggcagcaggattctgcgcctacacgttcaactacaacccggaaacacatccgcttgtggaagccgctgagaccagcggcaacatctactccacggcagctttcatggcccacttcgttgacagagtgctcaaggcaaccggtgctcagaaggtcaacctcgtcggccattctcagggcggcggccccctgccgcgcgcgtacatcaaatattacggggcgccaagaaagtcctcatctcgtcggtttggttccttccaacaggggaacacgcatgctcggcctggagaagttcctcaatgccagcggaaacccgctcagcactatcttcaatgctgcagcacagtttcgaaagctggaatccctgccccaacagttgcaagactccacatttctcagggaactcaacgcggatggaatgaccgtccccggcatcacatacaccgtcatcgccacccggttcgacaaccgagtatttccgtggactaataccttcatcaatgagcccggggtcaagaacatcgtcatccaagacgtctgtcccttggaccacagcgcccacacggatatccctaggacccgatgacccttcagattgtcatcaacgccttggaccccgagcgggccgccccggtcacctgcaccattcgcccattcaggcccagttag

(19) United States
(12) Patent Application Publication
(10) Pub. No.: US 2019/0336542 A1
(43) Pub. Date: Nov. 7, 2019

(54) SYNTHETIC BACTERIA AND METHODS OF USE

(71) Applicant: NAKED BIOME, INC., San Francisco, CA (US)

(72) Inventors: Emma TAYLOR, San Francisco, CA (US); David HANZEL, San Francisco, CA (US); Daniel J. NILSON, Livermore, CA (US)

(21) Appl. No.: 16/095,280

(22) PCT Filed: Apr. 21, 2017
(86) PCT No.: PCT/US2017/028918

§ 371 Date: Oct. 19, 2018

Related U.S. Application Data
(60) Provisional application No. 62/325,834, filed on Apr. 21, 2016.
(60) Provisional application No. 62/385,836, filed on Sep. 9, 2016.
(60) Provisional application No. 62/441,930, filed on Jan. 3, 2017.

(51) Int. Cl. A61K 35/74; C12N 15/74; A61K 38/22; A61K 38/47; C12N 15/52; C12N 9/18; C12N 9/26; A61P 17/06; A61P 17/08

(52) U.S. Cl. CPC A61K 35/74; C12N 15/74; A61K 38/2228; A61K 38/47; C12Y 302/01035; C12N 9/18; C12N 9/2474; A61P 17/06; A61P 17/08; C12N 15/52

(57) ABSTRACT

Disclosed are synthetic bacteria having de novo metabolic pathways for biosynthesis of select compounds. Further disclosed are methods of use to prevent and treat skin disorders and diseases.

### CROSS-REFERENCE TO RELATED APPLICATIONS

This is a U.S. National Phase of International Application No. PCT/US2017/028918 filed Apr. 21, 2017, which application claims the benefit of U.S. Provisional Ser. Nos. 62/325,834 filed on Apr. 21, 2016; 62/385,836 filed on Sep. 9, 2016; and 62/441,930 filed on Jan. 3, 2017 all of which are incorporated herein in their entirety.

### SEQUENCE LISTING

The instant application contains a Sequence Listing which has been filed electronically in ASCII format and is hereby incorporated by reference in its entirety. Said ASCII copy, created on Apr. 19, 2017, is named 48236-705_601_SL.txt and is 181,965 bytes in size.

### BACKGROUND

Acne vulgaris, or simply acne, is a relatively common chronic inflammatory skin disorder that occurs when follicles on the surface of the skin become blocked, forming a plug. Acne affects an estimated 80-90% of adolescents, as well as adults of all ages. A majority of current therapies to treat acne are formulations of generic compounds such as topical antibiotics, retinoids, benzoyl peroxide, and salicylic acid. Many of these formulations are irritating to the patient, limiting long-term compliance and therefore efficacy. Thus, there is a need for an effective and well-tolerated acne therapy to provide long-term maintenance and acne prevention.

### SUMMARY

Propionibacterium acnes is an important skin commensal, but it is also considered a pathogenic factor in several diseases including acne vulgaris. Type IA-2 (primarily ribotype 4 [RT4] and RT5) strains have been associated with acne, while type II strains, in particular RT6 and some RT2 strains, have rarely been found in acneic skin or are found to produce low pro-inflammatory metabolites and thus are defined as health-associated strains in the context of acne. Because P. acnes is the dominant bacteria in the human follicle, engineering and applying health-associated P. acnes strains to individuals with acne is a promising way to affect the microbiome of acneic skin. In particular, P. acnes are engineered to suppress acne at the molecular, metabolic, structural and ecological levels. In many cases, the microbial quorum-sensing mechanisms, microbial lipid synthesis and degradation pathways, and/or microbial communities are affected by the engineering. In this context, the phylogenetic and metabolic characteristics of a particular P. acnes strain can be, but is not necessarily, related to the activation or suppression of mechanisms associated with acne. In some cases, two P. acnes strains with identical 16S rDNA genes may have completely different metabolic capabilities as they relate to the causation of acne due to differences in regulation of their metabolic pathways.

Disclosed herein are synthetic bacteria that have been modified to affect metabolic pathways rendering the bacteria non-pathogenic. In certain instances these are modifications at the genetic level and involve deleting, in whole or in part, disrupting, inactivating or modifying a gene that affects a step in metabolic pathway, such as, for example the porphyrin pathway. In other instances, these modifications affect enzyme activity itself for example lipase or hyaluronidase. For example, genetic modification may be made that decrease expression or activity of these enzymes. In some instances an enzyme from a heterologous microbe may be introduced or swapped with an existing lipase or hyaluronidase. For example the hyaluronidase from Group B Streptococcus may be introduced into a bacteria and the endogenous hyaluronidase can be deleted, in whole or in part, inactivated, disrupted or replaced. In certain instances pathogenic type I lipases are deleted, in whole or in part, disrupted, inactivated r modified, or a lipase from a heterologous species may be introduced while the endogenous lipase is deleted, in whole or in part, inactivated, disrupted or replaced.

In one aspect of the disclosure, provided herein is a method for treating a skin disorder in a patient in need thereof, the method comprising applying from about 10² cfu/cm2 to about 10¹² cfu/cm2 of a synthetic bacteria to skin affected with the skin disorder; provided that the synthetic bacteria is engineered from a non-pathogenic bacteria and the synthetic bacteria: (a) produce one or more biomolecules comprising: (i) a pro-inflammatory metabolite produced at a tunable level to effect a first metabolic pathway of the synthetic bacteria; (ii) an enzyme produced at a tunable level to effect a second metabolic pathway of the synthetic bacteria; (iii) a lipase modified relative to a native lipase of the non-pathogenic bacteria; (iv) a sensor receptor specific for a first target molecule; (v) a sensor effector specific for a second target molecule; or (vi) a combination of any of (i) to (v); (b) mitigates induction of a patient inflammatory response to the applied synthetic bacteria; (c) comprises a genome modification that prevents the synthetic bacteria from acquiring an antibiotic resistance gene; or (d) a combination of any of (a) to (c).

In some embodiments, the enzyme is ST2S, ST4S, ST6S, bmpA, PTS-Mtl-EIIABC, MFS ST1, MFS ST2, ST1P, ST3P, ST4P, ST5P, ST6P, ST7P, PTS-Mtl-EIIA, ST1P1, ST3P1, ST4P1, ST6P1, FhuD, ST7A, manA, FhuC, FhuB, FhuD, COX15, talAB, hMuV, HtaA, HmuT, GAPDH, hemH, CS, IDH1, cobA-hemD, cysG, IDH1, IDH1, TGL, OGDH, narJ, narl, narH, narG, E1.7.2.1, norB, gdhA, gudB, rocG, MDT1, MDT2, MDT3, T2SF2, T2SF1, secA, secY, secF, secD, yajC, secE, ftsY, yidC, secG, clpX, clp2, clp1, or a combination thereof, and the enzyme is down-regulated. In some embodiments, the enzyme is ST3S; PTS-Nag-E1; ST2P; ST2A; FhuC; FhuB; FhuD; PFK; glpK; cyoE; fabZ; fabG; fabF; fabZ; fabG; fabF; fabZ; fabG; fabF; fabZ; fabG; fabF; fabZ; fabG; fabF; fabZ; fabG; fabF; fabZ; fabG; fabF; fabZ; fabG; fabF; fabD; fumC; DLST; AMT; CblO; cbiM; cbiQ; cbiN; cysG-cbiX; cobI-cbiL; cobM, cbiF; cobK, cbiJ; cobO, btuR; cobQ, cbiP; cbiB, cobD; cobS, cobV; MDT4; or a combination thereof, and the enzyme is up-regulated.

In some embodiments, the pro-inflammatory metabolite is a porphyrin. In some embodiments, the pro-inflammatory metabolite is produced at a threshold level about or less than about 4 micromolar. In some embodiments, the pro-inflammatory metabolite level is modulated by engineering a vitamin B12 metabolic pathway in the non-pathogenic bacteria. In some embodiments, engineering the vitamin B12 metabolic pathway comprises modifying one or more native molecules of the vitamin B12 metabolic pathway in the non-pathogenic bacteria. In some embodiments, the synthetic bacteria secretes an entity that increases or decreases production of another biomolecule in a bacteria neighboring the synthetic bacteria; and provided that the entity is selected from a chemical, nucleic acid, peptide, polypeptide, lipid, carbohydrate or small molecule. In some embodiments, the modified lipase has no lipase activity or a lipase activity less than about 50% of the activity of the native lipase. In some embodiments, the synthetic bacteria produces at least about 50% fewer free fatty acids from sebum than the non-pathogenic bacteria. In some embodiments, the first target molecule is a porphyrin, or a molecule involved in porphyrin metabolism. In some embodiments, the first target molecule is involved in a lipase reaction. In some embodiments, the first target molecule is involved in glucose production.

In some embodiments, the method further comprises monitoring the presence or absence of the first target molecule by monitoring the presence or absence, respectively, of binding between the sensor receptor and the first target molecule. In some embodiments, the presence or absence of the first target molecule indicates a pH level of the affected skin. In some embodiments, the pH level is less than about pH 4 or greater than about pH 7. In some embodiments, the pH level is not about pH 5.5. In some embodiments, the synthetic bacteria produces or sequesters an entity in the affected skin to alter the pH level of the affected skin. In some embodiments, the skin is altered to a pH between about pH 5 and pH 6. In some embodiments, the sensor effector affects production of the second target molecule by the synthetic bacteria. In some embodiments, the sensor effector attenuates or halts production of the second target molecule. In some embodiments, the second target molecule is a porphyrin. In some embodiments, the second target molecule is involved in a lipase reaction. In some embodiments, the second target molecule is involved in glucose production.

In some embodiments, the patient inflammatory response is mitigated by the production of a toll like receptor (TLR) ligand modified from a native TLR ligand of the non-pathogenic bacteria. In some embodiments, the modified TLR ligand has no binding affinity to a TLR of the patient, or the modified TLR ligand has a binding affinity to a TLR of the patient that is less than about 50% of the binding affinity of the native TLR ligand. In some embodiments, the TLR is present on a keratinocyte, inflammatory cell, other antigen presenting cell, or a combination thereof, in the patient. In some embodiments, the inflammatory cell is selected from a macrophage dendritic cell, and a Langerhans cell. In some embodiments, the modified TLR ligand is selected from a cell-wall component of the non-pathogenic bacteria, a lipoprotein from a gram-positive bacteria, lipoarabinomannan from mycobacteria, zymosan from a yeast cell wall, or a combination thereof. In some embodiments, the cell-wall component is selected from a peptidoglycan and lipoteichoic acid. In some embodiments, the patient inflammatory response is mitigated by replacing a toll like receptor (TLR) ligand present in the non-pathogenic bacteria with a human peptide. In some embodiments, the patient inflammatory response is mitigated by the absence of a toll like receptor (TLR) ligand present in the non-pathogenic bacteria. In some embodiments, the TLR ligand comprises TLR2 ligand, TLR4 ligand, or a combination thereof.

In some embodiments, the antibiotic is in a class of tetracycline antibiotics or erythromycin/clindamycin antibiotics. In some embodiments, the genome modification comprises a mutation in 16S ribosomal RNA of the non-pathogenic bacteria, 23S ribosomal RNA of the non-pathogenic bacteria, a mutation outside of the 16S or 23 S RNA, or a combination thereof, as compared to the non-pathogenic bacteria. In some embodiments, the 23 S ribosomal RNA of the synthetic bacteria has a mutation at base 2058, 2057, 2059, 1058, or a combination thereof as compared to the non-pathogenic bacteria. In some embodiments, a mutation comprises a base substitution, addition, deletion, or a combination thereof. In some embodiments, the synthetic bacteria is prevented from acquiring a mutation at base 2058, 2057, 2059, 1058, or a combination thereof, in the 23 S ribosomal RNA.

In some embodiments, the non-pathogenic bacteria is a gram positive bacteria. In some embodiments, the non-pathogenic bacteria is a gram negative bacteria. In some embodiments, the gram positive bacteria comprises Propionibacterium acnes, Staphylococcus epedermidis, Staphylococcus auereus, or a combination thereof. In some embodiments, the non-pathogenic bacteria is a Propionibacterium acnes strain belonging to Type clade II. In some embodiments, the Propionibacterium acnes belongs to ribotype 2. In some embodiments, the Propionibacterium acnes belongs to ribotype 6. In some embodiments, the Propionibacterium acnes strain has less than about 95% sequence homology to any one of SEQ ID NOS: 100, 101, 102 and 103. In some embodiments, the Propionibacterium acnes belongs to ribotype 8. In some embodiments, the non-pathogenic bacteria a Propionibacterium acnes strain associated with healthy skin. In some embodiments, the Propionibacterium acnes strain associated with healthy skin comprises ST0, ST7, ST25, ST26, ST27, ST28, ST30, ST58, ST59, ST60, ST61, ST62, ST63, ST64, ST65, ST66, ST67, ST68, ST69, ST71, ST72, ST79, ST6, ST7, ST25, ST26, ST27, ST28, ST30, ST58, ST59, ST60, ST61, ST62, ST63, ST64, ST65, ST66, ST67, ST68, ST69, ST71, ST72, ST79, ST32, ST33, ST73, ST74, ST75, ST76, ST77, ST81, ST90, ST12, ST32, ST33, ST51, ST53, ST73, ST74, ST75, ST76, ST77, ST81, or a combination thereof. In some embodiments, the synthetic bacteria do not induce or induce less human beta defensin (HBD) in patient keratinocytes as compared to the level of HBD induced by a disease-associated P. acnes strain; provided that the HBD comprise HBD-1, HBD-2, HBD-3 or a combination thereof; and provided that the disease-associated P. acnes strain is of clade IA, ribotype 4, ribotype 5, or a combination thereof. In some embodiments, the level of HBD induced in patient keratinocytes is less than about 50% of the amount of HBD induced in patient keratinocytes if the disease-associated P. acnes strain were applied to the affected skin in the same amount. In some embodiments, the synthetic bacteria do not induce or induce less than about 50% of one or more of: interleukin-8, interleukin-1, interleukin-6, TNF-alpha, and NFkB in patient keratinocytes as compared to levels produced by a disease-associated P. acnes strain; and provided that the disease-associated P. acnes strain is of clade IA, ribotype 4, ribotype 5, or a combination thereof. In some embodiments, the synthetic bacteria do not produce or produce less than about 50% of MMP as compared to levels of MMP produced by a disease-associated P. acnes strain; and provided that the disease-associated P. acnes strain is of clade IA, ribotype 4, ribotype 5, or a combination thereof. In some embodiments, the synthetic bacteria recruit at least about 50% fewer neutrophils and at least about 50% fewer polymorphonuclear leukocytes from the patient than the disease-associated P. acnes strain if the disease-associated P. acnes strain were applied to the affected skin in the same amount. In some embodiments, the synthetic bacteria recruit at least about 50% fewer dermal fibroblasts from the patient than the disease-associated P. acnes strain if the disease-associated P. acnes strain were applied to the affected skin in the same amount. In some embodiments, the MMP comprises MMP-8, MMP2 or both MMP-8 and MMP-2.

In some embodiments, the synthetic bacteria inhibits production of pro-inflammatory neuropeptides in the patient. In some embodiments, the one or more biomolecules produced by the synthetic bacteria is an antioxidant selected from vitamin A, vitamin C, and vitamin D. In some embodiments, the genome of the synthetic bacteria is stabilized to maintain engineered features of the synthetic bacteria. In some embodiments, the synthetic bacteria is applied in a topical composition at a concentration of at least about 0.1% by weight of the total composition. In some embodiments, the method further comprises administration of benzoyl peroxide, a laser treatment, peel treatment, antibiotic, retinoid, azaeilic acid, sulfur compound, other acne treatments conventional in the art, or a combination thereof, to the affected skin prior to application of the synthetic bacteria. In some embodiments, the method further comprises application of resveratrol to the affected skin prior to application of the synthetic bacteria. In some embodiments, the method further comprises exposing the affected skin to electromagnetic radiation at a wavelength from about 400 nm to about 700 nm prior to application of the synthetic bacteria. In some embodiments, the electromagnetic radiation is emitted from a light emitting diode. In some embodiments, the wavelength is between about 390 nm and about 420 nm. In some embodiments, the synthetic bacteria are applied in a topical oil-in-water emulsion or a topical water-in-oil emulsion. In some embodiments, the synthetic bacteria are lyophilized. In some embodiments, the synthetic bacteria are incorporated in a biologic stability platform.

In some embodiments, the patient has acne. In some embodiments, the method further comprises administering to the patient an additional acne treatment. In some embodiments, the additional acne treatment is configured to prevent acne. In some embodiments, the additional acne treatment is administered prior to application of the synthetic bacteria. In some embodiments, the addition acne treatment is administered after application of the synthetic bacteria. In some embodiments, the additional acne treatment comprises blue light therapy, red light therapy, a peel, an oral antibiotic, oral isotretinoin, oral hormonal therapy, or a combination thereof. In some embodiments, the oral hormonal therapy comprises spironolactone, an estrogenic compound, a progestational compound, a gonadotropin releasing hormone, or a combination thereof. In some embodiments, the additional acne treatment comprises applying to the affected skin: salicylic acid, glycolic acid, benzoyl peroxide, azaleic acid, a retinoid, a pimecrolimu, tacrolimus, topical dapsone, topical erythromycin/clindamycin, a topical anti-hormonal, or a combination thereof.

In some embodiments, the synthetic bacteria produces omiganan. In some embodiments, the synthetic bacteria produces epinecidin-1 (22-42) peptide. In some embodiments, the synthetic bacteria produces SALF (55-76) cyclic peptide. In some embodiments, the synthetic bacteria produces SALF (55-76) linear peptide. In some embodiments, the synthetic bacteria produces granulysin or a granulysin derivative. In some embodiments, the granulysin derivative comprises a helix-loop-helix motif. In some embodiments, the granulysin derivative comprises peptide 31-50 having a V44W mutation.

In some embodiments, the synthetic bacteria acquire a deleterious molecule from the affected skin to decrease the relative amount of the deleterious molecule from the affected skin after application of the synthetic bacteria. In some embodiments, the synthetic bacteria metabolizes the deleterious molecule. In some embodiments, the deleterious molecule comprises: a sulfur oxide, a nitrogen oxide, carbon monoxide, a volatile organic compound, particulate matter, a persistent free radical, toxic metal, a chlorofluorocarbon, ammonia, an odorous molecule, a radioactive pollutant, secondary pollutant, ground level ozone, peroxyacetyl nitrate, hazardous air pollutant, persistent organic pollutant, or a combination thereof. In some embodiments, the sulfur oxide comprises sulfur monoxide, disulfur dioxide, disulfur monoxide, sulfur dioxide, sulfur trioxide, of a combination thereof. In some embodiments, the sulfur oxide is sulfur dioxide. In some embodiments, the nitrogen oxide comprises nitrogen monoxide, nitrogen dioxide, nitrous oxide, nitrosylazide, oxatetrazole, dinitrogen trioxide, dinitrogen tetraoxide, dinitrogen pentoxide, trinitramide, nitrite, nitrate, nitronium, nitrosonium, peroxonitrite, or a combination thereof. In some embodiments, the nitrogen oxide is nitrogen dioxide. In some embodiments, the volatile organic compound is methane. In some embodiments, the volatile organic compound comprises benzene, toluene, xylene, 1,3-butadiene, isoprene, terpene, aliphatic hydrocarbon, ethyl acetate, glycol ether, acetone, chlorofluorocarbon, tetrachloroethene, methylene chloride, perchloroethylene, methyl tert-butyl ether, formaldehyde, or a combination thereof. In some embodiments, the particulate matter is a solid or liquid suspended in a gas; and provided that the particulate matter is derived from one or more of the following: volcanoes, dust storms, forest and grassland fires, living vegetation, sea spray, and burning of fossil fuels in vehicles, power plants and industrial processes. In some embodiments, the persistent free radical comprises Gomberg's triphenylmethyl radical, Fremy's salt (potassium nitrosodisulfonate), nitroxide, TEMPO (2,2,6,6-Tetramethylpiperidin-1-yl)oxyl), 4-Hydroxy-TEMPO (4-hydroxy-2,2,6,6-tetramethylpiperidin-1-oxyl), nitronyl nitroxide, azephenylenyl, perchlorophenylmethyl radical, TTM (tris(2,4,6-trichlorophenyl)methyl radical), or a derivative or combination thereof. In some embodiments, the toxic metal comprises mercury, lead, cadmium, manganese, or an alloy or combination thereof. In some embodiments, the chlorofluorocarbon comprises trichlorofluoromethane; dichlorodifluoromethane; difluoromethane/pentafluoroethane; chlorotrifluoromethane; chlorodifluoromethane; dichlorofluoromethane; chlorofluoromethane; bromochlorodifluoromethane; 1,1,2-trichloro-1,2,2-trifluoroethane; 1,1,1-trichloro-2,2,2-trifluoroethane; 1,2-dichloro-1,1,2,2-tetrafluoroethane; 1-chloro-1,1,2,2,2-pentafluoroethane; 2-chloro-1,1,1,2-tetrafluoroethane; 1,1-dichloro-1-fluoroethane; 1-chloro-1,1-difluoroethane; tetrachloro-1,2-difluoroethane; tetrachloro-1,1-difluoroethane; 1,1,2-trichlorotrifluoroethane; 1-bromo-2-chloro-1,1,2-trifluoroethane; 2-bromo-2-chloro-1,1,1-trifluoroethane; 1,1-dichloro-2,2,3,3,3-pentafluoropropane; 1,3-dichloro-1,2,2,3,3-pentafluoropropane or a combination thereof. In some embodiments, the radioactive pollutant is produced by a nuclear explosion, nuclear event, nuclear explosive device, radioactive decay of radon, or a combination thereof. In some embodiments, the secondary pollutant comprises particulates created from gaseous primary pollutants and compounds in photochemical smog. In some embodiments, the hazardous air pollutant comprises carbon monoxide, cyanide, glycol ether, polycyclic aromatic hydrocarbon, or a combination thereof. In some embodiments, the persistent organic pollutant comprises acetaldehyde; acetamide; acetonitrile; acetophenone; acrolein; acrylamide; acrylic acid; acrylonitrile 4-aminobiphenyl; aniline o-anisidine; m-anisidine; p-anisidine; asbestos; benzene; 1,3-butadiene; carbon disulfide; carbon monoxide; carbon tetrachloride; carbonyl sulfide; chlorine; chlorobenzene; chloroethane; chloroform; chloromethane; chloroprene; cresol; o-cresol; cumene; 1,2-dibromoethane; 1,2-dichloroethane; dichloromethane; ethylbenzene; ethylene glycol; ethylene oxide; fluidized bed concentrator; formaldehyde; hexachlorobenzene; hexane; hydrazine; hydrogen chloride; hydrogen fluoride; methanol; methyl isobutyl ketone; methyl isocyanide; methyl methacrylate; methyl tert-butyl ether; naphthalene; 4-nitroaniline; nitrogen dioxide; phenol; polychlorinated biphenyl; propionaldehyde; quinoline; sodium selenite; styrene; sulfur trioxide; tetrachloroethylene; toluene; 1,1,1-trichloroethane; trichloroethylene; vinyl acetate; vinyl chloride; xylene; chemicals regulated by the US EPA via maximum achievable control technology standards; or a combination thereof.

In some embodiments, the synthetic bacteria produces water or other moist environment on the affected skin. In some embodiments, the water or other moist environment alleviates irritation of the skin. In some embodiments, the patient has acne and the affected skin is irritated by an acne associated treatment. In some embodiments, the acne treatment comprises application of a retinoid, benzoyl peroxide, isotretinoin, salicylic acid, glycociazeilic acid, or a combination thereof, to the affected skin. In some embodiments, the water or other moist environment treats a disorder exacerbated with dry skin. In some embodiments, the disorder exacerbated with dry skin comprises eczema, xerosis, cheilitis, stasis dermatitis, seborrheic dermatitis, rosacea, psoriasis, or a combination thereof. In some embodiments, the synthetic bacteria produces a ceramide. In some embodiments, the ceramide treats xerosis, eczema, stasis dermatitis, psoriasis, cheilitis, retinoid dermatitis, an additional dry skin disorder, or a combination thereof. In some embodiments, the synthetic bacteria produces filagagrin. In some embodiments, the filagagrin treats xerosis, eczema, stasis dermatitis, psoriasis, cheilitis, retinoid dermatitis, an additional dry skin disorder, or a combination thereof.

In another aspect of the disclosure, provided herein is a synthetic bacteria derived from a non-pathogenic bacteria, the synthetic bacteria comprising a vitamin B12 metabolic pathway comprising a biomolecule introduced, removed, or modified relative to the vitamin B12 metabolic pathway of the non-pathogenic bacteria; provided that the synthetic bacteria produces a high level of intracellular vitamin B12 as compared to the non-pathogenic bacteria. In some embodiments, the biomolecule is an enzyme encoded by one or more of the following genes: cysG-cbiX, cobI-cbiL, cobM, cbiF, cobK, cbiJ, cobH, cbiC, cobB-cbiA, cobO, btuR, cobQ, cbiP, cbiB, cobD, cobS, and cobV. In some embodiments, the non-pathogenic bacteria is Propionibacterium acnes. In some embodiments, the biomolecule is heterologous to the non-pathogenic bacteria. In some embodiments, the synthetic bacteria is cultured in a substantially anaerobic culture medium. Further provided herein is use of the synthetic bacteria for the treatment of acne in a subject in need thereof.

In another aspect of the disclosure, provided herein is a synthetic bacteria derived from a non-pathogenic bacteria, the synthetic bacteria comprising a porphyrin pathway comprising a biomolecule introduced, removed, or modified relative to the porphyrin pathway of the non-pathogenic bacteria; provided that porphyrin is not produced by the synthetic bacteria or is produced at a level less than about 4 micromolar of porphyrin in the synthetic bacteria. In some embodiments, the non-pathogenic bacteria is Propionibacterium acnes. In some embodiments, the biomolecule is an enzyme encoded by one or more of the following genes: COX15, cyoE, and HemB, HemC, HemD, HemE, HemF, HemG, HemH, HemY, or PPA2095 protoporphyrinogen oxidase (HemY homologue). In some embodiments, the biomolecule is heterologous to the non-pathogenic bacteria. In some embodiments, the synthetic bacteria is cultured in a substantially anaerobic culture medium. Further provided herein is use of the synthetic bacteria for the treatment of acne in a subject in need thereof.

In another aspect of the disclosure, provided herein is a synthetic bacteria derived from a non-pathogenic bacteria, the synthetic bacteria comprising a citric acid pathway comprising a biomolecule introduced, removed, or modified relative to the citric acid pathway of the non-pathogenic bacteria; provided that the synthetic bacteria produces glycine. In some embodiments, the non-pathogenic bacteria is Propionibacterium acnes. In some embodiments, the biomolecule is an enzyme encoded by one or more of the following genes: CS, IDH1, OGDH, DLST, and fumC. In some embodiments, the at least one gene is heterologous to the non-pathogenic bacteria. In some embodiments, the synthetic bacteria is cultured in a substantially anaerobic culture medium. In some embodiments, the amount of glycine produced in the synthetic bacteria is greater than the amount of glycine produced in the non-pathogenic bacteria or in a pathogenic bacteria strain in the same genus and species as the non-pathogenic bacteria. In some embodiments, the amount of glycine produced in the synthetic bacteria is less than the amount of glycine produced in the non-pathogenic bacteria or in a pathogenic bacteria strain in the same genus and species as the non-pathogenic bacteria. Further provided herein is use of the synthetic bacteria for the treatment of acne in a subject in need thereof.

In another aspect of the disclosure, provided herein is a synthetic bacteria derived from a non-pathogenic bacteria, the synthetic bacteria comprising a nitrous oxide pathway comprising a biomolecule introduced, removed, or modified relative to the nitrous oxide pathway of the non-pathogenic bacteria; provided that the synthetic bacteria produces nitrous oxide. In some embodiments, the non-pathogenic bacteria is Propionibacterium acnes. In some embodiments, the biomolecule is an enzyme encoded by one or more of the following genes: narJ, narI, narH, narG, E.1.7.2.1, norB, gdhA, gudB, and rocG. In some embodiments, the at least one gene is heterologous to the non-pathogenic bacteria. In some embodiments, the synthetic bacteria is cultured in a substantially anaerobic culture medium. In some embodiments, the amount of nitrous oxide produced in the synthetic bacteria is greater than the amount of nitrous oxide produced in the non-pathogenic bacteria or in a pathogenic bacteria strain in the same genus and species as the non-pathogenic bacteria. In some embodiments, the amount of nitrous oxide produced in the synthetic bacteria is less than the amount of nitrous oxide produced in the non-pathogenic bacteria or in a pathogenic bacteria strain in the same genus and species as the non-pathogenic bacteria. Further provided herein is use of the synthetic bacteria for the treatment of acne in a subject in need thereof.

In another aspect of the disclosure, provided herein is a synthetic bacteria derived from a non-pathogenic bacteria, the synthetic bacteria comprising a fatty acid synthesis pathway comprising a biomolecule introduced, removed, or modified relative to the fatty acid synthesis pathway of the non-pathogenic bacteria; provided that the synthetic bacteria produces fatty acids. In some embodiments, the non-pathogenic bacteria is Propionibacterium acnes. In some embodiments, the biomolecule is an enzyme encoded by fadD. In some embodiments, the synthetic bacteria is cultured in a substantially anaerobic culture medium. Further provided herein is use of the synthetic bacteria for the treatment of acne in a subject in need thereof.

In another aspect of the disclosure, provided herein is a synthetic bacteria derived from a non-pathogenic bacteria, the synthetic bacteria adapted to secrete a corticotropin releasing hormone (CRH), a corticotropin releasing hormone receptor (CRHR), a corticotropin releasing hormone binding protein (CRHBP), or a combination thereof. In some embodiments, the non-pathogenic bacteria is Propionibacterium acnes. Further provided herein is use of the synthetic bacteria for the treatment of acne in a subject in need thereof. Further provided herein is a method of producing the synthetic bacteria comprising engineering the non-pathogenic bacteria to produce to the synthetic bacteria.

In another aspect of the disclosure, provided herein is a synthetic bacteria derived from a non-pathogenic bacteria, the synthetic bacteria adapted to secrete a interleukin-1 inhibitor. In some embodiments, the non-pathogenic bacteria is Propionibacterium acnes. Further provided herein is use of the synthetic bacteria for the treatment of acne in a subject in need thereof. Further provided herein is a method of producing the synthetic bacteria comprising engineering the non-pathogenic bacteria to produce to the synthetic bacteria.

In another aspect of the disclosure, provided herein is a synthetic bacteria derived from a non-pathogenic bacteria, the synthetic bacteria adapted to secrete a TNF-alpha inhibitor. In some embodiments, the non-pathogenic bacteria is Propionibacterium acnes. Further provided herein is use of the synthetic bacteria for the treatment of acne in a subject in need thereof. Further provided herein is a method of producing the synthetic bacteria comprising engineering the non-pathogenic bacteria to produce to the synthetic bacteria.

In another aspect of the disclosure, provided herein is a synthetic bacteria derived from a non-pathogenic bacteria, the synthetic bacteria adapted to secrete a TNF-alpha inhibitor and an interleukin-8 inhibitor. In some embodiments, the non-pathogenic bacteria is Propionibacterium acnes. Further provided herein is use of the synthetic bacteria for the treatment of acne in a subject in need thereof. Further provided herein is a method of producing the synthetic bacteria comprising engineering the non-pathogenic bacteria to produce to the synthetic bacteria.

In another aspect of the disclosure, provided herein is a synthetic bacteria derived from a non-pathogenic bacteria, the synthetic bacteria adapted to secrete a tumor neutrophil chemotaxis inhibitor. In some embodiments, the non-pathogenic bacteria is Propionibacterium acnes. Further provided herein is use of the synthetic bacteria for the treatment of acne in a subject in need thereof. Further provided herein is a method of producing the synthetic bacteria comprising engineering the non-pathogenic bacteria to produce to the synthetic bacteria.

In another aspect of the disclosure, provided herein is a synthetic bacteria derived from a non-pathogenic bacteria, the synthetic bacteria adapted to secrete a interleukin-6 inhibitor. In some embodiments, the non-pathogenic bacteria is Propionibacterium acnes. Further provided herein is use of the synthetic bacteria for the treatment of acne in a subject in need thereof. Further provided herein is a method of producing the synthetic bacteria comprising engineering the non-pathogenic bacteria to produce to the synthetic bacteria.

In another aspect of the disclosure, provided herein is a synthetic bacteria derived from a non-pathogenic bacteria, the synthetic bacteria adapted to secrete a NFkB inhibitor. In some embodiments, the non-pathogenic bacteria is Propionibacterium acnes. Further provided herein is use of the synthetic bacteria for the treatment of acne in a subject in need thereof. Further provided herein is a method of producing the synthetic bacteria comprising engineering the non-pathogenic bacteria to produce to the synthetic bacteria.

In another aspect of the disclosure, provided herein is a synthetic bacteria derived from a non-pathogenic bacteria, the synthetic bacteria adapted to secrete one or more of: human beta defensing-1 inhibitor, human beta defensing-2 inhibitor, and human beta defensing-3 inhibitor. In some embodiments, the non-pathogenic bacteria is Propionibacterium acnes. Further provided herein is use of the synthetic bacteria for the treatment of acne in a subject in need thereof. Further provided herein is a method of producing the synthetic bacteria comprising engineering the non-pathogenic bacteria to produce to the synthetic bacteria.

In another aspect of the disclosure, provided herein is a synthetic bacteria derived from a non-pathogenic bacteria, the synthetic bacteria adapted to secrete an antiantroden. In some embodiments, the non-pathogenic bacteria is Propionibacterium acnes. Further provided herein is use of the synthetic bacteria for the treatment of acne in a subject in need thereof. Further provided herein is a method of producing the synthetic bacteria comprising engineering the non-pathogenic bacteria to produce to the synthetic bacteria.

In another aspect of the disclosure, provided herein is a synthetic bacteria derived from a non-pathogenic bacteria, the synthetic bacteria adapted to secrete testosterone, a DHT inhibitor, and derivatives and combinations thereof. In some embodiments, the non-pathogenic bacteria is Propionibacterium acnes. Further provided herein is use of the synthetic bacteria for the treatment of acne in a subject in need thereof. Further provided herein is a method of producing the synthetic bacteria comprising engineering the non-pathogenic bacteria to produce to the synthetic bacteria.

In another aspect of the disclosure, provided herein is a synthetic bacteria derived from a non-pathogenic bacteria, the synthetic bacteria adapted to secrete an AP-1 inhibitor. In some embodiments, the non-pathogenic bacteria is Propionibacterium acnes. Further provided herein is use of the synthetic bacteria for the treatment of acne in a subject in need thereof. Further provided herein is a method of producing the synthetic bacteria comprising engineering the non-pathogenic bacteria to produce to the synthetic bacteria.

In another aspect of the disclosure, provided herein is a synthetic bacteria derived from a non-pathogenic bacteria, the synthetic bacteria adapted to secrete a retinoid or a derivative thereof. In some embodiments, the retinoid or derivative thereof comprise retinol, adapalene, tretinoin, tazarotene, retinoic acid, or a combination thereof. In some embodiments, the non-pathogenic bacteria is Propionibacterium acnes. Further provided herein is use of the synthetic bacteria for the treatment of acne in a subject in need thereof. Further provided herein is a method of producing the synthetic bacteria comprising engineering the non-pathogenic bacteria to produce to the synthetic bacteria.

In another aspect of the disclosure, provided herein is a synthetic bacteria derived from a non-pathogenic bacteria, the synthetic bacteria adapted to secrete a compound with a binding affinity for retinoid binding protein; and provided that binding of the compound with the retinoid binding protein activates the retinoid binding protein. In some embodiments, the non-pathogenic bacteria is Propionibacterium acnes. Further provided herein is use of the synthetic bacteria for the treatment of acne in a subject in need thereof. Further provided herein is a method of producing the synthetic bacteria comprising engineering the non-pathogenic bacteria to produce to the synthetic bacteria.

In another aspect of the disclosure, provided herein is a synthetic bacteria derived from a non-pathogenic bacteria, the synthetic bacteria adapted to secrete a PPAR-ligand inhibitor. In some embodiments, the non-pathogenic bacteria is Propionibacterium acnes. Further provided herein is use of the synthetic bacteria for the treatment of acne in a subject in need thereof. Further provided herein is a method of producing the synthetic bacteria comprising engineering the non-pathogenic bacteria to produce to the synthetic bacteria.

In another aspect of the disclosure, provided herein is a synthetic bacteria derived from a non-pathogenic bacteria, the synthetic bacteria adapted to secrete one or more molecules heterologous to the non-pathogenic bacteria. In some embodiments, the one or more molecules comprise a human hormone, interleukin, antibody, or a combination thereof. In some embodiments, the non-pathogenic bacteria is Propionibacterium acnes. Further provided herein is use of the synthetic bacteria for the treatment of acne in a subject in need thereof. Further provided herein is a method of producing the synthetic bacteria comprising engineering the non-pathogenic bacteria to produce to the synthetic bacteria.

In another aspect of the disclosure, provided herein is a synthetic bacteria derived from a non-pathogenic bacteria, the synthetic bacteria adapted to secrete a single domain antibody fragment specific for TNF-alpha. In some embodiments, the non-pathogenic bacteria is Propionibacterium acnes. Further provided herein is use of the synthetic bacteria for the treatment of acne in a subject in need thereof. Further provided herein is a method of producing the synthetic bacteria comprising engineering the non-pathogenic bacteria to produce to the synthetic bacteria.

In another aspect of the disclosure, provided herein is a synthetic bacteria derived from a non-pathogenic bacteria, the synthetic bacteria adapted to express human Trefoil Factor 1. In some embodiments, the non-pathogenic bacteria is Propionibacterium acnes. Further provided herein is use of the synthetic bacteria for the treatment of acne in a subject in need thereof. Further provided herein is a method of producing the synthetic bacteria comprising engineering the non-pathogenic bacteria to produce to the synthetic bacteria.

In another aspect of the disclosure, provided herein is a synthetic bacteria derived from a non-pathogenic bacteria, the synthetic bacteria adapted to express a non-toxic level of an adhesion antibody specific for a cell surface protein of a keratinocyte. In some embodiments, the non-pathogenic bacteria is Propionibacterium acnes. Further provided herein is use of the synthetic bacteria for the treatment of acne in a subject in need thereof. Further provided herein is a method of producing the synthetic bacteria comprising engineering the non-pathogenic bacteria to produce to the synthetic bacteria.

In another aspect of the disclosure, provided herein is a method of generating a synthetic bacteria from a non-pathogenic bacteria, the method comprising engineering the non-pathogenic bacteria with a transcription activator-like effector nuclease (TALEN) and a clustered regulatory interspaced palindromic repeat (CRISPR)/Cas9 endonuclease. In some embodiments, the non-pathogenic bacteria is Propionibacterium acnes. Further provided herein is the synthetic bacteria. Further provided herein is a method of using the synthetic bacteria for the treatment of acne in a subject in need thereof.

In another aspect of the disclosure, provided herein is a method of generating a synthetic bacteria from a non-pathogenic bacteria, the method comprising introducing an exogenous biomolecule into the non-pathogenic bacteria. In some embodiments, the exogenous biomolecule is introduced using a transient delivery system. In some embodiments, the transient delivery system comprises a type III secretion system from a bacteria. In some embodiments, the non-pathogenic bacteria is Propionibacterium acnes. Further provided herein is the synthetic bacteria. Further provided herein is a method of using the synthetic bacteria for the treatment of acne in a subject in need thereof.

In another aspect of the disclosure, provided herein is a method of generating a synthetic bacteria from a non-pathogenic bacteria, the method comprising engineering the non-pathogenic bacteria to deliver a therapeutic biomolecule to a mammalian cell. In some embodiments, the therapeutic biomolecule is a vaccine. In some embodiments, the therapeutic biomolecule is a peptide or protein. In some embodiments, the therapeutic biomolecule is an enzyme. In some embodiments, the therapeutic biomolecule is a transcription factor. In some embodiments, the transcription factor is MyoD. In some embodiments, the therapeutic biomolecule is a TALEN. In some embodiments, the non-pathogenic bacteria is Propionibacterium acnes. Further provided herein is the synthetic bacteria. Further provided herein is a method of using the synthetic bacteria for the treatment of acne in a subject in need thereof.

In another aspect of the disclosure, provided herein is a method of generating a synthetic bacteria from a non-pathogenic bacteria, the method comprising engineering the non-pathogenic bacteria to have controlled expression of a payload protein. In some embodiments, the non-pathogenic bacteria is Propionibacterium acnes. Further provided herein is the synthetic bacteria. Further provided herein is a method of using the synthetic bacteria for the treatment of acne in a subject in need thereof.

In another aspect of the disclosure, provided herein is a method of generating a synthetic bacteria from a non-pathogenic bacteria, the method comprising engineering the non-pathogenic bacteria to express a programmable adhesion molecule specific for a target surface or cell. In some embodiments, the non-pathogenic bacteria is Propionibacterium acnes. Further provided herein is the synthetic bacteria. Further provided herein is a method of using the synthetic bacteria for the treatment of acne in a subject in need thereof.

In another aspect of the disclosure, provided herein is a method of generating a synthetic bacteria from a non-pathogenic bacteria, the method comprising engineering the non-pathogenic bacteria to have a stable memory to detect the presence of a small molecule. In some embodiments, the non-pathogenic bacteria is Propionibacterium acnes. Further provided herein is the synthetic bacteria. Further provided herein is a method of using the synthetic bacteria for the treatment of acne in a subject in need thereof.

In another aspect of the disclosure, provided herein is a method of generating a synthetic bacteria from a non-pathogenic bacteria, the method comprising engineering the non-pathogenic bacteria to exhibit tropism-bacterial chemotaxis toward a pathogen. In some embodiments, the non-pathogenic bacteria is engineered to comprise a chemoreceptor and/or chemoeffector. In some embodiments, the non-pathogenic bacteria is Propionibacterium acnes. Further provided herein is the synthetic bacteria. Further provided herein is a method of using the synthetic bacteria for the treatment of acne in a subject in need thereof.

In another aspect of the disclosure, provided herein is a method of generating a synthetic bacteria from a non-pathogenic bacteria, the method comprising engineering the non-pathogenic bacteria to secrete a phenolic compound. In some embodiments, the phenolic compound comprises a flavonol, flavone, flavanone, flavanol, isoflavone, antocyjanidin, hydroxycinnamic acid, hydroxybenzoic acid, tannin, stilbene, lignin, or a combination thereof. In some embodiments, the non-pathogenic bacteria is Propionibacterium acnes. Further provided herein is the synthetic bacteria. Further provided herein is a method of using the synthetic bacteria for the treatment of acne in a subject in need thereof.

In another aspect of the disclosure, provided herein is a method of generating a synthetic bacteria from a non-pathogenic bacteria, the method comprising engineering the non-pathogenic bacteria to secrete an acne medicament. In some embodiments, the acne medicament comprises benzoyl peroxide, salicylic acid, glycolic acid, clindamycin, erythromycin, Bactrim, doxycycline, tetracycline, minioctcline, spironolactone, retinoids, tacrolimus, pimecrolimus, a steroid, aspirin, ibuprofen, dapsone, azaleic acid, an alphahyroxy acid, a keratolytic, sulfacetamide sulfur, or a combination thereof. In some embodiments, the non-pathogenic bacteria is Propionibacterium acnes. Further provided herein is the synthetic bacteria. Further provided herein is a method of using the synthetic bacteria for the treatment of acne in a subject in need thereof.

In another aspect of the disclosure, provided herein is a method of generating a synthetic bacteria from a non-pathogenic bacteria, the method comprising engineering the non-pathogenic bacteria to secrete a plant derived extract. In some embodiments, the plant derived extract is from Aloe vera, Azadirachta indica, Curcuma ionga, Hemidesmus indicus, Terminalia chebula, Withania somnifera, Butyrospermum paradoxum, Camellia sinensis L., Commiphora mukul, Hippophae rhamnoides L., Lens culinaris, Aloe barbadensis, Vitex negundo, Andrographis paniculata, Salmalia malabarica, Melaleuca alternifolia, or a combination thereof. In some embodiments, the non-pathogenic bacteria is Propionibacterium acnes. Further provided herein is the synthetic bacteria. Further provided herein is a method of using the synthetic bacteria for the treatment of acne in a subject in need thereof.

In another aspect of the disclosure, provided herein is a method of generating a synthetic bacteria from a non-pathogenic bacteria, the method comprising engineering the non-pathogenic bacteria to produce lactoferrin. In some embodiments, the non-pathogenic bacteria is Propionibacterium acnes. Further provided herein is the synthetic bacteria. Further provided herein is a method of using the synthetic bacteria for the treatment of acne in a subject in need thereof.

In another aspect of the disclosure, provided herein is a method of generating a synthetic bacteria from a non-pathogenic bacteria, the method comprising engineering the non-pathogenic bacteria to produce an active agent from an herb. In some embodiments, the herb comprises Forsythia suspensa (Thunb.) Vahl., Taraxacum mongolicum Hand.-Mazz., Lonicera japonica Thunb., Lonicera hypoglauca Miq., Lonicera confusa D.C., Lonicera dasystyla Rehd., Coix lacryma-jobi L. var. ma-yuen (Roman.) Stapf, Rheum palmatum L., Rheum tanguticum Maxim. Ex Balf., Rheum officinale Baill., Angelica dahurica Benth. Et Hood. F., Angelica dahurica Benth. Et Hook. F. var. formosana Shan et Yuan, Scutellaria baicalensis Georgi, Paeonia suffruticosa Andr., Salvia miltiorrhiza Bge., Morus alba L., or a combination thereof. In some embodiments, the herb comprises Lian Qiao, Pu Gong Ying, Jin Yin Hua, Yi Yi Ren, Da Huang, Bai Zhi, Huang Qin, Mu Dan Pi, Dan Shen, Sang Bai Pi, Qing-Shang-Fang-Feng-Tang, Zhen-Ren-Huo-Ming-Yin, Jia-Wei-Xiao-Yao-San, Wu-Wei-Xiao-Du-Yin, Huang-Lian-Jie-Du-Tang, or a combination thereof. In some embodiments, the non-pathogenic bacteria is Propionibacterium acnes. Further provided herein is the synthetic bacteria. Further provided herein is a method of using the synthetic bacteria for the treatment of acne in a subject in need thereof.

In another aspect of the disclosure, provided herein is a method of generating a synthetic bacteria from a non-pathogenic bacteria, the method comprising engineering the non-pathogenic bacteria to produce an antioxidant, niacinamide, alpha-hydroxy acid, salicylic acid, lipo-hydroxy acid, retinol, linoleic acid, lauric acid, retinaldehyde, zinc, zinc salt, alpha-linolenic, eicosapentaenoic acid, docosahexaenoic acid, tea tree oil, fatty acid, glycolic acid, lauric acid, benzoyl peroxide, undecyl-rhamnoside, SIG1273 gel, oat plantlet extract, or a derivative or combination thereof. In some embodiments, the non-pathogenic bacteria is Propionibacterium acnes. Further provided herein is the synthetic bacteria. Further provided herein is a method of using the synthetic bacteria for the treatment of acne in a subject in need thereof.

In another aspect of the disclosure, provided herein is a method of generating a synthetic bacteria from a non-pathogenic bacteria, the method comprising engineering the non-pathogenic bacteria to produce a sensor effector specific for a target molecule. In some embodiments, the sensor effector is an RNA molecule that modulates expression in the bacteria producing the target molecule. In some embodiments, the sensor effector is a transcription factor that modulates expression in the bacteria producing the target molecule. In some embodiments, the sensor effector modulates metabolism of the synthetic bacteria in response to the target molecule. In some embodiments, the sensor effector modulates metabolism of the synthetic bacteria in response to a threshold level of a target molecule. In some embodiments, the target molecule is a porphyrin. In some embodiments, the target molecule is a porphyrin and the threshold level is about 4 micromolar or greater. In some embodiments, the synthetic bacteria is adapted to kill or attenuate a bacteria producing the target molecule. In some embodiments, the synthetic bacteria is adapted to kill or attenuate a bacteria producing a level of the target molecule above a threshold level. In some embodiments, the target molecule is a porphyrin. In some embodiments, the target molecule is a porphyrin and the threshold level is about or less than about 4 micromolar. In some embodiments, the non-pathogenic bacteria is Propionibacterium acnes. Further provided herein is the synthetic bacteria. Further provided herein is a method of using the synthetic bacteria for the treatment of acne in a subject in need thereof.

In another aspect of the disclosure, provided herein is a method of generating a synthetic bacteria from a non-pathogenic bacteria, the method comprising engineering the non-pathogenic bacteria to produce a sensor effector to coordinate an activity between itself and one or more additional organisms. In some embodiments, the activity is to regulate cell lysis of the synthetic bacteria to regulate density of the synthetic bacteria. In some embodiments, the activity is to limit or prevent growth of a target bacteria. In some embodiments, the target bacteria is a disease-associated strain of a bacteria. In some embodiments, the target bacteria is an antibiotic resistant bacteria. In some embodiments, the activity is to treat a disease. In some embodiments, the activity is to prevent the disease. In some embodiments, the activity is coordination of chemical exchange and metabolism to produce a desired compound. In some embodiments, the non-pathogenic bacteria is Propionibacterium acnes. Further provided herein is the synthetic bacteria. Further provided herein is a method of using the synthetic bacteria for the treatment of acne in a subject in need thereof.

In another aspect of the disclosure, provided herein is a synthetic bacteria, the synthetic bacteria adapted to exhibit increased expression or activity of a deoR repressor operon. In certain embodiments, the synthetic bacteria was derived from a non-pathogenic bacteria. In certain embodiments, the synthetic bacteria was derived from a Propionibacterium acnes strain. In certain embodiments, the synthetic bacteria is a Propionibacterium acnes ribotype 1 or ribotype 3 strain. In certain embodiments, the synthetic bacteria is a Propionibacterium acnes ribotype 1 strain. In certain embodiments, described herein is a composition comprising the synthetic bacteria formulated in a cream, emulsion, gel, ointment, liposome, or nanoparticle. In certain embodiments, the composition further comprises a biological stabilizer effective to prevent death of any or more synthetic bacteria. In certain embodiment the synthetic bacteria is for use in the treatment of acne. In certain embodiment the synthetic bacteria is for use in the treatment of eczema. In certain embodiment the synthetic bacteria is for use in the treatment of psoriasis.

In another aspect of the disclosure, provided herein is a method to treat an individual with a skin disease comprising administering a compound comprising a synthetic bacteria, the synthetic bacteria adapted to exhibit increased expression or activity of a deoR repressor operon. In certain embodiments, the synthetic bacteria was derived from a non-pathogenic bacteria. In certain embodiments, the synthetic bacteria was derived from a Propionibacterium acnes strain. In certain embodiments, the synthetic bacteria is Propionibacterium acnes ribotype 1 or ribotype 3 strain. In certain embodiments, the synthetic bacteria is a Propionibacterium acnes ribotype 1 strain. In certain embodiments, the synthetic bacteria is formulated as a composition for topical application, wherein the composition for topical application is formulated as a lotion, cream, emulsion, gel, ointment, liposome, or nanoparticle. In certain embodiments, the composition further comprises a biological stabilizer effective to prevent death of any or more synthetic bacteria. In certain embodiments, the skin disease is acne. In certain embodiments, the skin disease is eczema. In certain embodiments, the skin disease is psoriasis.

In another aspect of the disclosure, provided herein is a synthetic bacteria, the synthetic bacteria adapted to exhibit increased expression or activity of a type II lipase. In certain embodiments, the synthetic bacteria was derived from a non-pathogenic bacteria. In certain embodiments, the synthetic bacteria was derived from a Propionibacterium acnes strain. In certain embodiments, the synthetic bacteria is a Propionibacterium acnes ribotype 1 or ribotype 3 strain. In certain embodiments, the synthetic bacteria is a Propionibacterium acnes ribotype 1 strain. In certain embodiments, described herein is a composition comprising the synthetic bacteria formulated in a cream, emulsion, gel, ointment, liposome, or nanoparticle. In certain embodiments, the composition further comprises a biological stabilizer effective to prevent death of any or more synthetic bacteria. In certain embodiment the synthetic bacteria is for use in the treatment of acne. In certain embodiment the synthetic bacteria is for use in the treatment of eczema. In certain embodiment the synthetic bacteria is for use in the treatment of psoriasis.

In another aspect of the disclosure, provided herein is a method to treat an individual with a skin disease comprising administering a compound comprising a synthetic bacteria, the synthetic bacteria adapted to exhibit increased expression or activity of a type II lipase. In certain embodiments, the synthetic bacteria was derived from a non-pathogenic bacteria. In certain embodiments, the synthetic bacteria was derived from a Propionibacterium acnes strain. In certain embodiments, the synthetic bacteria is Propionibacterium acnes ribotype 1 or ribotype 3 strain. In certain embodiments, the synthetic bacteria is a Propionibacterium acnes ribotype 1 strain. In certain embodiments, the synthetic bacteria is formulated as a composition for topical application, wherein the composition for topical application is formulated as a lotion, cream, emulsion, gel, ointment, liposome, or nanoparticle. In certain embodiments, the composition further comprises a biological stabilizer effective to prevent death of any or more synthetic bacteria. In certain embodiments, the skin disease is acne. In certain embodiments, the skin disease is eczema. In certain embodiments, the skin disease is psoriasis.

In another aspect of the disclosure, provided herein is a synthetic bacteria, the synthetic bacteria adapted to exhibit decreased expression or activity of a type I lipase. In certain embodiments, the synthetic bacteria was derived from a non-pathogenic bacteria. In certain embodiments, the synthetic bacteria was derived from a Propionibacterium acnes strain. In certain embodiments, the synthetic bacteria is a Propionibacterium acnes ribotype 1 or ribotype 3 strain. In certain embodiments, the synthetic bacteria is a Propionibacterium acnes ribotype 1 strain. In certain embodiments, described herein is a composition comprising the synthetic bacteria formulated in a cream, emulsion, gel, ointment, liposome, or nanoparticle. In certain embodiments, the composition further comprises a biological stabilizer effective to prevent death of any or more synthetic bacteria. In certain embodiment the synthetic bacteria is for use in the treatment of acne. In certain embodiment the synthetic bacteria is for use in the treatment of eczema. In certain embodiment the synthetic bacteria is for use in the treatment of psoriasis.

In another aspect of the disclosure, provided herein is a method to treat an individual with a skin disease comprising administering a compound comprising a synthetic bacteria, the synthetic bacteria adapted to exhibit decreased expression or activity of a type I lipase. In certain embodiments, the synthetic bacteria was derived from a non-pathogenic bacteria. In certain embodiments, the synthetic bacteria was derived from a Propionibacterium acnes strain. In certain embodiments, the synthetic bacteria is Propionibacterium acnes ribotype 1 or ribotype 3 strain. In certain embodiments, the synthetic bacteria is a Propionibacterium acnes ribotype 1 strain. In certain embodiments, the synthetic bacteria is formulated as a composition for topical application, wherein the composition for topical application is formulated as a lotion, cream, emulsion, gel, ointment, liposome, or nanoparticle. In certain embodiments, the composition further comprises a biological stabilizer effective to prevent death of any or more synthetic bacteria. In certain embodiments, the skin disease is acne. In certain embodiments, the skin disease is eczema. In certain embodiments, the skin disease is psoriasis.

In another aspect of the disclosure, provided herein is a synthetic bacteria, the synthetic bacteria adapted to exhibit decreased expression, activity or deletion of a dermatin-sulfate adhesin (DSA1 or DSA2). In certain embodiments, the synthetic bacteria was derived from a non-pathogenic bacteria. In certain embodiments, the synthetic bacteria was derived from a Propionibacterium acnes strain. In certain embodiments, the synthetic bacteria is a Propionibacterium acnes ribotype 1 or ribotype 3 strain. In certain embodiments, the synthetic bacteria is a Propionibacterium acnes ribotype 1 strain. In certain embodiments, described herein is a composition comprising the synthetic bacteria formulated in a cream, emulsion, gel, ointment, liposome, or nanoparticle. In certain embodiments, the composition further comprises a biological stabilizer effective to prevent death of any or more synthetic bacteria. In certain embodiment the synthetic bacteria is for use in the treatment of acne. In certain embodiment the synthetic bacteria is for use in the treatment of eczema. In certain embodiment the synthetic bacteria is for use in the treatment of psoriasis.

In another aspect of the disclosure, provided herein is a method to treat an individual with a skin disease comprising administering a compound comprising a synthetic bacteria, the synthetic bacteria adapted to exhibit decreased expression, activity or deletion of a dermatin-sulfate adhesin (DSA1 or DSA2). In certain embodiments, the synthetic bacteria was derived from a non-pathogenic bacteria. In certain embodiments, the synthetic bacteria was derived from a Propionibacterium acnes strain. In certain embodiments, the synthetic bacteria is Propionibacterium acnes ribotype 1 or ribotype 3 strain. In certain embodiments, the synthetic bacteria is a Propionibacterium acnes ribotype 1 strain. In certain embodiments, the synthetic bacteria is formulated as a composition for topical application, wherein the composition for topical application is formulated as a lotion, cream, emulsion, gel, ointment, liposome, or nanoparticle. In certain embodiments, the composition further comprises a biological stabilizer effective to prevent death of any or more synthetic bacteria. In certain embodiments, the skin disease is acne. In certain embodiments, the skin disease is eczema. In certain embodiments, the skin disease is psoriasis.

In another aspect of the disclosure, provided herein is a synthetic bacteria, the synthetic bacteria adapted to exhibit decreased loss or deletion of a pIMPLE plasmid virulence factor. In certain embodiments, the synthetic bacteria was derived from a non-pathogenic bacteria. In certain embodiments, the synthetic bacteria was derived from a Propionibacterium acnes strain. In certain embodiments, the synthetic bacteria is a Propionibacterium acnes ribotype 1 or ribotype 3 strain. In certain embodiments, the synthetic bacteria is a Propionibacterium acnes ribotype 1 strain. In certain embodiments, described herein is a composition comprising the synthetic bacteria formulated in a cream, emulsion, gel, ointment, liposome, or nanoparticle. In certain embodiments, the composition further comprises a biological stabilizer effective to prevent death of any or more synthetic bacteria. In certain embodiment the synthetic bacteria is for use in the treatment of acne. In certain embodiment the synthetic bacteria is for use in the treatment of eczema. In certain embodiment the synthetic bacteria is for use in the treatment of psoriasis.

In another aspect of the disclosure, provided herein is a method to treat an individual with a skin disease comprising administering a compound comprising a synthetic bacteria, the synthetic bacteria adapted to exhibit decreased loss or deletion of a pIMPLE plasmid virulence factor. In certain embodiments, the synthetic bacteria was derived from a non-pathogenic bacteria. In certain embodiments, the synthetic bacteria was derived from a Propionibacterium acnes strain. In certain embodiments, the synthetic bacteria is Propionibacterium acnes ribotype 1 or ribotype 3 strain. In certain embodiments, the synthetic bacteria is a Propionibacterium acnes ribotype 1 strain. In certain embodiments, the synthetic bacteria is formulated as a composition for topical application, wherein the composition for topical application is formulated as a lotion, cream, emulsion, gel, ointment, liposome, or nanoparticle. In certain embodiments, the composition further comprises a biological stabilizer effective to prevent death of any or more synthetic bacteria. In certain embodiments, the skin disease is acne. In certain embodiments, the skin disease is eczema. In certain embodiments, the skin disease is psoriasis.

In another aspect of the disclosure, provided herein is a synthetic bacteria, the synthetic bacteria adapted to exhibit loss or deletion of a thiopepeptide encoding island. In certain embodiments, the synthetic bacteria was derived from a non-pathogenic bacteria. In certain embodiments, the synthetic bacteria was derived from a Propionibacterium acnes strain. In certain embodiments, the synthetic bacteria is a Propionibacterium acnes ribotype 1 or ribotype 3 strain. In certain embodiments, the synthetic bacteria is a Propionibacterium acnes ribotype 1 strain. In certain embodiments, described herein is a composition comprising the synthetic bacteria formulated in a cream, emulsion, gel, ointment, liposome, or nanoparticle. In certain embodiments, the composition further comprises a biological stabilizer effective to prevent death of any or more synthetic bacteria. In certain embodiment the synthetic bacteria is for use in the treatment of acne. In certain embodiment the synthetic bacteria is for use in the treatment of eczema. In certain embodiment the synthetic bacteria is for use in the treatment of psoriasis.

In another aspect of the disclosure, provided herein is a method to treat an individual with a skin disease comprising administering a compound comprising a synthetic bacteria, the synthetic bacteria adapted to exhibit loss or deletion of a thiopepeptide encoding island. In certain embodiments, the synthetic bacteria was derived from a non-pathogenic bacteria. In certain embodiments, the synthetic bacteria was derived from a Propionibacterium acnes strain. In certain embodiments, the synthetic bacteria is Propionibacterium acnes ribotype 1 or ribotype 3 strain. In certain embodiments, the synthetic bacteria is a Propionibacterium acnes ribotype 1 strain. In certain embodiments, the synthetic bacteria is formulated as a composition for topical application, wherein the composition for topical application is formulated as a lotion, cream, emulsion, gel, ointment, liposome, or nanoparticle. In certain embodiments, the composition further comprises a biological stabilizer effective to prevent death of any or more synthetic bacteria. In certain embodiments, the skin disease is acne. In certain embodiments, the skin disease is eczema. In certain embodiments, the skin disease is psoriasis.

In another aspect of the disclosure, provided herein, is a composition comprising a therapeutically effective amount of a synthetic bacteria, the synthetic bacteria adapted to exhibit increased or decreased expression or activity of hyaluronidase. In certain embodiments, the synthetic bacteria comprises an insertion, deletion, or frameshift in a hyaluronidase gene that reduces or eliminates hyaluronidase activity. In certain embodiments, the synthetic bacteria comprises a P. acnes strain. In certain embodiments, the synthetic bacteria comprises an RT1 or RT2 ribotype. In certain embodiments, the synthetic bacteria comprises the DeoR gene. In certain embodiments, the synthetic bacteria comprises a type II lipase gene. In certain embodiments, the type II lipase gene comprises gehA or gehB. In certain embodiments, the synthetic bacteria lacks the PIMPLE plasmid. In certain embodiments, the synthetic bacteria comprises a CRISPR locus or portion thereof. In certain embodiments, the composition comprises an excipient or biological stabilizer. In certain embodiments, this excipient or biologic stabilizer is glycerol. In certain embodiments, the composition is stable at room temperature. In certain embodiments, the composition is formulated for topical administration. In certain embodiments, described herein, is a method of treating an individual with acne comprising administering the composition comprising the synthetic bacteria

In a certain aspect, described herein is a synthetic bacteria derived from a non-pathogenic bacteria, the synthetic bacteria comprising: a) a porphyrin pathway comprising a biomolecule introduced, removed, or modified relative to the porphyrin pathway of the non-pathogenic bacteria; provided that porphyrin is not produced by the synthetic bacteria or is produced at a level less than about 4 micromolar of porphyrin in the synthetic bacteria; b) a fatty acid synthesis pathway comprising a biomolecule introduced, removed, or modified relative to the fatty acid synthesis pathway of the non-pathogenic bacteria; provided that the synthetic bacteria produces fatty acids; c) a vitamin B12 metabolic pathway comprising a biomolecule introduced, removed, or modified relative to the vitamin B12 metabolic pathway of the non-pathogenic bacteria; provided that the synthetic bacteria produces a high levels of intracellular vitamin B12 as compared to the non-pathogenic bacteria; d) a modified lipase compared to a native lipase; e) a hyaluronidase from a heterologous species as the non-pathogenic bacteria; f) a nitrous oxide pathway comprising a biomolecule introduced, removed, or modified relative to the nitrous oxide pathway of the non-pathogenic bacteria; provided that the synthetic bacteria produces nitrous oxide; g) a biomolecule introduced, removed, or modified relative to the citric acid pathway of the non-pathogenic bacteria; provided that the synthetic bacteria produces glycine; h) a biomolecule introduced, wherein the biomolecule is adapted to produce an enzyme at a tunable level to effect a second metabolic pathway of the synthetic bacteria; i) a biomolecule introduced, wherein the biomolecule is adapted to secrete a corticotropin releasing hormone (CRH), a corticotropin releasing hormone receptor (CRHR), a corticotropin releasing hormone binding protein (CRHBP), or a combination thereof, j) a biomolecule introduced, wherein the biomolecule is adapted to secrete a TNF-alpha inhibitor, an interleukin-8 inhibitor, a tumor neutrophil chemotaxis inhibitor, an interleukin-6 inhibitor, an NFkB inhibitor, human beta defensing-1 inhibitor, human beta defensing-2 inhibitor, or a human beta defensing-3 inhibitor; or k) any combination of a), b), c), d), e), f), g), h), i), or j). In certain embodiments, the non-pathogenic bacteria is Propionibacterium acnes. In certain embodiments, the Propionibacterium acnes bacteria comprises Ribotype 1 or Ribotype 2. In certain embodiments, the non-pathogenic bacteria has been engineered or selected to comprise at least one gene encoding at least one of a deoxyribose operon repressor and a type II lipase. In certain embodiments, a Cas 5 protein is absent from the non-pathogenic bacteria. In certain embodiments, the non-pathogenic bacteria comprises less than about 10% pIMPLE plasmid. In certain embodiments, the non-pathogenic bacteria does not comprise an RT6 genotype. In certain embodiments, the non-pathogenic bacteria is a strain selected from at least one of HP3A11, HP3B4, HP4G1, and HP5G4. In certain embodiments, the non-pathogenic bacteria expresses an ATP binding cassette transporter. In certain embodiments, the non-pathogenic bacteria does not express a DNA binding response regulator or a phosphoglycerate kinase. In certain embodiments, the biomolecule comprises an enzyme encoded by one or more of the following genes: cysG-cbiX, cobI-cbiL, cobM, cbiF, cobK, cbiJ, cobH, cbiC, cobB-cbiA, cobO, btuR, cobQ, cbiP, cbiB, cobD, cobS, and cobV. In certain embodiments, the biomolecule comprises an enzyme encoded by fadD. In certain embodiments, the biomolecule comprises an enzyme encoded by one or more of the following genes: COX15, cyoE, and HemB, HemC, HemD, HemE, HemF, HemG, HemH, HemY, or PPA2095 protoporphyrinogen oxidase (HemY homologue). In certain embodiments, the biomolecule comprises a stop codon or truncation in any one or more of the following genes: COX15, cyoE, and HemB, HemC, HemD, HemE, HemF, HemG, HemH, HemY, or PPA2095 protoporphyrinogen oxidase (HemY homologue). In certain embodiments, the biomolecule comprises an enzyme encoded by one or more of the following genes: narJ, narI, narH, narG, E.1.7.2.1, norB, gdhA, gudB, and rocG. In certain embodiments, the biomolecule comprises an enzyme encoded by one or more of the following genes: CS, IDH1, OGDH, DLST, and fumC. In certain embodiments, the biomolecule comprises an enzyme wherein the enzyme comprises ST2S, ST4S, ST6S, bmpA, PTS-Mtl-EIIABC, MFS ST1, MFS ST2, ST1P, ST3P, ST4P, ST5P, ST6P, ST7P, PTS-Mtl-EIIA, ST1P1, ST3P1, ST4P1, ST6P1, FhuD, ST7A, manA, FhuC, FhuB, FhuD, COX15, talAB, hMuV, HtaA, HmuT, GAPDH, hemH, CS, IDH1, cobA-hemD, cysG, IDH1, IDH1, TGL, OGDH, narJ, narl, narH, narG, E1.7.2.1, norB, gdhA, gudB, rocG, MDT1, MDT2, MDT3, T2SF2, T2SF1, secA, secY, secF, secD, yajC, secE, ftsY, yidC, secG, clpX, clp2, clpi. In certain embodiments, the modified lipase has no lipase activity or a lipase activity less than about 50% of the activity of the native lipase. In certain embodiments, the modified lipase comprises a disruption to a gene selected from HMPREF0675_4855, HMPREF0675_4856, HMPREF0675_4479, HMPREF0675_4480, HMPREF0675_4481, HMPREF0675_3655/3657, HMPREF0675_4816, HMPREF0675_4817, HMPREF0675_5205, HMPREF0675_5206, HMPREF0675_5014, HMPREF0675_5101, HMPREF0675_5159, HMPREF0675_4093/4094, HMPREF0675_4163, HMPREF0675_5031, HMPREF0675_5390, HMPREF0675_3037, or a homolog thereof having greater than 90%, homology. In certain embodiments, the hyaluronidase from a heterologous species comprises a hyaluronidase from a Group B Streptococcus. In certain embodiments, the synthetic bacteria of any one of claims and an excipient or biological stabilizer are formulated as a composition In certain embodiments, the composition is formulated for topical application. In certain embodiments, the composition is for use in treating a skin disorder. In certain embodiments, the skin disorder comprises acne, psoriasis, eczema, or atopic dermatitis, or seborrheic dermatitis. In certain embodiments, the composition is administered to the skin of an individual in need, wherein the composition comprises from about 10² cfu/cm² to about 10¹² cfu/cm² of the synthetic bacteria. In certain embodiments, the skin disorder comprises acne, psoriasis, eczema, or atopic dermatitis, or seborrheic dermatitis. In certain embodiments, is a method of making a synthetic bacteria described herein the method comprising introducing a Cas9, CRISPR RNA (crRNA), trans-activating RNA (tracrRNA), and a homology directed repair cassette (HDR) greater than 200 base pairs in length into a non-pathogenic bacteria. In certain embodiments, the HDR is greater than 500 basepairs in length. In certain embodiments, the HDR is greater than 900 basepairs in length.

### BRIEF DESCRIPTION OF THE DRAWINGS

The novel features of the disclosure are set forth with particularity in the appended claims. A better understanding of the features and advantages of the present disclosure will be obtained by reference to the following detailed description that sets forth illustrative embodiments, in which the principles of the disclosure are utilized, and the accompanying drawings of which:

FIG. 1 is a schematic showing the relationship between P. acnes and host inflammatory response.

FIG. 2 is another schematic showing the relationship between P. acnes and host inflammatory response.

FIG. 3 is a third schematic showing the relationship between P. acnes and host inflammatory response.

FIG. 4 is a graph showing differential expression of pro-inflammatory porphyrins between acne-associated and health-associated strains of P. acnes.

FIG. 5 is a scheme of a synthetic bacteria as generally described herein in certain embodiments.

FIG. 6 is a schematic showing biomolecules involved in the metabolic pathways of P. acnes and suitable for engineering to produce a synthetic bacteria as described herein.

FIG. 7 is a schematic showing strategies for combating disease by a pathogen using a synthetic bacteria.

FIG. 8 shows the percentage of reads from a sample of a healthy volunteer (free of acne) that map to P. acnes to ribotype RT1, which are both deoR+ and type II lipase positive, versus the percentage of reads from the samples that map to P. acnes RT2.

FIG. 9 shows results of an assay for P. acnes viability under different preservation conditions.

FIG. 10 shows a portion of a 23S, ribosomal RNA sequence from bacteria commonly found on the human face that enables characterization of a subject's skin microbiome. Numbers listed to the left of sequences correspond to bacterial strains as follows: (1) P. acnes_KPA171202_RT12; (2) P. acnes_KPA171202_RT1_3; (3) P. acnes ATCC 11828_RT2_1; (4) P. acnes ATCC 11828_RT2_2; (5) P. avidum 44067; (6) P. acidipropionici ATCC 4875; (7) S. aureus 04-02981; (8) S. aureus Bmb9393; (9) S. aureus FDA209P; (10) S. epidermidis ATCC 12228; and (11) S. epidermidis PM221. Sequences 1-5 correspond to SEQ ID NOs: 114 to 118. Sequence 6 corresponds to SEQ ID NO: 119. Sequences 7-11 correspond to SEQ ID NOs: 120-124.

FIG. 11 shows a standard curve generated with serial dilutions of a combination of health-associated P. acnes and S. epidermidis that can be used to quantitate a percentage of health-associated P. acnes in a collected sample.

FIG. 12 shows qPCR of successful CRISPR editing in P. acnes.

FIG. 13 shows mutations in a gene encoding a P. acnes type I lipase that result in a gene encoding a P. acnes type II lipase. Type I lipase Intergenic Region corresponds to SEQ ID NO: 125. Type I lipase Second Lipase (region) (HMPREF0675_4856) corresponds to SEQ ID NO: 126. Type II lipase Intergenic Region corresponds to SEQ ID NO: 127. Type II lipase Second Lipase (region) (HMPREF0675_4856) corresponds to SEQ ID NO: 128.

FIG. 14 shows exemplary packaging for compositions disclosed herein.

FIG. 15 shows bacteria viability of composition disclosed herein after being packaged under several conditions.

### DETAILED DESCRIPTION OF THE INVENTION

The present disclosure describes methods and systems for generating synthetic bacteria. These synthetic bacteria are useful for the treatment of a variety of disorders and diseases, including skin-related disorders such as acne, psoriasis, eczema, rosacea, and seborrheic dermatitis. Additional uses include preventing and treating age-associated changes to skin and skin health maintenance. Such use includes prevention of photo-aging, photodamage leading to rhytids, loss of dermal elasticity, and evolution of skin cancers.

Synthetic bacteria are generated from non-pathogenic bacteria to comprise one or more non-naturally occurring metabolic pathways. Non-naturally occurring metabolic pathways in a synthetic bacteria function to: express an entity of interest such as a biomolecule or compound; eliminate, attenuate and/or inactivate an entity of interest such as a biomolecule or compound; produce low levels of pro-inflammatory metabolites; modulate activity of an enzyme in the pathway; alter pathway ligands to prevent induction of host inflammatory response; act as a sensor effector and/or sensor receptor; synergize with other organisms in a consortium; attenuate or eliminate acquisition of antibiotic resistance in the synthetic bacteria; provide a therapeutic and/or a cosmetic affect when the synthetic bacteria is administered to a patient in need thereof; and any combination thereof. Further provided are methods of cloning, expressing, purifying and manufacturing synthetic bacteria. In various methods and systems, synthetic bacteria are generated from a non-pathogenic bacteria present in the human microbiome, such as Propionibacterium acnes found on human skin. Described herein are methods of preparing and using these synthetic bacteria to treat a skin disorder or disease.

Methods for generating synthetic bacteria generally involve generating a non-naturally occurring metabolic pathway in bacteria to affect production of an entity of interest such as a biomolecule or compound. In one aspect of the disclosure, provided are systems and methods for de novo synthesis of entities of interest by generating non-naturally occurring metabolic pathways within an organism to create said entities of interest. Entities include biomolecules and compounds. Biomolecules include peptides, proteins, nucleic acids, small molecules, carbohydrates, and lipids. In some cases, the affect is to produce a desired level of a biomolecule. For bacteria used in a treatment, this includes producing low levels of pro-inflammatory metabolites. A non-limiting example of a pro-inflammatory metabolite is a porphyrin. In some cases, a biomolecule produced is a hormone and/or hormone ligand inhibitor. For example, a hormone is corticotropin releasing hormone (CRH) and a hormone ligand inhibitor is corticotropin releasing hormone ligand (CRHL) inhibitor. In some cases, a biomolecule produced is a sensor receptor for detecting levels of another biomolecule. As a non-limiting example, a sensor receptor detects bacteria having high levels of the biomolecule porphyrin. For example, a sensor receptor detects bacteria having about 4 micromolar porphyrin or higher. Further examples of biomolecules sensed by a sensor receptor include lipase and glucose. Similarly, a biomolecule produced may be a sensor effector that modulates production and/or activity of another biomolecule. In some cases, a sensor effector modulates porphyrin production in the synthetic bacteria. In some cases, a sensor effector modulates lipase activity of a biomolecule. In some cases, a sensor effector modulates glucose metabolism in the synthetic bacteria.

In various embodiments, a biomolecule comprises a nucleic acid, DNA, RNA, a gene, an open reading frame, a promoter or enhancer, an operon, mRNA, rRNA, tRNA, a polypeptide, protein, enzyme, or an organic molecule able to exert an effect on a biological system.

In some embodiments, a metabolic pathway is engineered to attenuate or eliminate production of a biomolecule. In some embodiments, a biomolecule of interest produced is a mutated and/or otherwise altered from its native composition in non-synthetic bacteria. In some cases, the biomolecule is an enzyme and the biomolecule produced has attenuated or no enzymatic activity. As a non-limiting example, a biomolecule is an enzyme lacking lipase activity. In some embodiments, a biomolecule of interest produced by a non-naturally occurring metabolic pathway prevents and/or mitigates induction of a host inflammatory response. Examples of such biomolecules include ligands involved in host inflammatory response, such as toll like receptor (TLR) ligands TLR2 and TLR4. In some cases, the synthetic bacteria evade the host inflammatory response when applied to an individual.

Various synthetic bacteria described herein have biomolecules that prevent the acquisition of antibiotic resistance. For example, the 16S and/or 23S ribosomal RNA are mutated from their native sequence to prevent acquisition of antibiotic resistance.

In certain embodiments, a synthetic bacteria described herein has an ability to remove chemicals and/or deleterious molecules from a surface on which the bacteria is applied. In some cases, this removal involves the bacteria taking up the chemicals and/or deleterious molecules. In some cases, this removal involves destroying the chemicals and/or deleterious molecules. Non-limiting examples of chemicals and deleterious molecules include environmental toxins and pollutants.

In general, synthetic bacteria are sensitive to antibiotics and are free of mobile elements such as transposons and plasmids. Stable integration of recombinant DNA in the chromosome is the simplest way to minimize gene flow, but there are other strategies available, like a mutually dependent host-plasmid platform based on conditional origins of replication, auxotrophies and toxin anti-toxin pairs. In addition, synthetic bacteria generally have containment strategies resistant to environmental supplementation, mutagenic drift and horizontal gene transfer. Such safeguards avoid the spread of these bacteria into the environment as well as the proliferation of deleterious bacteria. Classically, biocontainment has been achieved through either engineered auxotrophies (e.g., strains deficient of thymidylate synthase) or induced lethality.

Minimal genomes encoding only the genes needed to sustain life might preclude unexpected evolution of synthetic bacteria. These minimal genomes could be generated through genome reduction techniques known to those of skill in the art. However, the definitive firewall for biocontainment might be the use of artificial genetic languages, such as those that incorporate a non-standard amino acid in the core of essential proteins, and replacing the synthetic thymine analog 5-chlorouracil instead of the natural thymine nucleotide in the bacterial DNA. Such strains may exhibit strong resistance to evolutionary escape through mutagenesis or horizontal gene transfer, and cannot be supplemented with natural compounds. Strains of type II and III P. acnes have clustered regularly interspaced short palindromic repeats (CRISPRs) and associated CAS genes which confer resistance to mobile genetic elements such as phages, plasmids and transposons, making these suitable candidates for synthetic bacteria production. In addition, they lack pili, which are noted in type IA strains and possibly related to virulence. Type IA strains also had upregulated lipase and immunogenic iron sequestration which increases virulence in these strains, in addition to differential CAMP expression and adhesion proteins, increase involucrin and decrease IL-8 induction of expression, all which may contribute to virulence of type IA strains.

In the following description, certain specific details are set forth in order to provide a thorough understanding of various embodiments. However, one skilled in the art will understand that the embodiments provided may be practiced without these details. Unless the context requires otherwise, throughout the specification and claims which follow, the word “comprise” and variations thereof, such as, “comprises” and “comprising” are to be construed in an open, inclusive sense, that is, as “including, but not limited to.” As used in this specification and the appended claims, the singular forms “a,” “an,” and “the” include plural referents unless the content clearly dictates otherwise. It should also be noted that the term “or” is generally employed in its sense including “and/or” unless the content clearly dictates otherwise. Further, headings provided herein are for convenience only and do not interpret the scope or meaning of the claimed embodiments.

As used herein the term “about” refers to an amount that is near the stated amount by about 10%, 5%, or 1%.

Various aspects now will be described more fully hereinafter. Such aspects may, however, be embodied in many different forms and should not be construed as limited to the embodiments set forth herein; rather, these embodiments are provided so that this disclosure will be thorough and complete, and will fully convey its scope to those skilled in the art.

Where a range of values is provided, it is intended that each intervening value between the upper and lower limit of that range and any other stated or intervening value in that stated range is encompassed within the disclosure. For example, if a range of 1 to 8 is stated, it is intended that 2, 3, 4, 5, 6, and 7 are also explicitly disclosed, as well as the range of values greater than or equal to 1 and the range of values less than or equal to 8.

Provided are definitions to commonly used terms herein, however, such definitions are not intended to limit the scope of the disclosure and in cases wherein a common interpretation of the term is broader than described herein, the definition herein is not meant to be limiting. As such, “amphiphilic” generally refers to a molecule combining hydrophilic and lipophilic (hydrophobic) properties. A “gel” generally refers to a colloid in which the dispersed phase has combined with the continuous phase to produce a semisolid material, such as jelly. “Hydrophilic” generally refers to substances that have strongly polar groups that readily interact with water. “Hydrophobic” generally refers to substances that lack an affinity for water; tending to repel and not absorb water as well as not dissolve in or mix with water. “Lipid soluble” generally refers to substances that have a solubility of greater than or equal to 5 g/100 mL in a hydrophobic liquid, such as castor oil. “Lipophilic” generally refers to compounds having an affinity for lipids. Examples of lipophilic substances include but are not limited to naturally occurring and synthetic oils, fats, fatty acids, lecithins, triglycerides and combinations thereof. An “oil” generally refers to a composition containing at least 95% wt of a lipophilic substance. “Skin” generally refers to the epidermis of an individual, such as a human, and in some embodiments includes, where specified, particular regions of the skin, such as the face, neck, arms, legs, abdomen, hands, back, buttocks, and/or feet. “Water soluble” generally refers to substances that have a solubility of greater than or equal to 5 g/100 mL water.

### Synthetic Bacteria

In one aspect of the disclosure, provided herein are synthetic bacteria engineered to produce one or more biomolecules effective in modulating the environment on which the synthetic bacteria are applied. In many cases, the environment is one of the skin, such as a follicle, and the synthetic bacteria are engineered from a non-pathogenic bacteria. FIG. 5 provides exemplary features of a synthetic bacteria, wherein a synthetic bacteria herein comprises any combination of said features. One feature is a chemotactic module that controls bacterial migration in response to environmental signals of interest. Another feature is a sensory module that detects environmental signals and responds by activating the transcription of a biomolecule and/or reporter modules. Another feature is an adhesion module that facilitates binding of the synthetic bacteria to a specific target cell or tissue. Another feature is a delivery module that allows for the release of a therapeutic molecule. Another feature is a containment module that prevents the environmental spread of the synthetic bacteria.

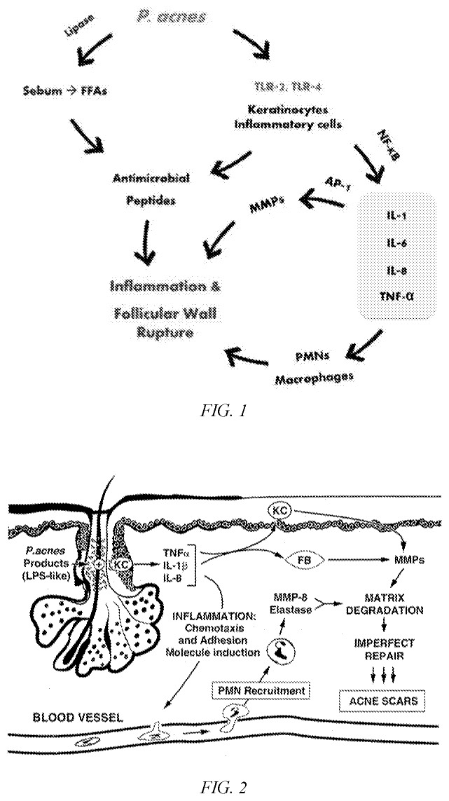

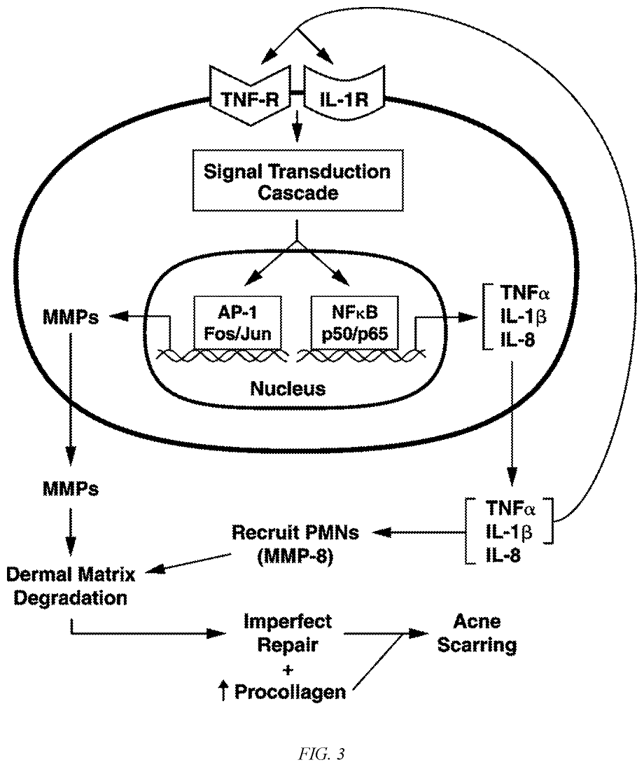

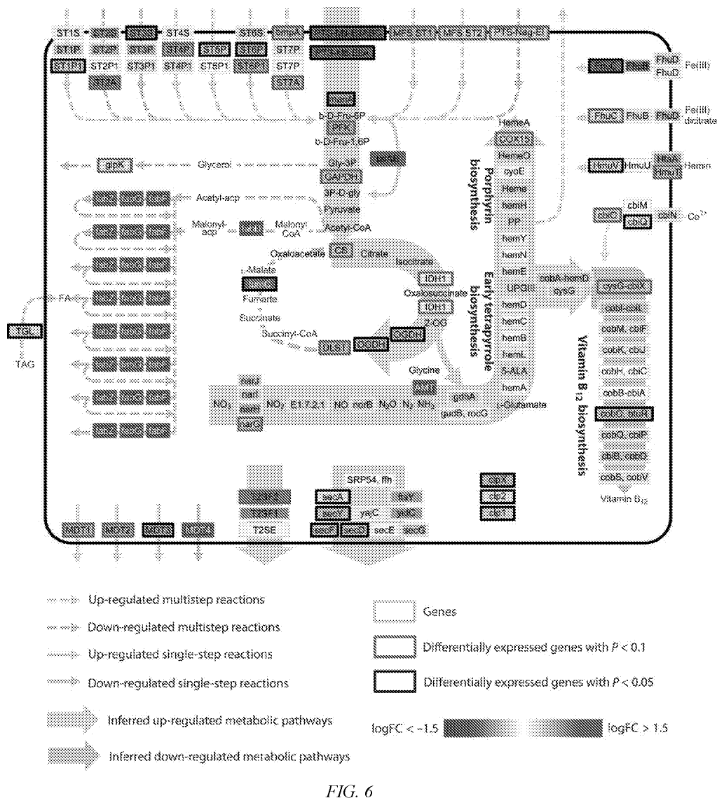

In various embodiments, a synthetic bacteria is engineered from a strain of P. acnes. FIGS. 1-3 provide schematics indicating the relationship between P. acnes and the host inflammatory response. Accordingly, biomolecules of any of the pathways and systems shown in the schematics of FIGS. 1-3 are suitable targets for introduction, manipulation, and/or removal in a health-associated P. acnes strain to produce a synthetic bacteria with desired characteristics, such as reduced inflammation and other indications related to a skin disorder like acne. FIG. 6 is a schematic showing metabolic pathways of P. acnes targeted for engineering to produce a synthetic bacteria as described herein. Table 1 provides a list of genes within P. acnes RT4 and RT5, loci 1 and 2, that are suitable targets for engineering to produce a synthetic bacteria. Table 2 provides a list of genes within P. acnes RT4 and RT5, locus 6, that are suitable targets for engineering to produce a synthetic bacteria. Table 3 provides a list of genes within P. acnes RT8, locus 4, that are suitable targets for engineering to produce a synthetic bacteria. Accordingly, it is provided herein that a disease-associated P. acnes bacteria is engineered to produce a synthetic bacteria having desired features as described elsewhere herein. For example, for use as a healthy probiotic such as a mimetic of a health-associated bacteria, a sensor detector, sensor effector, and the like.

TABLE 1
Target Genes in Loci 1 and 2 of P. acnes RT4 and RT5.

| Locus | ID | Description |
|---|---|---|
| Locus 1 | GM131 | ABC transporter ATP-binding protein |
| Locus 1 | GM132/GM133 ^{*2} | Site-specific recombinase |
| Locus 1 | GM134 | Site-specific recombinase |
| Locus 1 | GM135 | Hypothetical protein |
| Locus 1 | GM136 | Hypothetical protein |
| Locus 1 | GM137 | N-acetylmuramoyl-L-alanine amidase |
| Locus 2 | GM171 | Hypothetical protein |
| Locus 2 | GM172 | Hypothetical protein |
| Locus 2 | GM173 | Single-strand binding family protein |
| Locus 2 | GM174 | CobQ/CobB/MinD/ParA nucleotide |
| | | binding domain protein |
| Locus 2 | GM175 | Hypothetical protein |
| Locus 2 | GM176 | Hypothetical protein |
| Locus 2 | GM177 | Hypothetical protein |
| Locus 2 | GM178 | Hypothetical protein |
| Locus 2 | GM179 | Hypothetical protein |
| Locus 2 | GM180 | Hypothetical protein |
| Locus 2 | GM181 | CAXX amino protease family protein |
| Locus 2 | GM182 | Hypothetical protein |
| Locos 2 | GM183 | YcaO-like protein |
| Locus 2 | GM184 | Hypothetical protein |
| Locus 2 | GM185 | SagB-type dehydrogenase domain protein |
| Locus 2 | GM186 | Hypothetical protein |
| Locus 2 | GM187 | ABC transporter, ATP-binding protein |
| Locus 2 | GM188 | ABC-2 type transporter |
| Locus 2 | GM189 | Hypothetical protein |
| Locus 2 | GM196 | Hypothetical protein |

TABLE 2
Target Genes in Locus 3 of P. acnes RT4 and RT5.

| Locus | ID | Description |
|---|---|---|
| Locus 3 | PAGK_2319 | hypothetical protein |
| Locus 3 | PAGK_2320 | hypothetical protein |
| Locus 3 | PAGK_2321 | hypothetical protein |
| Locus 3 | PAGK_2322 | plasmid stabilization system protein |
| Locus 3 | PAGK_2323 | hypothetical protein |
| Locus 3 | PAGK_2324 | hypothetical protein |
| Locus 3 | PAGK_2325 | hypothetical protein |
| Locus 3 | PAGK_2326 | CobQ/CobB/MinD/ParA nucleotide binding |
| | | domain |
| Locus 3 | PAGK_2327 | hypothetical protein |
| Locus 3 | PAGK_2328 | hypothetical protein |
| Locus 3 | PAGK_2329 | hypothetical protein |
| Locus 3 | PAGK_2330 | hypothetical protein |
| Locus 3 | PAGK_2331 | hypothetical protein (similar to PPA1279) |
| Locus 3 | PAGK_2332 | plasmid partition protein ParA |
| Locus 3 | PAGK_2333 | hypothetical protein |
| Locus 3 | PAGK_2334 | hypothetical protein |
| Locus 3 | PAGK_2335 | hypothetical protein |
| Locus 3 | PAGK_2336 | putative ribbon helix helix protein oopG family |
| Locus 3 | PAGK_2337 | putative ribonscience E |
| Locus 3 | PAGK_2338 | hypothetical protein (similar to PPA1284) |
| Locus 3 | PAGK_2339 | hypothetical protein (similar to PPA1286) |
| Locus 3 | PAGK_2340 | putative permeases |
| Locus 3 | PAGK_2341 | hypothetical protein (similar to PPA1297) |
| Locus 3 | PAGK_2342 | hypothetical protein (similar to PPA1296) |
| Locus 3 | PAGK_2343 | hypothetical protein (similar to PPA1286) |
| Locus 3 | PAGK_2344 | hypothetical protein (similar to |
| | | CLOLEP_00122) |
| Locus 3 | PAGK_2345 | hypothetical protein (similar to |
| | | CLOLEP_00123) |
| Locus 3 | PAGK_2346 | hypothetical protein (similar to |
| | | CLOLEP_00124) |
| Locus 3 | PAGK_2347 | hypothetical protein (similar to |
| | | CLOLEP_00125) |
| Locus 3 | PAGK_2348 | hypothetical protein (similar to |
| | | CLOLEP_00126) |
| Locus 3 | PAGK_2349 | hypothetical protein (similar to |
| | | CLOLEP_00127) |
| Locus 3 | PAGK_2350 | hypothetical protein |
| Locus 3 | PAGK_2351 | hypothetical protein (similar to |
| | | CLOLEP_00129) |
| Locus 3 | PAGK_2352 | hypothetical protein (similar to |
| | | CLOLEP_00130) |
| Locus 3 | PAGK_2353 | hypothetical protein (similar to |
| | | CLOLEP_00131) |
| Locus 3 | PAGK_2354 | hypothetical protein (similar to |
| | | CLOLEP_00132) |
| Locus 3 | PAGK_2355 | hypothetical protein (similar to |
| | | CLOLEP_00134) |
| Locus 3 | PAGK_2356 | hypothetical protein (similar to |
| | | CLOLEP_00135) |
| Locus 3 | PAGK_2357 | hypothetical protein (similar to |
| | | CLOLEP_00141) |
| Locus 3 | PAGK_2358 | hypothetical protein (similar to |
| | | CLOLEP_00142) |
| Locus 3 | PAGK_2359 | hypothetical protein (similar to |
| | | CLOLEP_00143) |
| Locus 3 | PAGK_2360 | hypothetical protein (similar to |
| | | CLOLEP_00144, RepC) |
| Locus 3 | PAGK_2361 | hypothetical protein (similar to |
| | | CLOLEP_00145, TactZ) |
| Locus 3 | PAGK_2362 | hypothetical protein (similar to |
| | | CLOLEP_00146, TactA) |
| Locus 3 | PAGK_2363 | hypothetical protein (similar to |
| | | CLOLEP_00147, TactB) |
| Locus 3 | PAGK_2364 | hypothetical protein (similar to |
| | | CLOLEP_00148, TactC) |
| Locus 3 | PAGK_2365 | hypothetical protein (similar to |
| | | CLOLEP_00149, Hp-1) |
| Locus 3 | PAGK_2366 | hypothetical protein (similar to |
| | | CLOLEP_00151, TactE) |
| Locus 3 | PAGK_2367 | hypothetical protein (similar to |
| | | CLOLEP_00152, TactE) |
| Locus 3 | PAGK_2368 | hypothetical protein (similar to |
| | | CLOLEP_00153, TactE) |
| Locus 3 | PAGK_2369 | hypothetical protein (similar to |
| | | CLOLEP_00154) |
| Locus 3 | PAGK_2370 | hypothetical protein (similar to |
| | | CLOLEP_00157) |
| Locus 3 | PAGK_2371 | hypothetical protein (similar to |
| | | CLOLEP_00158) |
| Locus 3 | PAGK_2372 | hypothetical protein (similar to |
| | | CLOLEP_00159) |
| Locus 3 | PAGK_2373 | hypothetical protein (similar to |
| | | CLOLEP_00160) |
| Locus 3 | PAGK_2374 | hypothetical protein |
| Locus 3 | PAGK_2375 | hypothetical protein (similar to |
| | | CLOLEP_00162) |
| Locus 3 | PAGK_2376 | hypothetical protein (similar to |
| | | CLOLEP_00163) |
| Locus 3 | PAGK_2377 | hypothetical protein (similar to |
| | | CLOLEP_00164) |
| Locus 3 | PAGK_2378 | hypothetical protein (similar to |
| | | CLOLEP_00165) |
| Locus 3 | PAGK_2379 | repA |
| Locus 3 | PAGK_2380 | CobQ/CobB/MinD/ParA nucleotide binding |
| | | domain |
| Locus 3 | PAGK_2381 | hypothetical protein |
| Locus 3 | PAGK_2382 | hypothetical protein |
| Locus 3 | PAGK_2383 | YagtE |
| Locus 3 | PAGK_2384 | hypothetical protein |
| Locus 3 | PAGK_2385 | hypothetical protein |
| Locus 3 | PAGK_2386 | hypothetical protein |
| Locus 3 | PAGK_2387 | hypothetical protein |
| Locus 3 | PAGK_2388 | hypothetical protein |
| Locus 3 | PAGK_2389 | hypothetical protein |
| Locus 3 | PAGK_2390 | hypothetical protein |
| Locus 3 | PAGK_2391 | hypothetical protein |
| Locus 3 | PAGK_2392 | RestA |

TABLE 3
Target Genes in Locus 4 of P. acnes RT8.

| Locus | ID | Description |
|---|---|---|
| Locus 4 | HMPREF9676_00292 | tRNA adenyfyltransferase |
| Locus 4 | HMPREF9676_00293 | conserved hypothetical protein |
| Locus 4 | HMPREF9676_00294 | conserved domain protein |
| Locus 4 | HMPREF9676_00295 | response regulator receiver domain |
| | | protein |
| Locus 4 | HMPREF9676_00296 | his-Idine kinese |
| Locus 4 | HMPREF9676_00297 | hypothetical protein |
| Locus 4 | HMPREF9676_00298 | hypothetical protein |
| Locus 4 | HMPREF9676_00299 | hypothetical protein |
| Locus 4 | HMPREF9676_00300 | hypothetical protein |
| Locus 4 | HMPREF9676_00301 | hypothetical protein |
| Locus 4 | HMPREF9676_00302 | drug resistance MFS transporter, |
| | | drug H + antiporter-2 |
| | | (14 Spanner) (DHA2) family protein |
| Locus 4 | HMPREF9676_00303 | hypothetical protein |
| Locus 4 | HMPREF9676_00304 | conserved domain protein |
| Locus 4 | HMPREF9676_00305 | beta-kedoncyl synthase, N-terminal |
| | | domain protein |
| Locus 4 | HMPREF9676_00306 | hypothetical protein |
| Locus 4 | HMPREF9676_00307 | acetyltransferase, GNAY family |
| Locus 4 | HMPREF9676_00308 | putative (3R)-hydrosymyristoyl-ACP |
| | | dehydratase |
| Locus 4 | HMPREF9676_00309 | putative acryl carrier protein |
| Locus 4 | HMPREF9676_00310 | putative 3-ketcacyl-(acryl carrier |
| | | protein) reductase |
| Locus 4 | HMPREF9676_00311 | ornithine cryclodeaminasolnu |
| | | crytalin family protein |
| Locus 4 | HMPREF9676_00312 | pyridonal phosphate depondent |
| | | enzyme |
| Locus 4 | HMPREF9676_00313 | lantbiotic dehydratase, C-terminus |
| Locus 4 | HMPREF9676_00314 | Aminotransferase, class III |
| Locus 4 | HMPREF9676_00315 | acyl carrier domain protein |
| Locus 4 | HMPREF9676_00316 | AMP-binding enzyme |
| Locus 4 | HMPREF9676_00317 | Melonyl CoA-acyl carrier protein |
| | | transacytase family protein |
| Locus 4 | HMPREF9676_00318 | ABC-2 type transporter |
| Locus 4 | HMPREF9676_00319 | ABC transporter, ATP-binding protein |
| Locus 4 | HMPREF9676_00320 | hypothetical protein |

In some embodiments, synthetic bacteria are engineered to affect the pH of the microenvironment on which the synthetic bacteria are applied. In a non-limiting example, synthetic bacteria maintain a pH in the microenvironment between about pH 4 and pH 7, between about pH 4.5 and pH 6.5, or between about pH 5 and about pH 6. In some cases, synthetic bacteria maintain a pH in the microenvironment at about pH 5.5. Synthetic bacteria may affect the pH by a number of methods, such as producing and/or sequestering a pH modulating biomolecule and/or chemical. In some cases, the synthetic bacteria chemically alter an acid or base to affect the pH of its microenvironment. As a non-limiting embodiment, a synthetic bacteria detects a pH greater than about 7, less than about 4, or a pH that is not about 5.5, and affects the microenvironment to alter the pH to a value between about 4 and 7, or at about 5.5.

In some embodiments, synthetic bacteria are engineered to produce a biomolecule, such as a pro-inflammatory metabolite, produced at a tunable level optionally different from that of the another bacteria. As a non-limiting example, the biomolecule is ST2S, ST4S, ST6S, bmpA, PTS-Mtl-EIIABC, MFS ST1, MFS ST2, ST1P, ST3P, ST4P, ST5P, ST6P, ST7P, PTS-Mtl-EIIA, ST1P1, ST3P1, ST4P1, ST6P1, FhuD, ST7A, manA, FhuC, FhuB, FhuD, COX15, talAB, hMuV, HtaA, HmuT, GAPDH, hemH, CS, IDH1, cobA-hemD, cysG, IDH1, IDH1, TGL, OGDH, narJ, narl, narH, narG, E1.7.2.1, norB, gdhA, gudB, rocG, MDT1, MDT2, MDT3, T2SF2, T2SF1, secA, secY, secF, secD, yajC, secE, ftsY, yidC, secG, clpX, clp2, clp1, or a combination thereof, and expression of the biomolecule is down-regulated. As a non-limiting example, the biomolecule is ST3S; PTS-Nag-E1; ST2P; ST2A; FhuC; FhuB; FhuD; PFK; glpK; cyoE; fabZ; fabG; fabF; fabZ; fabG; fabF; fabZ; fabG; fabF; fabZ; fabG; fabF; fabZ; fabG; fabF; fabZ; fabG; fabF; fabZ; fabG; fabF; fabZ; fabG; fabF; fabD; fumC; DLST; AMT; CblO; cbiM; cbiQ; cbiN; cysG-cbiX; cobI-cbiL; cobM, cbiF; cobK, cbiJ; cobO, btuR; cobQ, cbiP; cbiB, cobD; cobS, cobV; MDT4; or a combination thereof, and expression of the biomolecule is up-regulated. In some cases, up- and/or down-regulation is compared to expression of the biomolecule in a bacteria from which the synthetic was derived and/or a related bacteria, including disease-associated bacteria.

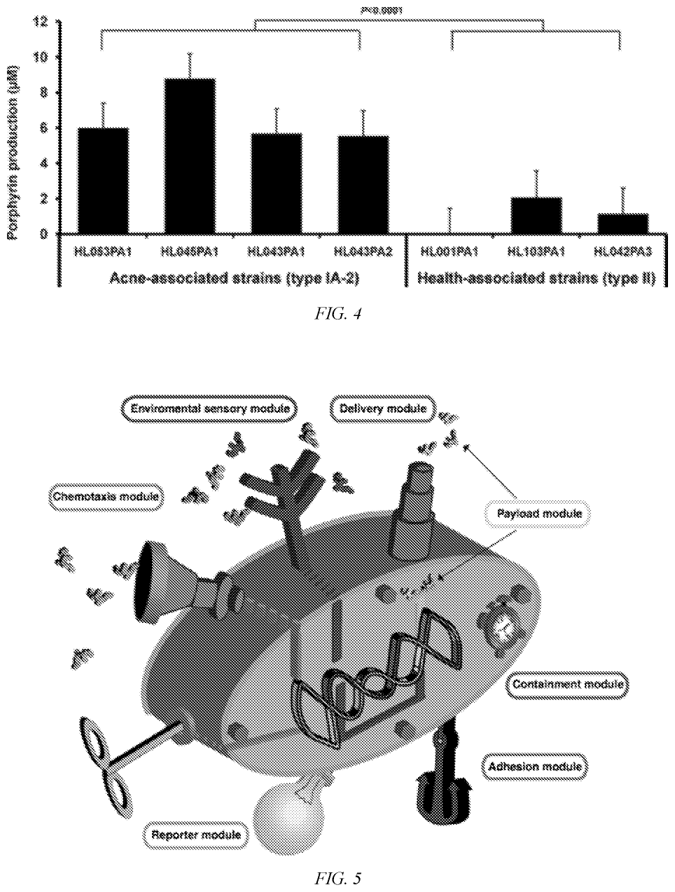

In some embodiments, the biomolecule is the metabolite porphyrin, which includes, without limitation, coproporphyrin III and protoporphyrin IX. In some embodiments, the biomolecule is porphyrin and it is produced at a level at or below about 1, 2, 3, or 4 micromolar. In some embodiments, the pro-inflammatory metabolite level is modulated by engineering a vitamin B12 metabolic pathway in the non-pathogenic bacteria. Some such engineering includes modifying one or more native molecules of the vitamin B12 metabolic pathway in the non-pathogenic bacteria. In some embodiments, a synthetic bacteria secretes an agent, such as a chemical, nucleic acid, peptide, polypeptide, lipid, carbohydrate, and/or small molecule, whereby the secreted agent affects production of another biomolecule in a bacteria neighboring or within the same environment as the synthetic bacteria. In some such cases, the another biomolecule is a porphyrin, where the porphyrin levels in neighboring bacteria are reduced upon agent secretion by the synthetic bacteria. In many implementations of the disclosure, the non-pathogenic bacteria is a strain of P. acnes. FIG. 4 is a graph showing the relationship between porphyrin production in P. acnes strains associated with acne or healthy skin. Accordingly, to mimic health-associated P. acnes, it is often desirable to produce a synthetic bacteria with decrease porphyrin levels.

In a certain embodiment, the synthetic bacteria comprises a porphyrin pathway that comprises a biomolecule introduced, removed, or modified relative to the porphyrin pathway of the non-pathogenic bacteria. In a certain embodiment the biomolecule comprises a gene selected from COX15, cyoE, and HemB, HemC, HemD, HemE, HemF, HemG, HemH, HemY, or PPA2095 protoporphyrinogen oxidase (HemY homologue), or any combination thereof. In some embodiments, the biomolecule comprises the insertion of a stop codon or truncation into the coding sequence of the gene. Truncations can be achieved using a CRISPR/Cas9 system. In other embodiments, the gene comprises a disruption to an intergenic region that reduces expression of the lipase in the synthetic bacteria. In certain embodiments, the synthetic bacteria produces a decrease in porphyrin production that is greater than 10%, 20%, 30%, 40%, 50%, 60%, 70%, 80%, o 90% when compared to the unmodified bacteria. In certain embodiments, porphyrin is not produced by the synthetic bacteria or is produced at a level less than about 4, 3, 2, or 1 micromolar. In certain embodiments, porphyrin is produced at a level less than about 400, 300, 200, or 100 nanomolar. In certain embodiments, the synthetic bacteria produce porphyrin at a level less than or about equal to that of P. acnes strains HL042PA3, HL103PA1, or HL001PA1.

In some embodiments, a biomolecule produced by synthetic bacteria is an antioxidant. For example, vitamin A, vitamin C, and/or vitamin D. In some cases, a synthetic bacteria produces one or more of the following biomolecules: omiganan, epinecidin-1 (22-42) peptide, SALF (55-76) cyclic peptide, SALF (55-76) linear peptide, granulysin or a granulysin derivative such as one having a helix-loop-helix motif or a peptide 31-50 having a V44W mutation.

In some embodiments, synthetic bacteria are engineered to produce a modified lipase having an activity less than about 25%, 50%, 75%, 85%, or 90% of an activity of the native lipase in the bacteria from which the synthetic bacteria was derived and/or another optionally related bacteria. In some cases, this reduction in activity attenuates neutrophil recruitment to the site in which the synthetic bacteria is applied. In some cases, the synthetic bacteria produces at least about 25%, 50%, 75%, 85%, or 90% fewer free fatty acids from sebum than the bacteria from which the synthetic bacteria was derived and/or another optionally related bacteria. In some embodiments, the lipase is modified by deletion in whole or in part. In some embodiments, the modified lipase comprises the insertion of a stop codon or truncation into the coding sequence of a lipase gene. Truncations can be achieved using a CRISPR/Cas9 system. in other embodiments, the lipase has a disruption to an intergenic region that reduces expression of the lipase in the synthetic bacteria. In certain embodiments, the lipase that s modified is HMPREF0675_4855, HMPREF0675_4856, HMPREF0675_4479, HMPREF0675_4480, HMPREF0675_4481, HMPREF0675_3655/3657, HMPREF0675_4816, HMPREF0675_4817, HMPREF0675_5205, HMPREF0675_5206, HMPREF0675_5014, HMPREF0675_5101, HMPREF0675_5159, HMPREF0675_4093/4094, HMPREF0675_4163, HMPREF0675_5031, HMPREF0675_5390, HMPREF0675_3037, or a homolog thereof having greater than 90%, 95%, 96%, 97%, 98%, 99% homology.

In some embodiments, synthetic bacteria are engineered with a heterologous hyaluronidase gene from another species or strain of bacteria. In certain embodiments, the heterologous hyaluronidase is from a group B Streptococcus.

In certain embodiments, the synthetic bacteria has been engineered to disrupt, delete, or have lower activity or expression of any of the following proteins: Adhesion (NCBI Accession No. 50843565 or 50843645); Cell wall hydrolase (NCBI Accession No. 50843410); Lipase/acylhydrolase (NCBI Accession No. 50843480); NPL/P60 protein (NCBI Accession No. 50842209); Peptide ABC transporter (NCBI Accession No. 50843590); Protein PPA1197 (NCBI Accession No. 50842677); Protein PPA1281 (NCBI Accession No. 50842762); Protein PPA1715 (NCBI Accession No. 50843175); Protein PPA1939 (NCBI Accession No. 50843388); Protein PPA2239 (NCBI Accession No. 50843674); Rare lipoprotein A rlpa (NCBI Accession No. 50843612); or Triacylglycerol lipase (NCBI Accession No. 50843543). In certain embodiments, the bacteria has been selected, transformed, or engineered with a nucleotide to delete or disrupt a gene encoding any of the following proteins: Adhesion (NCBI Accession No. 50843565 or 50843645); Cell wall hydrolase (NCBI Accession No. 50843410); Lipase/acylhydrolase (NCBI Accession No. 50843480); NPL/P60 protein (NCBI Accession No. 50842209); Peptide ABC transporter (NCBI Accession No. 50843590); Protein PPA1197 (NCBI Accession No. 50842677); Protein PPA1281 (NCBI Accession No. 50842762); Protein PPA1715 (NCBI Accession No. 50843175); Protein PPA1939 (NCBI Accession No. 50843388); Protein PPA2239 (NCBI Accession No. 50843674); Rare lipoprotein A rlpa (NCBI Accession No. 50843612); or Triacylglycerol lipase (NCBI Accession No. 50843543).

In certain embodiments, the synthetic bacteria has been engineered to disrupt, delete, or have lower activity or expression of any of the following proteins: HMPREF0675_4855; HMPREF0675_4856; HMPREF0675_4479; HMPREF0675_4480; HMPREF0675_4481; HMPREF0675_3655/3657; HMPREF0675_4816; HMPREF0675_4817; HMPREF0675_5205; HMPREF0675_5206; HMPREF0675_5014; HMPREF0675_5101; HMPREF0675_5159; HMPREF0675_4093/4094; HMPREF0675_4163; HMPREF0675_5031; HMPREF0675_5390; HMPREF0675_3037. In certain embodiments, the bacteria have been selected, transformed, or engineered with a nucleotide to delete or disrupt a gene encoding any of the following proteins: HMPREF0675_4855; HMPREF0675_4856; HMPREF0675_4479; HMPREF0675_4480; HMPREF0675_4481; HMPREF0675_3655/3657; HMPREF0675_4816; HMPREF0675_4817; HMPREF0675_5205; HMPREF0675_5206; HMPREF0675_5014; HMPREF0675_5101; HMPREF0675_5159; HMPREF0675_4093/4094; HMPREF0675_4163; HMPREF0675_5031; HMPREF0675_5390; HMPREF0675_3037.

In some embodiments, synthetic bacteria are engineered to produce a sensor receptor specific for a first target molecule. In some cases, the first target molecule is intracellular to bacteria and/or a host cell. In some cases, the first target molecule is extracellular to bacteria and/or a host cell. In some embodiments, the first target molecule is a porphyrin, or a molecule involved in porphyrin metabolism. In some cases, the first target molecule is involved in a lipase reaction. In some cases, the first target molecule is involved in glucose production. In some cases, the presence or absence of the first target molecule is evaluated by monitoring the presence or absence, respectively, of binding between the sensor receptor and the first target molecule.

In some embodiments, synthetic bacteria are engineered to produce a sensor effector specific for a second target molecule. In some cases, the second target molecule is intracellular to bacteria and/or a host cell. In some cases, the second target molecule is extracellular to bacteria and/or a host cell. In some embodiments, the sensor effector affects production of the second target molecule by the synthetic bacteria. In some embodiments, the sensor effector attenuates or halts production of the second target molecule. In some embodiments, the second target molecule is a porphyrin. In some embodiments, the second target molecule is involved in a lipase reaction. In some embodiments, the second target molecule is involved in glucose production.

In some embodiments, synthetic bacteria are engineered to mitigate induction of an individual's inflammatory response to the synthetic bacteria when administered to the individual, for example, by topical or other means. In some embodiments, the individual's inflammatory response is mitigated by the production of a toll like receptor (TLR) ligand modified from a native TLR ligand of the non-pathogenic bacteria. In some cases, the modified TLR ligand has no binding affinity to a TLR of the patient, or the modified TLR ligand has a binding affinity to a TLR of the patient that is less than about 25%, 50%, 75%, 85%, or 90% of the binding affinity of the native TLR ligand. TLR ligands include those present on a keratinocyte, inflammatory cell, other antigen presenting cell, or a combination thereof, in the individual. Inflammatory cells include, without limitation, macrophage dendritic cells and a langerhan cells. In some cases, the modified TLR ligand is selected from a cell-wall component of the non-pathogenic bacteria, a lipoprotein from a gram-positive bacteria, lipoarabinomannan from mycobacteria, zymosan from a yeast cell wall, or a combination thereof. Cell-wall components include peptidoglycans and lipoteichoic acid. In some embodiments, the individual's inflammatory response is mitigated by replacing a toll like receptor (TLR) ligand present in the non-pathogenic bacteria with a human peptide. In some embodiments, the individual's inflammatory response is mitigated by the absence of a toll like receptor (TLR) ligand present in the non-pathogenic bacteria. Non-limiting examples of TLR ligands include TLR2 ligands and TLR4 ligands.

In some embodiments, synthetic bacteria are engineered to comprise a genome or other modification that prevents the synthetic bacteria from acquiring an antibiotic resistance gene. Accordingly, some synthetic bacteria described herein are not resistant to antibiotics such as those in the class of tetracycline antibiotics, erythromycin antibiotics, and/or clindamycin antibiotics. In some cases, the genome modification comprises a mutation in 16S ribosomal RNA of the non-pathogenic bacteria or 23 S ribosomal RNA of the non-pathogenic bacteria. In some cases, the genome modification comprises a mutation outside of the 16S or 23S RNA of the non-pathogenic bacteria. Non-limiting examples of 23 S ribosomal RNA mutations within a synthetic bacteria include those occurring at base 2058, 2057, 2059, 1058, or a combination thereof. In some cases, a base mutation is made relative to the base of the non-pathogenic bacteria. Mutations include base substitutions, additions, deletions, or a combination thereof. In some cases, a synthetic bacteria is engineered to prevent the synthetic bacteria from acquiring a mutation at base 2058, 2057, 2059, 1058, or a combination thereof, in its 23S ribosomal RNA as compared to the base in the non-pathogenic bacteria. Table 4 provides 16S and 23S ribosomal RNA mutations within P. acnes that confer resistance to tetracycline and clindamycin/erythromycin antibiotics, respectively.

TABLE 4
Antibiotic resistance in P. acnes.

| Erythromycin | Mutations in the genes encoding 23S ribosomal RNA |
|---|---|
| Group I | A→G transition at E. coli equivalent base 2058 |
| | Highly resistant to erythromycin |
| | Variable for other macrolides and clindamycin |
| Group III | G→A transition at E. coli equivalent base 2057 |
| | Low level erythromycin resistance |
| Group IV | A→G transition at E. coli equivalent base 2059 |
| | Highly resistant to erythromycin and all macrolides |
| | Elevated but variable resistance to clindamycin |
| Tetracycline | Mutation in the gene encoding 16S ribosomal RNA |
| | G→W transition at E. coli equivalent base 1058 |
| | Variable resistance to tetracycline, doxycycline and |
| | minocycline |

In some embodiments, synthetic bacteria are engineered to acquire a deleterious molecule from an area of an individual to which the synthetic bacteria are applied, to decrease the relative amount of the deleterious molecule from the area after application of the synthetic bacteria. In some embodiments, the synthetic bacteria metabolizes the deleterious molecule. Non-limiting examples of a deleterious molecule include a sulfur oxide, a nitrogen oxide, carbon monoxide, a volatile organic compound, particulate matter, a persistent free radical, toxic metal, a chlorofluorocarbon, ammonia, an odorous molecule, a radioactive pollutant, secondary pollutant, ground level ozone, peroxyacetyl nitrate, hazardous air pollutant, and persistent organic pollutant. Sulfur oxide includes, without limitation, sulfur monoxide, disulfur dioxide, disulfur monoxide, sulfur dioxide, and sulfur trioxide. In some cases, the deleterious molecule is sulfur dioxide. Nitrogen oxide includes, without limitation, nitrogen monoxide, nitrogen dioxide, nitrous oxide, nitrosylazide, oxatetrazole, dinitrogen trioxide, dinitrogen tetraoxide, dinitrogen pentoxide, trinitramide, nitrite, nitrate, nitronium, nitrosonium, and peroxonitrite. In some cases, the deleterious molecule is nitrogen dioxide. In some cases, the volatile organic compound is methane. Further non-limiting examples of volatile organic compounds include benzene, toluene, xylene, 1,3-butadiene, isoprene, terpene, aliphatic hydrocarbon, ethyl acetate, glycol ether, acetone, chlorofluorocarbon, tetrachloroethene, methylene chloride, perchloroethylene, methyl tert-butyl ether, and formaldehyde. Non-limiting examples of particulate matter include a solid or liquid suspended in a gas; such as those derived from one or more of the following: volcanoes, dust storms, forest and grassland fires, living vegetation, sea spray, and burning of fossil fuels in vehicles, power plants and industrial processes. Non-limiting examples of a persistent free radical include Gomberg's triphenylmethyl radical, Fremy's salt (potassium nitrosodisulfonate), nitroxide, TEMPO (2,2,6,6-Tetramethylpiperidin-1-yl)oxyl), 4-Hydroxy-TEMPO (4-hydroxy-2,2,6,6-tetramethylpiperidin-1-oxyl), nitronyl nitroxide, azephenylenyl, perchlorophenylmethyl radical, and TTM (tris(2,4,6-trichlorophenyl)methyl radical). In some cases, the deleterious molecule is a toxic metal such as mercury, lead, cadmium, manganese, or an alloy or combination thereof. Non-limiting examples of a chlorofluorocarbon include trichlorofluoromethane; dichlorodifluoromethane; difluoromethane/pentafluoroethane; chlorotrifluoromethane; chlorodifluoromethane; dichlorofluoromethane; chlorofluoromethane; bromochlorodifluoromethane; 1,1,2-trichloro-1,2,2-trifluoroethane; 1,1,1-trichloro-2,2,2-trifluoroethane; 1,2-dichloro-1,1,2,2-tetrafluoroethane; 1-chloro-1,1,2,2,2-pentafluoroethane; 2-chloro-1,1,1,2-tetrafluoroethane; 1,1-dichloro-1-fluoroethane; 1-chloro-1,1-difluoroethane; tetrachloro-1,2-difluoroethane; tetrachloro-1,1-difluoroethane; 1,1,2-trichlorotrifluoroethane; 1-bromo-2-chloro-1,1,2-trifluoroethane; 2-bromo-2-chloro-1,1,1-trifluoroethane; 1,1-dichloro-2,2,3,3,3-pentafluoropropane; and 1,3-dichloro-1,2,2,3,3-pentafluoropropane. Non-limiting examples of radioactive pollutants include those produced by a nuclear explosion, nuclear event, nuclear explosive device, and radioactive decay of radon. Non-limiting examples of secondary pollutants include those comprising particulates created from gaseous primary pollutants and compounds in photochemical smog. Non-limiting examples of a hazardous air pollutant include carbon monoxide, cyanide, glycol ether, and polycyclic aromatic hydrocarbon. Non-limiting examples of persistent organic pollutants include acetaldehyde; acetamide; acetonitrile; acetophenone; acrolein; acrylamide; acrylic acid; acrylonitrile 4-aminobiphenyl; aniline o-anisidine; m-anisidine; p-anisidine; asbestos; benzene; 1,3-butadiene; carbon disulfide; carbon monoxide; carbon tetrachloride; carbonyl sulfide; chlorine; chlorobenzene; chloroethane; chloroform; chloromethane; chloroprene; cresol; o-cresol; cumene; 1,2-dibromoethane; 1,2-dichloroethane; dichloromethane; ethylbenzene; ethylene glycol; ethylene oxide; fluidized bed concentrator; formaldehyde; hexachlorobenzene; hexane; hydrazine; hydrogen chloride; hydrogen fluoride; methanol; methyl isobutyl ketone; methyl isocyanide; methyl methacrylate; methyl tert-butyl ether; naphthalene; 4-nitroaniline; nitrogen dioxide; phenol; polychlorinated biphenyl; propionaldehyde; quinoline; sodium selenite; styrene; sulfur trioxide; tetrachloroethylene; toluene; 1,1,1-trichloroethane; trichloroethylene; vinyl acetate; vinyl chloride; xylene; and any chemicals regulated by the US EPA via maximum achievable control technology standards.

In some embodiments, synthetic bacteria are supplemented with and/or engineered to produce a biomolecule selected from one or more of: UV-screening/observing amino acid-like molecules, flavonoids, betalanines, UV-screening/observing pigments (e.g. carotenoids/cartenoproteins, xanthopylls and porphyrin-based/heme-porphyrin based (prior art mentions porphyrins), UV-screening/observing co-factors (e.g. tetrahydrobiopterin), phenylpropanoids, polyphenol (e.g. tannins), pycnogenol, tyrosinases (and its substrates and products), alpha hydroxy acids (AHAs), polysaccharides (e.g. glycosaminoglycans, (GAGs) or mucopolysaccharides), skin related cofactors, vitamin E, polymers, and additional skin related natural compounds, such as: collagen, keratin, elastin, linoleic acid, laminin, tretinoin, tazarotene, sargaquinoic acid, sargachromenol, fucoxanthin, retinoid, anti-inflammatory cytokines (as II-2), cortisone, tacrolimus, ciclosporin, resveratrol, gallocatechol, gallocatechin, epigallocatechin gallate, retinoid, vitamin A, vitamin A derivatives, beta-carotene, vitamin D, vitamin A derivatives, moisture compounds; cortisone, tacrolimus and ciclosporin, DNA repair enzymes; photolyase, endonuclease and glycosylase.

In various aspects, synthetic bacteria are engineered, or derived from, non-pathogenic bacteria. However, use of pathogenic bacteria is envisioned by manipulating the pathogenic bacteria to attenuate virulence, for example, by removing and/or modifying one or more sections of its genome. In some cases, the non-pathogenic bacteria is a gram positive bacteria. In some cases, the non-pathogenic bacteria is a gram negative bacteria. Non-limiting examples of gram positive bacteria include Propionibacterium acnes, Staphylococcus epedermidis, and Staphylococcus auereus. In some cases, the non-pathogenic bacteria is a P. acnes strain belonging to the Type clade II. P. acnes Type clade II strains include ribotype 2, ribotype 8, and ribotype 6. In some embodiments, the P. acnes strain is one associated with healthy skin in an individual. In some embodiments, the P. acnes strain has sequence identity or homology to any one of SEQ ID NOS: 100, 101, 102 and 103. In some cases, the P. acnes strain has a sequence at least about 75%, 80%, 85%, 90%, 95%, 98%, 99% or 100% identical to any of SEQ ID NOS: 100, 101, 102 and 103. In some cases, the P. acnes strain has a sequence less than about 75%, 80%, 85%, 90%, 95%, 98%, or 99% identical to any of SEQ ID NOS: 100, 101, 102 and 103. The terms “homologous,” “homology,” or “percent homology” when used herein to describe to an amino acid sequence or a nucleic acid sequence, relative to a reference sequence, can be determined using the formula such as incorporated into the basic local alignment search tool (BLAST) programs. Percent homology and identity of sequences can be determined using the most recent version of BLAST, as of the filing date of this application. In some embodiments, a health-associated strain of P. acnes is a Type II strain, for example, ST0, ST7, ST25, ST26, ST27, ST28, ST30, ST58, ST59, ST60, ST61, ST62, ST63, ST64, ST65, ST66, ST67, ST68, ST69, ST71, ST72, ST79, ST6, ST7, ST25, ST26, ST27, ST28, ST30, ST58, ST59, ST60, ST61, ST62, ST63, ST64, ST65, ST66, ST67, ST68, ST69, ST71, ST72, and ST79. In some embodiments, a health-associated strain of P. acnes is a Type III strain, for example, ST32, ST33, ST73, ST74, ST75, ST76, ST77, ST81, ST90, ST12, ST32, ST33, ST51, ST53, ST73, ST74, ST75, ST76, ST77, ST81, and ST90. In some embodiments, features engineered from the non-pathogenic bacteria to produce the synthetic bacteria are stabilized. As a non-limiting example, by eliminating stress-inducible error-prone DNA polymerases in the synthetic bacteria.

Non-limiting examples of bacteria from which a synthetic bacteria described herein are derived from include one or more of: Actinomycetales, Anaerococcus, Bacillales, Bifidobacterium, Enhydrobacter, Finegoldia, Carnobacterium, Coryneobacterium, Lactobacillus, Lactococcus, Leunconostoc, Macrooccus, Micrococcineae, Oenococcus, Pediococcus, Peptoniphilus, Propionibacterium, Salinicoccus, Sphingomonas, Streptococcus, Tetragenoccus, and Weissella. In some embodiments, a synthetic bacteria is not derived from Propionibacterium acnes, a pathogenic strain of Coryneobacterium, S. aureus, or S. epidermidis. In other embodiments, a synthetic bacteria is derived from Propionibacterium acnes, a pathogenic strain of Coryneobacterium, S. aureus, or S. epidermidis. In some embodiments, a synthetic bacteria is derived from one or more of: Lactobacillus casei, Lactobacillus reuteri, Lactobacillus acidophilus, Lactobacillus jensenii, Bifidobacterium lognum, Bifidobacterium reuteri, Bifidobacterium lactis, Bifidobacterium breve, Bifidobacterium animalis, Propionibacterium acidipropionici, Propionibacterium freudenreichii, Propionibacterium thoenii, and Propionibacterium jensenii. In some embodiments, a synthetic bacteria is derived from bacteria in a phylum such as gamma-proteobacteria, alpha-proteobacteria, and bacteriodetes.

In various aspects, the effect of a synthetic bacteria on the immune system of the individual to which the synthetic bacteria is administered is minimized as compared to the effect caused by a disease-associated bacteria. For some cases wherein the non-pathogenic bacteria from which the synthetic bacteria is derived is a health-associated or other non-pathogenic P. acnes strain, the synthetic bacteria derived therefrom have a different effect on the individual's immune system than a disease-associated P. acnes strain such as those of clade IA, and ribotypes 4 and 5. In some embodiments, synthetic bacteria do not induce or induces less human beta defensin (HBD) in individual keratinocytes as compared to the level of HBD induced by a disease-associated P. acnes strain. HBD includes HBD-1, HBD-2, and HBD-3. As non-limiting examples, the level of HBD induced in individual keratinocytes is less than about 25%, 50%, 75%, 85%, or 90% of the amount of HBD induced in patient keratinocytes if the disease-associated P. acnes strain were applied to the affected skin in the same amount. In some embodiments, synthetic bacteria do not induce or induce fewer of one or more of: interleukin-8, interleukin-1, interleukin-6, TNF-alpha, and NFkB in individual keratinocytes as compared to levels produced by a disease-associated P. acnes strain. As non-limiting examples, the synthetic bacteria produce less than about 25%, 50%, 75%, 85%, or 90% of one or more of: interleukin-8, interleukin-1, interleukin-6, TNF-alpha, and NFkB in individual keratinocytes as compared to levels produced by a disease-associated P. acnes strain. In some embodiments, synthetic bacteria do not induce or induce fewer MMP as compared to levels of MMP produced by a disease-associated P. acnes strain. As non-limiting examples, the synthetic bacteria produce less than about 25%, 50%, 75%, 85%, or 90% of MMP as compared to levels of MMP produced by a disease-associated P. acnes strain. MMP includes MMP-8 and MMP2. In some embodiments, synthetic bacteria recruit fewer neutrophils from an individual than a disease-associated P. acnes strain if the disease-associated P. acnes strain were applied to the affected skin in the same amount. As non-limiting examples, the synthetic bacteria produce less than about 25%, 50%, 75%, 85%, or 90% of neutrophils from an individual than the disease-associated P. acnes strain if the disease-associated P. acnes strain were applied to the affected skin in the same amount. In some embodiments, synthetic bacteria recruit fewer polymorphonuclear leukocytes from an individual than a disease-associated P. acnes strain if the disease-associated P. acnes strain were applied to the affected skin in the same amount. As non-limiting examples, the synthetic bacteria produce less than about 25%, 50%, 75%, 85%, or 90% of polymorphonuclear leukocytes from an individual than the disease-associated P. acnes strain if the disease-associated P. acnes strain were applied to the affected skin in the same amount. In some embodiments, synthetic bacteria recruits at least about 25%, 50%, 75%, 85%, or 90% fewer dermal fibroblasts from an individual than a disease-associated P. acnes strain if the disease-associated P. acnes strain were applied to the affected skin in the same amount. In some embodiments, synthetic bacteria inhibits production of pro-inflammatory neuropeptides in an individual.

In another aspect of the disclosure, synthetic bacteria are provided that comprise an engineered vitamin B12 metabolic pathway that modulates production of intracellular vitamin B12 in the synthetic bacteria to levels suitable for therapeutic use. As a non-limiting example, for P. acnes strains, vitamin B12 production is sufficient so that the strain produces a lower concentration of porphyrin as compared to the non-engineered strain or to a disease-associated strain of P. acnes. In some embodiments, a non-pathogenic bacteria from which the synthetic bacteria was derived is engineered by modifying, supplementing, and/or removing one or more biomolecules of the native vitamin B12 metabolic pathway of the non-pathogenic bacteria. In some embodiments, the biomolecule is an enzyme encoded by one or more of the following genes: cysG-cbiX, cobI-cbiL, cobM, cbiF, cobK, cbiJ, cobH, cbiC, cobB-cbiA, cobO, btuR, cobQ, cbiP, cbiB, cobD, cobS, and cobV. In some embodiments, at least one of the vitamin B12 metabolic pathway biomolecules is encoded by a gene exogenous to the non-pathogenic bacteria. In some embodiments, at least one of the vitamin B12 metabolic pathway biomolecules is encoded by a gene heterologous to the non-pathogenic bacteria. In some embodiments, the synthetic bacteria is cultured in a substantially anaerobic culture medium. In some embodiments, the synthetic bacteria is useful for the treatment of acne in an individual in need thereof. In some embodiments, a synthetic bacteria comprises a vitamin B12 metabolic pathway comprising enzymes encoded by one or more of the following genes: cysG-cbiX, cobI-cbiL, cobM, cbiF, cobK, cbiJ, cobH, cbiC, cobB-cbiA, cobO, btuR, cobQ, cbiP, cbiB, cobD, cobS, and cobV; wherein at least one of the vitamin B12 metabolic pathway enzymes is encoded by a gene exogenous to the non-pathogenic bacteria; and the exogenous gene is expressed in the synthetic bacteria in an amount sufficient to produce a high level of intracellular vitamin B12 in the synthetic bacteria.

In another aspect of the disclosure, synthetic bacteria are provided that comprise an engineered porphyrin pathway that modulates production of porphyrin in the synthetic bacteria to levels suitable for therapeutic use. As a non-limiting example, for synthetic bacteria derived from health-associated P. acnes strains, the synthetic bacteria produces a lower concentration of porphyrin as compared to the non-engineered strain or to a disease-associated strain of P. acnes. In some cases, the synthetic bacteria produces less than about 4 micromolar of porphyrin. In some embodiments, a non-pathogenic bacteria from which the synthetic bacteria was derived is engineered by modifying, supplementing, and/or removing one or more biomolecules of the native porphyrin pathway of the non-pathogenic bacteria. In some embodiments, the biomolecule is an enzyme encoded by one or more of the following genes: COX15, cyoE, and HemB, HemC, HemD, HemE, HemF, HemG, HemH, HemY, or PPA2095 protoporphyrinogen oxidase (HemY homologue). In some embodiments, at least one of the porphyrin pathway biomolecules is encoded by a gene exogenous to the non-pathogenic bacteria. In some embodiments, at least one of the porphyrin pathway biomolecules is encoded by a gene heterologous to the non-pathogenic bacteria. In some embodiments, the synthetic bacteria is cultured in a substantially anaerobic culture medium. In some embodiments, the synthetic bacteria is useful for the treatment of acne in an individual in need thereof. In some embodiments, a synthetic bacteria comprises a porphyrin pathway comprising enzymes encoded one or more of the following genes: COX15, cyoE, and HemB, HemC, HemD, HemE, HemF, HemG, HemH, HemY, or PPA2095 protoporphyrinogen oxidase (HemY homologue); wherein at least one of the porphyrin pathway enzymes is encoded by a gene exogenous to the non-pathogenic bacteria; and the exogenous gene is expressed in the synthetic bacteria in an amount sufficient to not produce porphyrin, or to produce less than about 4 micromolar of porphyrin in the synthetic bacteria.

In another aspect of the disclosure, synthetic bacteria are provided that comprise an engineered citric acid pathway that modulates production of glycine in the synthetic bacteria to levels suitable for therapeutic use. As a non-limiting example, for synthetic bacteria derived from health-associated P. acnes strains, the synthetic bacteria produces a lower concentration of glycine as compared to the non-engineered strain or to a disease-associated strain of P. acnes. As a non-limiting example, for synthetic bacteria derived from health-associated P. acnes strains, the synthetic bacteria produces a higher concentration of glycine as compared to the non-engineered strain or to a disease-associated strain of P. acnes. In some embodiments, a non-pathogenic bacteria from which the synthetic bacteria was derived is engineered by modifying, supplementing, and/or removing one or more biomolecules of the native citric acid pathway of the non-pathogenic bacteria. In some embodiments, the biomolecule is an enzyme encoded by one or more of the following genes: CS, IDH1, OGDH, DLST, and fumC. In some embodiments, at least one of the citric acid pathway biomolecules is encoded by a gene exogenous to the non-pathogenic bacteria. In some embodiments, at least one of the citric acid pathway biomolecules is encoded by a gene heterologous to the non-pathogenic bacteria. In some embodiments, the synthetic bacteria is cultured in a substantially anaerobic culture medium. In some embodiments, the synthetic bacteria is useful for the treatment of acne in an individual in need thereof. In some embodiments, a synthetic bacteria comprises a citric acid pathway comprising enzymes encoded by one or more of the following genes: CS, IDH1, OGDH, DLST, and fumC; wherein at least one of the citric acid pathway enzymes is encoded by a gene exogenous to the non-pathogenic bacteria; and the exogenous gene is expressed in the synthetic bacteria in an amount sufficient produce glycine in the synthetic bacteria.

In another aspect of the disclosure, synthetic bacteria are provided that comprise an engineered nitrous oxide pathway that modulates production of nitrous oxide in the synthetic bacteria to levels suitable for therapeutic use. As a non-limiting example, for synthetic bacteria derived from health-associated P. acnes strains, the synthetic bacteria produces a lower concentration of nitrous oxide as compared to the non-engineered strain or to a disease-associated strain of P. acnes. As a non-limiting example, for synthetic bacteria derived from health-associated P. acnes strains, the synthetic bacteria produces a higher concentration of nitrous oxide as compared to the non-engineered strain or to a disease-associated strain of P. acnes. In some embodiments, a non-pathogenic bacteria from which the synthetic bacteria was derived is engineered by modifying, supplementing, and/or removing one or more biomolecules of the native nitrous oxide pathway of the non-pathogenic bacteria. In some embodiments, the biomolecule is an enzyme encoded by one or more of the following genes: narJ, narI, narH, narG, E.1.7.2.1, norB, gdhA, gudB, and rocG. In some embodiments, at least one of the nitrous oxide pathway biomolecules is encoded by a gene exogenous to the non-pathogenic bacteria. In some embodiments, at least one of the nitrous oxide pathway biomolecules is encoded by a gene heterologous to the non-pathogenic bacteria. In some embodiments, the synthetic bacteria is cultured in a substantially anaerobic culture medium. In some embodiments, the synthetic bacteria is useful for the treatment of acne in an individual in need thereof. In some embodiments, a synthetic bacteria comprises a nitrous oxide pathway comprising enzymes encoded by one or more of the following genes: narJ, narI, narH, narG, E.1.7.2.1, norB, gdhA, gudB, and rocG; wherein at least one of the nitrous oxide pathway enzymes is encoded by a gene exogenous to the non-pathogenic bacteria; and the exogenous gene is expressed in the synthetic bacteria in an amount sufficient produce nitrous oxide in the synthetic bacteria.

In another aspect of the disclosure, synthetic bacteria are provided that comprise an engineered fatty acid synthesis pathway that modulates production of fatty acid synthesis in the synthetic bacteria to levels suitable for therapeutic use. As a non-limiting example, for synthetic bacteria derived from health-associated P. acnes strains, the synthetic bacteria produces a lower concentration of fatty acids as compared to the non-engineered strain or to a disease-associated strain of P. acnes. As a non-limiting example, for synthetic bacteria derived from health-associated P. acnes strains, the synthetic bacteria produces a higher concentration of fatty acids as compared to the non-engineered strain or to a disease-associated strain of P. acnes. In some embodiments, a non-pathogenic bacteria from which the synthetic bacteria was derived is engineered by modifying, supplementing, and/or removing one or more biomolecules of the native fatty acid synthesis pathway of the non-pathogenic bacteria. In some embodiments, the biomolecule is an enzyme encoded by fadD. In some embodiments, at least one of the fatty acid synthesis pathway biomolecules is encoded by a gene exogenous to the non-pathogenic bacteria. In some embodiments, at least one of the fatty acid synthesis pathway biomolecules is encoded by a gene heterologous to the non-pathogenic bacteria. In some embodiments, the synthetic bacteria is cultured in a substantially anaerobic culture medium. In some embodiments, the synthetic bacteria is useful for the treatment of acne in an individual in need thereof. In some embodiments, a synthetic bacteria comprises a fatty acid synthesis pathway comprising an enzyme encoded by fabD; wherein at least one of the fatty acid synthesis pathway enzymes is encoded by a gene exogenous to the non-pathogenic bacteria; and the exogenous gene is expressed in the synthetic bacteria in an amount sufficient produce free fatty acids in the synthetic bacteria.

In another aspect of the disclosure, synthetic bacteria derived from a non-pathogenic bacteria are provided, the synthetic bacteria adapted to secrete a corticotropin releasing hormone (CRH), a corticotropin releasing hormone receptor (CRHR), a corticotropin releasing hormone binding protein (CRHBP), or a combination thereof. Further provided are methods of engineering the non-pathogenic bacteria to produce the synthetic bacteria. The synthetic bacteria provided herein are useful for the methods of treatment further described elsewhere in this disclosure. In many cases, the non-pathogenic bacteria is a health-associated P. acnes strain. As a non-limiting example, for synthetic bacteria derived from health-associated P. acnes strains, the synthetic bacteria produces a level of CRH, CRHR, and/or CRHBP different from that produced by the non-pathogenic bacteria or a disease-associated strain of P. acnes. In some embodiments, the non-pathogenic bacteria from which the synthetic bacteria was derived is engineered by modifying, supplementing, and/or removing one or more biomolecules of the non-pathogenic bacteria to modulate secretion levels of CRH, CRHR, and/or CRHBP in the synthetic bacteria. Biomolecules include nucleic acids such as genes and those effecting expression of said genes.

In another aspect of the disclosure, synthetic bacteria derived from a non-pathogenic bacteria are provided, wherein the synthetic bacteria are adapted to secrete a interleukin-1 inhibitor. Further provided are methods of engineering the non-pathogenic bacteria to produce the synthetic bacteria. The synthetic bacteria provided herein are useful for the methods of treatment further described elsewhere in this disclosure. In many cases, the non-pathogenic bacteria is a health-associated P. acnes strain. As a non-limiting example, for synthetic bacteria derived from health-associated P. acnes strains, the synthetic bacteria produces a level of interleukin-1 inhibitor different from that produced by the non-pathogenic bacteria or a disease-associated strain of P. acnes. In some embodiments, the non-pathogenic bacteria from which the synthetic bacteria was derived is engineered by modifying, supplementing, and/or removing one or more biomolecules of the non-pathogenic bacteria to modulate secretion levels of interleukin-1 inhibitor in the synthetic bacteria. Biomolecules include nucleic acids such as genes and those effecting expression of said genes.

In another aspect of the disclosure, synthetic bacteria derived from a non-pathogenic bacteria are provided, where the synthetic bacteria are adapted to secrete a TNF-alpha inhibitor. Further provided are methods of engineering the non-pathogenic bacteria to produce the synthetic bacteria. The synthetic bacteria provided herein are useful for the methods of treatment further described elsewhere in this disclosure. In many cases, the non-pathogenic bacteria is a health-associated P. acnes strain. As a non-limiting example, for synthetic bacteria derived from health-associated P. acnes strains, the synthetic bacteria produces a level of TNF-alpha inhibitor different from that produced by the non-pathogenic bacteria or a disease-associated strain of P. acnes. In some embodiments, the non-pathogenic bacteria from which the synthetic bacteria was derived is engineered by modifying, supplementing, and/or removing one or more biomolecules of the non-pathogenic bacteria to modulate secretion levels of TNF-alpha inhibitor in the synthetic bacteria. Biomolecules include nucleic acids such as genes and those effecting expression of said genes.

In another aspect of the disclosure, synthetic bacteria derived from a non-pathogenic bacteria are provided, wherein the synthetic bacteria are adapted to secrete a TNF-alpha inhibitor and an interleukin-8 inhibitor. Further provided are methods of engineering the non-pathogenic bacteria to produce the synthetic bacteria. The synthetic bacteria provided herein are useful for the methods of treatment further described elsewhere in this disclosure. In many cases, the non-pathogenic bacteria is a health-associated P. acnes strain. As a non-limiting example, for synthetic bacteria derived from health-associated P. acnes strains, the synthetic bacteria produces a level of TNF-alpha inhibitor and an interleukin-8 inhibitor different from that produced by the non-pathogenic bacteria or a disease-associated strain of P. acnes. In some embodiments, the non-pathogenic bacteria from which the synthetic bacteria was derived is engineered by modifying, supplementing, and/or removing one or more biomolecules of the non-pathogenic bacteria to modulate secretion levels of TNF-alpha inhibitor and an interleukin-8 inhibitor in the synthetic bacteria. Biomolecules include nucleic acids such as genes and those effecting expression of said genes.

In another aspect of the disclosure, synthetic bacteria derived from a non-pathogenic bacteria are provided, wherein the synthetic bacteria are adapted to secrete a tumor neutrophil chemotaxis inhibitor. Further provided are methods of engineering the non-pathogenic bacteria to produce the synthetic bacteria. The synthetic bacteria provided herein are useful for the methods of treatment further described elsewhere in this disclosure. In many cases, the non-pathogenic bacteria is a health-associated P. acnes strain. As a non-limiting example, for synthetic bacteria derived from health-associated P. acnes strains, the synthetic bacteria produces a level of tumor neutrophil chemotaxis inhibitor different from that produced by the non-pathogenic bacteria or a disease-associated strain of P. acnes. In some embodiments, the non-pathogenic bacteria from which the synthetic bacteria was derived is engineered by modifying, supplementing, and/or removing one or more biomolecules of the non-pathogenic bacteria to modulate secretion levels of tumor neutrophil chemotaxis inhibitor in the synthetic bacteria. Biomolecules include nucleic acids such as genes and those effecting expression of said genes.

In another aspect of the disclosure, synthetic bacteria derived from a non-pathogenic bacteria are provided, wherein the synthetic bacteria are adapted to secrete a interleukin-6 inhibitor. Further provided are methods of engineering the non-pathogenic bacteria to produce the synthetic bacteria. The synthetic bacteria provided herein are useful for the methods of treatment further described elsewhere in this disclosure. In many cases, the non-pathogenic bacteria is a health-associated P. acnes strain. As a non-limiting example, for synthetic bacteria derived from health-associated P. acnes strains, the synthetic bacteria produces a level of interleukin-6 inhibitor different from that produced by the non-pathogenic bacteria or a disease-associated strain of P. acnes. In some embodiments, the non-pathogenic bacteria from which the synthetic bacteria was derived is engineered by modifying, supplementing, and/or removing one or more biomolecules of the non-pathogenic bacteria to modulate secretion levels of interleukin-6 inhibitor in the synthetic bacteria. Biomolecules include nucleic acids such as genes and those effecting expression of said genes.

In another aspect of the disclosure, synthetic bacteria derived from a non-pathogenic bacteria are provided, wherein the synthetic bacteria are adapted to secrete a NFkB inhibitor. Further provided are methods of engineering the non-pathogenic bacteria to produce the synthetic bacteria. The synthetic bacteria provided herein are useful for the methods of treatment further described elsewhere in this disclosure. In many cases, the non-pathogenic bacteria is a health-associated P. acnes strain. As a non-limiting example, for synthetic bacteria derived from health-associated P. acnes strains, the synthetic bacteria produces a level of NFkB inhibitor different from that produced by the non-pathogenic bacteria or a disease-associated strain of P. acnes. In some embodiments, the non-pathogenic bacteria from which the synthetic bacteria was derived is engineered by modifying, supplementing, and/or removing one or more biomolecules of the non-pathogenic bacteria to modulate secretion levels of NFkB inhibitor in the synthetic bacteria. Biomolecules include nucleic acids such as genes and those effecting expression of said genes.

In another aspect of the disclosure, synthetic bacteria derived from a non-pathogenic bacteria are provided, wherein the synthetic bacteria are adapted to secrete one or more of: human beta defensing-1 inhibitor, human beta defensing-2 inhibitor, and human beta defensing-3 inhibitor. Further provided are methods of engineering the non-pathogenic bacteria to produce the synthetic bacteria. The synthetic bacteria provided herein are useful for the methods of treatment further described elsewhere in this disclosure. In many cases, the non-pathogenic bacteria is a health-associated P. acnes strain. As a non-limiting example, for synthetic bacteria derived from health-associated P. acnes strains, the synthetic bacteria produces a level of human beta defensing-1 inhibitor, human beta defensing-2 inhibitor, and/or human beta defensing-3 inhibitor different from that produced by the non-pathogenic bacteria or a disease-associated strain of P. acnes. In some embodiments, the non-pathogenic bacteria from which the synthetic bacteria was derived is engineered by modifying, supplementing, and/or removing one or more biomolecules of the non-pathogenic bacteria to modulate secretion levels of human beta defensing-1 inhibitor, human beta defensing-2 inhibitor, and/or human beta defensing-3 inhibitor in the synthetic bacteria. Biomolecules include nucleic acids such as genes and those effecting expression of said genes.

In another aspect of the disclosure, synthetic bacteria derived from a non-pathogenic bacteria are provided, wherein the synthetic bacteria are adapted to secrete an antiantroden. Further provided are methods of engineering the non-pathogenic bacteria to produce the synthetic bacteria. The synthetic bacteria provided herein are useful for the methods of treatment further described elsewhere in this disclosure. In many cases, the non-pathogenic bacteria is a health-associated P. acnes strain. As a non-limiting example, for synthetic bacteria derived from health-associated P. acnes strains, the synthetic bacteria produces a level of antiantroden different from that produced by the non-pathogenic bacteria or a disease-associated strain of P. acnes. In some embodiments, the non-pathogenic bacteria from which the synthetic bacteria was derived is engineered by modifying, supplementing, and/or removing one or more biomolecules of the non-pathogenic bacteria to modulate secretion levels of antiantroden in the synthetic bacteria. Biomolecules include nucleic acids such as genes and those effecting expression of said genes.

In another aspect of the disclosure, synthetic bacteria derived from a non-pathogenic bacteria are provided, wherein the synthetic bacteria are adapted to secrete testosterone, a DHT inhibitor, and derivatives and combinations thereof. Further provided are methods of engineering the non-pathogenic bacteria to produce the synthetic bacteria. The synthetic bacteria provided herein are useful for the methods of treatment further described elsewhere in this disclosure. In many cases, the non-pathogenic bacteria is a health-associated P. acnes strain. As a non-limiting example, for synthetic bacteria derived from health-associated P. acnes strains, the synthetic bacteria produces a level of testosterone, a DHT inhibitor, and/or derivatives thereof different from that produced by the non-pathogenic bacteria or a disease-associated strain of P. acnes. In some embodiments, the non-pathogenic bacteria from which the synthetic bacteria was derived is engineered by modifying, supplementing, and/or removing one or more biomolecules of the non-pathogenic bacteria to modulate secretion levels of testosterone, a DHT inhibitor, and/or derivatives thereof in the synthetic bacteria. Biomolecules include nucleic acids such as genes and those effecting expression of said genes.

In another aspect of the disclosure, synthetic bacteria derived from a non-pathogenic bacteria are provided, wherein the synthetic bacteria are adapted to secrete an AP-1 inhibitor. Further provided are methods of engineering the non-pathogenic bacteria to produce the synthetic bacteria. The synthetic bacteria provided herein are useful for the methods of treatment further described elsewhere in this disclosure. In many cases, the non-pathogenic bacteria is a health-associated P. acnes strain. As a non-limiting example, for synthetic bacteria derived from health-associated P. acnes strains, the synthetic bacteria produces a level of AP-1 inhibitor different from that produced by the non-pathogenic bacteria or a disease-associated strain of P. acnes. In some embodiments, the non-pathogenic bacteria from which the synthetic bacteria was derived is engineered by modifying, supplementing, and/or removing one or more biomolecules of the non-pathogenic bacteria to modulate secretion levels of AP-1 inhibitor in the synthetic bacteria. Biomolecules include nucleic acids such as genes and those effecting expression of said genes.

In another aspect of the disclosure, synthetic bacteria derived from a non-pathogenic bacteria are provided, wherein the synthetic bacteria are adapted to secrete a retinoid or a derivative thereof. Further provided are methods of engineering the non-pathogenic bacteria to produce the synthetic bacteria. The synthetic bacteria provided herein are useful for the methods of treatment further described elsewhere in this disclosure. In many cases, the non-pathogenic bacteria is a health-associated P. acnes strain. As a non-limiting example, for synthetic bacteria derived from health-associated P. acnes strains, the synthetic bacteria produces a level of retinoid or a derivative thereof different from that produced by the non-pathogenic bacteria or a disease-associated strain of P. acnes. In some embodiments, the non-pathogenic bacteria from which the synthetic bacteria was derived is engineered by modifying, supplementing, and/or removing one or more biomolecules of the non-pathogenic bacteria to modulate secretion levels of retinoid or a derivative thereof in the synthetic bacteria. Biomolecules include nucleic acids such as genes and those effecting expression of said genes. Non-limiting examples of retinoids or derivatives thereof include retinol, adapalene, tretinoin, tazarotene, and retinoic acid.

In another aspect of the disclosure, synthetic bacteria derived from a non-pathogenic bacteria are provided, wherein the synthetic bacteria are adapted to secrete a compound with a binding affinity for retinoid binding protein; wherein binding of the compound with the retinoid binding protein activates the retinoid binding protein. Further provided are methods of engineering the non-pathogenic bacteria to produce the synthetic bacteria. The synthetic bacteria provided herein are useful for the methods of treatment further described elsewhere in this disclosure. In many cases, the non-pathogenic bacteria is a health-associated P. acnes strain. As a non-limiting example, for synthetic bacteria derived from health-associated P. acnes strains, the synthetic bacteria produces a level of a compound with a binding affinity for retinoid binding protein different from that produced by the non-pathogenic bacteria or a disease-associated strain of P. acnes. In some embodiments, the non-pathogenic bacteria from which the synthetic bacteria was derived is engineered by modifying, supplementing, and/or removing one or more biomolecules of the non-pathogenic bacteria to modulate secretion levels of a compound with a binding affinity for retinoid binding protein in the synthetic bacteria. Biomolecules include nucleic acids such as genes and those effecting expression of said genes.

In another aspect of the disclosure, synthetic bacteria derived from a non-pathogenic bacteria are provided, wherein the synthetic bacteria are adapted to secrete a PPAR-ligand inhibitor. Further provided are methods of engineering the non-pathogenic bacteria to produce the synthetic bacteria. The synthetic bacteria provided herein are useful for the methods of treatment further described elsewhere in this disclosure. In many cases, the non-pathogenic bacteria is a health-associated P. acnes strain. As a non-limiting example, for synthetic bacteria derived from health-associated P. acnes strains, the synthetic bacteria produces a level of PPAR-ligand inhibitor different from that produced by the non-pathogenic bacteria or a disease-associated strain of P. acnes. In some embodiments, the non-pathogenic bacteria from which the synthetic bacteria was derived is engineered by modifying, supplementing, and/or removing one or more biomolecules of the non-pathogenic bacteria to modulate secretion levels of PPAR-ligand inhibitor in the synthetic bacteria. Biomolecules include nucleic acids such as genes and those effecting expression of said genes.

In another aspect of the disclosure, synthetic bacteria derived from a non-pathogenic bacteria are provided, wherein the synthetic bacteria are adapted to secrete one or more molecules heterologous to the non-pathogenic bacteria. In many cases, the non-pathogenic bacteria is a health-associated P. acnes strain. Further provided are methods of engineering the non-pathogenic bacteria to produce the synthetic bacteria. The synthetic bacteria provided herein are useful for the methods of treatment further described elsewhere in this disclosure. As non-limiting examples, a heterologous molecule includes a human hormone, interleukin, and antibody. In another aspect of the disclosure, synthetic bacteria derived from a non-pathogenic bacteria are provided, the synthetic bacteria adapted to secrete an antibody specific for TNF-alpha. In another aspect of the disclosure, synthetic bacteria derived from a non-pathogenic bacteria are provided, the synthetic bacteria adapted to express human Trefoil Factor 1. In another aspect of the disclosure, synthetic bacteria derived from a non-pathogenic bacteria are provided, the synthetic bacteria adapted to express a non-toxic level of an adhesion antibody specific for a cell surface protein of a keratinocyte.

### Non-Pathogenic Bacteria

Provided herein are compositions for treating and preventing skin disorders, wherein the compositions comprise a synthetic bacteria wherein the synthetic bacteria is derived from a non-pathogenic. In certain embodiments, the non-pathogenic bacteria is a “health-associated microbe.” Generally, the term, “health-associated microbe,” as used herein, refers to a microbe that is more prevalent in healthy or individuals free of a skin disease than in individuals diagnosed with the skin disease. In some embodiments, health-associated microbes disclosed herein are associated with desirable or optimal oral health. In some embodiments, health-associated microbes disclosed herein are associated with desirable or optimal gastrointestinal health.

In certain embodiments, health-associated microbes disclosed herein are associated with desirable health, optimal health or improved health relative to the health of a subject with a disease, disorder or condition disclosed herein. Desirable health, optimal health or improved health may be characterized as free of a condition, disorder or disease. Desirable health, optimal health or improved health may be characterized as free of one or more symptoms of a condition, disorder or disease. Desirable health, optimal health or improved health may be characterized as free of all symptoms of a condition, disorder or disease. Desirable health, optimal health or improved health may be characterized as improved health relative to health with a disease, disorder or condition. In certain embodiments, the health-associated microbe is associated with optimal, desirable or improved skin health. In certain embodiments, the health-associated microbe is associated with optimal, desirable or improved oral health. In certain embodiments, the health-associated microbe is associated with optimal, desirable or improved digestive health. In certain embodiments, the health-associated microbe is a P. acnes strain associated with skin health, oral health, digestive health, or any combination thereof, that is optimal, desirable or improved relative to respective health associated with a condition, disorder or disease.

In certain embodiments, there is a statistically significant difference in the presence of the health-associated microbe on the skin of an individual free of a disease when compared to an individual with the disease. In certain embodiments, there is at least about a 10% greater quantity of a health-associated microbe on the skin of an individual free of a disease when compared to an individual with the disease. In certain embodiments, there is at least about a 50% greater quantity of a health-associated microbe on the skin of an individual free of a disease when compared to an individual with the disease. In certain embodiments, there is at least about a 100% greater quantity of a health-associated microbe on the skin of an individual free of a disease when compared to an individual with the disease. In certain embodiments, there is at least about a 2-fold greater presence of the health-associated microbe on the skin of an individual free of a disease when compared to an individual with the disease. In certain embodiments, there is at least about a 3-fold greater presence of the health-associated microbe on the skin of an individual free of a disease when compared to an individual with the disease. In certain embodiments, there is at least about a 5-fold greater presence of the health-associated microbe on the skin of an individual free of a disease when compared to an individual with the disease. In certain embodiments, there is at least about a 10-fold greater presence of the health-associated microbe on the skin of an individual free of a disease when compared to an individual with the disease.

In certain embodiments, the health-associated microbe is an isolated species of bacteria. In certain embodiments, the health-associated microbe is a purified species of bacteria. In certain embodiments, the health-associated microbe is an isolated and purified species of bacteria. In certain embodiments, the health-associated microbe is an isolated strain of bacteria. In certain embodiments, the health-associated microbe is a purified strain of bacteria. In certain embodiments, the health-associated microbe is an isolated and purified strain of bacteria. In certain embodiments, the health-associated microbe is an isolated species of Propionibacterium. In certain embodiments, the health-associated microbe is a purified species of Propionibacterium. In certain embodiments, the health-associated microbe is an isolated and purified species of Propionibacterium. In certain embodiments, the health-associated microbe is an isolated strain of P. acnes. In certain embodiments, the health-associated microbe is a purified strain of P. acnes. In certain embodiments, the health-associated microbe is an isolated and purified strain of P. acnes.

As described herein, some strains of P. acnes are associated with acne and some strains of P. acnes are associated with skin free of acne or disease. These P. acnes strains can be differentiated at the genetic level by using nucleic acid sequence determination methods known in the art such as PCR, restriction mapping, Sanger sequencing, and next-generation sequencing. In some instances, a health-associated microbe disclosed herein is beneficial for the treatment of a specific skin disorder, but not all skin disorders. In some instances, a health-associated microbe disclosed herein is beneficial for the treatment of a plurality of skin disorder, but not all skin disorders. In some instances, a health-associated microbe disclosed herein is beneficial for the treatment any skin disorder. By way of non-limiting example, a health-associated microbe disclosed herein may be beneficial only for acne, but not for eczema, seborrheic dermatitis, or psoriasis. In another instance, a health-associated microbe disclosed herein is beneficial only for eczema, but not for acne, seborrheic dermatitis, or psoriasis. In another instance, a health-associated microbe disclosed herein is beneficial only for psoriasis, but not for acne, seborrheic dermatitis, or eczema. In another instance, a health-associated microbe disclosed herein is beneficial only for seborrheic dermatitis, but not for psoriasis, acne, or eczema. In some instances, a health-associated microbe disclosed herein is beneficial for eczema, acne and psoriasis. In some instances, a health-associated microbe disclosed herein is beneficial for acne and a condition selected from eczema, seborrheic dermatitis. In some instances, a health-associated microbe disclosed herein is beneficial for eczema, seborrheic dermatitis, acne and psoriasis.

In certain embodiments, non-pathogenic bacteria disclosed herein comprise at least one health-associated microbe, wherein the health-associated microbe is a strain of P. acnes or bacteria that is associated with healthy or normal skin. In certain embodiments, compositions disclosed herein comprise at least one health-associated microbe, wherein the health-associated microbe is a strain of P. acnes or bacteria that produces low levels of inflammatory mediators when incubated with a subject's own keratinocytes or pooled keratinocytes from multiple subjects.

In certain embodiments, non-pathogenic bacteria comprise an isolated P. acnes strain. In some embodiments, the isolated P. acnes strain is a purified strain. In certain embodiments, compositions comprise a mixture of about 2 to about 10 isolated P. acnes strains. In certain embodiments, compositions comprise a mixture of about 3 to about 8 isolated P. acnes strains. In certain embodiments, compositions comprise a mixture of about 2 to about 5 isolated P. acnes strains. In certain embodiments, compositions comprise a mixture of about 3 to about 6 isolated P. acnes strains. In certain embodiments, the isolated strain is isolated based on its phylotype or ribotype.

In certain embodiments, non-pathogenic bacteria disclosed herein comprise at least one P. acnes strain having a health-associated phylotype. In certain embodiments, the health-associated phylotype is selected from type I, type II, and type III. In some embodiments, compositions disclosed herein comprise at least two P. acnes strains having health-associated phylotypes, wherein the health-associated phylotypes are a combination of type I, type II, and type III. In certain embodiments, the type I phylotype is selected from type IA, type IB, and type IC. In certain embodiments, the type IA phylotype is selected from type IA₁ and type IA₂. Strains can be phylotyped as in McDowell et al. (PLoS ONE 8(9): e70897 (2013)).

In some embodiments, non-pathogenic bacteria disclosed herein comprise a combination of health-associated microbes, wherein the health-associated microbes comprise a combination of healthy strains of P. acnes. In some embodiments, combinations of healthy strains of P. acnes comprise a combination of strains of P. acnes of a plurality of ribotypes. In some embodiments, the plurality of ribotypes comprises at least two ribotypes selected from RT1, RT2, RT3, RT7, RT8, RT9, and RT10. In some embodiments, the plurality of ribotypes comprises at least two ribotypes selected from RT1, RT2 and RT3. In certain embodiments, the plurality of ribotypes comprises at least two ribotypes selected from RT1, RT2, RT3 and not RT6. In certain embodiments, the plurality of ribotypes comprises ribotypes selected from RT1 and RT2. In certain embodiments, the plurality of ribotypes comprises ribotypes selected from RT1 and RT3. In certain embodiments, the plurality of ribotypes comprises ribotypes selected from RT2 and RT3. In certain embodiments, the plurality of ribotypes comprises RT1, but not RT6. In certain embodiments, the plurality of ribotypes comprises RT2, but not RT6.

In some embodiments, compositions disclosed herein comprise a combination of health-associated microbes, wherein the health-associated microbes comprise a combination of healthy strains of P. acnes. In some embodiments, the combination comprises a first strain of P. acnes and a second strain of P. acnes. In some embodiments, the first strain of P. acnes is of a first ribotype and a second strain of P. acnes is of a second ribotype. In some embodiments, the first ribotype and the second ribotype are the same. In some embodiments, the first ribotype and the second ribotype are different. In some embodiments, the first ribotype is RT1 and the second ribotype is RT1. In some embodiments, the first ribotype is RT2 and the second ribotype is RT2. In some embodiments, the first ribotype is RT1 and the second ribotype is RT2. In some embodiments, the first ribotype is RT1 and the second ribotype is RT3. In some embodiments, the first ribotype is RT2 and the second ribotype is RT3. In some embodiments, the first ribotype is RT1 and the second ribotype is not RT6. In some embodiments, the first ribotype is RT2 and the second ribotype is not RT6. In some embodiments, the first ribotype is RT3 and the second ribotype is not RT6.

In some embodiments, compositions disclosed herein comprise healthy strains of P. acnes, and do not comprise any other type of microbe or bacteria. In some embodiments, health-associated microbes disclosed herein comprise at least one health-associated strain of P. acnes, wherein the health-associated strain of P. acnes has a ribotype of RT1 or RT2. In some embodiments, health-associated microbes disclosed herein do not comprise a strain of P. acnes, other than a health-associated strain of P. acnes disclosed herein that has a ribotype selected from RT1 and RT2. In some embodiments, the health-associated microbes do not comprise a strain of P. acnes that has a ribotype RT6. In some embodiments, the health-associated microbes do not comprise a strain of P. acnes that expresses DNA binding response regulator or phosphoglycerate kinase, as described herein. In some embodiments, the health-associated microbes comprise a strain of P. acnes that expresses an ATP binding cassette transporter, as described herein.

In certain embodiments, compositions disclosed herein comprise a health-associated microbe, wherein the health-associated microbe is Lactobacillus reuteri (L. reuteri), or a strain thereof. In certain embodiments, compositions disclosed herein comprise at least one L. reuteri strain selected from Korean Collection for Type Cultures (KCTC) deposited strains, such as KCTC 3679, KCTC 3594, KCTC 3678, and any combination thereof.

In certain embodiments, compositions disclosed herein comprise a health-associated microbe, wherein the health-associated microbe is Staphylococcus epidermidis (S. epidermidis). In certain embodiments, compositions disclosed herein comprise at least one S. epidermidis strain selected from 14.1.R1, AS1, AU 10, AU16, AU21, AU23, AU24, AU35, AU36, AU39, AU40, AU 44, AU48, AU53, AU60, AU73, AU81, FS1, G53, IS2, and a combination thereof.

### Selected, Transformed, or Engineered Bacteria

In certain embodiments, the non-pathogenic bacteria described herein comprise one or more strains of bacteria that is selected, transformed or engineered with a gene or gene mutation that is beneficial for a skin disorder. Thus, the bacteria have been transformed into a “non-pathogenic” form, or a health-associated form from a disease-associated form. In certain embodiments, a gene that contributes to pathogenesis of a skin disorder is deleted or mutated to inactivate or reduce the corresponding gene product. In certain embodiments, a gene that reduces the pathogenesis of a skin disorder is added, or mutated to activate or increase levels of the corresponding gene product. In certain embodiments, the bacteria are grown and selected from culture or selected from healthy disease free individuals.

In certain embodiments, selected, transformed, or engineered bacteria are to be delivered as a probiotic via compositions and methods disclosed herein. In certain embodiments, selected, transformed, or engineered bacteria disclosed herein comprise a gene encoding a deoxyribose operon repressor (deoR). In certain embodiments, selected, transformed, or engineered bacteria disclosed herein express a deoxyribose operon repressor. In certain embodiments, selected, transformed, or engineered bacteria disclosed herein comprise a gene encoding a Type II lipase. In certain embodiments, selected, transformed, or engineered bacteria disclosed herein express Type II lipase. By way of non-limiting example, the Type II lipase may be a glycerol-ester hydrolase B (GehB). In certain embodiments, selected, transformed, or engineered bacteria disclosed herein do not comprise a gene encoding Type I lipase. In certain embodiments, selected, transformed, or engineered bacteria disclosed herein do not express a Type I lipase. By way of non-limiting example, the type I lipase may be a glycerol-ester hydrolase A (GehA). In certain embodiments, selected, transformed, or engineered bacteria do not comprise a pIMPLE plasmid. In certain embodiments, selected, transformed, or engineered bacteria disclosed herein comprise a gene encoding an ABC transporter. In certain embodiments, selected, transformed, or engineered bacteria expresses an ABC transporter. In certain embodiments, selected, transformed, or engineered bacteria disclosed herein do not comprise a gene encoding a phosphoglycerate kinase. In certain embodiments, selected, transformed, or engineered bacteria do not comprise a phosphoglycerate kinase. In certain embodiments, selected, transformed, or engineered bacteria do not comprise a DNA binding response regulator.

In certain embodiments, selected, transformed, or engineered bacteria disclosed herein do not express a dermatin-sulfate adhesin (e.g., DSA1, DSA2). In certain embodiments, selected, transformed, or engineered bacteria disclosed herein do comprise a nucleic acid encoding a dermatin-sulfate adhesin (e.g., DSA1, DSA2). The absence or deletion of dermatin-sulfate adhesins may disable adhesion of microbes to keratinocytes.

In certain embodiments, selected, transformed, or engineered bacteria disclosed herein express a hyaluronidase. In certain embodiments, selected, transformed, or engineered bacteria disclosed herein do not express a hyaluronidase. In certain embodiments, selected, transformed, or engineered bacteria disclosed herein comprise a nucleic acid encoding a hyaluronidase. In certain embodiments, selected, transformed, or engineered bacteria disclosed herein do not comprise a nucleic acid encoding a hyaluronidase. In certain embodiments, a strain of bacteria that is selected, transformed, or engineered bacteria is present or has increased expression of a hyaluronidase gene relative to the strain of bacteria when it is not selected, transformed, or engineered. In certain embodiments, a strain of bacteria that is selected, transformed, or engineered bacteria is present or has increased hyaluronidase activity relative to the strain of bacteria when it is not selected, transformed, or engineered. In certain embodiments, a strain of bacteria that is selected, transformed, or engineered bacteria is present or has reduced hyaluronidase activity relative to the strain of bacteria when it is not selected, transformed, or engineered. In certain embodiments, selected, transformed, or engineered bacteria disclosed herein lack a hyaluronidase gene.

In certain embodiments, selected, transformed, or engineered bacteria disclosed herein confer an antibiotic sensitivity to macrolide and tetracycline antibiotics. In certain embodiments, selected, transformed, or engineered bacteria disclosed herein have an absence or deletion of a thiopeptide encoding island, respectively. In certain embodiments, selected, transformed, or engineered bacteria disclosed herein have a presence or addition of a tyrosine decarboxylase island (which increases intracellular pH under stress to tolerate acidic environments), respectively. In certain embodiments, selected, transformed, or engineered bacteria disclosed herein have reduced transposase 2 enzyme activity. In certain embodiments, selected, transformed, or engineered bacteria disclosed herein lack transposase 2 enzyme activity. In certain embodiments, a selected, transformed, or engineered strain of bacteria disclosed herein has reduced activity relative to the strain when it is not selected, transformed, or engineered, respectively.

In certain embodiments, selected, transformed, or engineered bacteria comprise selected, transformed, or engineered S. epidermis, respectively. In certain embodiments, the selected, transformed, or engineered bacteria comprise selected, transformed, or engineered P. acnes.

In certain embodiments, the non-pathogenic bacteria are transformed using recombinant DNA techniques known in the art. In certain embodiments, the bacteria are transformed by growing bacteria under selective pressure to acquire or lose a gene, gene product, or expression thereof. In a certain embodiment, the bacteria are transformed using CRISPR technology. In a certain embodiment, the bacteria are transformed using homologous recombination.

In certain embodiments, the selected, transformed, or engineered bacteria comprise a selected, transformed, or engineered P. acnes strain. In certain embodiments, the selected, transformed, or engineered bacteria comprise a selected, transformed, or engineered P. acnes strain, wherein the selected, transformed, or engineered P. acnes strain has a ribotype selected from RT1, RT2, RT3, RT4, RT5, RT7, RT8, RT9, and RT10. In certain embodiments, the selected, transformed, or engineered bacteria comprise a selected, transformed, or engineered P. acnes strain, wherein the selected, transformed, or engineered P. acnes strain has a ribotype selected from RT1, RT2, RT3, RT7, RT8, RT9, and RT10. In certain embodiments, the selected, transformed, or engineered bacteria comprise a selected, transformed, or engineered P. acnes strain, wherein the selected, transformed, or engineered P. acnes strain has a ribotype selected from RT1, RT2, RT3, RT7, RT8, RT9, and RT10. In certain embodiments, the selected, transformed, or engineered bacteria comprise a selected, transformed, or engineered P. acnes strain, wherein the selected, transformed, or engineered P. acnes strain has a ribotype selected from RT1, RT2, RT3, and not RT6. In certain embodiments, the selected, transformed, or engineered bacteria comprise a selected, transformed, or engineered P. acnes strain, wherein the selected, transformed, or engineered P. acnes strain has a ribotype selected from RT1 and RT2. In certain embodiments, the selected, transformed, or engineered bacteria comprise a selected, transformed, or engineered P. acnes strain, wherein the selected, transformed, or engineered P. acnes strain has a ribotype of RT1. In certain embodiments, the selected, transformed, or engineered bacteria comprise a selected, transformed, or engineered P. acnes strain, wherein the selected, transformed, or engineered P. acnes strain has a ribotype of RT2. In certain embodiments, the selected, transformed, or engineered bacteria are an RT1 strain of P. acnes. In certain embodiments, the selected, transformed, or engineered bacteria are an RT2 strain of P. acnes. In certain embodiments, the selected, transformed, or engineered bacteria are an RT3 strain of P. acnes. In certain embodiments, the selected, transformed, or engineered bacteria are not an RT6 strain of P. acnes.

### Markers

Disclosed herein are compositions that comprise synthetic bacteria. Synthetic bacteria disclosed herein are generally derived from at least one strain of bacteria, wherein the at least one strain of bacteria exhibits a health-associated presence, health-associated absence or health-associated expression level of at least one marker. In some instances, health-associated expression of the at least one marker is a lack of expression. In some instances, health-associated expression of the at least one marker is expression that is low as compared to expression of the at least one marker in a reference strain. In some instances, the reference strain is a pathogenic strain. In some instances, the reference strain is not a health-associated strain. In some instances, health-associated expression of the at least one marker is expression that is high as compared to expression of the at least one marker in a reference strain. In some embodiments, the marker is a nucleic acid. In some embodiments, the nucleic acid comprises a gene encoding the marker or a portion thereof. In some embodiments, the nucleic acid is a gene encoding the marker or a portion thereof. In some embodiments, the marker is a protein. In some embodiments, the marker is a peptide (e.g., less than or equal to 100 amino acids). In some embodiments, the marker is not a nucleic acid or a protein. Non-limiting examples of a marker that neither comprises a nucleic acid or protein include glycans and lipids.

Exemplary markers of health-associated microbes useful for the creation of the synthetic bacteria disclosed herein include, but are not limited to, a deoxyribose operon repressor, a CRISPR associated protein (Cas), a lipase, an ATP binding cassette transporter, a DNA binding response regulator, a phosphoglycerate kinase, dermatin-sulfate adhesin, and hyaluronidase. In some instances, the at least one strain of bacteria comprises a plasmid. In some instances, presence or absence of the plasmid is a marker. By way of non-limiting example, the plasmid may be a pIMPLE plasmid disclosed herein. As further described herein, presence of a deoR, a type II lipase, an ABC transporter, or a Cas5, or a combination thereof, is generally associated with probiotics and health-associated microbes disclosed herein. In contrast, probiotics and health-associated microbes disclosed herein are generally associated with an absence or low expression of a pIMPLE plasmid, a type I lipase, a DNA binding response regulator, a phosphoglycerate kinase, or dermatin-sulfate adhesin, or a combination thereof. However, it would be understood to one of skill in the art that nature presents exceptions to such generalities. Therefore, expression patterns of these markers that are alternative or contrary to those described herein are contemplated as well. Compositions comprising one or more strains characterized by such markers are further characterized herein, including the description as follows.

Table 5 below provides a summary of non-limiting examples of non-pathogenic P. acnes bacteria, or populations of bacteria comprising P. acnes bacteria, that can be distinguished by analysis of different genetic markers. The + symbol indicates the presence of deoR or Cas5 (or nucleic acids encoding deoR or Cas5) in the columns labeled deoR and Cas5, respectively. The − symbol indicates absence of deoR or Cas5 or nucleic acids encoding deoR or Cas5) in the columns labeled deoR and Cas5, respectively. % pIMPLE plasmid refers to the number of reads aligned/number of reads tested when bacteria is sequenced for pIMPLE plasmid.

TABLE 5
Sequences of P. acnes genetic elements.

| | | | | | % | | | | |
|---|---|---|---|---|---|---|---|---|---|
| | | | | | pIMPLE | ABC | | | Exemplary |
| Group | RT | deoR | Lipase | Cas5 | plasmid | XP | DBRR | PGK | strains |
| A | 1 | − | I | − | <5% | + | − | − | |
| B | 1 | + | I | − | <5% | + | − | − | |
| C | 1 | + | II | − | <5% | + | − | − | HP3A11 |
| D | 2 | + | II | + | <5% | + | − | − | HP4G1, HP5G4 |
| E | 4 | − | I | − | >1% | + | − | − | HL045PA1 |
| F | 5 | − | I | − | >1% | + | − | − | HL043PA1 |
| G | 6 | + | II | + | >5% | − | + | + | HL110PA3, HL110PA4 |

ABC XP = ATP binding cassette transporter
DBRR = DNA binding response regulator
PGK = phosphoglycerate kinase

In certain embodiments, the composition comprises at least one strain of a P. acnes microbe that corresponds to group A of Table 5. In certain embodiments, the composition comprises at least one strain of a P. acnes microbe that corresponds to group B of Table 5. In certain embodiments, the composition comprises at least one strain of a P. acnes microbe that corresponds to group C of Table 5. In certain embodiments, the composition comprises at least one strain of a P. acnes microbe that corresponds to group D of Table 5.

In certain embodiments, the composition comprises at least one strain of a P. acnes microbe that corresponds to groups A, B, C or D of Table 5. In certain embodiments, the composition comprises at least two strains of a P. acnes microbe that correspond to groups A, B, C or D of Table 5. In certain embodiments, the composition comprises at least three strains of a P. acnes microbe that correspond to groups A, B, C or D of Table 5. In certain embodiments, the composition comprises at least four strains of a P. acnes microbe that correspond to groups A, B, C or D of Table 5. In certain embodiments, the composition comprises at least five strains of a P. acnes microbe that correspond to groups A, B, C, or D of Table 5.

In some embodiments, the composition does not comprise a strain of P. acnes that corresponds to group E of Table 5. In some embodiments, the composition does not comprise a strain of P. acnes that corresponds to group F of Table 5. In some embodiments, the composition does not comprise a strain of P. acnes that corresponds to group G of Table 5.

In some embodiments, the composition comprises at least one strain of P. acnes that corresponds to group A of Table 5, but does not comprise a strain of P. acnes that corresponds to groups E, F or G of Table 5. In some embodiments, the composition comprises at least one strain of P. acnes that corresponds to group B of Table 5, but does not comprise a strain of P. acnes that corresponds to groups E, F or G of Table 5. In some embodiments, the composition comprises at least one strain of P. acnes that corresponds to group C of Table 5, but does not comprise a strain of P. acnes that corresponds to groups E, F or G of Table 5. In some embodiments, the composition comprises at least one strain of P. acnes that corresponds to group D of Table 5, but does not comprise a strain of P. acnes that corresponds to groups E, F or G of Table 5. In some embodiments, the composition comprises at least one strain of P. acnes that corresponds to group C of Table 5 and at least one strain of P. acnes that corresponds to group D of Table 5, but does not comprise a strain of P. acnes that corresponds to groups E, F or G of Table 5.

deoR

In some embodiments, compositions disclosed herein comprise synthetic bacteria derived from at least one strain of non-pathogenic bacteria, wherein the at least one strain of non-pathogenic bacteria comprises a deoxyribose operon repressor (deoR). In some embodiments, the deoR is a deoR family transcriptional regulator expressed in Propionibacterium acnes subsp. defendens (ATCC 11828, GenBank: AER05724.1). In some embodiments, compositions disclosed herein comprise at least one strain of bacteria, wherein the at least one strain of bacteria comprises a nucleic acid encoding a deoxyribose operon repressor (deoR). In certain embodiments, the at least one strain of bacteria has been selected, transformed, or engineered to acquire the presence of the deoR. In certain embodiments, the deoR has a sequence that is at least 80% homologous to SEQ ID NO: 104. In certain embodiments, the deoR has a sequence that is at least 90% homologous to SEQ ID NO: 104. In certain embodiments, the deoR has a sequence that is at least 95% homologous to SEQ ID NO: 104. In certain embodiments, the deoR has a sequence that is at least 97% homologous to SEQ ID NO: 104. In certain embodiments, the deoR has a sequence that is at least 98% homologous to SEQ ID NO: 104. In certain embodiments, the deoR has a sequence that is at least 99% homologous to SEQ ID NO: 104. In certain embodiments, the deoR has a sequence that is 100% homologous to SEQ ID NO: 104. In certain embodiments, the at least one strain of bacteria has greater expression or activity of a deoxyribose operon repressor than a reference strain (e.g., pathogenic strain, not a health-associated strain). In certain embodiments, the at least one strain has at least about 1.5-fold, at least about 2-fold, at least about 3-fold, at least about 5-fold or at least about 10-fold greater expression or activity of the deoxyribose operon repressor as compared to the reference strain.

pIMPLE Plasmid

In some embodiments, compositions disclosed herein comprise synthetic bacteria derived from at least one strain of non-pathogenic bacteria, wherein the at least one strain of non-pathogenic bacteria does not comprise a pIMPLE plasmid. In certain embodiments, the pIMPLE plasmid has a sequence that is at least about 80% homologous to SEQ ID NO: 105. In certain embodiments, the pIMPLE plasmid has a sequence that is at least about 90% homologous to SEQ ID NO: 105. In certain embodiments, the pIMPLE plasmid has a sequence that is at least about 95% homologous to SEQ ID NO: 105. In certain embodiments, the pIMPLE plasmid has a sequence that is at least about 97% homologous to SEQ ID NO: 105. In certain embodiments, the pIMPLE plasmid has a sequence that is at least about 98% homologous to SEQ ID NO: 105. In certain embodiments, the pIMPLE plasmid has a sequence that is at least about 99% homologous to SEQ ID NO: 105. In certain embodiments, the pIMPLE plasmid has a sequence that is 100% homologous to SEQ ID NO: 105. In certain embodiments, a plasmid with at least 80%, 90%, 95%, 98%, 99%, or 100% homology to SEQ ID NO: 105 is partially or completely deleted from the at least one strain of bacteria. In certain embodiments, a plasmid with at least 80%, 90%, 95%, 98%, 99%, or 100% homology to SEQ ID NO: 105 is disrupted by an insertion of one or more nucleotides or a introduction of a frameshift mutation in the a selected, transformed, or engineered strain of bacteria. In certain embodiments, the at least one strain of bacteria contains portions of a complete pIMPLE plasmid (SEQ ID NO: 105). In certain embodiments, the at least one strain of bacteria may comprise less than about 25%, 20%, 15%, 14%, 13%, 12%, 11%, 10%, 9%, 8%, 7%, 6% of the complete pIMPLE sequence set forth in SEQ ID NO: 105. In certain embodiments, the at least one strain of bacteria does not comprise a specific portion of the pIMPLE plasmid that is present in an RT6 strain or any other disease associated strain. In certain embodiments, the at least one strain of bacteria comprises a low copy number of a pIMPLE plasmid (SEQ ID NO: 105). In certain embodiments, the at least one strain of bacteria comprises less than 5 copies of pIMPLE plasmid per bacterial genome. In certain embodiments, the at least one strain of bacteria comprises less than 4 copies of pIMPLE plasmid per bacterial genome. In certain embodiments, the at least one strain of bacteria comprises less than 3 copies of pIMPLE plasmid per bacterial genome. In certain embodiments, the at least one strain of bacteria comprises less than 2 copies of pIMPLE plasmid per bacterial genome. In certain embodiments, the at least one strain of bacteria comprises 1 copy of pIMPLE plasmid per bacterial genome. In certain embodiments, the at least one strain of bacteria comprises a low percentage of pIMPLE plasmid (SEQ ID NO: 105). In certain embodiments, the bacteria comprise less than about 15%, 14%, 13%, 12%, 11%, 10%, 9%, 8%, 7%, 6%, 5%, 4%, 3%, 2%, 1% pIMPLE plasmid. In certain embodiments, the at least one strain of bacteria comprises less than about 1.0%, 0.9%, 0.8%, 0.7%, 0.6%, 0.5%, 0.4%, 0.3%, 0.2%, 0.1% pIMPLE plasmid. pIMPLE plasmid percentage can be determined by next-generation sequencing of P. acnes bacteria, as % reads aligned. In certain embodiments, the pIMPLE percentage is percentage of total sequencing reads that align to pIMPLE from HL096PA1 (an RT5).

### Lipases

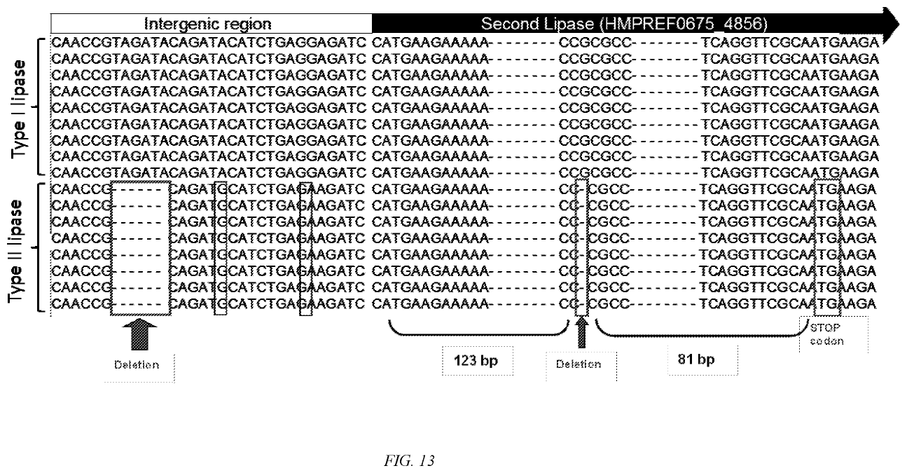

In some embodiments, compositions disclosed herein comprise synthetic bacteria derived from at least one strain of non-pathogenic bacteria, wherein the at least one strain of non-pathogenic bacteria comprises a strain of bacteria that expresses a lipase. In some embodiments, the lipase is Type I lipase. In some embodiments, the lipase is Type II lipase. In some embodiments, the compositions disclosed herein comprise a strain of bacteria that does not express a lipase. In some embodiments, the compositions disclosed herein comprise a strain of bacteria that does not express a Type I lipase. In some embodiments, the compositions disclosed herein comprise a strain of bacteria that does not express a Type II lipase. In some embodiments, the strain of bacteria comprises a nucleic acid encoding a Type I lipase. In some embodiments, the strain of bacteria comprises a nucleic acid encoding a Type II lipase. Type I lipase and Type II lipase, as described herein, may be encoded by a similar nucleic acid. For example, a gene encoding Type I lipase will encode a Type II lipase upon a 6 bp deletion in the intergenic region and a single base deletion at position 124, the latter causing a frameshift that creates premature STOP codon, see, e.g., FIG. 13.

### Type I Lipase

In some embodiments, compositions disclosed herein comprise synthetic bacteria derived from at least one strain of non-pathogenic bacteria, wherein the at least one strain of non-pathogenic bacteria does not express a Type I lipase. In some embodiments, compositions disclosed herein comprise at least one strain of bacteria, wherein the at least one strain of bacteria does not comprise a nucleic acid encoding a Type I lipase. In certain embodiments, compositions disclosed herein comprise at least one strain of bacteria, wherein the at least one strain of bacteria has been selected, transformed, or engineered for absence of Type I lipase expression or activity. In certain embodiments, compositions disclosed herein comprise at least one strain of bacteria, wherein the at least one strain of bacteria has been selected, transformed, or engineered for the presence of Type I lipase expression or activity. In certain embodiments, compositions disclosed herein comprise at least one strain of bacteria, wherein the strain has been selected, transformed, or engineered for lower expression or activity of Type I lipase relative to a reference strain (e.g., pathogenic strain, not a health-associated strain). In certain embodiments, at least one strain of bacteria has at least about 1.5-fold lower expression or activity of Type I lipase compared to the reference strain. In certain embodiments, at least one strain of bacteria has at least about 2-fold lower expression or activity of Type I lipase compared to the reference strain. In certain embodiments, at least one strain of bacteria has at least about 3-fold lower expression or activity of Type I lipase compared to the reference strain. In certain embodiments, at least one strain of bacteria has at least about 5-fold lower expression or activity of Type I lipase compared to the reference strain. In certain embodiments, at least one strain of bacteria has at least about 10-fold lower expression or activity of Type I lipase compared to the reference strain.

In some embodiments, compositions disclosed herein comprise synthetic bacteria derived from at least one strain of non-pathogenic bacteria, wherein the at least one strain of non-pathogenic bacteria expresses a Type I lipase. In certain embodiments, compositions disclosed herein comprise at least one strain of bacteria, wherein the at least one strain of bacteria has been selected, transformed, or engineered for the presence of Type I lipase expression or activity. In certain embodiments, compositions disclosed herein comprise at least one strain of bacteria, wherein the strain has been selected, transformed, or engineered for greater expression or activity of Type I lipase relative to the reference strain.

In some embodiments, at least a portion of the Type I lipase is encoded by a sequence of SEQ ID NO: 113. In some embodiments, at least a portion of the Type I lipase is encoded by a sequence that has at least 80% homology to SEQ ID NO: 113. In some embodiments, at least a portion of the Type I lipase is encoded by a sequence that has at least 90% homology to SEQ ID NO: 113. In some embodiments, at least a portion of the Type I lipase is encoded by a sequence that has at least 95% homology to SEQ ID NO: 113. In some embodiments, at least a portion of the Type I lipase is encoded by a sequence that has at least 97% homology to SEQ ID NO: 113. In some embodiments, at least a portion of the Type I lipase is encoded by a sequence that has at least 98% homology to SEQ ID NO: 113. In some embodiments, at least a portion of the Type I lipase is encoded by a sequence that has at least 99% homology to SEQ ID NO: 113. In some embodiments, the Type I lipase is encoded by a sequence that has at least 80% homology to SEQ ID NO: 113. In some embodiments, at least a portion of the Type I lipase is encoded by a sequence of SEQ ID NO: 130. In some embodiments, at least a portion of the Type I lipase is encoded by a sequence that has at least 80% homology to SEQ ID NO: 130. In some embodiments, at least a portion of the Type I lipase is encoded by a sequence that has at least 90% homology to SEQ ID NO: 130. In some embodiments, at least a portion of the Type I lipase is encoded by a sequence that has at least 95% homology to SEQ ID NO: 130. In some embodiments, at least a portion of the Type I lipase is encoded by a sequence that has at least 97% homology to SEQ ID NO: 130. In some embodiments, at least a portion of the Type I lipase is encoded by a sequence that has at least 98% homology to SEQ ID NO: 130. In some embodiments, at least a portion of the Type I lipase is encoded by a sequence that has at least 99% homology to SEQ ID NO: 130. In some embodiments, at least a portion of the Type I lipase is encoded by a sequence that has at least 80% homology to SEQ ID NO: 130.

In certain embodiments, a nucleic acid with at least 80%, 90%, 95%, 98%, 99%, or 100% homology to SEQ ID NO: 113 is partially or completely deleted from the at least one strain. In certain embodiments, the nucleic acid is deleted by at least about 5%, at least about 10%, at least about 15%, at least about 20%, at least about 25%, at least about 30%, at least about 35%, at least about 40%, at least about 45%, at least about 50%, at least about 55%, at least about 60%, at least about 65%, at least about 70%, at least about 75%, at least about 80%, at least about 85%, at least about 90%, at least about 95% or more from the 3 prime end of the nucleic acid. In certain embodiments, the nucleic acid is deleted by at least about 5%, at least about 10%, at least about 15%, at least about 20%, at least about 25%, at least about 30%, at least about 35%, at least about 40%, at least about 45%, at least about 50%, at least about 55%, at least about 60%, at least about 65%, at least about 70%, at least about 75%, at least about 80%, at least about 85%, at least about 90%, at least about 95% or more from the 5 prime end of the nucleic acid.

In certain embodiments, a nucleic acid with at least 80%, 90%, 95%, 98%, 99%, or 100% homology to SEQ ID NO: 113 is disrupted by an insertion of one or more nucleotides or a introduction of a frameshift mutation in the at least one strain. In certain embodiments, at least about 5%, at least about 10%, at least about 15%, at least about 20%, at least about 25%, at least about 30%, at least about 35%, at least about 40%, at least about 45%, at least about 50%, at least about 55%, at least about 60%, at least about 65%, at least about 70%, at least about 75%, at least about 80%, at least about 85%, at least about 90%, or at least about 95% of the nucleic acid with at least 80%, 90%, 95%, 98%, 99%, or 100% homology to SEQ ID NO: 113 is disrupted.

### Type II Lipase

In some embodiments, compositions disclosed herein comprise synthetic bacteria derived from at least one strain of non-pathogenic bacteria, wherein the at least one strain of non-pathogenic bacteria expresses a Type II lipase. A non-limiting example of a Type II lipase is Lipase ADE00051, HMPREF0675_4856. In some embodiments, compositions disclosed herein comprise at least one strain of bacteria, wherein the at least one strain of bacteria comprises a nucleic acid encoding a Type II lipase. In certain embodiments, the at least one strain of bacteria has been selected, transformed, or engineered to express a Type II lipase. In certain embodiments, the at least one strain of bacteria has at least about 1.5-fold greater expression or activity of Type II lipase compared to a reference strain (e.g., pathogenic strain, not a health-associated strain). In certain embodiments, the at least one strain of bacteria has at least about 2-fold greater expression or activity of Type II lipase compared to the reference strain. In certain embodiments, the at least one strain of bacteria has at least about 3-fold greater expression or activity of Type II lipase compared to the reference strain. In certain embodiments, the at least one strain of bacteria has at least about 5-fold greater expression or activity of Type II lipase compared to the reference strain. In certain embodiments, the at least one strain of bacteria has at least about 10-fold greater expression or activity of Type II lipase compared to the reference strain.

In some embodiments, at least a portion of the type II lipase is expressed from a nucleic acid comprising SEQ ID NO: 131. SEQ ID NO: 131 is found in the complete circular genome of Propionibacterium acnes ATCC 11828 and starts at position 390,423 of ATCC 11828. The following subsequent positions are in reference to the first nucleotide of SEQ ID NO:131. The lipase coding sequence is bases 22-1032, referred to herein as ADE0051, HMPREF0675_4856, and SEQ ID NO: 106. Bases 1-21 is an intergenic region. Type II Lipase has a G in position 7 and an A in position 16. In some embodiments, at least a portion of the type I lipase is expressed from a nucleic acid comprising SEQ ID NO: 130. In contrast, relative to SEQ ID NO: 131, SEQ ID NO: 130 has a 6 bp sequence TAGATA inserted between base pairs 1 and 2, an A in position 7, a G in position 16, and a G between base pairs 145 and 146. SEQ ID NO: 130 and SEQ ID NO: 131 are shown in Table 14. FIG. 13 also illustrates the differences between sequences encoding Type I lipase and Type II lipase

In certain embodiments, the Type II lipase is encoded by a nucleic acid with at least about 90% homology to SEQ ID NO: 106. In certain embodiments, at least a portion of the Type II lipase is encoded by a nucleic acid with at least about 95% homology to SEQ ID NO: 106. In certain embodiments, at least a portion of the Type II lipase is encoded by a nucleic acid with at least about 97% homology to SEQ ID NO: 106. In certain embodiments, the at least a portion of Type II lipase is encoded by a nucleic acid with at least about 97% homology to SEQ ID NO: 106. In certain embodiments, at least a portion of the Type II lipase is encoded by a nucleic acid with at least about 99% homology to SEQ ID NO: 106. In certain embodiments, at least a portion of the Type II lipase is encoded by a nucleic acid with 100% homology to SEQ ID NO: 106. In certain embodiments, at least a portion of the Type II lipase is encoded by a nucleic acid with at least about 90% homology to SEQ ID NO: 131. In certain embodiments, at least a portion of the Type II lipase is encoded by a nucleic acid with at least about 95% homology to SEQ ID NO: 131. In certain embodiments, at least a portion of the Type II lipase is encoded by a nucleic acid with at least about 97% homology to SEQ ID NO: 131. In certain embodiments, at least a portion of the Type II lipase is encoded by a nucleic acid with at least about 97% homology to SEQ ID NO: 131. In certain embodiments, at least a portion of the Type II lipase is encoded by a nucleic acid with at least about 99% homology to SEQ ID NO: 131. In certain embodiments, at least a portion of the Type II lipase is encoded by a nucleic acid with 100% homology to SEQ ID NO: 131.

### CRISPR Cas5

In some embodiments, compositions disclosed herein comprise synthetic bacteria derived from at least one strain of non-pathogenic bacteria, wherein the at least one strain of non-pathogenic bacteria comprises a CRISPR locus or a portion of a CRISPR locus. In some embodiments, compositions disclosed herein comprise at least one strain of bacteria, wherein the at least one strain of bacteria expresses a CRISPR-associated protein (Cas). By way of non-limiting example, the CRISPR-associated proteins include Cas5, Cas9, Cpf1, Cas3, Cas8a, Cas8b, Cas8c, Cas10d, Cse1, Cse2, Csy1, Csy2, Csy3, GSU0054, Cas10, Csm2, Cmr5, Cas10, Csx11, Csx10, Csf1, Csn2, Cas4, C2c1, C2c3, and C2c2.

In some embodiments, compositions disclosed herein comprise synthetic bacteria derived from at least one strain of non-pathogenic bacteria, wherein the at least one strain of non-pathogenic bacteria expresses a Cas5. In some embodiments, compositions disclosed herein comprise at least one strain of bacteria, wherein the at least one strain of bacteria comprises a nucleic acid encoding a Cas5. In certain embodiments, the at least one strain of bacteria has been selected, transformed, or engineered to express a Cas5. In certain embodiments, the at least one strain of bacteria has at least about 1.5-fold greater expression or activity of Cas5 compared to a reference strain (e.g., pathogenic strain, not a health-associated strain). In certain embodiments, the at least one strain of bacteria has at least about 2-fold greater expression or activity of Cas5 compared to the reference strain. In certain embodiments, the at least one strain of bacteria has at least about 3-fold greater expression or activity of Cas5 compared to the reference strain. In certain embodiments, the at least one strain of bacteria has at least about 5-fold greater expression or activity of Cas5 compared to the reference strain. In certain embodiments, the at least one strain of bacteria has at least about 10-fold greater expression or activity of Cas5 compared to the reference strain.

In some instances, the at least one strain of bacteria expresses Cas5. In some embodiments, a strain of P. acnes is characterized as a health-associated P. acnes or a disease-associated P. acnes based on the presence of Cas5. In some embodiments, Cas5 is found in P. acnes strain ATCC 11828. In some embodiments, Cas5 is encoded by a sequence as set forth in SEQ ID NO: 111. In some embodiments, Cas5 is encoded by a sequence that is at least about 70%, 80%, 85%, 90%, 91%, 92%, 93%, 94%, 95%, 96%, 97%, 98%, 99%, or 100% homologous to SEQ ID NO: 111. n some embodiments, Cas5 is encoded by a sequence that is about 70%, 80%, 85%, 90%, 91%, 92%, 93%, 94%, 95%, 96%, 97%, 98%, 99%, or 100% sequence identity to SEQ ID NO: 111. In some embodiments, Cas5 is encoded by a sequence that is about 95% homologous to SEQ ID NO: 111. In some embodiments, Cas5 is encoded by a sequence that is about 97% homologous to SEQ ID NO: 111. In some embodiments, Cas5 is encoded by a sequence that is about 99% homologous to SEQ ID NO: 111. In some embodiments, Cas5 is encoded by a sequence that is about 100% homologous to SEQ ID NO: 111.

In some instances, the at least one strain of bacteria comprises a nucleic acid encoding Cas5, wherein the nucleic acid comprises at least about 10, at least about 20, at least about 30, at least about 40, at least about 50, at least about 60, at least about 70, at least about 80, at least about 90, at least about 100, at least about 110, at least about 120, at least about 130, at least about 140, at least about 150, at least about 160, at least about 170, at least about 180, at least about 190, at least about 200, at least about 210, at least about 220, at least about 230, at least about 240, at least about 250, at least about 260, at least about 270, at least about 280, at least about 290, at least about 300, at least about 310, at least about 320, at least about 330, at least about 340, at least about 350, at least about 360, at least about 370, at least about 380, at least about 390, at least about 400, at least about 410, at least about 420, at least about 430, at least about 440, at least about 450, at least about 460, at least about 470, at least about 480, at least about 490, at least about 500, at least about 550, at least about 650, at least about 700, or more than about 700 consecutive bases of SEQ ID NO: 111. In some instances, the at least one strain of bacteria comprises a nucleic acid encoding Cas5, wherein the nucleic acid comprises about 10, about 20, about 30, about 40, about 50, about 60, about 70, about 80, about 90, about 100, about 110, about 120, about 130, about 140, about 150, about 160 about 170, about 180, about 190, about 200, about 210, about 220, about 230, about 240, about 250, about 260, about 270, about 280, about 290, about 300, about 310, about 320, about 330, about 340, about 350, about 360, about 370, about 380, about 390, about 400, about 410, about 420, about 430, about 440, about 450, about 460, about 470, about 480, about 490, about 500, about 550, about 650, about 700, or more than about 700 consecutive bases of SEQ ID NO: 111.

### ABC Transporter

In some embodiments, compositions disclosed herein comprise synthetic bacteria derived from at least one strain of non-pathogenic bacteria, wherein the at least one strain of non-pathogenic bacteria comprises an ATP-binding cassette transporter. In certain embodiments, the at least one strain of bacteria comprises a nucleic acid encoding an ATP-binding cassette transporter (ABC transporter). In certain embodiments, the at least one strain of bacteria is selected for expression or overexpression of a nucleic acid encoding an ABC transporter. In certain embodiments, the at least one strain of bacteria is selected for increased activity of an ABC transporter. In certain embodiments, the at least one strain of bacteria is selected for the presence of a nucleic acid encoding an ABC transporter. In certain embodiments, the at least one strain of bacteria is transformed for overexpression of a nucleic acid encoding an ABC transporter. In certain embodiments, the at least one strain of bacteria is transformed for increased activity of an ABC transporter. In certain embodiments, the at least one strain of bacteria is transformed for the presence of a nucleic acid encoding an ABC transporter. In certain embodiments, the at least one strain of bacteria is engineered for overexpression of a nucleic acid encoding an ABC transporter. In certain embodiments, the at least one strain of bacteria is engineered for increased activity of an ABC transporter. In certain embodiments, the at least one strain of bacteria is engineered for the presence of a nucleic acid encoding an ABC transporter. In some embodiments, the ABC transporter is a portion of a known ABC transporter. In some embodiments, the ABC transporter is a portion of a known ABC transporter, wherein the portion of the known ABC transporter can perform an activity of the known ABC transporter. In some embodiments, the ABC transporter is a portion of a known ABC transporter, wherein the portion of the known ABC transporter can perform an enzymatic activity of the known ABC transporter. In some embodiments, the ABC transporter is a portion of a known ABC transporter, wherein the portion of the known ABC transporter can perform a transport activity of the known ABC transporter.

In certain embodiments, the at least one strain has at least about 1.5-fold greater expression or activity of ABC transporter compared to a reference strain (e.g., pathogenic strain, not a health-associated strain). In certain embodiments, the at least one strain has at least about 2-fold greater expression or activity of ABC transporter compared to the reference strain. In certain embodiments, the at least one strain has at least about 3-fold greater expression or activity of ABC transporter compared to the reference strain. In certain embodiments, the at least one strain has at least about 5-fold greater expression or activity of ABC transporter compared to the reference strain. In certain embodiments, the at least one strain has at least about 10-fold greater expression or activity of ABC transporter compared to the reference strain.

In some embodiments, ABC transporters disclosed herein are encoded by a sequence of SEQ ID NO: 109 or a sequence that is homologous to SEQ ID NO: 109. In some embodiments, the ABC transporter is encoded by a sequence that is at least about 80% homologous to SEQ ID NO: 109. In some embodiments, the ABC transporter is encoded by a sequence that is at least about 90% homologous to SEQ ID NO: 109. In some embodiments, the ABC transporter is encoded by a sequence that is at least about 95% homologous to SEQ ID NO: 109. In some embodiments, the ABC transporter is encoded by a sequence that is at least about 96% homologous to SEQ ID NO: 109. In some embodiments, the ABC transporter is encoded by a sequence that is at least about 97% homologous to SEQ ID NO: 109. In some embodiments, the ABC transporter is encoded by a sequence that is at least about 98% homologous to SEQ ID NO: 109. In some embodiments, the ABC transporter is encoded by a sequence that is at least about 99% homologous to SEQ ID NO: 109. In some embodiments, the ABC transporter is encoded by a sequence that is 100% homologous to SEQ ID NO: 109. In certain embodiments, the at least one strain is (completely or partially) selected, transformed, or engineered with a nucleic acid that is at least about 80%, 90%, 95%, 98%, 99%, or 100% homology to SEQ ID NO: 109 is partially or completely present in the at least one strain.

### DNA Binding Response Regulator

In some embodiments, compositions disclosed herein comprise synthetic bacteria derived from at least one strain of non-pathogenic bacteria, wherein the at least one strain of non-pathogenic bacteria does not comprise a DNA binding response regulator. In certain embodiments, the at least one strain does not comprise a gene encoding a DNA binding response regulator. In certain embodiments, the at least one strain has been selected for reduced expression or activity of a DNA binding response regulator. In certain embodiments, the at least one strain has been selected for absence of a DNA binding response regulator. In certain embodiments, bacteria disclosed herein have been transformed for reduced expression or activity of a DNA binding response regulator. In certain embodiments, bacteria disclosed herein have been transformed for absence of a DNA binding response regulator. In certain embodiments, bacteria disclosed herein have been engineered for reduced expression or activity of a DNA binding response regulator. In certain embodiments, bacteria disclosed herein have been engineered for absence of a DNA binding response regulator.

In certain embodiments, the at least one strain of bacteria has at least about 1.5-fold less expression or activity of a DNA binding response regulator relative to a reference strain (e.g., pathogenic strain, not a health-associated strain). In certain embodiments, the at least one strain has at least about 2-fold less expression or activity of a DNA binding response regulator relative to the reference strain. In certain embodiments, the at least one strain has at least about 3-fold less expression or activity of a DNA binding response regulator relative to the reference strain. In certain embodiments, the at least one strain has at least about 5-fold less expression or activity of a DNA binding response regulator relative to the reference strain. In certain embodiments, the at least one strain has at least about 10-fold less expression or activity of a DNA binding response regulator relative to the reference strain.

In certain embodiments, the at least one strain has been selected, transformed, or engineered to remove a nucleic acid with at least 90% homology to SEQ ID NO: 110. In certain embodiments, the at least one strain has been selected, transformed, or engineered to express a nucleic acid with at least 90% homology to SEQ ID NO: 110 at a lower level relative to the reference strain. In certain embodiments, the at least one strain has been selected, transformed, or engineered to remove a nucleic acid with at least 95% homology to SEQ ID NO: 110. In certain embodiments, the at least one strain has been selected, transformed, or engineered to express a nucleic acid with at least 95% homology to SEQ ID NO: 110 at a lower level relative to the reference strain. In certain embodiments, the at least one strain has been selected, transformed, or engineered to remove a nucleic acid with at least 97% homology to SEQ ID NO: 110. In certain embodiments, the at least one strain has been selected, transformed, or engineered to express a nucleic acid with at least 97% homology to SEQ ID NO: 110 at a lower level relative to the reference strain. In certain embodiments, the at least one strain has been selected, transformed, or engineered to remove a nucleic acid with at least 99% homology to SEQ ID NO: 110. In certain embodiments, the at least one strain has been selected, transformed, or engineered to express a nucleic acid with at least 99% homology to SEQ ID NO: 110 at a lower level relative to the reference strain. In certain embodiments, the at least one strain has been selected, transformed, or engineered to remove a nucleic acid with 100% homology to SEQ ID NO: 110. In certain embodiments, the at least one strain has been selected, transformed, or engineered to express a nucleic acid with 100% homology to SEQ ID NO: 110 at a lower level relative to the reference strain.

In certain embodiments, the at least one strain comprises a nucleic acid that is disrupted by an insertion of one or more nucleotides or a introduction of a frameshift mutation, wherein the nucleic acid has 80%, 90%, 95%, 98%, 99%, or 100% homology to SEQ ID NO: 110, before being disrupted. For example, in certain embodiments, a nucleic acid with at least 80%, 90%, 95%, 98%, 99%, or 100% homology to SEQ ID NO: 110 is deleted by at least about 5%, at least about 10%, at least about 15%, at least about 20%, at least about 25%, at least about 30%, at least about 35%, at least about 40%, at least about 45%, at least about 50%, at least about 55%, at least about 60%, at least about 65%, at least about 70%, at least about 75%, at least about 80%, at least about 85%, at least about 90%, at least about 95% or more from the 3 prime end of the nucleic acid. In certain embodiments, the nucleic acid with at least 80%, 90%, 95%, 98%, 99%, or 100% homology to SEQ ID NO: 110 is deleted by at least about 5%, at least about 10%, at least about 15%, at least about 20%, at least about 25%, at least about 30%, at least about 35%, at least about 40%, at least about 45%, at least about 50%, at least about 55%, at least about 60%, at least about 65%, at least about 70%, at least about 75%, at least about 80%, at least about 85%, at least about 90%, at least about 95% or more from the 5 prime end of the nucleic acid.

### Phosphoglycerate Kinase

In some embodiments, compositions disclosed herein comprise synthetic bacteria derived from at least one strain of non-pathogenic bacteria, wherein the at least one strain of non-pathogenic bacteria, wherein the at least one strain of bacteria does not comprise a phosphoglycerate kinase. In certain embodiments, the at least one strain does not comprise a nucleic acid encoding a phosphoglycerate kinase. In certain embodiments, the at least one strain has been selected for reduced expression or activity of a phosphoglycerate kinase. In certain embodiments, the at least one strain has been selected for an absence of a phosphoglycerate kinase. In certain embodiments, bacteria disclosed herein have been transformed for reduced expression or activity of a phosphoglycerate kinase. In certain embodiments, bacteria disclosed herein have been transformed for an absence of a phosphoglycerate kinase. In certain embodiments, bacteria disclosed herein have been engineered for reduced expression or activity of a phosphoglycerate kinase. In certain embodiments, bacteria disclosed herein have been engineered for an absence of a phosphoglycerate kinase.

In certain embodiments, the at least one strain of bacteria has at least about 1.5-fold less expression or activity of a phosphoglycerate kinase relative to a reference strain (e.g., pathogenic strain, not a health-associated strain). In certain embodiments, the at least one strain has at least about 2-fold less expression or activity of a phosphoglycerate kinase relative to the reference strain. In certain embodiments, the at least one strain has at least about 3-fold less expression or activity of a phosphoglycerate kinase relative to the reference strain. In certain embodiments, the at least one strain has at least about 5-fold less expression or activity of a phosphoglycerate kinase relative to the reference strain. In certain embodiments, the at least one strain has at least about 10-fold less expression or activity of a phosphoglycerate kinase relative to the reference strain.

In certain embodiments, the at least one strain has been selected, transformed, or engineered to remove a nucleic acid with at least 90% homology to SEQ ID NO: 112. In certain embodiments, the at least one strain has been selected, transformed, or engineered to express a nucleic acid with at least 90% homology to SEQ ID NO: 112 at a lower level relative to the reference strain. In certain embodiments, the at least one strain has been selected, transformed, or engineered to remove a nucleic acid with at least 95% homology to SEQ ID NO: 112. In certain embodiments, the at least one strain has been selected, transformed, or engineered to express a nucleic acid with at least 95% homology to SEQ ID NO: 112 at a lower level relative to the reference strain. In certain embodiments, the at least one strain has been selected, transformed, or engineered to remove a nucleic acid with at least 97% homology to SEQ ID NO: 112. In certain embodiments, the at least one strain has been selected, transformed, or engineered to express a nucleic acid with at least 97% homology to SEQ ID NO: 112 at a lower level relative to the reference strain. In certain embodiments, the at least one strain has been selected, transformed, or engineered to remove a nucleic acid with at least 99% homology to SEQ ID NO: 112. In certain embodiments, the at least one strain has been selected, transformed, or engineered to express a nucleic acid with at least 99% homology to SEQ ID NO: 112 at a lower level relative to the reference strain. In certain embodiments, the at least one strain has been selected, transformed, or engineered to remove a nucleic acid with 100% homology to SEQ ID NO: 112. In certain embodiments, the at least one strain has been selected, transformed, or engineered to express a nucleic acid with 100% homology to SEQ ID NO: 112 at a lower level relative to the reference strain.

In certain embodiments, the at least one strain comprises a nucleic acid that is disrupted by an insertion of one or more nucleotides or a introduction of a frameshift mutation, wherein the nucleic acid has 80%, 90%, 95%, 98%, 99%, or 100% homology to SEQ ID NO: 112, before being disrupted. For example, in certain embodiments, a nucleic acid with at least 80%, 90%, 95%, 98%, 99%, or 100% homology to SEQ ID NO: 9 is deleted by at least about 5%, at least about 10%, at least about 15%, at least about 20%, at least about 25%, at least about 30%, at least about 35%, at least about 40%, at least about 45%, at least about 50%, at least about 55%, at least about 60%, at least about 65%, at least about 70%, at least about 75%, at least about 80%, at least about 85%, at least about 90%, at least about 95% or more from the 3 prime end of the nucleic acid. In certain embodiments, the nucleic acid with at least 80%, 90%, 95%, 98%, 99%, or 100% homology to SEQ ID NO: 112 is deleted by at least about 5%, at least about 10%, at least about 15%, at least about 20%, at least about 25%, at least about 30%, at least about 35%, at least about 40%, at least about 45%, at least about 50%, at least about 55%, at least about 60%, at least about 65%, at least about 70%, at least about 75%, at least about 80%, at least about 85%, at least about 90%, at least about 95% or more from the 5 prime end of the nucleic acid.

### Dermatin-Sulfate Adhesin

In some embodiments, compositions disclosed herein comprise synthetic bacteria derived from at least one strain of non-pathogenic bacteria, wherein the at least one strain of non-pathogenic bacteria, wherein the at least one strain of bacteria does not comprise a dermatin-sulfate adhesin (DSA1 and DSA2). In certain embodiments, the at least one strain does not comprise a gene encoding a dermatin-sulfate adhesin. In certain embodiments, the at least one strain has been selected for reduced expression or activity of a dermatin-sulfate adhesin. In certain embodiments, the at least one strain has been selected for absence of a dermatin-sulfate adhesin. In certain embodiments, bacteria disclosed herein have been transformed for reduced expression or activity of a dermatin-sulfate adhesin. In certain embodiments, bacteria disclosed herein have been transformed for absence of a dermatin-sulfate adhesin. In certain embodiments, bacteria disclosed herein have been engineered for reduced expression or activity of a dermatin-sulfate adhesin. In certain embodiments, bacteria disclosed herein have been engineered for absence of a dermatin-sulfate adhesin.

In certain embodiments, the bacteria have been selected, transformed, or engineered for lower expression or activity or deletion of a dermatin-sulfate adhesin. In certain embodiments, the selected, transformed, or engineered bacteria has 1.5-fold, 2-fold, 3-fold, or 10-fold less expression or activity of the DSA 1 or DSA 2 compared to a reference strain. In certain embodiments, DSA1 or DSA2 is partially or completely deleted from the genome of the selected, transformed, or engineered, or engineered bacteria. In certain embodiments, DSA1 or DSA2 is disrupted by an insertion of one or more nucleotides or an introduction of a frameshift mutation in the genome of the selected, transformed, or engineered, or engineered bacteria.

### Hyaluronidase

In some embodiments, compositions disclosed herein comprise synthetic bacteria derived from at least one strain of non-pathogenic bacteria, wherein the at least one strain of non-pathogenic bacteria does not comprise a hyaluronidase. Hyaluronidase is also known as hyaluronate lyase (locus tag PPA_RS01930). In some embodiments, compositions disclosed herein comprise at least one strain of bacteria, wherein the at least one strain of bacteria does not have hyaluronidase activity. In some embodiments, compositions disclosed herein comprise at least one strain of bacteria, wherein the at least one strain of bacteria does not comprise a nucleic acid encoding a hyaluronidase. In certain embodiments, the bacteria have been selected, transformed, or engineered for lower expression or activity of hyaluronidase relative to the bacteria before selecting, transforming or engineering, respectively.

In some embodiments, compositions disclosed herein comprise at least one strain of bacteria, wherein the at least one strain of bacteria comprises a hyaluronidase. In some embodiments, compositions disclosed herein comprise at least one strain of bacteria, wherein the at least one strain of bacteria has hyaluronidase activity. In some embodiments, compositions disclosed herein comprise at least one strain of bacteria, wherein the at least one strain of bacteria has a nucleic acid encoding a hyaluronidase. In certain embodiments, the bacteria have been selected, transformed, or engineered for greater expression or activity of hyaluronidase relative to the bacteria before selecting, transforming or engineering, respectively.

In certain embodiments, the selected, transformed, or engineered bacteria have 1.5-fold greater or lower expression or activity of hyaluronate lyase compared to a non-selected, transformed, or engineered strain. In certain embodiments, the selected, transformed, or engineered bacteria have 2-fold greater or lower expression or activity of hyaluronate lyase compared to a non-selected, transformed, or engineered strain. In certain embodiments, the selected, transformed, or engineered bacteria have 3-fold greater or lower expression or activity of hyaluronate lyase compared to a non-selected, transformed, or engineered strain. In certain embodiments, the selected, transformed, or engineered bacteria have 5-fold greater or lower expression or activity of hyaluronate lyase compared to a non-selected, transformed, or engineered strain. In certain embodiments, the selected, transformed, or engineered bacteria have 10-fold greater or lower expression or activity of hyaluronate lyase compared to a non-selected, transformed, or engineered strain. In certain embodiments, the bacteria have been selected, transformed, or engineered to acquire or express at a greater level, a nucleic acid with at least 90% homology to SEQ ID NO: 107. In certain embodiments, the bacteria have been selected, transformed, or engineered to acquire or express at a greater level, a nucleic acid with at least 95% homology to SEQ ID NO: 107. In certain embodiments, the bacteria have been selected, transformed, or engineered to acquire or express at a greater level, a nucleic acid with at least 97% homology to SEQ ID NO: 107. In certain embodiments, the bacteria have been selected, transformed, or engineered to acquire or express at higher level, a nucleic acid with at least 97% homology to SEQ ID NO: 107. In certain embodiments, the bacteria have been selected, transformed, or engineered to acquire or express at a higher level, a nucleic acid with at least 99% homology to SEQ ID NO: 107. In certain embodiments, the bacteria have been selected, transformed, or engineered to acquire or express at a higher level, a nucleic acid with 100% homology to SEQ ID NO: 107. In certain embodiments, the bacteria is a P. acnes bacteria. In certain embodiments, a gene with at least 80%, 90%, 95%, 98%, 99%, or 100% homology to SEQ ID NO: 107 is partially or completely deleted from the genome of the synthetic bacteria. In certain embodiments, a gene with at least 80%, 90%, 95%, 98%, 99%, or 100% homology to SEQ ID NO: 107 is disrupted by an insertion of one or more nucleotides or a introduction of a frameshift mutation in the genome of the selected, transformed, or engineered, or engineered bacteria. In certain embodiments, the selected, transformed, or engineered or selected bacteria are deoR+, Type II lipase positive, pIMPLE negative, or CRISPR Cas5 positive. In certain embodiments, the selected, transformed, or engineered or selected bacteria comprise P. acnes of ribotype RT1 and/or RT2.

In certain embodiments, the bacteria have been selected, transformed, or engineered for lesser expression or absence of hyaluronate lyase. In certain embodiments, the bacteria are selected, transformed, or engineered, or engineered to acquire the presence of a hyaluronidase gene. Hyaluronidase is also known as hyaluronate lyase (locus tag PPA_RS01930). In certain embodiments, the selected, transformed, or engineered, or engineered bacteria have 1.5-fold greater or lower expression or activity of hyaluronate lyase compared to a non-selected, transformed, or engineered strain. In certain embodiments, the selected, transformed, or engineered bacteria have 2-fold greater or lower expression or activity of hyaluronate lyase compared to a non-selected, transformed, or engineered strain. In certain embodiments, the selected, transformed, or engineered bacteria have 3-fold greater or lower expression or activity of hyaluronate lyase compared to a non-selected, transformed, or engineered strain. In certain embodiments, the selected, transformed, or engineered bacteria have 5-fold greater or lower expression or activity of hyaluronate lyase compared to a non-selected, transformed, or engineered strain. In certain embodiments, the selected, transformed, or engineered bacteria have 10-fold greater or lower expression or activity of hyaluronate lyase compared to a non-selected, transformed, or engineered strain. In certain embodiments, the bacteria have been selected, transformed, or engineered to acquire or express at a greater level, a nucleic acid with at least 90% homology to SEQ ID NO: 107. In certain embodiments, the bacteria have been selected, transformed, or engineered to acquire or express at a greater level, a nucleic acid with at least 95% homology to SEQ ID NO: 107. In certain embodiments, the bacteria have been selected, transformed, or engineered to acquire or express at a greater level, a nucleic acid with at least 97% homology to SEQ ID NO: 107. In certain embodiments, the bacteria have been selected, transformed, or engineered to acquire or express at higher level, a nucleic acid with at least 97% homology to SEQ ID NO: 107. In certain embodiments, the bacteria have been selected, transformed, or engineered to acquire or express at a higher level, a nucleic acid with at least 99% homology to SEQ ID NO: 107. In certain embodiments, the bacteria have been selected, transformed, or engineered to acquire or express at a higher level, a nucleic acid with 100% homology to SEQ ID NO: 107. In certain embodiments, the bacteria is a P. acnes bacteria. In certain embodiments, a nucleic acid with at least 80%, 90%, 95%, 98%, 99%, or 100% homology to SEQ ID NO: 107 is partially or completely deleted from the genome of the synthetic bacteria. In certain embodiments, a nucleic acid with at least 80%, 90%, 95%, 98%, 99%, or 100% homology to SEQ ID NO: 107 is disrupted by an insertion of one or more nucleotides or a introduction of a frameshift mutation in the genome of the selected, transformed, or engineered, or engineered bacteria. In certain embodiments, the selected, transformed, or engineered or selected bacteria are deoR+, Type II lipase positive, pIMPLE negative, or CRISPR Cas5 positive. In certain embodiments, the selected, transformed, or engineered or selected bacteria comprise P. acnes of ribotype RT1 and/or RT2.

### Alanine Dehydrogenase

In some embodiments, compositions disclosed herein comprise synthetic bacteria derived from at least one strain of non-pathogenic bacteria, wherein the at least one strain of non-pathogenic bacteria does not comprise an alanine dehydrogenase. In some embodiments, compositions disclosed herein comprise at least one strain of bacteria, wherein the at least one strain of bacteria does not have alanine dehydrogenase activity. In some embodiments, compositions disclosed herein comprise at least one strain of bacteria, wherein the at least one strain of bacteria does not comprise a nucleic acid encoding an alanine dehydrogenase. In certain embodiments, the bacteria have been selected, transformed, or engineered for lower expression or activity of alanine dehydrogenase relative to the bacteria before selection, transformation or engineering, respectively.

In certain embodiments, bacteria are selected, transformed, or engineered for the absence or deletion of a nucleic acid encoding alanine dehydrogenase or a portion thereof. In some embodiments, the alanine dehydrogenase is encoded by a sequence of SEQ ID NO: 108. In some embodiments, the alanine dehydrogenase is encoded by a sequence that is at least 80% homology to SEQ ID NO: 108. In certain embodiments, the bacteria are selected, transformed, or engineered for the absence or deletion of an alanine dehydrogenase or a portion thereof with at least 90% homology to SEQ ID NO: 108. In certain embodiments, the bacteria are selected, transformed, or engineered for the absence or deletion of an alanine dehydrogenase or a portion thereof with at least 95% homology to SEQ ID NO: 108. In certain embodiments, the bacteria are selected, transformed, or engineered for the absence or deletion of an alanine dehydrogenase or a portion thereof with at least 97% homology to SEQ ID NO: 108. In certain embodiments, the bacteria are selected, transformed, or engineered for the absence or deletion of an alanine dehydrogenase or a portion thereof with at least 98% homology to SEQ ID NO: 108. In certain embodiments, the bacteria are selected, transformed, or engineered for the absence or deletion of an alanine dehydrogenase or a portion thereof with at least 99% homology to SEQ ID NO: 108. In certain embodiments, a nucleic acid with at least 80%, 90%, 95%, 98%, 99%, or 100% homology to SEQ ID NO: 108 is partially or completely deleted from the selected, transformed, or engineered, or engineered bacteria. In certain embodiments, a nucleic acid with at least 80%, 90%, 95%, 98%, 99%, or 100% homology to SEQ ID NO: 108 is disrupted by an insertion of one or more nucleotides or a introduction of a frameshift mutation in the selected, transformed, or engineered bacteria. In certain embodiments, the nucleic acid is deleted by at least about 5%, at least about 10%, at least about 15%, at least about 20%, at least about 25%, at least about 30%, at least about 35%, at least about 40%, at least about 45%, at least about 50%, at least about 55%, at least about 60%, at least about 65%, at least about 70%, at least about 75%, at least about 80%, at least about 85%, at least about 90%, at least about 95% or more from the 3 prime end of the nucleic acid. In certain embodiments, the nucleic acid is deleted by at least about 5%, at least about 10%, at least about 15%, at least about 20%, at least about 25%, at least about 30%, at least about 35%, at least about 40%, at least about 45%, at least about 50%, at least about 55%, at least about 60%, at least about 65%, at least about 70%, at least about 75%, at least about 80%, at least about 85%, at least about 90%, at least about 95% or more from the 5 prime end of the nucleic acid.

### Transposase 2

In some embodiments, compositions disclosed herein comprise synthetic bacteria derived from at least one strain of non-pathogenic bacteria, wherein the at least one strain of non-pathogenic bacteria does not comprise a transposase 2. In some embodiments, compositions disclosed herein comprise at least one strain of bacteria, wherein the at least one strain of bacteria does not have transposase 2 activity. In some embodiments, compositions disclosed herein comprise at least one strain of bacteria, wherein the at least one strain of bacteria does not comprise a nucleic acid encoding a transposase 2. In certain embodiments, the bacteria have been selected, transformed, or engineered for lower expression or activity of transposase 2 relative to the bacteria before selection, transformation or engineering, respectively. In some embodiments, compositions disclosed herein comprise at least one strain of bacteria, wherein the at least one strain of bacteria does not have transposase 2 activity, expresses deoR and is of ribotype RT1.

In certain embodiments, bacteria are selected, transformed, or engineered for the absence or deletion of a nucleic acid encoding transposase 2 or a portion thereof. In some embodiments, the transposase 2 is encoded by a sequence of SEQ ID NO: 129. In some embodiments, the transposase 2 is encoded by a sequence that is at least 80% homology to SEQ ID NO: 129. In certain embodiments, the bacteria are selected, transformed, or engineered for the absence or deletion of a transposase 2 or a portion thereof with at least 90% homology to SEQ ID NO: 129. In certain embodiments, the bacteria are selected, transformed, or engineered for the absence or deletion of a transposase 2 or a portion thereof with at least 95% homology to SEQ ID NO: 129. In certain embodiments, the bacteria are selected, transformed, or engineered for the absence or deletion of a transposase 2 or a portion thereof with at least 97% homology to SEQ ID NO: 129. In certain embodiments, the bacteria are selected, transformed, or engineered for the absence or deletion of a transposase 2 or a portion thereof with at least 98% homology to SEQ ID NO: 129. In certain embodiments, the bacteria are selected, transformed, or engineered for the absence or deletion of a transposase 2 or a portion thereof with at least 99% homology to SEQ ID NO: 129. In certain embodiments, a nucleic acid with at least 80%, 90%, 95%, 98%, 99%, or 100% homology to SEQ ID NO: 129 is partially or completely deleted from the selected, transformed, or engineered, or engineered bacteria. In certain embodiments, a nucleic acid with at least 80%, 90%, 95%, 98%, 99%, or 100% homology to SEQ ID NO: 129 is disrupted by an insertion of one or more nucleotides or a introduction of a frameshift mutation in the selected, transformed, or engineered bacteria. In certain embodiments, the nucleic acid is deleted by at least about 5%, at least about 10%, at least about 15%, at least about 20%, at least about 25%, at least about 30%, at least about 35%, at least about 40%, at least about 45%, at least about 50%, at least about 55%, at least about 60%, at least about 65%, at least about 70%, at least about 75%, at least about 80%, at least about 85%, at least about 90%, at least about 95% or more from the 3 prime end of the nucleic acid. In certain embodiments, the nucleic acid is deleted by at least about 5%, at least about 10%, at least about 15%, at least about 20%, at least about 25%, at least about 30%, at least about 35%, at least about 40%, at least about 45%, at least about 50%, at least about 55%, at least about 60%, at least about 65%, at least about 70%, at least about 75%, at least about 80%, at least about 85%, at least about 90%, at least about 95% or more from the 5 prime end of the nucleic acid.

### Additional Markers

In some embodiments, compositions disclosed herein comprise synthetic bacteria derived from at least one strain of non-pathogenic bacteria, wherein the at least one strain of non-pathogenic bacteria comprises at least one protein selected from a protein that mediates biosynthesis of a polysaccharide, a protein that mediates biosynthesis of the cell wall, a protein that mediates biosynthesis of amino acids, a protein that mediates carbohydrate metabolism, and a protein that mediates glycerol transportation. In some embodiments, compositions disclosed herein comprise at least one strain of bacteria, wherein the at least one strain of bacteria comprises at least one nucleic acid that encodes a protein, wherein the protein mediates biosynthesis of a polysaccharide, a protein that mediates biosynthesis of cell wall, a protein that mediates biosynthesis of amino acids, a protein that mediates carbohydrate metabolism, and a protein that mediates glycerol transportation. In some embodiments, the protein that mediates biosynthesis of a polysaccharide is a glycosyl transferase. In some embodiments, the protein that mediates biosynthesis of cell-wall is a D-alanine-D-alanine ligase. In some embodiments, the protein that mediates amino acid biosynthesis is a cobalamin-independent methionine synthase. In some embodiments, the protein is a glycerol uptake facilitator protein. In some embodiments, the protein is a protoporphyrinogen oxidase. In some embodiments, the protoporphyrinogen oxidase is encoded by a hemY gene.

In some embodiments, compositions disclosed herein comprise synthetic bacteria derived from at least one strain of non-pathogenic bacteria, wherein the at least one strain of non-pathogenic bacteria comprises at least one nucleic acid encoding a protein that is selected from a glycosyl transferase, a D-alanin-D-alanine ligase, and a cobalamin-independent methionine synthase.

In certain embodiments, the bacteria have been selected, transformed, or engineered for greater expression or activity of a protein, wherein the protein is selected from a protein that mediates biosynthesis of a polysaccharide, a protein that mediates biosynthesis of cell wall, and a protein that mediates biosynthesis of amino acids. In certain embodiments, the bacteria have been selected, transformed, or engineered for greater expression or activity of a protein selected from a glycosyl transferase, a D-alanin-D-alanine ligase, and a cobalamin-independent methionine synthase.

In some embodiments, compositions disclosed herein comprise synthetic bacteria derived from at least one strain of non-pathogenic bacteria, wherein the at least one strain of non-pathogenic bacteria does not comprise a Christie-Atkins-Munch-Petersen (CAMP) protein. In some embodiments, compositions disclosed herein comprise at least one strain of bacteria, wherein the at least one strain of bacteria does not comprise a CAMP1 protein. In some embodiments, compositions disclosed herein comprise at least one strain of bacteria, wherein the at least one strain of bacteria does not comprise a CAMP2 protein. In some embodiments, compositions disclosed herein comprise at least one strain of bacteria, wherein the at least one strain of bacteria does not comprise a CAMP3 protein.

In some embodiments, compositions disclosed herein comprise synthetic bacteria derived from at least one strain of non-pathogenic bacteria, wherein the at least one strain of non-pathogenic bacteria does not comprise a nucleic acid encoding a CAMP protein. In certain embodiments, the bacteria have been selected, transformed, or engineered for less expression or activity of a CAMP protein relative to the bacteria that is not selected, transformed or engineered. In certain embodiments, the bacteria have been selected, transformed, or engineered for no expression or activity of a CAMP protein. In certain embodiments, the bacteria have been mutated to remove at least a portion of a nucleic acid encoding a CAMP protein. In some embodiments, the CAMP protein is selected from CAMP1, CAMP2, and CAMP3.

In some embodiments, compositions disclosed herein comprise synthetic bacteria derived from at least one strain of non-pathogenic bacteria, wherein the at least one strain of non-pathogenic bacteria does not comprise a sialidase. In some embodiments, compositions disclosed herein comprise at least one strain of bacteria, wherein the at least one strain of bacteria does not comprise a nucleic acid encoding a sialidase. In certain embodiments, the bacteria have been selected, transformed, or engineered for less expression or activity of a sialidase relative to the bacteria that is not selected, transformed or engineered. In certain embodiments, the bacteria have been selected, transformed, or engineered for no expression or activity of a sialidase. In certain embodiments, the bacteria have been mutated to remove at least a portion of a nucleic acid encoding a sialidase.

In some embodiments, compositions disclosed herein comprise synthetic bacteria derived from at least one strain of non-pathogenic bacteria, wherein the at least one strain of non-pathogenic bacteria does not comprise a neuramidase. In some embodiments, compositions disclosed herein comprise at least one strain of bacteria, wherein the at least one strain of bacteria does not comprise a nucleic acid encoding a neuramidase. In certain embodiments, the bacteria have been selected, transformed, or engineered for less expression or activity of a neuramidase relative to the bacteria that is not selected, transformed or engineered. In certain embodiments, the bacteria have been selected, transformed, or engineered for no expression or activity of a neuramidase. In certain embodiments, the bacteria have been mutated to remove at least a portion of a nucleic acid encoding a neuramidase.

In certain embodiments, the non-pathogenic bacteria has been selected, transformed, or engineered for higher activity or expression of any of the following proteins: Adhesion (NCBI Accession No. 50842581); CAMP factor (NCBI Accession No. 50842175, 50842711, 50842820, 50843546); Endoglycoceramidase (NCBI Accession No. 50842131); Iron transport lipoprotein (NCBI Accession No. 50841911); Lysozyme M1 (NCBI Accession No. 50843125); Protein PAGK_237 (NCBI Accession No. 482891444); Protein PPA0532 (NCBI Accession No. 50842016); Protein PPA0533 (NCBI Accession No. 50842017); or Protein PPA1498 (NCBI Accession No. 50842976). In certain embodiments, the bacteria has been selected, transformed, or engineered with a nucleic acid encoding any of the following protein any of the following proteins: Adhesion (NCBI Accession No. 50842581); CAMP factor (NCBI Accession No. 50842175, 50842711, 50842820, 50843546); Endoglycoceramidase (NCBI Accession No. 50842131); Iron transport lipoprotein (NCBI Accession No. 50841911); Lysozyme M1 (NCBI Accession No. 50843125); Protein PAGK_237 (NCBI Accession No. 482891444); Protein PPA0532 (NCBI Accession No. 50842016); Protein PPA0533 (NCBI Accession No. 50842017); or Protein PPA1498 (NCBI Accession No. 50842976).

In certain embodiments, the non-pathogenic bacteria has been selected, transformed, or engineered for lower activity or expression of any of the following proteins: Adhesion (NCBI Accession No. 50843565 or 50843645); Cell wall hydrolase (NCBI Accession No. 50843410); Lipase/acylhydrolase (NCBI Accession No. 50843480); NPL/P60 protein (NCBI Accession No. 50842209); Peptide ABC transporter (NCBI Accession No. 50843590); Protein PPA1197 (NCBI Accession No. 50842677); Protein PPA1281 (NCBI Accession No. 50842762); Protein PPA1715 (NCBI Accession No. 50843175); Protein PPA1939 (NCBI Accession No. 50843388); Protein PPA2239 (NCBI Accession No. 50843674); Rare lipoprotein A rlpa (NCBI Accession No. 50843612); or Triacylglycerol lipase (NCBI Accession No. 50843543). In certain embodiments, the bacteria has been selected, transformed, or engineered with a nucleotide to delete or disrupt a gene encoding any of the following proteins: Adhesion (NCBI Accession No. 50843565 or 50843645); Cell wall hydrolase (NCBI Accession No. 50843410); Lipase/acylhydrolase (NCBI Accession No. 50843480); NPL/P60 protein (NCBI Accession No. 50842209); Peptide ABC transporter (NCBI Accession No. 50843590); Protein PPA1197 (NCBI Accession No. 50842677); Protein PPA1281 (NCBI Accession No. 50842762); Protein PPA1715 (NCBI Accession No. 50843175); Protein PPA1939 (NCBI Accession No. 50843388); Protein PPA2239 (NCBI Accession No. 50843674); Rare lipoprotein A rlpa (NCBI Accession No. 50843612); or Triacylglycerol lipase (NCBI Accession No. 50843543).

In certain embodiments, non-pathogenic bacteria disclosed herein have been selected, transformed, or engineered for lower activity or expression of any of the following proteins: HMPREF0675_4855; HMPREF0675_4856; HMPREF0675_4479; HMPREF0675_4480; HMPREF0675_4481; HMPREF0675_3655/3657; HMPREF0675_4816; HMPREF0675_4817; HMPREF0675_5205; HMPREF0675_5206; HMPREF0675_5014; HMPREF0675_5101; HMPREF0675_5159; HMPREF0675_4093/4094; HMPREF0675_4163; HMPREF0675_5031; HMPREF0675_5390; HMPREF0675_3037. In certain embodiments, the bacteria have been selected, transformed, or engineered with a nucleotide to delete or disrupt a gene encoding any of the following proteins: HMPREF0675_4855; HMPREF0675_4856; HMPREF0675_4479; HMPREF0675_4480; HMPREF0675_4481; HMPREF0675_3655/3657; HMPREF0675_4816; HMPREF0675_4817; HMPREF0675_5205; HMPREF0675_5206; HMPREF0675_5014; HMPREF0675_5101; HMPREF0675_5159; HMPREF0675_4093/4094; HMPREF0675_4163; HMPREF0675_5031; HMPREF0675_5390; HMPREF0675_3037.

In certain embodiments, the non-pathogenic bacteria have been selected, transformed, or engineered for higher activity or expression of any of the following proteins HMPREF0675_4855; HMPREF0675_4856; HMPREF0675_4479; HMPREF0675_4480; HMPREF0675_4481; HMPREF0675_3655/3657; HMPREF0675_4816; HMPREF0675_4817; HMPREF0675_5205; HMPREF0675_5206; HMPREF0675_5014; HMPREF0675_5101; HMPREF0675_5159; HMPREF0675_4093/4094; HMPREF0675_4163; HMPREF0675_5031; HMPREF0675_5390; HMPREF0675_3037. In certain embodiments, the bacteria has been selected, transformed, or engineered with a nucleic acid encoding any of the following proteins: HMPREF0675_4855; HMPREF0675_4856; HMPREF0675_4479; HMPREF0675_4480; HMPREF0675_4481; HMPREF0675_3655/3657; HMPREF0675_4816; HMPREF0675_4817; HMPREF0675_5205; HMPREF0675_5206; HMPREF0675_5014; HMPREF0675_5101; HMPREF0675_5159; HMPREF0675_4093/4094; HMPREF0675_4163; HMPREF0675_5031; HMPREF0675_5390; HMPREF0675_3037.

In certain embodiments, the selected, transformed, or engineered bacteria do not comprise an antibiotic resistance gene. In certain embodiments, the selected, transformed, or engineered bacteria lack an antibiotic resistance gene to any one or more of aminoglycoside, beta-lactam, colistin, fluoroquinolone, fosfomycin, fusidic acid, macrolide, lincosamide, streptogramin B, nitroimidazole, oxazolidinone, phenicol, rifampicin, sulphonamide, tetracycline, trimethoprim, or glycopeptide. In certain embodiments, an antibiotic can be applied to halt treatment with selected, transformed, or engineered bacteria disclosed herein. In certain embodiments, the antibiotic is aminoglycoside, beta-lactam, colistin, fluoroquinolone, fosfomycin, fusidic acid, macrolide, lincosamide, streptogramin B, nitroimidazole, oxazolidinone, phenicol, rifampicin, sulphonamide, tetracycline, trimethoprim, or glycopeptide.

In certain embodiments, the bacteria are selected, transformed, or engineered in order to reduce expression or release of pro-inflammatory mediators by human cells of which the bacteria contact. Bacteria may either directly or indirectly contact human cells (e.g., human skin cells). For instance, bacteria may indirectly contact human cells via factors secreted or released from the bacteria. Non-limiting example of pro-inflammatory mediators from human cells are IL-8, IL-1, IL-6, TNF-alpha, INF-alpha, and human beta defensin.

### Mixtures of Different Microbes

Provided herein are compositions of a plurality of synthetic bacteria. The composition of synthetic bacteria may be a mixture of a plurality of different synthetic bacteria. In a certain embodiment, the mixture comprises at least one selected, transformed, or engineered bacteria. In a certain embodiment, the mixture comprises at least one selected, transformed, or engineered P. acnes. In certain embodiments, the mixture comprises 2, 3, 4, 5, 6, 7, 8, 9, 10 or more isolated and purified species, strains, ribotypes, or phylotypes of bacteria. In a certain embodiment, the mixture comprises at least one strain of bacteria that normally colonizes a tissue or body area other than the skin. In a certain embodiment, the mixture comprises at least one strain of bacteria that normally colonizes the oral cavity. In certain embodiments, the at least one bacteria that normally colonizes the oral cavity is S. salivarius. In a certain embodiment, the mixture comprises at least one strain of bacteria that normally colonizes the lumen of the gastrointestinal system. In a certain embodiment, the mixture comprises at least one bacteria that normally colonizes the lumen of the gastrointestinal system is a Lactobacillus or a Bifidobacterium. In certain embodiments, the Bifidobacterium is Bidifobacterium lactis Bb-12, Bifidobacterium animalis, Bifidobacterium breve, Bifidobacterium bifidum, or any combination thereof. In certain embodiments, the Lactobacillus is Lactobacillus acidophilus, Lactobacillus bulgaricus, Lactobacillus rhamnosus GG, Lactobacillus fermentumi, Lactobacillus sakei, Lactobacillus casei, Lactobacillus salivarius, L rhamnosus LC705, Lactobacillus F19 L, Lactobacillus acidophilus La-5, or any combination thereof. In a certain embodiment, the mixture comprises at least 2, 3, 4, 5, 6, 7, 8, 9, 10, or more different bacterial species. In a certain embodiment, the mixture comprises a mixture of at least 2, 3, 4, 5, 6, 7, 8, 9, 10, or more different bacterial strains. In a certain embodiment, the mixture contains at least one non-bacterial microbe such as a fungus, virus, or bacteriophage. Any defined mixture of a plurality of probiotic strains may be recited to “consist essentially of.” This means that the mixture includes only the specified strains plus any non-active ingredient necessary for proper administration as a topical or oral formulation, such as an excipient or diluent.

In some embodiments, compositions disclosed herein comprise synthetic bacteria useful for treating eczema. In some embodiments, compositions for treating eczema disclosed herein comprise Staphylococcus aureus. In some embodiments, compositions for treating eczema disclosed herein comprise dead Staphylococcus aureus bacteria. In some embodiments, compositions for treating eczema disclosed herein comprise Staphylococcus hominis. In other embodiments, the probiotic comprises of one more of a Dermacoccus, Methlyobacterium or Propionibacterium as they have a negative correlation with S. aureus. In other embodiments, a topical probiotic composition of S. mitis, S. sanguinis or S. cristatus are included in the probiotic.

### Exemplary Bacteria

Synthetic bacteria described herein are engineered or otherwise generally derived from one or more source bacteria, including non-pathogenic bacteria and pathogenic bacteria rendered non-pathogenic by genetic or other means. In many instances, synthetic bacteria are genetically modified to express, produce and/or secrete a biomolecule and/or compound of interest, have modified enzymatic activity, and/or are configured to reduce or otherwise eliminate induction of a host inflammatory response. In some embodiments, source bacteria are non-pathogenic and non-invasive microorganisms. In some embodiments, a non-pathogenic bacteria is gram-positive food grade bacterial strain. In some embodiments, a source bacteria is one that occurs naturally in the human skin microbiome. In certain embodiments, the synthetic bacteria has a genetic background of the source bacteria but for modifications introduced per the methods of this disclosure.

Human skin is populated with microorganisms that reside on the skin, referred to as the skin microbiome. The bacterial microorganisms resident on the skin (in a healthy, non-diseased human) are usually non-pathogenic and commensal and/or mutualistic. The bacteria commonly resident on the human skin are set forth herein, and are indicated by phylogenetic levels, described with their phylogenetic lineage, down to the genus level. In various aspects, synthetic bacteria are combined in a composition with one or more other bacteria, synthetic or otherwise naturally derived. In some embodiments, a population of bacteria in a composition includes a group of individuals of one bacterial species in an area that is separate from other groups of bacteria, apart from rare migration events. In some embodiments, bacteria refers to a community of bacteria, or a collection of populations of different bacteria species that occur together in space and time. In some cases, the community of bacteria includes one or more of all species (that is, across all trophic levels and/or phylogenetic levels), or, alternatively, includes all trophically similar species (for example, all the plants in a rainforest). In another embodiment, bacteria comprises a metapopulation, intending a group of populations that are perceived to exist as a series of local populations that are linked by migration between them. In some embodiments, bacteria are a metacommunity, intending an assemblage of trophically similar individuals and species, each of which is perceived to exist as a series of local communities, linked by the dispersal of potentially interacting species.

Bacteria resident on the skin of healthy humans include bacterial species typically resident on the face of humans, such as Actinobacteria, including bacterial in the genus Corynebacterium and in the genus Propionibacterium. In some embodiments, bacteria resident on the skin of healthy human subjects include bacterial species typically resident on skin other than the face, including for example bacteria in the genus Bacteroidetes and proteobacteria.

In some embodiments, a synthetic bacteria is derived from a bacteria from the genus Propionibacterium, including but not limited to, Propionibacterium acidifaciens, Propionibacterium acidipropionici, Propionibacterium acidipropionici strain 4900, Propionibacterium acnes, Propionibacterium australiense, Propionibacterium avidum, Propionibacterium cyclohexanicum, Propionibacterium freudenreichii subsp. freudenreichii, P. freudenreichii ssp. freudenreichii strain 20271, Propionibacterium freudenreichii subsp. shermanii, P. freudenreichii ssp. shermanii strain 4902, P. freudenreichii ssp. shermanii strain 4902, Propionibacterium granulosum, Propionibacterium innocuum, jensenii, P. jensenii strain 20278, Propionibacterium lymphophilum, Propionibacterium microaerophilum, Propionibacterium propionicum, and/or Propionibacterium thoenii, and P. thoenii strain 20277. In some embodiments, a synthetic bacteria is derived from a bacteria from a population of bacteria classified as “generally regarded as safe” (GRAS) in the Propionibacterium genus, including but not limited to Propionibacterium acidipropionici, Propionibacterium freudenreichii subsp. freudenreichii, Propionibacterium freudenreichii subsp. shermanii, Propionibacterium jensenii, and Propionibacterium thoenii. In some embodiments, a bacteria source is not Propionibacterium acnes. In some embodiments, a bacteria source is Propionibacterium acnes.

In some embodiments, a synthetic bacteria is derived from a bacteria from the genus Corynebacterium, including but not limited to, C. accolens, C. afermentan, C. amycolatum, C. argentoratense, C. aquaticum, C. auris, C. bovis, C. diphtheria, C. equi (now Rhodococcus equi), C. flavescens, C. glucuronolyticum, C. glutamicum, C. granulosum, C. haemolyticum, C, halofytica, C. jeikeium (group JK), C. macginleyi, C. matruchotii, C. minutissimum, C. parvum (Propionibacterium acnes), C. propinquum, C. pseudodiphtheriticum (C. hofinannii), C. pseudotuberculosis, (C. ovis), C. pyogenes, C. urealyticum (group D2), C. renale, C. spec, C. striatum, C. tenuis, C. ulcerans, C. urealyticum, and C. xerosis. Bacteria with lipophilic and nonlipophilic groups are contemplated, and the nonlipophilic bacteria may include fermentative corynebacteria and nonfermentative corynebacteria. In some embodiments, a synthetic bacteria is derived from a bacteria from a population of bacteria classified as “generally regarded as safe” (GRAS) in the Corynebacterium genus, including but not limited to Corynebacterium ammoniagenes, Corynebacterium casei, Corynebacterium flavescens, and Corynebacterium variabile. In some embodiments, a bacteria source is not one of C. diphtheria C. amicolatum, C. striatum, C. jeikeium, C. urealyticum, and C. xerosis, C. pseudotuberculosis, C. tenuis, C. striatum, or C. minutissimum. In some embodiments, a bacteria source is one of C. diphtheria C. amicolatum, C. striatum, C. jeikeium, C. urealyticum, and C. xerosis, C. pseudotuberculosis, C. tenuis, C. striatum, or C. minutissimum.

In some embodiments, a synthetic bacteria is derived from a bacteria from the suborder Micrococcineae, including but not limited to the GRAS bacteria species: Arthrobacter arilaitensis, Arthrobacter bergerei, Arthrobacter globiformis, Arthrobacter nicotianae, Kocuria rhizophila, Kocuria varians, Micrococcus luteus, Micrococcus lylae, Microbacterium gubbeenense, Brevibacterium aurantiacum, Brevibacterium casei, Brevibacterium linens, Brachybacterium alimentarium, and Brachybacterium tyrofermentans. In some embodiments, a bacteria source is not from the order of Actinomycetales, including but not limited to the GRAS bacteria species Streptomyces griseus subsp. griseus. In some embodiments, a bacteria source is from the order of Actinomycetales. In some cases, the bacteria Streptomyces griseus will not express tyrosinase.

In some embodiments, a synthetic bacteria is derived from a bacteria from the genus Staphylococcus, including but not limited to, Staphylococcus agnetis, S. arlettae, S. auricularis, S. capitis, S. caprae, S. camosus, Staphylococcus caseolyticus, S. chromogenes, S. cohnii, S. condiment, S. delphini, S. devriesei, S. equorum, S. felis, S. fleurettii, S. gallinarum, S. haemolyticus, S. hominis, S. hyicus, S. intermedius, S. kloosii, S. leei, S. lentus, S. lugdunensis, S. lutrae, S. massiliensis, S. microti, S. muscae, S. nepalensis, S. pasteuri, S. pettenkoferi, S. piscifermentans, S. pseudintermedius, S. pseudolugdunensis, S. pulvereri, S. rostra, S. saccharolyticus, S. saprophyticus, S. schleiferi, S. sciuri, S. simiae, S. simulans, S. stepanovicii, S. succinus, S. vitulinus, S. wameri, and S. xylosus. In some embodiments, a synthetic bacteria is derived from bacteria classified as “generally regarded as safe” (GRAS) in the order of Staphylococcus, including but not limited to, Staphylococcus camosus subsp. camosus, Staphylococcus camosus subsp. utilis, Staphylococcus cohnii, Staphylococcus condimenti, Staphylococcus equorum subsp. equorum, Staphylococcus equorum subsp. linens, Staphylococcus fleurettii, Staphylococcus piscifermentans, Staphylococcus saprophyticus, Staphylococcus sduri subsp. sduri, Staphylococcus succinus subsp succinus, Staphylococcus succinus subsp. casei, Staphylococcus vitulinus, Staphylococcus wameri, and Staphylococcus xylosus. In some cases, a source bacteria is not S. aureus or S. epidermidis. In some cases, a source bacteria is S. aureus or S. epidermidis.

In some embodiments, a synthetic bacteria is derived from a bacteria from the genus Streptococcus, including but not limited to, Streptococcus acidominimus, Streptococcus adjacens, Streptococcus agalactiae, Streptococcus alactolyticus, Streptococcus anginosus, Streptococcus australis, Streptococcus bovis, Streptococcus cabali, Streptococcus canis, Streptococcus caprinus, Streptococcus castoreus, Streptococcus cecorum, Streptococcus constellatus, Streptococcus constellatus subsp. constellatus, Streptococcus constellatus subsp. pharyngis, Streptococcus cremoris, Streptococcus criceti, Streptococcus cristatus, Streptococcus danieliae, Streptococcus defectives, Streptococcus dentapri, Streptococcus dentirousetti, Streptococcus didelphis, Streptococcus difficilis, Streptococcus durans, Streptococcus dysgalactiae, Streptococcus dysgalactiae subsp. dysgalactiae, Streptococcus dysgalactiae subsp. equisimilis, Streptococcus entericus, Streptococcus equi, Streptococcus equi subsp. equi, Streptococcus equi subsp. ruminatorum, Streptococcus equi subsp. zooepidemicus, Streptococcus equines, Streptococcus faecalis, Streptococcus faecium, Streptococcus ferus, Streptococcus gallinaceus, Streptococcus gallolyticus, Streptococcus gallolyticus subsp. gallolyticus, Streptococcus gallolyticus subsp. macedonicus, Streptococcus gallolyticus subsp. pasteurianus, Streptococcus garvieae, Streptococcus gordonii, Streptococcus halichoeri, Streptococcus hansenii, Streptococcus henryi, Streptococcus hyointestinalis, Streptococcus hyovaginalis, Streptococcus ictaluri, Streptococcus infantarius, Streptococcus infantarius subsp. coli, Streptococcus infantarius subsp. infantarius, Streptococcus infantis, Streptococcus iniae, Streptococcus intermedius, Streptococcus intestinalis, Streptococcus lactarius, Streptococcus lactis, Streptococcus lactis subsp. cremoris, Streptococcus lactis subsp. diacetilactis, Streptococcus lactis subsp. lactis, Streptococcus lutetiensis, Streptococcus macacae, Streptococcus macedonicus, Streptococcus marimammalium, Streptococcus massiliensis, Streptococcus merionis, Streptococcus minor, Streptococcus mitis, Streptococcus morbillorum, Streptococcus mutans, Streptococcus oligofermentans, Streptococcus oralis, Streptococcus orisratti, Streptococcus ovis, Streptococcus parasanguinis, Streptococcus parauberis, Streptococcus parvulus, Streptococcus pasteurianus, Streptococcus peroris, Streptococcus phocae, Streptococcus plantarum, Streptococcus pleomorphus, Streptococcus pluranimalium, Streptococcus plurextorum, Streptococcus pneumonia, Streptococcus porci, Streptococcus porcinus, Streptococcus porcorum, Streptococcus pseudopneumoniae, Streptococcus pseudoporcinus, Streptococcus pyogenes, Streptococcus raffinolactis, Streptococcus ratti, Streptococcus rupicaprae, Streptococcus saccharolyticus, Streptococcus salivarius, Streptococcus salivarius subsp. salivarius, Streptococcus salivarius subsp. thermophilus, Streptococcus sanguinis, Streptococcus shiloi, Streptococcus sinensis, Streptococcus sobrinus, Streptococcus suis, Streptococcus thermophilus, Streptococcus thoraltensis, Streptococcus tigurinus, Streptococcus troglodytae, Streptococcus troglodytidis, Streptococcus uberis, Streptococcus urinalis, Streptococcus vestibularis, and Streptococcus waius. In some embodiments, a synthetic bacteria is derived from a bacteria classified as “generally regarded as safe” (GRAS) in the genus Streptococcus, including but not limited to, Streptococcus thermophilus strain Th4, Streptococcus gallolyticus subsp. macedonicus, Streptococcus salivarius subsp. salivarius, and Streptococcus salivarius subsp. thermophilus.

In some embodiments, a synthetic bacteria is derived from a bacteria from the genus Lactobacillus, including but not limited to, Lactococcus garvieae, Lactococcus lactis, Lactococcus lactis subsp. cremoris, Lactococcus lactis subsp. hordniae, Lactococcus lactis, Lactococcus lactis subsp. lactis, Lactococcus piscium, Lactococcus plantarum, Lactococcus raffinolactis, Lactobacillus acetotolerans, Lactobacillus acidophilus, Lactobacillus agilis, Lactobacillus algidus, Lactobacillus alimentarius, Lactobacillus amylolyticus, Lactobacillus amylophilus, Lactobacillus amylovorus, Lactobacillus animalis, Lactobacillus aviarius, Lactobacillus aviarius subsp. araffinosus, Lactobacillus aviarius subsp. aviarius, Lactobacillus bavaricus, Lactobacillus bifermentans, Lactobacillus brevis, Lactobacillus buchneri, Lactobacillus bulgaricus, Lactobacillus camis, Lactobacillus casei, Lactobacillus casei subsp. alactosus, Lactobacillus casei subsp. casei, Lactobacillus casei subsp. pseudoplantarum, Lactobacillus casei subsp. rhamnosus, Lactobacillus casei subsp. tolerans, Lactobacillus catenaformis, Lactobacillus cellobiosus, Lactobacillus collinoides, Lactobacillus confusus, Lactobacillus coryniformis, Lactobacillus coryniformis subsp. coryniformis, Lactobacillus coryniformis subsp. torquens, Lactobacillus crispatus, Lactobacillus curvatus, Lactobacillus curvatus subsp. curvatus, Lactobacillus curvatus subsp. melibiosus, Lactobacillus delbrueckii, Lactobacillus delbrueckii subsp. bulgaricus, Lactobacillus delbrueckii subsp. delbrueckii, Lactobacillus delbrueckii subsp. lactis, Lactobacillus divergens, Lactobacillus farciminis, Lactobacillus fermenturn, Lactobacillus formicalis, Lactobacillus fructivorans, Lactobacillus fructosus, Lactobacillus gallinarum, Lactobacillus gasser Lactobacillus graminis, Lactobacillus halotolerans, Lactobacillus hamsteri, Lactobacillus helveticus, Lactobacillus heterohiochii, Lactobacillus hilgardii, Lactobacillus homohiochii, Lactobacillus iners, Lactobacillus intestinalis, Lactobacillus jensenii, Lactobacillus johnsonii, Lactobacillus kandleri, Lactobacillus kefiri, Lactobacillus kefuranofaciens, Lactobacillus kefirgranum, Lactobacillus kunkeei, Lactobacillus lactis, Lactobacillus leichmannii, Lactobacillus lindneri, Lactobacillus malefermentans, Lactobacillus mali, Lactobacillus maltaromicus, Lactobacillus manihotivorans, Lactobacillus minor, Lactobacillus minutus, Lactobacillus mucosae, Lactobacillus murinus, Lactobacillus nagelii, Lactobacillus oris, Lactobacillus panis, Lactobacillus parabuchneri, Lactobacillus paracasei, Lactobacillus paracasei subsp. paracasei, Lactobacillus paracasei subsp. tolerans, Lactobacillus parakefiri, Lactobacillus paralimentarius, Lactobacillus paraplantarum, Lactobacillus pentosus, Lactobacillus perolens, Lactobacillus piscicola, Lactobacillus plantarum, Lactobacillus pontis, Lactobacillus reuteri, Lactobacillus rhamnosus, Lactobacillus rhamnosus strain 55a, Lactobacillus rimae, Lactobacillus rogosae, Lactobacillus ruminis, Lactobacillus sakei, Lactobacillus sakei subsp. camosus, Lactobacillus sakei subsp. sakei, Lactobacillus salivarius, Lactobacillus salivarius subsp. salicinius, Lactobacillus salivarius subsp. salivarius, Lactobacillus sanfranciscensis, Lactobacillus sharpeae, Lactobacillus suebicus, Lactobacillus trichodes, Lactobacillus uli, Lactobacillus vaccinostercus, Lactobacillus vaginalis, Lactobacillus viridescens, Lactobacillus vitulinus, Lactobacillus xylosus, Lactobacillus yamanashiensis, Lactobacillus yamanashiensis subsp. mali, Lactobacillus yamanashiensis subsp. yamanashiensis and Lactobacillus zeae. In some embodiments, a synthetic bacteria is derived from bacteria classified as “generally regarded as safe” (GRAS) in the genus Lactobacillus, including but not limited to, Lactobacillus acidophilus strain NP 28, Lactobacillus acidophilus strain NP51, Lactobacillus subsp. lactis strain NP7, Lactobacillus reuteri strain NCIMB 30242, Lactobacillus casei strain Shirota, Lactobacillus reuteri strain DSM 17938, Lactobacillus reuteri strain NCIMB 30242, Lactobacillus acidophilus NCFM, Lactobacillus rhamnosus strain HN001, Lactobacillus rhamnosus strain HN001 produced in a milk-based medium, Lactobacillus reuteri strain DSM 17938, Lactobacillus casei subsp. rhamnosus strain GG, Lactobacillus acidophilus, Lactobacillus lactis, Lactobacillus acetotolerans, Lactobacillus acidifarinae, Lactobacillus acidipisds, Lactobacillus acidophilus, Lactobacillus alimenmrius, Lactobacillus amylolyticus, Lactobacillus amylovorus, Lactobacillus brevis, Lactobacillus buchneri, Lactobacillus cacaonum, Lactobacillus casei subsp. casei, Lactobacillus collinoides, Lactobacillus composti, Lactobacillus co-ryniformis subsp. coryniformis, Lactobacillus crispatus, Lactobacillus crustorum, Lactobacillus curvatus subps. curvatus, Lactobacillus delbrueckii subsp. bulgaricus, Lactobacillus delbrueckii subsp. delbrueckii, Lactobacillus delbrueckii subsp. lactis, Lactobacillus dextrinicus, Lactobacillus diolivorans, Lactobacillus fabifermentans, Lactobacillus farciminis, Lactobacillus fermentum, Lactobacillus fructivorans, Lactobacillus frumenti, Lactobacillus gasser Lactobacillus ghanensis, Lactobacillus hammesii, Lactobacillus harbinensis, Lactobacillus helveticus, Lactobacillus hilgardii, Lactobacillus homohiochii, Lactobacillus hordei, Lactobacillus jensenii, Lactobacillus johnsonii, Lactobacillus kefiri, Lactobacillus kefiranofadens subsp. kefiranofaciens, Lactobacillus kefiranofadens subsp. kefirgranum, Lactobacillus kimchii, Lactobacillus kisonensis, Lactobacillus mali, Lactobacillus manihotivorans, Lactobacillus mindensis, Lactobacillus mucosae, Lactobacillus nagelii, Lactobacillus namurensis, Lactobacillus nantensis, Lactobacillus nodensis, Lactobacillus oeni, Lactobacillus otakiensis, Lactobacillus panis, Lactobacillus parabrevis, Lactobacillus parabuchneri, Lactobacillus paracasei subsp. paracasei, Lactobacillus parakefiri, Lactobacillus paralimentarius, Lactobacillus paraplantarum, Lactobacillus pentosus, Lactobacillus perolens, Lactobacillus plantarum subsp. plantarum, Lactobacillus pobuzihii, Lactobacillus ponds, Lactobacillus rapi Lactobacillus reuteri, Lactobacillus rhamnosus, Lactobacillus rossiae, Lactobacillus sakei subsp carnosus, Lactobacillus sakei subsp. sakei, Lactobacillus sali varies subsp. salivarius, Lactobacillus sanfranciscensis, Lactobacillus satsumensis, Lactobacillus secaliphilus, Lactobacillus senmaizukei, Lactobacillus siliginis, Lactobacillus spicheri, Lactobacillus suebicus, Lactobacillus sunkii, Lactobacillus tucceti, Lactobacillus vacdnosterrus, Lactobacillus versmoldensis, and Lactobacillus yamanashiensis.

In some embodiments, a synthetic bacteria is derived from a bacteria from the genus Lactococcus, including but not limited to, Lactococcus schleifer, Lactococcus chungangensis, Lactococcus fujiensis, Lactococcus garvieae, Lactococcus lactis, Lactococcus lactis subsp. cremoris, Lactococcus lactis subsp. hordniae, Lactococcus lactis subsp. lactis, Lactococcus lactis subsp. tructae, Lactococcus piscium, Lactococcus plantarum, and Lactococcus raffinolacti. In some embodiments, a synthetic bacteria is derived from bacteria classified as “generally regarded as safe” (GRAS) in the genus Lactococcus, including but not limited to, Lactococcus lactis subsp. cremoris, Lactococcus lactis subsp. lactis, and Lactococcus raffinolactis.

In some embodiments, a synthetic bacteria is derived from a bacteria from the genus Enterococcus, including but not limited to, the GRAS bacteria species Enterococcus durans, Enterococcus faecalis, and Enterococcus faecium.

In some embodiments, a synthetic bacteria is derived from a bacteria from the genus Tetragenococcus, including but not limited to, Tetragenococcus halophilus and Tetragenococcus koreensis.

In some embodiments, a synthetic bacteria is derived from a bacteria from the genus Weissella, including but not limited to, the GRAS bacteria species Weissella koreensis, Weissella paramesenteroides, Weissella thailandensis, Weissella confusa, Weissella beninensis, Weissella cibaria, Weissella fabaria, Weissella ghanensis, and Weissella hellenica.

In some embodiments, a synthetic bacteria is derived from a bacteria from the genus Leuconostoc, including but not limited to, the GRAS bacteria species Leuconostoc carnosum, Leuconostoc citreum, Leuconostoc fallax, Leuconostoc holzapfelii, Leuconostoc inhae, Leuconostoc kimchii, Leuconostoc lactis, Leuconostoc mesenteroides subsp. cremoris, Leuconostoc mesenteroides subsp. dextranicum, Leuconostoc mesenteroides subsp. mesenteroides, Leuconostoc palmae, and Leuconostoc pseudomesenteroides.

In some embodiments, a synthetic bacteria is derived from a bacteria from the genus Oenococcus, including but not limited to, Oenococcus oeni.

In some embodiments, a synthetic bacteria is derived from a bacteria from the genus Salinicoccus, including but not limited to, Salinicoccus ventosa, Salinicoccus albus, Salinicoccus alkaliphilus, Salinicoccus carnicancri, Salinicoccus halodurans, Salinicoccus hispanicus, Salinicoccus iranensis, Salinicoccus jeotgali, Salinicoccus kunmingensis, Salinicoccus luteus, Salinicoccus qingdaonensis, Salinicoccus roseus, Salinicoccus salsiraiae, Salinicoccus sesuvii, and Salinicoccus siamensis.

In some embodiments, a synthetic bacteria is derived from a bacteria from the genus of Macrococcus, including but not limited to, Macrococcus caseolyticus.

In some embodiments, a synthetic bacteria is derived from a bacteria from the order Bacillales, including but not limited to, the GRAS bacteria species Bacillus amyloliquefaciens, Bacillus coagulans, and Bacillus subbtilis.

In some embodiments, a synthetic bacteria is not derived from Finegoldia magna. In some embodiments, a synthetic bacteria is derived from Finegoldia magna.

In some embodiments, a synthetic bacteria is derived from a bacteria from the genus of Anaerococcus, including but not limited to, the species Anaerococcus hydrogenalis, Anaerococcus lactolyticus, Anaerococcus murdochii, Anaerococcus octavius, Anaerococcus prevotii, Anaerococcus tetradius, and Anaerococcus vaginalis.

In some embodiments, a synthetic bacteria is derived from a bacteria from the genus of Peptoniphilus, including but not limited to, the species Peptoniphilus asaccharolyticus, Peptoniphilus coxii, Peptoniphilus duerdenii, Peptoniphilus gorbachii, Peptoniphilus harei, Peptoniphilus indolicus, Peptoniphilus ivorii, Peptoniphilus koenoeneniae, Peptoniphilus lacrimalis, Peptoniphilus methioninivorax, Peptoniphilus olsenii, and Peptoniphilus tyrrelliae.

In some embodiments, a synthetic bacteria is derived from a bacteria from the genus of Enhydrobacter, including but not limited to, the species Enhydrobacter aerosaccus.

In some embodiments, a synthetic bacteria is derived from a bacteria from the genus of Sphingomonas, including but not limited to, the species Sphingomonas abaci, Sphingomonas adhaesiva, Sphingomonas aerolata, Sphingomonas aestuarii, Sphingomonas alaskensis, Sphingomonas alpine, Sphingomonas aquatilis, Sphingomonas aromaticivorans, Sphingomonas asaccharolytica, Sphingomonas astaxanthinifaciens, Sphingomonas aurantiaca, Sphingomonas azotifigens, Sphingomonas capsulate, Sphingomonas changbaiensis, Sphingomonas chlorophenolica, Sphingomonas chungbukensis, Sphingomonas cloacae, Sphingomonas cynarae, Sphingomonas desiccabilis, Sphingomonas dokdonensis, Sphingomonas echinoides, Sphingomonas endophytica, Sphingomonas faeni, Sphingomonas fennica, Sphingomonas formosensis, Sphingomonas ginsengisoli, Sphingomonas ginsenosidimutans, Sphingomonas glacialis, Sphingomonas haloaromaticamans, Sphingomonas hankookensis, Sphingomonas herbicidovorans, Sphingomonas histidinilytica, Sphingomonas indica, Sphingomonas insulae, Sphingomonas japonica, Sphingomonas jaspsi, Sphingomonas jejuensis, Sphingomonas jinjuensis, Sphingomonas kaistensis, Sphingomonas koreensis, Sphingomonas laterariae, Sphingomonas leidyi, Sphingomonas macrogolitabida, Sphingomonas macrogoltabidus, Sphingomonas mall, Sphingomonas melonis, Sphingomonas molluscorum, Sphingomonas mucosissima, Sphingomonas natatoria, Sphingomonas oligophenolica, Sphingomonas oryziterrae, Sphingomonas panni, Sphingomonas parapaucimobilis, Sphingomonas paucimobilis, Sphingomonas phyllosphaerae, Sphingomonas pituitosa, Sphingomonas polyaromaticivorans, Sphingomonas pruni, Sphingomonas pseudosanguinis, Sphingomonas rosa, Sphingomonas roseiflava, Sphingomonas rubra, Sphingomonas sanguinis, Sphingomonas sanxanigenens, Sphingomonas sediminicola, Sphingomonas soli, Sphingomonas starnbergensis, Sphingomonas stygia, Sphingomonas subarctica, Sphingomonas suberifaciens, Sphingomonas subterranean, Sphingomonas taejonensis, Sphingomonas terrae, Sphingomonas trueperi, Sphingomonas ursincola, Sphingomonas wittichii, Sphingomonas xenophaga, Sphingomonas xinjiangensis, Sphingomonas yabuuchiae, Sphingomonas yanoikuyae, and Sphingomonas yunnanensis.

In some embodiments, a synthetic bacteria is derived from a bacteria from a GRAS species in the gamma-proteobacteria phylum, such as Halomonas elongata, Hafnia alvei, and in some cases, excluding Hafnia alvei.

In some embodiments, a synthetic bacteria is derived from a bacteria from the genus of Alpha-proteobacteria phylum, including but not limited to, the GRAS species Acetobacter aceti subsp. aceti, Acetobacterfabarum, Acetobacter lovaniensis, Acetobacter malorum, Acetobacter orientalis, Acetobacter pasteurianus subsp. pasteurianus, Acetobacter pornorum, Acetobacter syzygii, Acetobacter tropicalis Gluconacetobacter azotocaptans, Gluconacetobacter diazotrophicus, Gluconacetobacter entanii, Gluconacetobacter europaeus, Gluconacetobacter hansenii, Gluconacetobacter johannae, Gluconacetobacter oboediens, Gluconobacter oxydans, and Gluconacetobacter xylinus.

In some embodiments, a synthetic bacteria is derived from Zymomonas mobilis subsp. mobilis.

In some embodiments, a synthetic bacteria is derived from the Bacteriodetes phylum, including but not limited to, Bacteroides xylanisolvens strain DSM 23964.

In some embodiments, a synthetic bacteria is derived from a bacteria from the genus of Bifidobacterium, including but not limited to, Bifidobacterium adolescentis, Bifidobacterium adolescentis ATCC 15703, Bifidobacterium adolescentis L2-32, Bifidobacterium angulatum, Bifidobacterium, angulatum DSM 20098=JCM 7096, Bifidobacterium animalis, Bifidobacterium animalis subsp. animalis, Bifidobacterium animalis subsp. animalis ATCC 25527, Bifidobacterium animalis subsp. lactis, Bifidobacterium animalis subsp. lactis AD011, Bifidobacterium animalis subsp. lactis ATCC 27673, Bifidobacterium animalis subsp. lactis B420, Bifidobacterium animalis subsp. lactis BB-12, Bifidobacterium animalis subsp. lactis Bi-07, Bifidobacterium animalis subsp. lactis BI-04, Bifidobacterium animalis subsp. lactis BLC1, Bifidobacterium animalis subsp. lactis BS 01, Bifidobacterium animalis subsp. lactis CNCM I-2494, Bifidobacterium animalis subsp. lactis DSM 10140, Bifidobacterium animalis subsp. lactis HN019, Bifidobacterium animalis subsp. lactis V9, Bifidobacterium asteroids, Bifidobacterium asteroides PRL2011, Bifidobacterium biavatii, Bifidobacterium bifidum, Bifidobacterium bifidum ATCC 29521=JCM 1255, Bifidobacterium bifidum BGN4, Bifidobacterium bifidum CECT 7366, Bifidobacterium bifidum DSM 20215, Bifidobacterium bifidum IPLA 20015, Bifidobacterium bifidum JCM 1254, Bifidobacterium bifidum L MG 13195, Bifidobacterium bifidum NCIMB 41171, Bifidobacterium bifidum PRL2010, Bifidobacterium bifidum S17, Bifidobacterium bombi, Bifidobacterium boum, Bifidobacterium breve, Bifidobacterium breve ACS-071-V-Sch8b, Bifidobacterium breve CECT 7263, Bifidobacterium breve DPC 6330, Bifidobacterium breve DSM 20213=JCM 1192, Bifidobacterium breve EX336960VC18, Bifidobacterium breve EX336960VC19, Bifidobacterium breve EX336960VC21, Bifidobacterium breve EX533959VC21, Bifidobacterium breve HPH0326, Bifidobacterium breve JCP7499, Bifidobacterium breve S27, Bifidobacterium breve UCC2003, Bifidobacterium callitrichos, Bifidobacterium catenulatum, Bifidobacterium catenulatum DSM 16992=JCM 1194, Bifidobacterium choerinum, Bifidobacterium choerinum DSM 20434, Bifidobacterium coagulans, Bifidobacterium indicum, Bifidobacterium kashiwanohense, Bifidobacterium kashiwanohense JCM 15439, Bifidobacterium longum, Bifidobacterium longum 3. sub.--1. sub.--37DFAAB, Bifidobacterium longum AGR2137, Bifidobacterium longum BORI, Bifidobacterium longum D2957, Bifidobacterium longum DJO10A, Bifidobacterium longum NCC2705, Bifidobacterium longum subsp. infantis, Bifidobacterium longum subsp. infantis 157F, Bifidobacterium longum subsp. infantis ATCC 15697=JCM 1222, Bifidobacterium longum subsp. infantis CCUG 52486, Bifidobacterium longum subsp. longum, Bifidobacterium longum subsp. longum 1-6B, Bifidobacterium longum subsp. longum 2-2B, Bifidobacterium longum subsp. longum 35B, Bifidobacterium longum subsp. longum 44B, Bifidobacterium longum subsp. longum ATCC 55813, Bifidobacterium longum subsp. longum BBMN68, Bifidobacterium longum subsp. longum CECT 7347, Bifidobacterium longum subsp. longum CMCC P0001, Bifidobacterium longum subsp. longum F8, Bifidobacterium longum subsp. longum JCM 1217, Bifidobacterium longum subsp. longum JDM301, Bifidobacterium longum subsp. longum KACC 91563, Bifidobacterium longum subsp. suis, Bifidobacterium magnum, Bifidobacterium magnum DSM 20222, Bifidobacterium coryneforme, Bifidobacterium crudilactis, Bifidobacterium cuniculi, Bifidobacterium dentium, Bifidobacterium dentium ATCC 27678, Bifidobacterium dentium ATCC 27679, Bifidobacterium dentium Bd1, Bifidobacterium dentium JCM 1195, Bifidobacterium dentium JCVIHMP022, Bifidobacterium gallicum, Bifidobacterium gallicum DSM 20093, Bifidobacterium gallinarum, Bifidobacterium simiae, Bifidobacterium stellenboschense, Bifidobacterium stercoris, Bifidobacterium subtile, Bifidobacterium subtile DSM 20096, Bifidobacterium merycicum, Bifidobacterium minimum, Bifidobacterium minimum DSM 20102, Bifidobacterium mongoliense, Bifidobacterium pseudocatenulatum, Bifidobacterium pseudocatenulatum D2CA, Bifidobacterium pseudocatenulatum DSM 20438=JCM 1200, Bifidobacterium pseudolongum, Bifidobacterium pseudolongum AGR2145, Bifidobacterium pseudolongum subsp. globosum, Bifidobacterium pseudolongum subsp. pseudolongum, Bifidobacterium psychraerophilum, Bifidobacterium pullorum, Bifidobacterium pullorum ATCC 49618, Bifidobacterium reuteri, Bifidobacterium ruminantium, Bifidobacterium saeculare, Bifidobacterium saguini, Bifidobacterium scardovii, Bifidobacterium scardovii JC M 12489, Bifidobacterium thermacidophilum, Bifidobacterium thermacidophilum subsp. porcinum, Bifidobacterium thermacidophilum subsp. thermacidophilum, Bifidobacterium thermophilum, Bifidobacterium thermophilum RBL67, Bifidobacterium tsurumiense, Bifidobacterium tsurumiense DSM 17777, Bifidobacterium sp. Bifidobacterium breve M-16V, Bifidobacterium animalis subsp. lactis strains HN019, Bi-07, BI-04 and B420, Bifidobacterium animalis subsp. lactis strain Bf-6, Bifidobacterium longum strain BB536, and Bifidobacterium lactis strain Bb12.

In some embodiments, a synthetic bacteria is derived from a bacteria from the genus of Carnobacterium, including but not limited to, Carnobacterium alterfunditum, Carnobacterium divergens, Carnobacterium funditum, Carnobacterium gallinarum, Carnobacterium inhibens, Carnobacterium jeotgali, Carnobacterium maltaromaticum, Carnobacterium maltaromaticum 38b, Carnobacterium maltaromaticum ATCC 35586, Carnobacterium maltaromaticum LMA28, Carnobacterium mobile, Carnobacterium pleistocenium, Carnobacterium viridians, Carnobacterium sp., Carnobacterium sp. ‘eilaticum 021211’, Carnobacterium sp. 11-1, Carnobacterium sp. 12266/2009, Carnobacterium sp. 13-3, Carnobacterium sp. 17-4, Carnobacterium sp. 22-6, Carnobacterium sp. 2673, Carnobacterium sp. 27L, Carnobacterium sp. 35L, Carnobacterium sp. 37-3-1, Carnobacterium sp. 38ANA V Carnobacterium sp. 40L, Carnobacterium sp. 7196, Carnobacterium sp. A, Carnobacterium sp. A2S10L14, Carnobacterium sp. A4, Carnobacterium sp. A726, Carnobacterium sp. aG53, Carnobacterium sp. ARCTIC-P2, Carnobacterium sp. ARCTIC-P26, Carnobacterium sp. ARCTIC-P35, Carnobacterium sp. AT12, Carnobacterium sp. A T7, Carnobacterium sp. B, Carnobacterium sp. B5, Carnobacterium sp. BA-8I, Carnobacterium sp. BBDP54, Carnobacterium sp. BBDP71, Carnobacterium sp. BM-8, Carnobacterium sp. BM-8I, Carnobacterium sp. C-13, Carnobacterium sp. c58, Carnobacterium sp. cG53, Carnobacterium sp. CM 1, Carnobacterium sp. D35, Carnobacterium sp. D4, Carnobacterium sp. D5, Carnobacterium sp. EK-153, Carnobacterium sp. ES-11, Carnobacterium sp. FBT1-19, Carnobacterium sp. FBT1-22, Carnobacterium sp. FBT3-14, Carnobacterium sp. FBT3-9, Carnobacterium sp. FBT4-1, Carnobacterium sp. FBT4-18, Carnobacterium sp. G1516J1L, Carnobacterium sp. G4a-1, Carnobacterium sp. G5a-1, Carnobacterium sp. GCM1, Carnobacterium sp. H126a, Carnobacterium sp. Hg4-03, Carnobacterium sp. I-Bh20-14, Carnobacterium sp. I-Bh4-26, Carnobacterium sp. KA-2, Carnobacterium sp. KA-8, Carnobacterium sp. KH1, Carnobacterium sp. KOPRI80142, Carnobacterium sp. KOPRI80153, Carnobacterium sp. KOPRI80155, Carnobacterium sp. L02-6127, Carnobacterium sp. LIV10, Carnobacterium sp. LMG 26642, Carnobacterium sp. L V62: W, Carnobacterium sp. LV66, Carnobacterium sp. M7-CI 0, Carnobacterium sp. MARL15, Carnobacterium sp. MKJ37, Carnobacterium sp. NFU35-25, Carnobacterium sp. NJ-46, Carnobacterium sp. R-36982, Carnobacterium sp. R1234, Carnobacterium sp. S171, Carnobacterium sp. S181, Carnobacterium sp. Sd5t18, Carnobacterium sp. Sd5t5, Carnobacterium sp. Sd6t1, Carnobacterium sp. Sd6t15, Carnobacterium sp. Sd6t17, Carnobacterium sp. Sd6t18, Carnobacterium sp. SR2-31-I, Carnobacterium sp. St2, Carnobacterium sp. T301, Carnobacterium sp. U 149, Carnobacterium sp. UPAA77, Carnobacterium sp. UST050418-652, Carnobacterium sp. WFPIS001, Carnobacterium sp. WN1359, Carnobacterium sp. WN1370, Carnobacterium sp. WN1371, Carnobacterium sp. WN1372, Carnobacterium sp. WN1373, Carnobacterium sp. WN1374, Carnobacterium sp. Y6, Carnobacterium divergens, Carnobacterium maltaromaticum, Carnobacterium piscicola, Carnobacterium maltaromaticum strain CB1 (viable and heat-treated), and Carnobacterium maltaromaticum strain CB1.

In some embodiments, a synthetic bacteria is derived from a bacteria from the genus of Pediococcus, including but not limited to, Pediococcus acidilactici, Pediococcus acidilactici 7.sub.--4, Pediococcus acidilactici D3, Pediococcus acidilactici DSM 20284, Pediococcus acidilactici MA18/5M, Pediococcus argentinicus, Pediococcus cellicola, Pediococcus claussenii, Pediococcus claussenii ATCC BAA-344, Pediococcus damnosus, Pediococcus damnosus 9-6b, Pediococcus ethanolidurans, Pediococcus inopinatus, Pediococcus Pediococcus lolii NGRI 0510Q, Pediococcus parvulus, Pediococcus parvulus CIRM 750, Pediococcus pentosaceus, Pediococcus pentosaceus ATCC 25745, Pediococcus pentosaceus IE-3, Pediococcus siamensis, Pediococcus stilesii, Pediococcus sp. 14.8.17, Pediococcus sp. BGM59, Pediococcus sp. BZ-2005, Pediococcus sp. CAT-100BC, Pediococcus sp. CR-6S, Pediococcus sp. CRA51, Pediococcus sp. EDB-L14, Pediococcus sp. epsi2-MSE-E3-2, Pediococcus sp. epsi31-MSE-E3-2, Pediococcus sp. FUA 3137, Pediococcus sp. FUA 3140, Pediococcus sp. FUA 3226, Pediococcus sp. GS4, Pediococcus sp. IBUN 186, Pediococcus sp. IE3, Pediococcus sp. IJ-K1, Pediococcus sp. J-11, Pediococcus sp. KDLLL3-1, Pediococcus sp. L04, Pediococcus sp. LAB4012, Pediococcus sp. Lact10, Pediococcus sp. LQC 1953, Pediococcus sp. LQC 1957, Pediococcus sp. LQC 1963, Pediococcus sp. LQC 1966, Pediococcus sp. LQC 1972, Pediococcus sp. MB2C, Pediococcus sp. MB2D, Pediococcus sp. MFC1, Pediococcus sp. MMZ60A, Pediococcus sp. MUU10, Pediococcus sp. MUU13, Pediococcus sp. MUU2, Pediococcus sp. MUU3, Pediococcus sp. MUU4, Pediococcus sp. NBRC 106004, Pediococcus sp. NBRC 106014, Pediococcus sp. NBRC 106015, Pediococcus sp. NBRC 106028, Pediococcus sp. NBRC 106032, Pediococcus sp. NBRC 107178, Pediococcus sp. NBRC 107186, Pediococcus sp. NBRC 107193, Pediococcus sp. NBRC 107213, Pediococcus sp. NBRC 107218, Pediococcus sp. NBRC 107221, Pediococcus sp. NBRC 107222, Pediococcus sp. NBRC 107244, Pediococcus sp. NBRC 107250, Pediococcus sp. NBRC 107256, Pediococcus sp. NBRC 107260, Pediococcus sp. NBRC 107264, Pediococcus sp. NBRC 107299, Pediococcus sp. NBRC 107306, Pediococcus sp. NBRC 107309, Pediococcus sp. NBRC 107310, Pediococcus sp. NBRC 107331, Pediococcus sp. NBRC 107343, Pediococcus sp. NBRC 107346, Pediococcus sp. NBRC 107350, Pediococcus sp. N1R1, Pediococcus sp. N1R3, Pediococcus sp. omega41-FH-E3-2, Pediococcus sp. P14, Pediococcus sp. Por3, Pediococcus sp. Porm4, Pediococcus sp. Porn 7, Pediococcus sp. Pov5, Pediococcus sp. Pov7, Pediococcus sp. Pov8, Pediococcus sp. QCH-42, Pediococcus sp. QCH-66, Pediococcus sp. QCH-67, Pediococcus sp. QMA-03G, Pediococcus sp. QMA-06CH, Pediococcus sp. QMA-07G, Pediococcus sp. QMA-11, Pediococcus sp. QMA-21 BC, Pediococcus sp. QMA-23BC, Pediococcus sp. QMA-24BC, Pediococcus sp. QMA-27BC, Pediococcus sp. Rrt8, Pediococcus sp. Rrt9, Pediococcus sp. Rrvl, Pediococcus sp. Rrv3, Pediococcus sp. S17, Pediococcus sp. S18, Pediococcus sp. SD2, Pediococcus sp. shahsavar, Pediococcus sp. sigal, Pediococcus sp. T1RIC23, Pediococcus sp. T1R4C24, Pediococcus sp. Te6, Pediococcus sp. YCO-02, Pediococcus sp. YCO-04, Pediococcus sp. YCO-09, Pediococcus sp. YCO-10, Pediococcus sp. YCO-11, Pediococcus sp. YCO-12, Pediococcus sp. YCO-13, Pediococcus sp. YCO-16, Pediococcus sp. YCO-17, Pediococcus sp. YCO-18, Pediococcus sp. YCO-23, Pediococcus sp. YCO-25, Pediococcus sp. YCO-26, Pediococcus sp. YCO-28, Pediococcus sp. YXC-17, Pediococcus sp. Z-17, Pediococcus acidilactici strain NP3, Pediococcus acidilactici, Pediococcus acidilactici, Pediococcus parvulus, and Pediococcus pentosaceus.

In some embodiments, a bacteria that is pathogenic, and is not altered to render it non-pathogenic, is not a suitable source bacteria from which a synthetic bacteria is derived. In some embodiments, source bacteria include bacteria capable of existing on skin, in particular human skin, and more particularly bacteria that reside on human skin and are GRAS bacteria, and in some cases, excluding pathogenic and/or opportunistic bacteria.

### Target Entities Produced by Synthetic Bacteria

In various aspects, synthetic bacteria are designed to express or produce a biomolecule as generally described previously herein. In further aspects of the disclosure, synthetic bacteria are designed to produce one or more chemical compounds. In some cases, a chemical compound or biomolecule regulates or affects a pathway of the synthetic bacteria. In some cases, a chemical compound or biomolecule regulates or affects production of another biomolecule.

In some embodiments, a synthetic bacteria is produced for the treatment (including prevention and/or maintenance) of acne, wherein the synthetic bacteria produces the biomolecule porphyrin (including, but not limited to, coproporphyrin III, protoporphyrin IX) at a given level. In many cases, the level is at or below about 4 micromolar. In some embodiments, a synthetic bacteria produces a lipase biomolecule with low or absent activity. In some embodiments, a synthetic bacteria produces a level of an enzyme biomolecule from the vitamin B12 metabolic pathway of the synthetic bacteria that results in no or low levels of porphyrin production (including, but not limited to, coproporphyrin III, protoporphyrin IX).

In some embodiments, a synthetic bacteria is produced with a modified (including, without limitation, exogenous and/or heterogeneous) TLR2 or TLR4 ligand biomolecule to prevent the induction of a TLR2 or TLR2 host inflammatory response when the synthetic bacteria is administered to an individual. TLR2 and TLR4 ligands include but are not limited to, cell-wall components such as peptidoglycan, lipoteichoic acid and lipoprotein from gram-positive bacteria, lipoarabinomannan from mycobacteria, and zymosan from yeast cell wall. Toll-like receptor 2 (TLR2) is involved in the recognition of a wide array of microbial molecules representing broad groups of species such as Gram-positive and Gram-negative bacteria, as well as mycoplasma and yeast. In some embodiments, a synthetic bacteria inhibits induction of a host inflammatory response by attenuating or eliminating binding between a biomolecule of the synthetic bacteria and a host cell receptor. As a non-limiting example, the host cell receptor is a protease activated receptor.

In some embodiments, a synthetic bacteria produces one or more of: mycosporine, gadusols, oxo-mycosporines, imino-mycosporines and mycosporine-like amino acids (MAA; glycosylated or covalently bound to oligosaccharides, oligosaccharide-linked MAAS), gadusol, deoxygadusol, 4-deoxygadusol (s2), shinorine, porphyra-334, palythine, palythene, asterina-330, palythinol, mycosporine-glycine, mycosporine serinol, mycosporine-taurine, mycosporine-glycine-valine, mycosporine-2-glycine, mycosporine-glycine-glutamic acid, mycosporine-glutamic acid-glycine, mycosporine-methylamine-serine, mycosporine-methylamine-threonine, usujirene, dehydroxylusujirene, playthenic acid-337, playthenic acid-335, palythine-serine, palythine-threonine, palythine-threonine-sulphate, playthine-serine-sulphate, euhalothece, mycosporine-alanine (2-(e)-2,3-dihydroxipro-1-enylimino-mycosporine-alanine), scytonemin; molecules with sequence similarity to maas, such as dehydroquinate synthase homolog (dhqs homolog) and atp-grasp; melamines, including eumelanin-(or dihydroxyphenylalanine (dopa) melanins), pheomelanin allomelanins, pyomelanine, dopamelanin, neuromelanin; uv-screening/observing amino acids-like molecules, such as urocanic acid flavonoids, anthocyanines and anthoxantins, and anthocyanidins betalanines, such as betacyanin and betaxanthins; uv-screening/observing pigments, such as carotenoids/cartenoproteins, carotens, lycopene, xanthopylls, lutins, zeaxanthin, porphyrin-based/hemeporphyrin based, chlorophyll-ii; uv-screening/observing co-factors, such as tetrahydrobiopterin and phenylpropanoids polyphenol, tannins, phlorotannins, dieckol, eckol, flavan-3-ols or flavanols, pycnogenol sargaquinoic acid, sargachromenol, sphaerophorin (depside) pannarin (depsidone); and DNA repair enzymes, that repair damage caused by exposure to uv, like photolyase, endonuclease and DNA glycosylases, for UV protection or for a skin disorder or disease. In some embodiments, a synthetic bacteria composition comprises a divalent inorganic cation.

In some embodiments, a synthetic bacteria produces one or more of: retinoid, vitamin A, beta-caroten, vitamin D and it's derivatives, and anti-inflammatory cytokines such as interleukin-2 (il-2), for psoriasis or other skin disorder or disease.

In some embodiments, a synthetic bacteria produces one or more of: polymers, such as polyol and glycerol; skin related natural compounds, such as collagen, keratin, elastin, linoleic acid, laminin, tretinoin, tazarotene, sargaquinoic acid, sargachromenol, fucoxanthin, and retinoid for dry skin or other skin disorder or disease.

In some embodiments, a synthetic bacteria produces one or more of: tyrosinases (and its substrates and products); alpha hydroxy acids (AHAs), such as glycolic acid, lactic acid and citric acid for relief of oxidative stress caused by UV or for a skin disorder or disease.

In some embodiments, a synthetic bacteria produces one or more of: polysaccharides; glycosaminoglycans (GAGs) or mucopolysaccharides; hyaluronan (also called hyaluronic acid or hyaluronate or HA); skin related cofactors, such as vitamin A, vitamin C or L-ascorbic acid, or simply ascorbate; biopterin; coenzyme A (CoA, CoASH, or HSCoA); Coenzyme Q10, ubiquinone, ubidecarenone, coenzyme Q; CoQ10; molybdopterin; vitamin E; alpha, beta, gamma, delta-tocopherols and alpha, beta, gamma, delta-tocotrienols, polymers, such as polyol and glycerol, skin related natural compounds, such as collagen, keratin, elastin, linoleic acid, laminin, tretinoin, tazarotene, sargaquinoic acid, sargachromenol, fucoxanthin, retinoid, anti-inflammatory cytokines (such as IL-2), cortisone, tacrolimus, cyclosporine, resveratrol, gallocatechol, gallocatechin, epigallocatechin gallate; cortisone, tacrolimus, cyclosporine; anaerobic bacteria delivering oxygen; talcum, and starch for relief of oxidative stress (e.g., as caused by UV), use as an antioxidant, use as an anti-reactive oxygen species, use as an anti-aging, and/or use in a skin disorder or disease.

In some embodiments, a synthetic bacteria produces one or more entities from one or more of the following groups:

Group 1: (mycosporine, gadusols, oxo-mycosporines, imino-mycosporines and mycosporine-like Amino Acids (MAA; glycosylated or covalently bound to oligosaccharides, oligosaccharide-linked MAAs); and/or intracellular or extracellular gadusol, deoxygadusol, 4-Deoxygadusol (S2), shinorine, porphyra-334, palythine, palythene, asterina-330, palythinol, mycosporine-glycine, mycosporine serinol, mycosporine-taurine, mycosporine-glycine-valine, mycosporine-2-glycine, mycosporine-glycine-glutamic acid, mycosporine-glutamic acid-glycine, mycosporine-methylamine-serine, mycosporine-methylamine-threonine, usujirene, dehydroxylusujirene, playthenic acid-337, playthenic acid-335, palythine-serine, palythine-threonine, palythine-threonine-sulphate, playthine-serine-sulphate, euhalothece, mycosporine-alanine (2-(e)-2,3-dihydroxipro-1-enylimino-mycosporine-alanine));

Group 2: (scytonemin);

Group 3: (melanines: eumelanin- (or dihydroxyphenylalanine (DOPA) melanins), pheomelanin allomelanins, pyomelanine, dopamelanin, neuromelanin);

Group 4: (UV-screening/observing amino acids-like molecules: urocanic acid);

Group 5: (flavonoids: anthocyanines and anthoxantins, anthocyanidins);

Group 6: (betalanines: betacyanin, betaxanthins);

Group 7: (molecules with sequence similarity to MAAs: dehydroquinate synthase homolog (DHQS homolog), ATP-grasp);

Group 8: (UV-screening/observing pigments: carotenoids/cartenoproteins, carotens, lycopene, xanthopylls, lutins, zeaxanthin, porphyrin-based/heme-porphyrin based, chlorophyll-II);

Group 9: (UV-screening/observing co-factors, such as tetrahydrobiopterin and biopterin);

Group 10: (phenylpropanoids);

Group 11: (tannins: phlorotannins, dieckol, eckol);

Group 12: (sargaquinoic acid, sargachromenol, sphaerophorin (depside), pannarin (depsidone)); and

Group 13: (DNA repair enzymes that repair damage caused by exposure to UV, such as photolyase, endonuclease, and DNA glycosylase).

In some embodiments, a synthetic bacteria produces one or more entities from one or more of the following groups:

Group A: (tyrosinases (and its substrates and products));

Group B: (alpha hydroxy acids (AHAs): glycolic acid, lactic acid, and citric acid);

Group C: (polysaccharides: glycosaminoglycans (GAGs), mucopolysaccharides, hyaluronan (also called hyaluronic acid or hyaluronate or HA);

Group D: (skin related cofactors: vitamin C or L-ascorbic acid, or simply ascorbate, vitamin A, biopterin, coenzyme A (CoA, CoASH, or HSCoA), coenzyme 010 (ubiquinone, ubidecarenone, coenzyme Q, CoQ10), molybdopterin);

Group E: (vitamin E: alpha, beta, gamma, delta-tocopherols, alpha, beta, gamma, delta-tocotrienols);

Group F: (polyol, glycerol); and

Group G: (any additional skin related natural compounds, such as collagen, keratin, elastin, linoleic acid, laminin, tretinoin, tazarotene, sargaquinoic acid, sargachromenol, fucoxanthin, retinoid, anti-inflammatory cytokines (such as IL-2), cortisone, tacrolimus, ciclosporin, resveratrol, gallocatechol, gallocatechin, and epigallocatechin gallate).

In some embodiments, a synthetic bacteria producing one or more of the entities in any one or more of groups A-G and groups 1-13 is useful for the treatment of, for example, oxidative stress, cosmetic, and/or anti-aging effects. In some cases, the synthetic bacteria provides relief from UV exposure. In some cases, the synthetic bacteria is useful for treating any skin disorder and/or disease as described herein or readily envisioned by one of skill in the art. In various embodiments, synthetic bacteria producing one or entities in any one or more of groups 1-13 and/or groups A-G are useful for the treatment of acne. Further examples of skin disorders and/or diseases include active dermatitis, burns, insect bites, hives, dandruff and body odor.

Further provided herein are synthetic bacteria producing one or more entities of interest, the synthetic bacteria having or being derived from a bacteria of any one of SEQ ID NOS: 100, 101, 102 and 103. In some cases, the synthetic bacteria is derived from a bacteria having about or at least about a 50%, 60%, 70%, 80%, 90% or 100% identity to a bacteria of any one of SEQ ID NOS: 100, 101, 102 and 103. In some cases, the synthetic bacteria is derived from a bacteria having about or at least about a 50%, 60%, 70%, 80%, 90% or 100% identity to a P. acnes RT6 bacteria. In some cases, the synthetic bacteria is derived from a bacteria having about or at least about a 50%, 60%, 70%, 80%, 90% or 100% identity to a P. acnes RT2 bacteria. In some cases, an entity has about or at least about a 50%, 60%, 70%, 80%, 90% or 100% identity to a gene in any one of SEQ ID NOS: 100, 101, 102 and 103. In some embodiments, an entity has about or at least about a 50%, 60%, 70%, 80%, 90% or 100% identity to a gene from a P. acnes RT6 bacteria. Non-limiting examples of P. acnes RT6 bacteria include HL110PA4, HL110PA3, HL042PA3 and HL202PA1. In some embodiments, an entity has about or at least about a 50%, 60%, 70%, 80%, 90% or 100% identity to a gene in a P. acnes RT2 bacteria. Non-limiting examples of RT2 P. acnes bacteria include HL060PA1, HL103PA1, HL082PA2, HL001PA1, HL106PA1, J139 and ATCC11828.

Entities and bacteria exemplified herein are inclusive of entities and bacteria being homologous and/or substantially identical to the exemplified entities and bacteria, respectively. In certain instances, two sequences are said to be homologous, substantially identical or identical if the sequences have an identity of at least about 40%, 50%, 60%, 70%, 80%, 90%, 95% or more when aligned for maximum correspondence. Also included are proteins having conserved protein domains with domains of proteins embodied as a biomolecule or entity herein. Methods for aligning sequences for comparison are well-known in the art, and include, by way of example, BLAST (provided by the National Center for Biotechnology Information). Furthermore, because of the degeneracy of the genetic code, a large number of functionally identical nucleic acids encode any given protein. One of skill will recognize the individual codon usage to a nucleic acid, peptide, polypeptide, or protein sequence that alters, adds or deletes a single amino acid or a small percentage of amino acids in the encoded sequence to allow the coding of an entity or bacteria provided herein.

### Generation and Production of Synthetic Bacteria

Further provided herein are methods for generating and producing a synthetic bacteria as described herein. In many methods, the method comprises engineering a non-pathogenic bacteria by supplementing, removing, and/or modulating or otherwise mutating an entity to produce a metabolic pathway within the synthetic bacteria different from that of the non-pathogenic bacteria. Biomolecules include nucleic acids-both coding and non-coding, small molecules, lipids, carbohydrates, peptides and proteins. Regions of a non-pathogenic bacteria suitable for engineering to produce a synthetic bacteria as described herein are shown in the scheme of FIG. 5.

In some embodiments, a method for generating a synthetic bacteria comprises engineering the non-pathogenic bacteria with a transcription activator-like effector nuclease (TALEN) and a clustered regulatory interspaced palindromic repeat (CRISPR)/Cas9 endonuclease. In some cases, the exogenous biomolecule is introduced using a transient delivery system. In some cases, the transient delivery system comprises a type III secretion system from a bacteria. Further provided are the synthetic bacteria engineered using said method. Further provided are therapeutic methods using the synthetic bacteria engineered using said method.

In some embodiments, a method for generating a synthetic bacteria comprises engineering the non-pathogenic bacteria to deliver a therapeutic biomolecule to a mammalian cell. Non-limiting examples of biomolecules include vaccines, peptides, nucleic acids, proteins such as enzymes, and small molecules. In some embodiments, the therapeutic biomolecule is a transcription factor such as MyoD. In some embodiments, the therapeutic biomolecule is a TALEN. In some cases, the non-pathogenic bacteria is Propionibacterium acnes. Further provided are the synthetic bacteria engineered using said method. Further provided are therapeutic methods using the synthetic bacteria engineered using said method.

In some embodiments, a method for generating a synthetic bacteria comprises engineering the non-pathogenic bacteria to have controlled expression of a payload protein. In some embodiments, a method for generating a synthetic bacteria comprises engineering the non-pathogenic bacteria to express a programmable adhesion molecule specific for a target surface or cell. In some embodiments, a method for generating a synthetic bacteria comprises engineering the non-pathogenic bacteria to have a stable memory to detect the presence of a small molecule. In some embodiments, a method for generating a synthetic bacteria comprises engineering the non-pathogenic bacteria to produce lactoferrin. In some embodiments, a method for generating a synthetic bacteria comprises engineering the non-pathogenic bacteria to produce an antioxidant, niacinamide, alpha-hydroxy acid, salicylic acid, lipo-hydroxy acid, retinol, linoleic acid, lauric acid, retinaldehyde, zinc, zinc salt, alpha-linolenic, eicosapentaenoic acid, docosahexaenoic acid, tea tree oil, fatty acid, glycolic acid, lauric acid, benzoyl peroxide, undecyl-rhamnoside, SIG1273 gel, oat plantlet extract, or any combination thereof. In some cases, the non-pathogenic bacteria is Propionibacterium acnes. Further provided are the synthetic bacteria engineered using any of the methods described. Further provided are therapeutic methods using the synthetic bacteria engineered using any of the methods described.

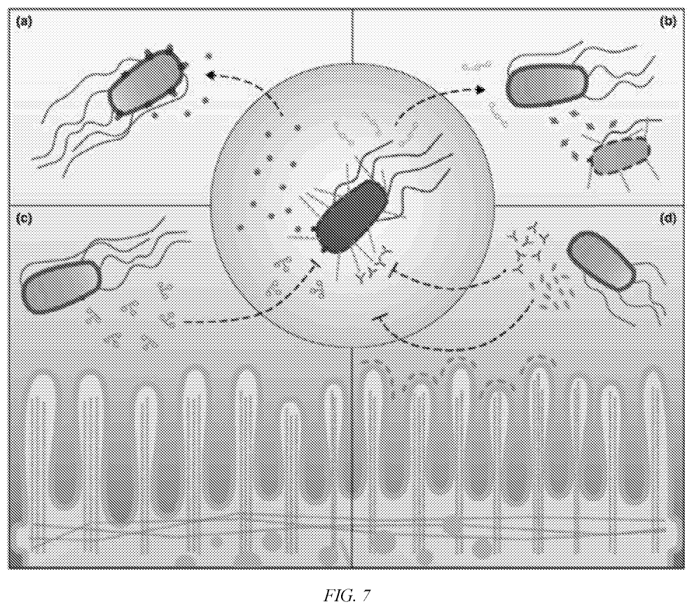

In some embodiments, a method for generating a synthetic bacteria comprises engineering the non-pathogenic bacteria to exhibit tropism-bacterial chemotaxis toward a pathogen. As a non-limiting example, the non-pathogenic bacteria comprises a chemoreceptor and/or chemoeffector. FIG. 7 shows non-limiting examples of mechanisms suitable for use by a synthetic bacteria to prevent pathogen infection, such as a viral or bacterial infection in a host treated with the synthetic bacteria. A pathogenic bacteria is represented in the center of the figure releasing toxins (stars) and quorum sensing signals (linear circles). Engineered bacteria shown in each quadrant can combat the pathogen in a variety of ways. In a first example, shown in panel A, the synthetic bacteria neutralizes the toxins using modified surface components. In a second example, shown in panel B, the synthetic bacteria produces antimicrobial factors (Y) upon detection of quorum sensing signals from the pathogen mediating killing of the pathogenic bacteria. In a third example, shown in panel C, the synthetic bacteria interferes with quorum sensing mechanisms of the pathogen by releasing alternative quorum sensing signals (circles in a T), thus triggering repression of virulence genes. In a fourth example, shown in panel D, synthetic bacteria prevent colonization by the pathogen by secreting antibodies and/or adhesion subunits that competitively inhibit pathogen adhesion to host cells.

In some embodiments, a method for generating a synthetic bacteria comprises engineering the non-pathogenic bacteria to secrete a phenolic compound. Non-limiting examples of phenolic compounds include flavonol, flavone, flavanone, flavanol, isoflavone, antocyjanidin, hydroxycinnamic acid, hydroxybenzoic acid, tannin, stilbene, and lignin. Phenolic compounds include flavonoids derived from the following plants: apples, oranges, grapefruits, black grapes, black elderberries, blueberries, cranberries, cabbage, lettuce, broccoli, radish, chives, onion paprica, chicory, green tea, red wine, Ginkgo biloba leaves, Morus alba leaves, selery, cayenne, pepper, red paprica, parsley, thyme, lemon, rose hip, peppermint, tomatoes, mint, nigella seeds, citrus fruit (e.g., oranges and grapefruits), tea, red wine, chocolate, apples, kiwi, soy, soy products, legumes, cherries, strawberries, grapes, red wine, black currant, black elderberries, chokeberries, blueberries, red cabbage, rhubarb, radish, and red onion. Phenolic compounds include phenolic acids derived from the following plants: apples, pears, plums, cherries, apricots, peaches, black currant, blueberries, Ginkgo biloba and Morus alba leaves, tobacco leaves, potatoes, spinach, lettuce, cabbage, bean, radish, potatoes, broccoli, curly kale, asparagus, olive oil, wine, coffee citrus juice, grains, grapes, black currant, blackberries, lingon berries, strawberries, raspberries, onion, tea, green and black tea, red wine, grapes, mulberries, peanuts, berries, Flaxseed, sunflower seeds, sezame seeds, grains, carrot, onion, chives, apples, cherries, blueberries, strawberries, nuts, tea, and coffee. In some cases, the non-pathogenic bacteria is Propionibacterium acnes. Further provided are the synthetic bacteria engineered using any of the methods described. Further provided are therapeutic methods using the synthetic bacteria engineered using any of the methods described.

In some embodiments, a method for generating a synthetic bacteria comprises engineering the non-pathogenic bacteria to secrete an acne medicament. Non-limiting examples of acne medicaments include benzoyl peroxide, salicylic acid, glycolic acid, clindamycin, erythromycin, Bactrim, doxycycline, tetracycline, minioctcline, spironolactone, retinoids, tacrolimus, pimecrolimus, a steroid, aspirin, ibuprofen, dapsone, azaleic acid, an alphahyroxy acid, a keratolytic, and sulfacetamide sulfur. In some embodiments, a method for generating a synthetic bacteria comprises engineering the non-pathogenic bacteria to secrete a plant derived extract. Non-limiting examples of plant derived extracts include, Aloe vera, Azadirachta indica, Curcuma ionga, Hemidesmus indicus, Terminalia chebula, Withania somnifera, Butyrospermum paradoxum, Camellia sinensis L., Commiphora mukul, Hippophae rhamnoides L., Lens culinaris, Aloe barbadensis, Vitex negundo, Andrographis paniculata, Salmalia malabarica, and Melaleuca alternifolia. In some embodiments, a method for generating a synthetic bacteria comprises engineering the non-pathogenic bacteria to produce an active agent from an herb. Non-limiting examples of herbs include those having the Latin name: Forsythia suspensa (Thunb.) Vahl., Taraxacum mongolicum Hand.-Mazz., Lonicera japonica Thunb., Lonicera hypoglauca Miq., Lonicera confusa D.C., Lonicera dasystyla Rehd., Coix lacryma-jobi L. var. ma-yuen (Roman.) Stapf, Rheum palmatum L., Rheum tanguticum Maxim. Ex Balf., Rheum officinale Baill., Angelica dahurica Benth. Et Hood. F., Angelica dahurica Benth. Et Hook. F. var. formosana Shan et Yuan, Scutellaria baicalensis Georgi, Paeonia suffruticosa Andr., Salvia miltiorrhiza Bge., Morus alba L, Sapashnikovia divaricata (Turcz.) Schisch., Coptis chinensis Franch., Coptis deltoidea C. Y. Cheng et Hsiao, or Coptis teeta Wall, Ligusticum chuanxiong Hort., Platycodon grandiflorum (Jacq.) A. DC, Forsythia suspensa (Thunb.) Vahl, Scutellaria baicalensis Georgi, Mentha haplocalyx Briq, Angelica dahurica Benth. et Hood. F. or Angelica dahurica Benth. et Hook. F. var. formosana Shan et Yuan, Schizonepeta tenuifolia (Benth.) Briq., Glycyrrhiza uralensis Fisch., Citrus aurantium L., Lonicera japonica Thunb, Lonicera hypoglauca Miq, Lonicera confusa DC, or Lonicera dasystyla Rehd, Sa-poshnikovia divaricata (Turcz.) Schisch, Angelica dahurica Benth. et Hook. F. var. formosana Shan et Yuan, Angelica sinensis (Oliv.) Diels, Paeonia lactiflora Pall. Boswellia carterii Birdw. Commiphora myrrha Engl, Fritillaria thunbergii Miq, Trichosanthes kirilowii Maxim. or Trichosanthes japonica Regel, Gleditsia sinensis Lam., Citrus reticulata Blanco, Glycyrrhiza uralensis Fisch, Paeonia lactiflora Pall., Bupleurum chinense DC., Atractylodes macrocephala Koidz, Poria cocos (Schw.) Wolf. Angelica sinensis (Oliv.) Diels, Mentha haplocalyx Briq., Glycyrrhiza uralensis Fisch, Zingiber officinale Rosc, Paeonia suffruticosa Andr., Gardenia jasminoides Ellis, Lonicera japonica Thunb, Lonicera hypoglauca Miq, Lonicera confusa DC, or Lonicera dasystyla Rehd., Chrysanthemum morifolium Ramat., Taraxacum mongolicum Hand.-Mazz., Viola prionantha Bunge, Gardenia jasminoides Ellis., Scutellaria baicalensis Georgi, Coptis chinensis Franch., Phellodendron chinense Schneid. and Phellodendron amurense Rupr. Non-limiting examples of herbs include those having the English name: Lian Qiao, Pu Gong Ying, Jin Yin Hua, Yi Yi Ren, Da Huang, Bai Zhi, Huang Qin, Mu Dan Pi, Dan Shen, Sang Bai Pi, Qing-Shang-Fang-Feng-Tang, Zhen-Ren-Huo-Ming-Yin, Jia-Wei-Xiao-Yao-San, Wu-Wei-Xiao-Du-Yin, and Huang-Lian-Jie-Du-Tang. In some cases, the non-pathogenic bacteria is Propionibacterium acnes. Further provided are the synthetic bacteria engineered using any of the methods described. Further provided are therapeutic methods using the synthetic bacteria engineered using any of the methods described.

In some embodiments, a method for generating a synthetic bacteria comprises engineering the non-pathogenic bacteria to produce a sensor effector specific for a target molecule. In some cases, the sensor effector is an RNA molecule that modulates expression in the bacteria producing the target molecule. In some cases, the sensor effector is a transcription factor that modulates expression in the bacteria producing the target molecule. In some cases, the sensor effector modulates metabolism of the synthetic bacteria in response to the target molecule. In some cases, the sensor effector modulates metabolism of the synthetic bacteria in response to a threshold level of a target molecule. In some cases, the synthetic bacteria is adapted to kill or attenuate a bacteria producing the target molecule. In some cases, the synthetic bacteria is adapted to kill or attenuate a bacteria producing a level of the target molecule above a threshold level. In some cases, the target molecule is a porphyrin. In some cases, the target molecule is a porphyrin and the threshold level is about 4 micromolar or greater. In some cases, the non-pathogenic bacteria is Propionibacterium acnes. Further provided are the synthetic bacteria engineered using any of the methods described. Further provided are therapeutic methods using the synthetic bacteria engineered using any of the methods described.

In some embodiments, a method for generating a synthetic bacteria comprises engineering the non-pathogenic bacteria to produce a sensor effector to coordinate an activity between itself and one or more additional organisms. In some cases, the activity is to regulate cell lysis of the synthetic bacteria to regulate density of the synthetic bacteria. In some cases, the activity is to limit or prevent growth of a target bacteria. In some cases, the target bacteria is a disease-associated strain of a bacteria. In some cases, the target bacteria is an antibiotic resistant bacteria. In some cases, the activity is to treat a disease. In some cases, the activity is to prevent the disease. In some cases, the activity is coordination of chemical exchange and metabolism to produce a desired compound. In some cases, the non-pathogenic bacteria is Propionibacterium acnes. Further provided are the synthetic bacteria engineered using any of the methods described. Further provided are therapeutic methods using the synthetic bacteria engineered using any of the methods described.

### Recombinant Generation of Synthetic Bacteria

In various embodiments, synthetic bacteria are recombinantly produced by genetically engineering a source bacteria, generally a non-pathogenic bacteria.

In general, for synthetic bacteria engineered to encode for a biomolecule of interest, the synthetic bacteria is sometimes prepared by cloning of an isolated nucleic acid molecule that encodes for the biomolecule into an appropriate vector. Vectors include those which are modular and allow independent design of each component in separate conditions and an easy exchange of all essential elements. In such a modular vector, essential elements typically include: replicon, promoter (constitutive or inducible with regulation system), gene of interest, marker or reporter, resistance or limiting factor, Multiple cloning site (MCS), shine-delgarno (ribosomal binding site), and terminators. Suitable vectors can be chosen or constructed, containing appropriate regulatory sequences, including promoter sequences, terminator fragments, polyadenylation sequences, enhancer sequences, and other sequences as appropriate. Appropriate bacterial expression vectors are generally known and available to those of skill in the art. Exemplary essential building blocks of a vector are listed in Table 6, allowing modular configuration of a backbone plasmid, with different combinations, for a suitable expression of the molecule of interest.

TABLE 6
Modular components of an expression vector.

| | Promoter- | Promoter- | Marker or | | |
|---|---|---|---|---|---|
| Replicon | inducible | constitutive | reporter | Terminator | Replicon |
| Ori + | Bacteriocin, | PermB, | cml- | LacZ | Ori + |
| repA, | dnaJ | PldhL, P1 | chloramphenicol, | termi- | repA, |
| p15A, | (from usp45; | (SPL), P10 | (air) alanin | nator, | p15A, |
| p353-1, | High Temp), | (SPL), P11 | racemase gene, | lollypop | p353-1, |
| p353-2, | FOS, gadC- | (SPL), P13 | Abr, amp (Ap)- | structure, | p353-2, |
| p8014-2, | GdR (low | (SPL), P14 | ampicillin, | T1T2, | p8014-2, |
| pA1- | pH), grac- | (SPL), P15 | amyS, ccpA, | Tcat194, | pA1- |
| derived, | lac (IPTG), | (SPL), P16 | cloxacillin, | term667, | derived, |
| pAl, | lacA/lacC/ | (SPL), P17 | Cmr, ermL, | term908, | pAl, |
| pAM- | lacR | (SPL), P20 | ery/ern- | TpepA, | pAM- |
| beta-1, | (Lactose), | (SPL), P21, | erythromycin | TpepN, | beta-1, |
| pBG10, | lacA/T7 | P21 (SPL), | resistance | TsaiA | pBG10, |
| pBM02, | (Lactose), | P22 (SPL), | marker, | | pBM02, |
| pC194, | lacF, lacG, | P23, P23 | estA, genes for | | pC194, |
| pCl305, | lacS-GalR | (SPL), P25 | TTFC, | | pCl305, |
| pCl528, | (Lactose), | (SPL), P27 | gentamycin, | | pCl528, |
| pD125, | lacZ, | (SPL), P29 | GusA (beta- | | pD125, |
| pFX1/3, | NICE system, | (SPL), P3 | glucuronidas), | | pFX1/3, |
| pG+, | nisA/F/R/K/P | (SPL), P30 | Kanamycin, | | pG+, |
| pGK12, | (Nisin), orfX | (SPL), P31 | LacZ, luxAB, | | pGK12, |
| pGT633, | of sakacin | (SPL), P32 | msmR, | | pGT633, |
| pLA106, | Pregulon, | (weak), P33 | neomycin, nisl, | | pLA106, |
| pLAB1000, | PA170 | (SPL), P34 | nsr, penicillin, | | pLAB1000, |
| pLB10, | (low pH, low | (SPL), P35 | PepN | | pLB10, |
| pLC2, | temp.), pgm, | (SPL), P38 | (aminopeptidase | | pLC2, |
| pLF1311, | phi31 (and | (SPL), P4 | N), pepO, ptsH, | | pLF1311, |
| | ori; | | | | |
| pLJ1, | phi infection), | (SPL), P40 | streptomycin, | | pLJ1, |
| pLP1, | Porf1, | (SPL), P41 | tetracycline | | pLP1, |
| pLP825, | Porf330, | (SPL), P42 | | | pLP825, |
| pLPE323, | PorfX, PpfkA, | (SPL), P43 | | | pLPE323, |
| pLUL631, | prtP or ptrM | (SPL), P44 | | | pLUL631, |
| pND302, | (absence of | (SPL), P44 | | | pND302, |
| pND324, | peptides), | (weak), P46 | | | pND324, |
| pOri+, | PsapA, PsapA | (SPL), P47 | | | pOri+, |
| pPM4, | (sakacin A), | (SPL), | | | pPM4, |
| pPSC, | PsapiP, PslpA, | P48(SPL), | | | pPSC, |
| pPSC20/22, | PspplP | P5 (SPL), | | | pPSC20/22, |
| pSH71, | (Sakacin P), | P59, P6 | | | pSH71, |
| pSK11L, | PsspA, PsspQ, | (SPL), P8 | | | pSK11L, |
| pVS40, | Ptuf (CDM), | (SPL), P9 | | | pVS40, |
| pWC1, | Pusp45, | (SPL), | | | pWC1, |
| pWS97, | rep/op phi rlt | Pami, | | | pWS97, |
| pWV01, | (Mitomycin | Ppgm, | | | pWV01, |
| pWV02, | C), repressor/ | Pspac, | | | pWV02, |
| rep256, | operator | Pveg, | | | rep256, |
| repD + E | phirlt | PrRNA1-a, | | | repD + E |
| | (Mytomycin | PrRNA1-b, | | | |
| | C), sodA | PrRNA2-b, | | | |
| | (Aeration), | PrRNA3-a, | | | |
| | tec-Rro12 | PrRNA3-b, | | | |
| | (high temp), | PrRNA4-a, | | | |
| | hyA, tre, | PrRNA4-b, | | | |
| | trpE (absence | PrRNA5-a, | | | |
| | of | PrRNA5-b, | | | |
| | tryptophan), | | | | |
| | xylA (Xylose) | Pslp | | | |

In general, the modular organization of an expression vector is one that allows for expression of a biomolecule by a bacterial host cell. This includes vector such as an expression vector or a chromosomal integration vector. Exemplary vectors include those having features such as SEQ ID NO: 15 and SEQ ID NO:14. In some embodiments, features include DNA elements and/or encode for proteins in SEQ ID NOS: 1-13. In some embodiments, such features are amplified using primers, for example, those having SEQ ID NOS: 16 or 17. In some embodiments, a vector comprises a compatible backbone origin of replication to the bacteria strain in use, a compatible promoter for the expression of the molecule of interest, and a compatible resistance gene. In various cases, the plasmid has restriction enzymes, for example, for cloning purposes. Sequences corresponding to the restriction enzyme recognition sites indicated in the vector of SEQ ID NO: 15 include those of SEQ ID NOS: 19-91.

In some embodiments, a vector is derived from and/or comprises a sequence of nucleic acids from chromosome HL096PA1 of P. acnes RT5. As a non-limiting example, the vector is plasmid pIMPLE-HL096PA1 from P. acnes, Accession number CP003293 or CP003294. Referring to exemplary plasmid pIMPLE-HL096PA1, the plasmid has 74 open reading frames (PAGK 2319-PAGK2392); an origin of replication (RepA); and encodes for plasmid partition related proteins such as ParA (PAGK 2332), and plasmid stabilization system toxin and antitoxin proteins (PAGK 2321 and PAGK 2322). pIMPLE-HL096PA1 further comprises a translation initiation region (SEQ ID NO: 92). In some embodiments, a vector for engineering a synthetic bacteria encodes for one or more genes listed in Table 7. In some cases, one or more genes from Table 7 are modified in a vector and expressed in a source bacteria to generate a synthetic bacteria.

TABLE 7
Genes Encoded in Locus 3 of P. acnes RT4 and RT5.

| Locus | ID | Description |
|---|---|---|
| Locus 3 | PAGK_2319 | hypothetical protein |
| Locus 3 | PAGK_2320 | hypothetical protein |
| Locus 3 | PAGK_2321 | hypothetical protein |
| Locus 3 | PAGK_2322 | plasmid stabilization system protein |
| Locus 3 | PAGK_2323 | hypothetical protein |
| Locus 3 | PAGK_2324 | hypothetical protein |
| Locus 3 | PAGK_2325 | hypothetical protein |
| Locus 3 | PAGK_2326 | CobQ/CobB/MinD/ParA nucleotide binding |
| | | domain |
| Locus 3 | PAGK_2327 | hypothetical protein |
| Locus 3 | PAGK_2328 | hypothetical protein |
| Locus 3 | PAGK_2329 | hypothetical protein |
| Locus 3 | PAGK_2330 | hypothetical protein |
| Locus 3 | PAGK_2331 | hypothetical protein (similar to PPA1279) |
| Locus 3 | PAGK_2332 | plasmid partition protein ParA |
| Locus 3 | PAGK_2333 | hypothetical protein |
| Locus 3 | PAGK_2334 | hypothetical protein |
| Locus 3 | PAGK_2335 | hypothetical protein |
| Locus 3 | PAGK_2336 | putative ribbon helix helix protein oopG family |
| Locus 3 | PAGK_2337 | putative ribonscience E |
| Locus 3 | PAGK_2338 | hypothetical protein (similar to PPA1284) |
| Locus 3 | PAGK_2339 | hypothetical protein (similar to PPA1286) |
| Locus 3 | PAGK_2340 | putative permeases |
| Locus 3 | PAGK_2341 | hypothetical protein (similar to PPA1297) |
| Locus 3 | PAGK_2342 | hypothetical protein (similar to PPA1296) |
| Locus 3 | PAGK_2343 | hypothetical protein (similar to PPA1286) |
| Locus 3 | PAGK_2344 | hypothetical protein (similar to |
| | | CLOLEP_00122) |
| Locus 3 | PAGK_2345 | hypothetical protein (similar to |
| | | CLOLEP_00123) |
| Locus 3 | PAGK_2346 | hypothetical protein (similar to |
| | | CLOLEP_00124) |
| Locus 3 | PAGK_2347 | hypothetical protein (similar to |
| | | CLOLEP_00125) |
| Locus 3 | PAGK_2348 | hypothetical protein (similar to |
| | | CLOLEP_00126) |
| Locus 3 | PAGK_2349 | hypothetical protein (similar to |
| | | CLOLEP_00127) |
| Locus 3 | PAGK_2350 | hypothetical protein |
| Locus 3 | PAGK_2351 | hypothetical protein (similar to |
| | | CLOLEP_00129) |
| Locus 3 | PAGK_2352 | hypothetical protein (similar to |
| | | CLOLEP_00130) |
| Locus 3 | PAGK_2353 | hypothetical protein (similar to |
| | | CLOLEP_00131) |
| Locus 3 | PAGK_2354 | hypothetical protein (similar to |
| | | CLOLEP_00132) |
| Locus 3 | PAGK_2355 | hypothetical protein (similar to |
| | | CLOLEP_00134) |
| Locus 3 | PAGK_2356 | hypothetical protein (similar to |
| | | CLOLEP_00135) |
| Locus 3 | PAGK_2357 | hypothetical protein (similar to |
| | | CLOLEP_00141) |
| Locus 3 | PAGK_2358 | hypothetical protein (similar to |
| | | CLOLEP_00142) |
| Locus 3 | PAGK_2359 | hypothetical protein (similar to |
| | | CLOLEP_00143) |
| Locus 3 | PAGK_2360 | hypothetical protein (similar to |
| | | CLOLEP_00144, RepC) |
| Locus 3 | PAGK_2361 | hypothetical protein (similar to |
| | | CLOLEP_00145, TactZ) |
| Locus 3 | PAGK_2362 | hypothetical protein (similar to |
| | | CLOLEP_00146, TactA) |
| Locus 3 | PAGK_2363 | hypothetical protein (similar to |
| | | CLOLEP_00147, TactB) |
| Locus 3 | PAGK_2364 | hypothetical protein (similar to |
| | | CLOLEP_00148, TactC) |
| Locus 3 | PAGK_2365 | hypothetical protein (similar to |
| | | CLOLEP_00149, Hp-1) |
| Locus 3 | PAGK_2366 | hypothetical protein (similar to |
| | | CLOLEP_00151, TactE) |
| Locus 3 | PAGK_2367 | hypothetical protein (similar to |
| | | CLOLEP_00152, TactE) |
| Locus 3 | PAGK_2368 | hypothetical protein (similar to |
| | | CLOLEP_00153, TactE) |
| Locus 3 | PAGK_2369 | hypothetical protein (similar to |
| | | CLOLEP_00154) |
| Locus 3 | PAGK_2370 | hypothetical protein (similar to |
| | | CLOLEP_00157) |
| Locus 3 | PAGK_2371 | hypothetical protein (similar to |
| | | CLOLEP_00158) |
| Locus 3 | PAGK_2372 | hypothetical protein (similar to |
| | | CLOLEP_00159) |
| Locus 3 | PAGK_2373 | hypothetical protein (similar to |
| | | CLOLEP_00160) |
| Locus 3 | PAGK_2374 | hypothetical protein |
| Locus 3 | PAGK_2375 | hypothetical protein (similar to |
| | | CLOLEP_00162) |
| Locus 3 | PAGK_2376 | hypothetical protein (similar to |
| | | CLOLEP_00163) |
| Locus 3 | PAGK_2377 | hypothetical protein (similar to |
| | | CLOLEP_00164) |
| Locus 3 | PAGK_2378 | hypothetical protein (similar to |
| | | CLOLEP_00165) |
| Locus 3 | PAGK_2379 | repA |
| Locus 3 | PAGK_2380 | CobQ/CobB/MinD/ParA nucleotide binding |
| | | domain |
| Locus 3 | PAGK_2381 | hypothetical protein |
| Locus 3 | PAGK_2382 | hypothetical protein |
| Locus 3 | PAGK_2383 | YagtE |
| Locus 3 | PAGK_2384 | hypothetical protein |
| Locus 3 | PAGK_2385 | hypothetical protein |
| Locus 3 | PAGK_2386 | hypothetical protein |
| Locus 3 | PAGK_2387 | hypothetical protein |
| Locus 3 | PAGK_2388 | hypothetical protein |
| Locus 3 | PAGK_2389 | hypothetical protein |
| Locus 3 | PAGK_2390 | hypothetical protein |
| Locus 3 | PAGK_2391 | hypothetical protein |
| Locus 3 | PAGK_2392 | RestA |

Vectors used to produce synthetic bacteria described herein include plasmids, viral vectors, cosmids, and artificial chromosomes. In general, common to all engineered vectors are an origin of replication, a multiple cloning site, and a selectable marker. The vectors further comprise sequences for expression of the biomolecules of interest, which can be expressed within the synthetic bacteria and/or integrated into the bacterial genome. For expression, in various instances, the copy number of the plasmid is between about 5 and 500 copy numbers per cell. Non-limiting examples of plasmids and expression vectors include p252, p256, p353-2, p8014-2, pA1, pACYC, pAJ01, pAlI-derived, pall, pAM-beta-1,2,3,5,8, pAR1411, pBG10, pBK, pBM02, pBR322, pBR328, pBS-sIpGFP, pC194, PC194/PUB110, pC30i1, pC30iI, pCD034-1, pCD034-2, pCD256, p012000, pC1305, pCI528, pCIS3, pCL2.1, pCT1138, pD125, pE194, pE194/PLS1, pEGFP-C1, pEH, pF8801, pFG2, pFK-series, pGK-series, pGK12, pGK13, pIA, pIAV1,5,6,7,9, pIL.CatT, pIL252/3, pIL253, pIL7, pISA, pJW563, pKRV3, pLAB1000, pLB4, pLBS, pLE16, pLEB124, pLEB590, pLEB591, pLEB600, pLEB604, pLEP24Mcop, pLJ1, pLKS, pLTK2, pWCFS101 and pMD5057, pLP1/18/30, pLP18, pLP317, pLP317cop, pLP3537, pLP3537xyl, pLP402, pLP825, pLP825 and pLPE323, pLP82H, pLPC37, pLPE23M, pLPE323, pLPE350, pLPI, pLS1, pLS1 and pE194, plu1631, pLUL631 from L. reuteri carrying an erythromycin-resistance gene, pM3, pM4, pMD5057, pMG36e, pND324, pNZ-series, pPSC series, pSH71 (de vos, 1987), pSIP-series, pSK11L, pSL2, PSN2, pSN2, pT181, pT181, pC194, pE194, pT181, pE194/pLS1, pC194/pUB110 and pSN2, pTL, pTRK family, pTRT family, pTUAT35, pUBII0 and pC194, pUCL22, pULP8/9, pVS40, pWC1, pWCFS101, pWV02, pWVO4, pWV05, RepA, and system BetL.

Further non-limiting examples of vectors and expression systems useful for preparing a synthetic bacteria include: the lactose phosphotransferase system, optionally linked to the E. coli bacteriophage T7 promoter; the L. lactis nisA promoter system; vectors comprising promoters regulated by environmental conditions, such as for example the P170 promoter that is only active at low pH; a cosmid, a hybrid plasmid (often used as a cloning vector) that contains a Lambda phage cos sequence (cos sites+plasmid); DNA sequences are originally from the lambda phage, and cosmids can be used to build genomic libraries; and a bacterial artificial chromosome (BAC), which is a DNA construct, based on a functional fertility plasmid (or F-plasmid), used for transforming and cloning in bacteria, usually E. coli. Suitable vectors can be chosen or constructed, containing appropriate regulatory sequences, including promoter sequences, terminator fragments, polyadenylation sequences, enhancer sequences, marker genes and other sequences as appropriate. In some embodiments, the mechanism of replication of the replicon is by RCR or by theta-replicating plasmids. In some embodiments, a resistance gene of a vector is based on, for example, antibiotics, bacterium marker, heat-shock, or sugar utilization abilities, such as: thymidylate synthase (thyA), lactose phosphotransferase (lacF), phosph-beta-galactosidase (lac G), or alanine recemase (alr). In some cases, a terminator is added at different positions to provide more efficient expression. Further, in some cases, a signal or anchor sequences is provided to direct expression of polypeptides to the membrane, extracellular space or the cell wall (e.g., by covalent attachment to peptidoglycan). Further systems, vectors, and modifications to those described herein are readily available and known to one of skill in the art.

In various embodiments, a synthetic bacteria is engineered from a source bacteria to have a heterologous biomolecule, that is, a biomolecule or gene expressing said biomolecule, is introduced into the source bacteria when the source bacteria does not normally made said biomolecule. In some cases, the biomolecule is a modification of a biomolecule homologous or similar to a native biomolecule of the source bacteria. In some embodiments, a gene encoding for a heterologous biomolecule is recombinantly produced. In some cases, a recombinant nucleic acid is introduced encoding for a biomolecule, heterologous or otherwise, and optionally another coding region, such as a promoter. In some embodiments, a vector comprises one or more components to regulate expression and localization of a biomolecule, such as a promoter element, sequences encoding signal sequence, a coding sequence for the biomolecule, terminator and anchor sequence. Promoters include those which are constitutive and inducible. Non-limiting examples of promoters include LdhL, SIp, ernB, and orfX. Further examples of promoters include, without limitation, P.sub.59, P.sub.23, Lactobacillus casei L(+)-lactate dehydrogenase promoter, Promoter of Bacillus amylase or xylose promoters; Lactococcus nisin promoter; p32 promoter, T7 gene 10 promoter, alpha amylase promoter sequence of Lactobacillus amylovirus, and promoters which control expression of: LdhL, SIp, ermB, orfX, p6 (pLA6), pLT71, T7, p11, lacTp, dltp, ccpAp, plp, and inducible lactobacillus as lac promoter, LdhL, SIp, ernB, and orfX.

In some embodiments, a synthetic bacteria is engineered to encode for another sequence element that facilitates production of a biomolecule to be expressed in the synthetic bacteria (heterologous or other biomolecules). Such sequence elements include, but are not limited to, promoter/regulatory sequences which facilitate constitutive or inducible expression of the biomolecule or which facilitate over-expression of the biomolecule in the bacterium. Additional sequence elements also include those that facilitate secretion of the biomolecule from the bacteria, accumulation of the biomolecule within the bacteria, and/or programmed lysis of the synthetic bacteria in order to release the protein from the same.

A sequence encoding for a biomolecule introduced or modified in a synthetic bacteria is cloned into an expression vector as described herein using any method known in the art of recombinant DNA technology. Known sequences of biomolecules for cloning into a vector can be identified in commercially available databases. In some embodiments, a coding sequence is inserted in the vector by de-novo sequencing or by PCR amplification. De-novo synthesis includes methods such as the Capillary Electrophoresis method, and Sanger sequencing techniques. In various embodiments, the introduced sequence is verified prior to transformation of the synthetic bacteria with said expression vector. As a non-limiting example, sequences are verified after cloning using, for example a chain termination method for sequencing double-stranded templates.

Expression of an introduced or modified biomolecule of a synthetic bacteria can be performed by any expression method known in the art. In various embodiments, the concentration of biomolecule introduced or manipulated in a synthetic bacteria is varied from 0.1 mM to 100 mM. This concentration can be controlled by various parameters, such as: the concentration of bacteria, the copy number of the plasmid, the activity of the promoter, and the kinetics of the molecule of interest. In some embodiments, a copy number of a vector is between about 5 and 500 copy numbers per cell.

In some embodiments, a biomolecule coding sequence is incorporated into the synthetic bacteria genome. This biomolecule can comprise nucleic acids such as DNA or RNA. In certain embodiments, the biomolecule comprises DNA.

Exemplary sequences of biomolecules within the expression vector for preparing a synthetic bacteria include those with genes coding for molecules for screening UV. As a non-limiting example, genes coding for molecules screening UV in the range of 100-500 nm are contemplated. Further exemplary biomolecule sequences include those coding for molecules reducing oxidative stress, such as genes coding for molecules reducing oxidative stress caused by UV; anti-oxidants, anti-reactive oxygen species (anti-ROS). Additional examples of biomolecule sequences include those encoding for scyA-F, from Cyanobacteria sp. Sun screen compounds, such as shinorine, such as those obtained from corals (Stylophora pistallata), fish (Scarus schlegeli and Chlorurus sordidus), algea (Porphyra umbilicalis), microalgea and, bacteria, as from cyanobacterium Nostoc spp., (like as Nostoc flagelliforme or Nostoc sp. PCC 7524) Lyngbya spp., Anabaena spp., and Nodularia spp. Nostoc punctiforme PCC 73102 Anabaena sp., Anabaena variabilis, Anabaena cylindrica PCC 7122, Cyanothece sp. PCC 7424, Cyanothece sp. PCC 8802, Rivularia sp. PCC 7116, Chroococcidiopsis thermalis PCC 7203, Cylindrospermum stagnale PCC 7417, Stanieria cyanosphaera PCC 7437, Crinalium epipsammum PCC 9333, Crinalium epipsammum PCC 9333, Anabaena sp. 90 chromosome chANA01, Gloeocapsa sp. PCC 7428, Chlorogloeopsis fritschii, Trichodesmium erythraeum IMS101, Microcystis aeruginosa PCC 7806, Microcystis aeruginosa strain UV027, Planktothrix rubescens NIVA-CYA 98, Microcystis sp. NIVA-CYA 172/5, Nostoc sp. GSV224, and Oscillatoria nigro-viridis PCC 7112.

In some embodiments, a vector includes an element such as a purification tag to purify an expressed biomolecule introduced into the cell via the vector. In some cases, an element for expressing the biomolecule in a membrane is included in a vector, for example, usp45. In some embodiments, a vector or any sequence thereof, including one encoding for a biomolecule, is codon optimized. Further improvements include optimization of percentage of GC content.

In some embodiments, a vector includes a sequence encoding for a limiting factor as described elsewhere herein. In some cases, the limiting factor encoding sequence is incorporated into the synthetic bacteria via homologous recombination.

In some embodiments, a source bacteria for transformation of an expression vector as described herein is selected for transformation ability and/or an ability for heterologous protein expression. Various methods for rendering the source bacteria competent include standard techniques such as the rubidium chloride method and electroporation. Various optimization methods for electroporation include optimizing culture media, cell growth stages, DNA concentration, wash or electroporation buffer composition, cuvette gap size, and voltage to improve transformation efficiency.

### Compositions and Methods of Treatment

In one aspect of the disclosure, provided herein are compositions for treating an individual with a therapeutically effective amount of a synthetic bacteria as provided herein. A therapeutically effective amount includes from about 10² cfu/cm² to about 10¹² cfu/cm². In some embodiments, synthetic bacteria are applied in a topical composition at a concentration of at least about 0.1%, or about 0.1% to about 2% by weight of the composition. In some embodiments, synthetic bacteria are formulated into the composition to provide at least about 10² bacteria per cm², 10³ bacteria per cm², 10⁴ bacteria per cm², 10⁵ bacteria per cm², 10⁶ bacteria per cm², or 10⁷ bacteria per cm². In some embodiments, synthetic bacteria are formulated into the composition to provide from about 10² bacteria per cm² to about 10²⁰ bacteria per cm². In some embodiments, synthetic bacteria are formulated into the composition to provide from about 10⁵ bacteria per cm² to about 10¹² bacteria per cm². In some embodiments, synthetic bacteria are formulated into the composition to provide from about 10⁶ bacteria per cm² to about 10⁹ bacteria per cm². In some embodiments, synthetic bacteria are formulated into the composition to provide from about 10⁷ bacteria per cm² to about 10⁸ bacteria per cm². In some embodiments, a synthetic bacteria composition intended to be administered topically comprises synthetic bacteria from about 10 to about 10¹⁵ cfu/g, from about 10⁵ to about 10¹⁵ cfu/g, or from about 10⁷ to about 10¹² cfu/g of bacteria per gram of composition or carrier. In some embodiments, a composition comprises at least about 0.0001% (expressed by dry weight) of synthetic bacteria. In some cases, a composition comprises from about 0.0001% to about 99%, from about 0.001% to about 90% by weight, from about 0.01% to about 80% by weight, or from about 0.1% to about 70% by weight, relative to the total weight of the composition synthetic bacteria.

Synthetic bacteria is provided in a composition in a live, attenuated, semi-active or inactivated, or dead form. In some cases, synthetic bacteria are used in a live form, and are capable of chronically expressing an entity of interest, have modified enzymatic activity or are modified to not induce a host inflammatory response upon topical application of the composition in which they are formulated. In some cases, a synthetic bacteria is in the form of a fraction, including a fraction comprising one or more produced entities. Synthetic bacteria and entities thereof may also be introduced in the form of a lyophilized powder, of a culture supernatant, of harvested compound, and/or where appropriate, in a concentrated form.

Provided herein, in some aspects, are compositions that comprise at least one synthetic bacteria disclosed herein, wherein the compositions are formulated for administration to a subject in need thereof. Generally, the subject is a human afflicted with acne, eczema, psoriasis, seborrheic dermatitis, rosacea, or any combination thereof. In some embodiments, a composition is formulated for topical administration to a subject in need thereof. In some embodiments, the compositions are formulated for topical administration to the skin of the subject. In some embodiments, the compositions are formulated for topical administration to the scalp of the subject. In some embodiments, a composition is formulated for oral administration. By way of non-limiting example, compositions disclosed herein comprising strains of Lactobacillus may be preferentially administered by oral administration. In some embodiments, a composition is formulated for transdermal administration. In some embodiments, a composition is formulated for injectable administration. In certain embodiments, the composition is a formulation selected from a gel, ointment, lotion, emulsion, paste, cream, foam, mousse, liquid, spray, suspension, dispersion and aerosol. In certain embodiments, the formulation comprises one or more excipients to provide a desired form and a desired viscosity, flow or other physical or chemical characteristic for effective application, coverage and adhesion to skin.

Compositions disclosed herein may be presented in a formulation that includes one or more excipients to improve any one or more of shelf-life, application, skin penetration, and therapeutic effect. In some embodiments, the excipient is necessary to improve any one or more of shelf-life, application, skin penetration, and therapeutic effect.

In certain embodiments, the synthetic bacteria compositions described herein are formulated for oral ingestion. The oral ingestion form may be a pill, tablet, capsule, paste, liquid suspension, colloid, or mixed with various foods such as candies, chews, yogurt, milk, cottage cheese or non-dairy based or lactose reduced substitutes. The formulation may contain additional non-active ingredients that improve flavor, smell, or texture of the edible composition. The formulation may also include binding agents, encapsulating films, or excipients that preserve shelf-life and bioavailability.

An emulsion may be described as a preparation of one liquid distributed in small globules throughout the body of a second liquid. In some embodiments, the dispersed liquid is the discontinuous phase, and the dispersion medium is the continuous phase. When oil is the dispersed liquid and an aqueous solution is the continuous phase, it is known as an oil-in-water emulsion, whereas when water or aqueous solution is the dispersed phase and oil or oleaginous substance is the continuous phase, it is known as a water-in-oil emulsion. The oil phase may consist at least in part of a propellant, such as an HFA propellant. Either or both of the oil phase and the aqueous phase may contain one or more surfactants, emulsifiers, emulsion stabilizers, buffers, and other excipients. Preferred excipients include surfactants, especially non-ionic surfactants; emulsifying agents, especially emulsifying waxes; and liquid non-volatile non-aqueous materials, particularly glycols such as propylene glycol. The oil phase may contain other oily pharmaceutically approved excipients. For example, materials such as hydroxylated castor oil or sesame oil may be used in the oil phase as surfactants or emulsifiers.

A lotion may be described as a low- to medium-viscosity liquid formulation. A lotion can contain finely powdered substances that are in soluble in the dispersion medium through the use of suspending agents and dispersing agents. Alternatively, lotions can have as the dispersed phase liquid substances that are immiscible with the vehicle and are usually dispersed by means of emulsifying agents or other suitable stabilizers. In one embodiment, the lotion is in the form of an emulsion having a viscosity of between 100 and 1000 centistokes. The fluidity of lotions permits rapid and uniform application over a wide surface area. Lotions are typically intended to dry on the skin leaving a thin coat of their medicinal components on the skin's surface.

A cream may be described as a viscous liquid or semi-solid emulsion of either the “oil-in-water” or “water-in-oil type”. Creams may contain emulsifying agents and/or other stabilizing agents. In one embodiment, the formulation is in the form of a cream having a viscosity of greater than 1000 centistokes, typically in the range of 20,000-50,000 centistokes. Creams are often time preferred over ointments as they are generally easier to spread and easier to remove.

The basic difference between a cream and a lotion is the viscosity, which is dependent on the amount/use of various oils and the percentage of water used to prepare the formulations. Creams are typically thicker than lotions, may have various uses and often one uses more varied oils/butters, depending upon the desired effect upon the skin. In a cream formulation, the water-base percentage is about 60-75% and the oil-base is about 20-30% of the total, with the other percentages being the emulsifier agent, preservatives and additives for a total of 100%.

An ointment may be described as a semisolid preparation containing an ointment base and optionally one or more active agents of this disclosure. Examples of suitable ointment bases include hydrocarbon bases (e.g., petrolatum, white petrolatum, yellow ointment, and mineral oil); absorption bases (hydrophilic petrolatum, anhydrous lanolin, lanolin, and cold cream); water-removable bases (e.g., hydrophilic ointment), and water-soluble bases (e.g., polyethylene glycol ointments). Pastes typically differ from ointments in that they contain a larger percentage of solids. Pastes are typically more absorptive and less greasy that ointments prepared with the same components.

A gel may be described as a semisolid system containing dispersions of small or large molecules in a liquid vehicle that is rendered semisolid by the action of a thickening agent or polymeric material dissolved or suspended in the liquid vehicle. The liquid may include a lipophilic component, an aqueous component or both. Some emulsions may be gels or otherwise include a gel component. Some gels, however, are not emulsions because they do not contain a homogenized blend of immiscible components. Suitable gelling agents include, but are not limited to, modified celluloses, such as hydroxypropyl cellulose and hydroxyethyl cellulose; Carbopol homopolymers and copolymers; and combinations thereof. Suitable solvents in the liquid vehicle include, but are not limited to, diglycol monoethyl ether; alklene glycols, such as propylene glycol; dimethyl isosorbide; alcohols, such as isopropyl alcohol and ethanol. The solvents are typically selected for their ability to dissolve the drug. Other additives, which improve the skin feel and/or emolliency of the formulation, may also be incorporated. Examples of such additives include, but are not limited, isopropyl myristate, ethyl acetate, C12-C15 alkyl benzoates, mineral oil, squalane, cyclomethicone, capric/caprylic triglycerides, and combinations thereof.

Foams may be described as an emulsion in combination with a gaseous propellant. The gaseous propellant consists primarily of hydrofluoroalkanes (HFAs). Suitable propellants include HFAs such as 1,1,1,2-tetrafluoroethane (HFA 134a) and 1,1,1,2,3,3,3-heptafluoropropane (HFA 227), but mixtures and admixtures of these and other HFAs that are currently approved or may become approved for medical use are suitable. The propellants preferably are not hydrocarbon propellant gases which can produce flammable or explosive vapors during spraying. Furthermore, the compositions preferably contain no volatile alcohols, which can produce flammable or explosive vapors during use.

Emollients may be described as externally applied agents that soften or soothe skin and are generally known in the art and listed in compendia, such as the “Handbook of Pharmaceutical Excipients”, 4.sup.th Ed., Pharmaceutical Press, 2003. In certain embodiments, the emollients are almond oil, castor oil, ceratonia extract, cetostearoyl alcohol, cetyl alcohol, cetyl esters wax, cholesterol, cottonseed oil, cyclomethicone, ethylene glycol palmitostearate, glycerin, glycerin monostearate, glyceryl monooleate, isopropyl myristate, isopropyl palmitate, lanolin, lecithin, light mineral oil, medium-chain triglycerides, mineral oil and lanolin alcohols, petrolatum, petrolatum and lanolin alcohols, soybean oil, starch, stearyl alcohol, sunflower oil, xylitol and combinations thereof. In one embodiment, the emollients are ethylhexylstearate and ethylhexyl palmitate.

Surfactants are surface-active agents that lower surface tension and thereby increase the emulsifying, foaming, dispersing, spreading and wetting properties of a product. In certain embodiments, suitable non-ionic surfactants include emulsifying wax, glyceryl monooleate, polyoxyethylene alkyl ethers, polyoxyethylene castor oil derivatives, polysorbate, sorbitan esters, benzyl alcohol, benzyl benzoate, cyclodextrins, glycerin monostearate, poloxamer, povidone and combinations thereof. In one embodiment, the non-ionic surfactant is stearyl alcohol.

Emulsifiers are surface active substances which promote the suspension of one liquid in another and promote the formation of a stable mixture, or emulsion, of oil and water. In certain embodiments, the emulsifiers are metallic soaps, certain animal and vegetable oils, and various polar compounds. Suitable emulsifiers include acacia, anionic emulsifying wax, calcium stearate, carbomers, cetostearyl alcohol, cetyl alcohol, cholesterol, diethanolamine, ethylene glycol palmitostearate, glycerin monostearate, glyceryl monooleate, hydroxpropyl cellulose, hypromellose, lanolin, hydrous, lanolin alcohols, lecithin, medium-chain triglycerides, methylcellulose, mineral oil and lanolin alcohols, monobasic sodium phosphate, monoethanolamine, nonionic emulsifying wax, oleic acid, poloxamer, poloxamers, polyoxyethylene alkyl ethers, polyoxyethylene castor oil derivatives, polyoxyethylene sorbitan fatty acid esters, polyoxyethylene stearates, propylene glycol alginate, self-emulsifying glyceryl monostearate, sodium citrate dehydrate, sodium lauryl sulfate, sorbitan esters, stearic acid, sunflower oil, tragacanth, triethanolamine, xanthan gum and combinations thereof. In one embodiment, the emulsifier is glycerol stearate. In one embodiment, the emulsifier is glycerol. In one embodiment, the emulsifier is glycerin.

In some embodiments, compositions disclosed herein are formulated to be applied to a subject's scalp. In some embodiments, the composition is formulated to be used as a product selected from a shampoo, a conditioner, a mousse, a gel, and a spray. Such compositions would be useful for the treatment of seborrheic dermatitis. Treatment of seborrheic dermatitis with such compositions may result in the reduction of a symptom selected from dandruff and cradle cap. However, compositions disclosed herein may be used to treat seborrheic dermatitis at other areas of the body besides the scalp. Non-limiting examples of other areas include the chest, stomach, skin folds, arms, legs, groin area and under breasts.

In some embodiments, compositions disclosed herein comprise a buffer, wherein the buffer controls a pH of the composition. Preferably, the buffers buffer the composition from a pH of about 4 to a pH of about 7.5, from a pH of about 4 to a pH of about 7, and from a pH of about 5 to a pH of about 7.

In some embodiments, compositions disclosed herein are formulated to provide or maintain a desirable skin pH. In some embodiments, the desirable skin pH is between about 4.5 and about 6.5. In some embodiments, the desirable skin pH is between about 5 and about 6. In some embodiments, the desirable skin pH is about 5.5. In some embodiments, compositions disclosed herein are formulated with a skin pH modulating agent. Non-limiting examples of pH modulating agents include salicylic acid, glycolic acid, trichloroacetic acid, azeilic acid, lactic acid, aspartic acid, hydrochloride, stearic acid, glyceryl stearate, cetyl palmitate, urea phosphate, and tocopheryl acetate.

In some embodiments, compositions disclosed herein are formulated to provide more oxygen to the skin. In some embodiments, compositions disclosed herein are formulated to provide more oxygen exposure to the skin. In some embodiments, compositions disclosed herein are formulated to provide more oxygen diffusion into the skin. In some embodiments, compositions disclosed herein are formulated to provide more oxygen diffusion through the skin. In some embodiments, compositions disclosed herein are formulated with an agent that provides more oxygen to the skin. In some embodiments, compositions disclosed herein are used with an agent that provides more oxygen to the skin. In some embodiments, compositions disclosed herein are used before use of an agent that provides more oxygen to the skin. In some embodiments, compositions disclosed herein are used after use of an agent that provides more oxygen to the skin. A non-limiting example of an agent that provides oxygen to the skin is chlorophyll.

Preservatives can be used to prevent the growth of fungi and microorganisms. Suitable antifungal and antimicrobial agents include, but are not limited to, benzoic acid, butylparaben, ethyl paraben, methyl paraben, propylparaben, sodium benzoate, sodium propionate, benzalkonium chloride, benzethonium chloride, benzyl alcohol, cetylpyridinium chloride, chlorobutanol, phenol, phenylethyl alcohol, and thimerosal. In one embodiment, a concentration of a preservative that is effective to prevent fungal growth is selected, without affecting the effectiveness of the composition for its intended purposed upon topical application.

Excipients in the formulation are selected based on the type of formulation intended. In certain embodiments, the excipients include gelatin, casein, lecithin, gum acacia, cholesterol, tragacanth, stearic acid, benzalkonium chloride, calcium stearate, glyceryl monostearate, cetostearyl alcohol, cetomacrogol emulsifying wax, sorbitan esters, polyoxyethylene alkyl ethers, polyoxyethylene castor oil derivatives, polyoxyethylene sorbitan fatty acid esters, polyethylene glycols, polyoxyethylene stearates, colloidol silicon dioxide, phosphates, sodium dodecyl sulfate, carboxymethylcellulose calcium, carboxymethylcellulose sodium, methylcellulose, hydroxyethylcellulose, hydroxypropylcellulose, hydroxypropylmethycellulose phthalate, noncrystalline cellulose, magnesium aluminum silicate, triethanolamine, polyvinyl alcohol, polyvinylpyrrolidone, sugars, and starches.

In some embodiments, compositions disclosed herein are formulated with glycerol. In some instances, a strain of bacteria in the composition ferments the glycerol, thereby producing short chain fatty acids. Non-limiting examples of short-chain fatty acids include acetic acid, lactic acid, and propionic acid. In some embodiments, the strain of bacteria is a Propionibacterium strain. In some embodiments, the strain of bacteria is a P. acnes strain.

Penetration enhancers are frequently used to promote transdermal delivery of drugs across the skin, in particular across the stratum corneum. Some penetration enhancers cause dermal irritation, dermal toxicity and dermal allergies. However, the more commonly used ones include urea, (carbonyldiamide), imidurea, N,N-diethylformamide, N-methyl-2-pyrrolidine, 1-dodecal-azacyclopheptane-2-one, calcium thioglycate, 2-pyyrolidine, N,N-diethyl-m-toluamide, oleic acid and its ester derivatives, such as methyl, ethyl, propyl, isopropyl, butyl, vinyl and glycerylmonooleate, sorbitan esters, such as sorbitan monolaurate and sorbitan monooleate, other fatty acid esters such as isopropyl laurate, isopropyl myristate, isopropyl palmitate, diisopropyl adipate, propylene glycol monolaurate, propylene glycol monooleatea and non-ionic detergents such as BRIJ® 76 (stearyl poly(10 oxyethylene ether), BRIJ® 78 (stearyl poly(20)oxyethylene ether), BRIJ® 96 (oleyl poly(10)oxyethylene ether), and BRIJ® 721 (stearyl poly (21) oxyethylene ether) (ICI Americas Inc. Corp.).

The composition can be formulated to comprise the health-associated microbe or probiotic at a particular concentration. For example, the composition can comprise an amount of probiotic such that the microorganisms may be delivered in effective amounts. In certain embodiments, the amount of probiotic delivered is at least 1×10³, 1×10⁴, 1×10⁵, 1×10⁶, 1×10⁷, 1×10⁸, 1×10⁹, 1×10¹⁰ colony forming units per unit dose. The composition may be formulated with the health-associated microbe or probiotic in a proportion of at least about 0.0001% (expressed by dry weight), from about 0.0001% to about 99%, from about 0.001% to about 90% by weight, from about 0.01% to about 80% by weight, and from about 0.1% to about 70% by weight, relative to the total weight of the composition. In general, a composition intended to be administered topically comprises at least 1×10³, 1×10⁴, 1×10⁵, 1×10⁶, 1×10⁷, 1×10⁸, 1×10⁹, 1×10¹⁰ microorganisms per gram of carrier, or at equivalent doses calculated for inactive or dead microorganisms or for bacterial fractions or for metabolites produced.

Synthetic bacteria disclosed herein may be delivered in effective amounts per unit dose, of at least about 1×10² colony forming units (cfu) to about 1×10²⁰ cfu. In the particular case of the compositions that have to be administered topically, the concentration of each bacterial strain and/or corresponding fraction and/or metabolite can be adjusted so as to correspond to doses (expressed as bacterial equivalent) ranging from about 1×10⁵ to about 1×10¹² cfu/dose.

Compositions disclosed herein for topical application generally comprise from about 1×10² to about 1×10¹⁵ cfu/g, from about 1×10⁵ to about 1×10¹² cfu/g, or from about 1×10⁶ to about 10×10¹² cfu/g of synthetic bacteria.

In certain embodiments, compositions disclosed herein are formulated in order to deliver at least 10⁶ synthetic bacteria per cm² of skin. In certain embodiments, the composition is formulated in order to deliver at least 10⁷ synthetic bacteria per cm² of skin. In certain embodiments, the composition is formulated in order to deliver at least 10⁸ synthetic bacteria per cm² of skin. In certain embodiments, the composition is formulated in order to deliver at least 10⁹ synthetic bacteria per cm² of skin. In certain embodiments, the composition is formulated in order to deliver less than 10⁹ synthetic bacteria per cm² of skin. In certain embodiments, the composition is formulated in order to deliver less than 10⁸ synthetic bacteria per cm² of skin. In certain embodiments, the composition is formulated in order to deliver less than 10⁷ synthetic bacteria per cm² of skin. In certain embodiments, the composition is formulated in order to deliver between about 10⁷ and 10⁸ synthetic bacteria per cm² of skin. In certain embodiments, the composition is formulated in order to deliver between about 10⁶ synthetic bacteria per cm² of skin and about 10¹⁰ synthetic bacteria per cm² of skin. In certain embodiments, the composition is formulated in order to deliver between about 10⁶ synthetic bacteria per cm² of skin and about 10⁹ synthetic bacteria per cm² of skin. In certain embodiments, the composition is formulated in order to deliver between about 10⁷ synthetic bacteria per cm² of skin and about 10¹⁰ synthetic bacteria per cm² of skin. In certain embodiments, the composition is formulated in order to deliver between about 10⁷ synthetic bacteria per cm² of skin and about 10⁹ synthetic bacteria per cm² of skin.

In certain embodiments, compositions disclosed herein are formulated at a concentration of about 10⁵ microbes per milliliter to about 10¹² microbes per milliliter. In certain embodiments, compositions disclosed herein are formulated at a concentration of about 10⁶ microbes per milliliter. In certain embodiments, compositions disclosed herein are formulated at a concentration of about 10⁷ microbes per milliliter. In certain embodiments, compositions disclosed herein are formulated at a concentration of about 10⁸ microbes per milliliter. In certain embodiments, compositions disclosed herein are formulated at a concentration of about 10⁹ microbes per milliliter. In certain embodiments, compositions disclosed herein are formulated at a concentration of about 10¹⁰ microbes per milliliter.

In certain embodiments, compositions disclosed herein for topical or oral use contain biologic stability compounds including but not limited to carbohydrates such as trehalose, mannose, fructose, glucose, sucrose, lactose, raffinose, stachyose, melezitose, dextran, and sugar alcohols; and/or cryopreservatives such as glycerol, bovine-free media, (e.g., tryptic soy broth), whey protein, NaCl, phosphate buffer, MgCl, lyophilized bacteria, or other inactive/killed bacteria.

After formulation, composition disclosed herein may be packaged in a manner suitable for delivery and use by an end user. In one embodiment, the composition is placed into an appropriate dispenser and shipped to the end user. Examples of a final container may include a pump bottle, squeeze bottle, jar, tube, capsule or vial.

In some embodiments, compositions disclosed herein can be added to an applicator before packaging. Non-limiting examples of applicators include a cotton pad, a polyester pad, a q-tip, a sponge, and a brush. In some embodiments, the applicator is placed in a package. Non-limiting examples of a package includes bags and foil or wax lined paper packets. The interior of the package may be sterile. In some embodiments, air in the package is removed with a vacuum before sealing. In some embodiments, the package is heat-sealed. In some embodiments, the package is sealed with adhesive.

In another embodiment, compositions disclosed herein are lyophilized or freeze dried, for reconstitution before application to the skin. In one embodiment, lyophilization or freeze drying is conducted with one or more excipients, such as glycerol or other sugar alcohols, to improve the shelf life of the selected, transformed, or engineered bacteria. In one embodiment, the lyophilized composition does not include trehalose (.alpha.-D-glucopyranosyl-1,1-.alpha.-D-glucopyranosyde). In some embodiments, the composition does not have to be frozen.

Compositions disclosed herein may be packaged in one or more containers. For example, a single bottle, tube, container, or capsule may be divided to two equal or unequal parts wherein one part contains the bacteria, in their packing form (freeze dried/inactive, etc.), and the other part contains an activation material, which can be a liquid or a gel. The single bottle or container can be designed so that an end user can dispense with a single force applied to the container all or a portion of the contents in the two container parts, to dispense onto the skin or other surface the selected, transformed, or engineered bacteria and the activation material. The kit may also be of the form that comprises two or more containers, one container with the population(s) of selected, transformed, or engineered bacteria and the other with a formulation for admixture with the populations of selected, transformed, or engineered bacteria. In another example, two or more containers, one container with the population of selected, transformed, or engineered bacteria, the other container with natural non pathogenic skin bacteria that are not selected, transformed, or engineered, and a third container with a formulation for admixture with the populations of selected, transformed, or engineered bacteria. In another example, the two or more containers composing the single bottle had one pump connected to two separate tubes, each draining from a different chamber. The kit may also include one or more complementary products, such as soaps, body washes or moisturizing lotions with certain pH, lotions or creams containing active compounds, bacteria and limiting factors etc. In another embodiment, the complementary product is a limiting factor that will enhance the growth, activity and/or expression of the compound of interest to provide a lasting or continuous expression of the compound. The complementary product may include any compound beneficial to the activity of the original product, and enhance its activity for lasting efficacy. Another contemplated packaging is one wherein the population of selected, transformed, or engineered bacteria is maintained as a layer on a bandage or film that is combined with a second layer of bandage/film that will allow activation of the bacteria, and that optionally may also limit reproduction/growth factors. In another embodiment, the final product is stored refrigerated, with the bacteria being in their active state. In another embodiment, the bacteria are stored in a small bead of water soluble cellulose. The beads can be mixed in any solution such as sunscreen/moisturizing/body wash or soap.

### Methods of Preserving Preparations of Synthetic Bacteria

Provided herein, in some aspects, are methods of producing desired preparations of synthetic bacteria. In some embodiments, the methods comprise producing a desired preparation of at least one strain of synthetic bacteria that is derived from a Propionibacterium. In some embodiments, the at least one strain is a strain of P. acnes. In some embodiments, the at least one strain is a strain of P. acnes subsp. acnes. In some embodiments, the at least one strain is a strain of P. Avidum. In some embodiments, the at least one strain is a strain of P. granulosum. In preferred embodiments, the strain of Propionibacterium is a health-associated strain, as described herein.

In some embodiments, methods comprise adding a sample of the synthetic bacteria to a glycerol solution to produce a synthetic bacteria glycerol stock, and storing the synthetic bacteria glycerol stock at a temperature of about 4° C. or less. Producing a desired preparation of synthetic bacteria may comprise at least one of cooling, freezing, and storing a synthetic bacteria sample, a composition thereof or a stock thereof.

By way of non-limiting example, methods are provided herein for producing a desired preparation of a synthetic bacteria comprising adding a sample of the synthetic bacteria to a glycerol solution to produce a synthetic bacteria glycerol stock, and storing the synthetic bacteria glycerol stock at a temperature of about 4° C. or less, wherein more than about 50% of the synthetic bacteria is viable when the synthetic bacteria in the glycerol solution is brought to ambient temperature.

Also by way of non-limited examples, methods are provided herein for producing a desired preparation of preserved synthetic bacteria, wherein about 90% of said v is viable after sixty days of storage, said method comprising: adding a sample of synthetic bacteria to a solution of about 50% glycerol to produce a synthetic bacteria stock, freezing the synthetic bacteria glycerol stock at −20° C., thereby forming said desired preparation wherein greater than about 90% of the sample of synthetic bacteria are viable after a thawing of the synthetic bacteria glycerol stock.

In some embodiments, methods comprise storing the synthetic bacteria, wherein at least about 50% of the synthetic bacteria is viable when the synthetic bacteria in the glycerol solution is brought to ambient temperature. In some embodiments, at least about 60% of the synthetic bacteria is viable when the synthetic bacteria in the glycerol solution is brought to ambient temperature. In some embodiments, at least about 70% of the synthetic bacteria is viable when the synthetic bacteria in the glycerol solution is brought to ambient temperature. In some embodiments, at least about 80% of the synthetic bacteria is viable when the synthetic bacteria in the glycerol solution is brought to ambient temperature. In some embodiments, more than about 90% of the synthetic bacteria are viable when the synthetic bacteria in the glycerol solution is brought to ambient temperature.

In some embodiments, methods comprise adding the synthetic bacteria to a glycerol solution, wherein the glycerol solution is between about 5% and about 75% glycerol. In some embodiments, methods comprise adding the synthetic bacteria to a glycerol solution, wherein the glycerol solution is between about 5% and about 65% glycerol. In some embodiments, methods comprise adding the synthetic bacteria to a glycerol solution, wherein the glycerol solution is between about 5% and about 55% glycerol. In some embodiments, methods comprise adding the synthetic bacteria to a glycerol solution, wherein the glycerol solution is between about 5% and about 45% glycerol. In some embodiments, methods comprise adding the synthetic bacteria to a glycerol solution, wherein the glycerol solution is between about 5% and about 35% glycerol. In some embodiments, methods comprise adding the synthetic bacteria to a glycerol solution, wherein the glycerol solution is between about 5% and about 25% glycerol. In some embodiments, methods comprise adding the synthetic bacteria to a glycerol solution, wherein the glycerol solution is between about 25% and about 75% glycerol. In some embodiments, the glycerol solution is between about 30% and about 70% glycerol. In some embodiments, the glycerol solution is between about 35% and about 65% glycerol. In some embodiments, the glycerol solution is between about 40% and about 60% glycerol. In some embodiments, the glycerol solution is between about 45% and about 50% glycerol. In some embodiments, the glycerol solution is about 25% glycerol. In some embodiments, the glycerol solution is about 30% glycerol. In some embodiments, the glycerol solution is about 35% glycerol. In some embodiments, the glycerol solution is about 40% glycerol. In some embodiments, the glycerol solution is about 45% glycerol. In some embodiments, the glycerol solution is about 50% glycerol. In some embodiments, the glycerol solution is about 55% glycerol. In some embodiments, the glycerol solution is about 60% glycerol.

In some embodiments, methods comprise storing synthetic bacteria, or a composition thereof, disclosed herein, at a selected temperature. In some embodiments, methods comprise storing the synthetic bacteria glycerol stock at a selected temperature. In some embodiments, the temperature is between about 30° C. and about −80° C. In some embodiments, the temperature is between about 25° C. and about −80° C. In some embodiments, the temperature is between about 25° C. and about −20° C. In some embodiments, the temperature is between about 30° C. and about 4° C. In some embodiments, the temperature is between about 10° C. and about −80° C. In some embodiments, the temperature is between about 10° C. and about −40° C. In some embodiments, the temperature is between about 10° C. and about −30° C. In some embodiments, the temperature is between about 10° C. and about −20° C. In some embodiments, the temperature is between about 4° C. and about −80° C. In some embodiments, the temperature is between about 4° C. and about −25° C. In some embodiments, the temperature is between about 4° C. and about −20° C. In some embodiments, the temperature is about 22° C. to about 28° C. In some embodiments, the temperature is about 25° C. In some embodiments, the temperature is about 4° C. In some embodiments, the temperature is about −20° C. In some embodiments, the temperature is between about −80° C.

In some embodiments, methods comprise thawing a composition of synthetic bacteria disclosed herein. In some embodiments, methods comprise warming a composition of synthetic bacteria disclosed herein. In some embodiments, methods comprise thawing a synthetic bacteria glycerol stock at room temperature. In some embodiments, methods comprise rapid thawing the synthetic bacteria glycerol stock in a bath. The bath temperature may be between about 25° C. and about 40° C. In some embodiments, methods comprise rapidly thawing a composition of synthetic bacteria disclosed herein. By way of non-limiting example, a subject may apply a composition disclosed herein, wherein the composition is frozen, directly to skin. In some embodiments, methods comprise slowly thawing a composition of synthetic bacteria disclosed herein. By way of non-limiting example, a subject may transfer a composition disclosed herein that is frozen to a refrigerator to reach a refrigerated temperature before being brought to room temperature, before being applied to skin, or before being combined with another composition (e.g., emollient, lotion, gel). The term “frozen” includes compositions at temperatures at which the composition is in a solid form or semi-solid form. Frozen may include compositions at temperatures of less than 0° C., and less than −15° C. The term “refrigerated temperature” refers to a temperature of about 0° C. to about 10° C., e.g., 4° C. A refrigerated temperature does not necessarily need to be achieved with a refrigerator. By non-limiting example, an ice bucket could similarly cool a sample.

In some embodiments, methods comprise storing a synthetic bacteria glycerol stock, wherein at least about 60% to at least about 90% of the synthetic bacteria sample is viable after the synthetic bacteria glycerol stock is brought to ambient temperature. In some embodiments, the at least about 70% to at least about 90% of the P. acnes sample is viable after the synthetic bacteria glycerol stock is brought to ambient temperature. In some embodiments, the at least about 80% to at least about 90% of the viable after the synthetic bacteria glycerol stock is brought to ambient temperature. In some embodiments, at least about 60% of synthetic bacteria sample is viable after the synthetic bacteria glycerol stock is brought to ambient temperature. In some embodiments, at least about 70% of the synthetic bacteria sample is viable after the Propionibacterium glycerol stock is brought to ambient temperature. In some embodiments, at least about 80% of the Propionibacterium sample is viable after the synthetic bacteria glycerol stock is brought to ambient temperature. In some embodiments, at least about 90% of the synthetic bacteria sample is viable after the synthetic bacteria glycerol stock is brought to ambient temperature. Ambient temperature is considered an acceptable room temperature. In some embodiments, the ambient temperature is between about 25° C. and about 35° C. In some embodiments, the ambient temperature is between about 20° C. and about 30° C. In some embodiments, the ambient temperature is between about 22° C. and about 28° C. In some embodiments, the ambient temperature is about 25° C.

In some embodiments, methods comprise storing a synthetic bacteria glycerol stock, wherein at least about 50% of the synthetic bacteria sample is viable after at least about 30 days of storing. In some embodiments, at least about 60% of the synthetic bacteria sample is viable after at least about 30 days of storing. In some embodiments, at least about 70% of the synthetic bacteria sample is viable after at least about 20 days of storing. In some embodiments, at least about 80% of the synthetic bacteria sample is viable after at least about 30 days of storing. In some embodiments, at least about 90% of the synthetic bacteria sample is viable after at least about 30 days of storing. In some embodiments, at least about 95% of the synthetic bacteria sample is viable after at least about 30 days of storing. In some embodiments, at least about 50% of the synthetic bacteria sample is viable after at least about 60 days of storing. In some embodiments, at least about 60% of the synthetic bacteria sample is viable after at least about 60 days of storing. In some embodiments, at least about 70% of the synthetic bacteria sample is viable after at least about 60 days of storing. In some embodiments, at least about 80% of the synthetic bacteria sample is viable after at least about 60 days of storing. In some embodiments, at least about 90% of the synthetic bacteria sample is viable after at least about 60 days of storing. In some embodiments, at least about 95% of the synthetic bacteria sample is viable after at least about 60 days of storing. In some embodiments, at least about 50% of the synthetic bacteria sample is viable after at least about 90 days of storing. In some embodiments, at least about 60% of the synthetic bacteria sample is viable after at least about 90 days of storing. In some embodiments, at least about 70% of the synthetic bacteria sample is viable after at least about 90 days of storing. In some embodiments, at least about 80% of the synthetic bacteria sample is viable after at least about 90 days of storing. In some embodiments, at least about 90% of the synthetic bacteria sample is viable after at least about 90 days of storing. In some embodiments, at least about 95% of the synthetic bacteria sample is viable after at least about 90 days of storing. In some embodiments, at least about 50% of the synthetic bacteria sample is viable after at least about 120 days of storing. In some embodiments, at least about 60% of the synthetic bacteria sample is viable after at least about 120 days of storing. In some embodiments, at least about 70% of the synthetic bacteria sample is viable after at least about 120 days of storing. In some embodiments, at least about 80% of the synthetic bacteria sample is viable after at least about 120 days of storing. In some embodiments, at least about 90% of the synthetic bacteria sample is viable after at least about 120 days of storing. In some embodiments, at least about 95% of the synthetic bacteria sample is viable after at least about 120 days of storing. In some embodiments, at least about 50% of the synthetic bacteria sample is viable after at least about 180 days of storing. In some embodiments, at least about 60% of the synthetic bacteria sample is viable after at least about 180 days of storing. In some embodiments, at least about 70% of the sample is viable after at least about 180 days of storing. In some embodiments, at least about 80% of the synthetic bacteria sample is viable after at least about 180 days of storing. In some embodiments, at least about 90% of the synthetic bacteria sample is viable after at least about 180 days of storing. In some embodiments, at least about 95% of the synthetic bacteria sample is viable after at least about 180 days of storing. In some embodiments, at least about 50% of the synthetic bacteria sample is viable after at least about a year of storing. In some embodiments, at least about 60% of the synthetic bacteria sample is viable after at least about a year of storing. In some embodiments, at least about 70% of the synthetic bacteria sample is viable after at least about a year of storing. In some embodiments, at least about 80% of the synthetic bacteria sample is viable after at least about a year of storing. In some embodiments, at least about 90% of the synthetic bacteria sample is viable after at least about a year of storing. In some embodiments, at least about 95% of the synthetic bacteria sample is viable after at least about a year of storing.

In some embodiments, methods comprise storing synthetic bacteria in a solution, wherein the solution is between about 10% glycerol v/v and about 90% glycerol v/v in solution. In some embodiments, the solution is between about 20% glycerol v/v and about 80% glycerol v/v in solution. In some embodiments, the solution is between about 25% glycerol v/v and about 75% glycerol v/v in solution. In some embodiments, the solution is between about 30% glycerol v/v and about 70% glycerol v/v in solution. In some embodiments, the solution is between about 35% glycerol v/v and about 65% glycerol v/v in solution. In some embodiments, the solution is between about 40% glycerol v/v and about 60% glycerol v/v in solution. In some embodiments, the solution is between about 45% glycerol v/v and about 55% glycerol v/v in solution. In some embodiments, the solution is between about 15% glycerol v/v and about 35% glycerol v/v in solution. In some embodiments, the solution is between about 20% glycerol v/v and about 30% glycerol v/v in solution. In some embodiments, the solution is about 20% glycerol v/v in solution. In some embodiments, the solution is about 25% glycerol v/v in solution. In some embodiments, the solution is about 30% glycerol v/v in solution. In some embodiments, the solution is about 35% glycerol v/v in solution. In some embodiments, the solution is about 40% glycerol v/v in solution. In some embodiments, the solution is about 45% glycerol v/v in solution. In some embodiments, the solution is about 50% glycerol v/v in solution. In some embodiments, the solution is about 50% glycerol v/v in solution. In some embodiments, the solution is about 55% glycerol v/v in solution. In some embodiments, the solution is about 60% glycerol v/v in solution. In some embodiments, the solution is about 65% glycerol v/v in solution. In some embodiments, the solution is about 75% glycerol v/v in solution.

In some embodiments, methods comprise storing synthetic bacteria in a solution, wherein the solution comprises glycerol and water. In some embodiments, the solution consists essentially of glycerol and water. In some embodiments, methods comprise storing synthetic bacteria in a solution, wherein the solution comprises glycerol and a saline solution. In some embodiments, the solution consists essentially of glycerol and a saline solution. In some embodiments, the solution comprises glycerol and a buffered saline solution. In some embodiments, the solution consists essentially of glycerol and a buffered saline solution. In some embodiments, the solution comprises glycerol and a phosphate buffered saline solution. In some embodiments, the solution consists essentially of glycerol and a phosphate buffered saline solution.

In some embodiments, methods comprise storing synthetic bacteria in a solution, wherein the solution has a pH of between about 3.5 and about 7. In some embodiments, the solution has a pH of between about 4 and about 6.5. In some embodiments, the solution has a pH of between about 4 and about 6. In some embodiments, the solution has a pH of between about 4 and about 5.5. In some embodiments, the solution has a pH of between about 4.5 and about 5.5. In some embodiments, the solution has a pH of between about 4.8 and about 5. In some embodiments, the solution has a pH of about 4. In some embodiments, the solution has a pH of about 4.2. In some embodiments, the solution has a pH of about 4.4. In some embodiments, the solution has a pH of about 4.6. In some embodiments, the solution has a pH of about 4.8. In some embodiments, the solution has a pH of about 5. In some embodiments, the solution has a pH of about 5.2. In some embodiments, the solution has a pH of about 5.4. In some embodiments, the solution has a pH of about 5.6. In some embodiments, the solution has a pH of about 5.8. In some embodiments, the solution has a pH of about 6.

In some embodiments, methods comprise storing synthetic bacteria in a solution, wherein the solution comprises a salt or ion thereof. In some embodiments, the solution comprises an ion selected from potassium, calcium, magnesium, sodium, and boron. In some embodiments, the solution comprises potassium. In some embodiments, the solution comprises potassium. In some embodiments, the concentration of the salt or ion thereof is between about 0.001 mM and about 1 mM. In some embodiments, the concentration of the salt or ion thereof is between about 0.001 mM and about 0.1 mM. In some embodiments, the concentration of the salt or ion thereof is between about 0.01 mM and about 0.1 mM. In some embodiments, the concentration of the salt or ion thereof is between about 0.05 mM and about 0.1 mM. In some embodiments, the concentration of the salt or ion thereof is between about 0.01 mM and about 1 mM. In some embodiments, the concentration of the salt or ion thereof is between about 0.1 mM and about 1 mM. In some embodiments, the concentration of the salt or ion thereof is between about 100 mM and about 250 mM. In some embodiments, the concentration of the salt or ion thereof is between about 125 mM and about 225 mM. In some embodiments, the concentration of the salt or ion thereof is between about 150 mM and about 200 mM. In some embodiments, the concentration of potassium is between about 100 mM and about 250 mM. In some embodiments, the concentration of potassium is between about 125 mM and about 225 mM. In some embodiments, the concentration of potassium is between about 150 mM and about 200 mM. In some embodiments, the solution comprises calcium at a concentration of about 0.001 mM to about 1 mM. In some embodiments, the solution comprises calcium at a concentration of about 0.01 mM to about 0.5 mM. In some embodiments, the solution comprises calcium at a concentration of about 0.05 mM to about 0.1 mM.

In some embodiments, methods comprise storing synthetic bacteria in a solution, wherein the solution comprises a prebiotic stabilizing agent. In some embodiments, the prebiotic stabilizing agent is selected from a polysaccharide or oligosaccharide. In some embodiments, the prebiotic stabilizing agent is inulin. In some embodiments, the stabilizing agent is present in the solution at a concentration of about 0.01% v/v to about 1% v/v. In some embodiments, the stabilizing agent is present in the solution at a concentration of about 0.01% v/v to about 0.5% v/v. In some embodiments, the stabilizing agent is present in the solution at a concentration of about 0.05% v/v to about 0.2% v/v. In some embodiments, the solution comprises inulin at a concentration of about 0.01% v/v to about 1% v/v. In some embodiments, the solution comprises inulin at a concentration of about 0.01% v/v to about 0.5% v/v. In some embodiments, the solution comprises inulin at a concentration of about 0.05% v/v to about 0.2% v/v. For clarity, the term % v/v, as used herein, represent the percentage of a total volume of a solution that is represented by a volume of a component of the solution.

In some embodiments, methods comprise storing synthetic bacteria in a solution, wherein the solution comprises an anti-acne agent, wherein the anti-acne agent is an agent that prevents, reduces or abolishes acne. In some embodiments, the anti-acne agent is selected from a retinoid, a vitamin, an antioxidant, a peroxide, an acid, an oil, an alcohol, an extract, and analogs thereof. For clarity, the term, “analog,” as used herein, refers to a compound having a structure similar to that of another one, but differing from it by less than about 10% of the total structure. In some embodiments, the retinoid is selected from tretinoin, tazarotene, adapalene, and retinol. In some embodiments, the vitamin or analog thereof is selected from a Vitamin A, Vitamin C, Vitamin D, Vitamin E, and calciptotriene. In some embodiments, the antioxidant is selected from Vitamin C and Vitamin E. peroxide is benzoyl peroxide. In some embodiments, the acid is selected from salicylic acid, azaelic acid, trichloracetic acid, and glycolic acid. In some embodiments, the alcohol is selected from cetyl alcohol, stearyl alcohol, and cetearyl alcohol. In some embodiments, the alcohol is selected from retinol (also known as Vitamin A₁) and resveratrol. In some embodiments, the oil is selected from lavender oil, clary sage oil, juniper berry oil, bergamot oil, jojoba oil, rosemary oil, coconut oil, avocado oil, peppermint oil, and tea tree oil. In some embodiments, the oil is tea tree oil. In some embodiments, the extract is selected from an extract of aloe, garlic, amaranth, neem, coriander, lemon, basil, grapefruit, cucumber, grape, beet, green tea or a combination thereof. In some embodiments, the extract is a green tea extract.

In some embodiments, methods comprise storing synthetic bacteria in a solution, wherein the solution is incorporated in a biologic stability platform. In some embodiments, the biologic stability platform eliminates a need for temperature control, e.g., cold chain storage. In some embodiments, the biologic storage platform comprises foam drying or foam formation of the solution or glycerol stock solution. In some embodiments, the biologic stability platform comprises at least one of a glyconanoparticle, a liposome, a nanoparticle, trehalose, sucrose, stachyose, hydroxyethyl starch, and a combination of glycine and mannitol.

In some embodiments, methods comprise storing or preserving a sample of synthetic bacteria that his derive from a P. acnes of at least one selected ribotype. the sample of P. acnes bacteria comprises P. acnes bacteria of ribotype RT1. In some embodiments, the sample of P. acnes bacteria comprises P. acnes bacteria of ribotype RT2. In some embodiments, the sample of P. acnes bacteria comprises P. acnes bacteria of ribotypes RT1 and RT2. In some embodiments, the sample of P. acnes bacteria consists essentially of P. acnes bacteria of ribotype RT1. In some embodiments, the sample of P. acnes bacteria consists essentially of P. acnes bacteria of ribotype RT2. In some embodiments, the sample of P. acnes bacteria consists essentially of P. acnes bacteria of ribotypes RT1 and RT2.

In some embodiments, methods may comprise culturing an initial culture of the sample of synthetic bacteria. The initial culture may be a smaller aliquot of the synthetic bacteria and the method may comprise proliferating the synthetic bacteria to obtain a desired amount. In some embodiments, methods may comprise culturing the initial culture in a culture medium. In some embodiments, the cell culture medium comprises reinforced clostridial medium. In some embodiments, the cell culture medium consists essentially of reinforced clostridial medium. In some embodiments, the cell culture medium comprises Luria broth. In some embodiments, the cell culture medium comprises tryptone broth. In some embodiments, methods comprise at least one step of splitting, diluting or passaging the initial culture or product thereof in the culture medium. In some embodiments, the methods comprise at least one step of washing the sample of the initial culture or product thereof. In some embodiments, the methods comprise at least one step of centrifuging or pelleting the initial culture or product thereof. In some embodiments, the culture is centrifuged at about 3500 rcf to about 4500 rcf. In some embodiments, the culture is centrifuged at about 3800 rcf to about 4200 rcf. In some embodiments, the culture is centrifuged at about 4000 rcf. In some embodiments, the methods comprise at least one step of vortexing the initial culture or product thereof. In some embodiments, the methods comprise at least one step of pipetting the initial culture of product thereof. Any one of the steps described herein may be performed at least one time. Any one of the steps described herein may be performed two times. Any one of the steps described herein may be performed three times. In some embodiments, methods comprise adding a sachet to a culture comprising the sample of synthetic bacteria or an initial culture thereof. In some embodiments, the sachet reduces oxygen exposure to the synthetic bacteria.

### Compositions of Preserved Synthetic Bacteria

Provided herein, in some aspects, are compositions that comprise a preserved sample of synthetic bacteria wherein the bacteria comprises Propionibacterium. In some embodiments, the Propionibacterium comprises P. acnes. In some embodiments, compositions disclosed herein comprise P. acnes bacteria of ribotype RT1. In some embodiments, the compositions comprise P. acnes bacteria of ribotype RT2. In some embodiments, the compositions comprise P. acnes bacteria of ribotype RT1 and RT2. In some embodiments, the bacteria of the compositions consist essentially of P. acnes bacteria of ribotype RT1. In some embodiments, the bacteria of the compositions consist essentially of P. acnes bacteria of ribotype RT2. In some embodiments, the compositions comprise P. acnes bacteria of ribotype RT1 and RT2.

In some embodiments, compositions disclosed herein comprise a sample of bacteria preserved in at least one cryopreservative agent. In some embodiments, the cryopreservative agent is a polyol. Non-limiting examples of polyols include DMSO, ethylene glycol, glycerol, propylene (PEG) glycol, sucrose, trehalose, and 2-Methyl-2,4-pentanediol (MPD). In various embodiments, the PEG may have a molecular weight between about 10 g/mol and about 10,000 g/mol. In various embodiments, the PEG may have a molecular weight between about 10 g/mol and about 5,000 g/mol. In various embodiments, the PEG may have a molecular weight between about 10 g/mol and about 1,000 g/mol. In various embodiments, the PEG may have a molecular weight between about 10 g/mol and about 500 g/mol.

In some embodiments, compositions disclose herein comprise polyethylene glycol. In some embodiments, a composition comprising polyethylene glycol allows for a reduced amount of glycerol, whilst maintaining viability of bacteria in the composition that is similar to viability of bacteria in compositions without polyethylene glycol and a greater amount of glycerol. In some embodiments, reducing or minimizing the amount of glycerol in a composition disclosed herein results in a formulation that has a texture that is more preferable to a subject. In some embodiments, reducing or minimizing the amount of glycerol in a composition disclosed herein results in a formulation that less comedogenic relative to a composition with a greater amount of glycerol.

In some embodiments, compositions disclosed herein comprise a sample of bacteria preserved in a mixture of a first polyol and a second polyol. In some embodiments, the first polyol or the second polyol is glycerol. In some embodiments, the first polyol or the second polyol is a polyethylene glycol. In some embodiments, compositions disclosed herein comprise a sample of bacteria preserved in a mixture of glycerol and polyethylene glycol. In some embodiments, the mixture is between about 1% glycerol v/v and about 50% glycerol v/v, and between about 1% polyethylene glycol w/v and about 50% w/v polyethylene glycol. In some embodiments, the mixture is between about 5% glycerol v/v and about 50% glycerol v/v, and between about 5% polyethylene glycol w/v and about 50% w/v polyethylene glycol. In some embodiments, the mixture is between about 5% glycerol v/v and about 30% glycerol v/v, and between about 5% polyethylene glycol w/v and about 40% w/v polyethylene glycol. In some embodiments, the mixture is between about 10% glycerol v/v and about 35% glycerol v/v, and between about 10% polyethylene glycol w/v and about 35% w/v polyethylene glycol.

In some embodiments, compositions disclosed herein comprise a sample of bacteria preserved in at least one cryopreservative agent. In some embodiments, the cryopreservative agent is a polyol. Non-limiting examples of polyols include DMSO, ethylene glycol, glycerol, propylene (PEG) glycol, sucrose, trehalose, and 2-Methyl-2,4-pentanediol (MPD). In various embodiments, the PEG may have a molecular weight between about 10 g/mol and about 10,000 g/mol. In various embodiments, the PEG may have a molecular weight between about 10 g/mol and about 5,000 g/mol. In various embodiments, the PEG may have a molecular weight between about 10 g/mol and about 1,000 g/mol. In various embodiments, the PEG may have a molecular weight between about 10 g/mol and about 500 g/mol.

In some embodiments, compositions disclose herein comprise polyethylene glycol. In some embodiments, a composition comprising polyethylene glycol allows for a reduced amount of glycerol, whilst maintaining viability of bacteria in the composition that is similar to viability of bacteria in compositions without polyethylene glycol and a greater amount of glycerol. In some embodiments, reducing or minimizing the amount of glycerol in a composition disclosed herein results in a formulation that has a texture that is more preferable to a subject. In some embodiments, reducing or minimizing the amount of glycerol in a composition disclosed herein results in a formulation that less comedogenic relative to a composition with a greater amount of glycerol.

In some embodiments, compositions disclosed herein comprise a sample of bacteria preserved in a mixture of a first polyol and a second polyol. In some embodiments, the first polyol or the second polyol is glycerol. In some embodiments, the first polyol or the second polyol is a polyethylene glycol. In some embodiments, compositions disclosed herein comprise a sample of bacteria preserved in a mixture of glycerol and polyethylene glycol. In some embodiments, the mixture is between about 1% glycerol v/v and about 50% glycerol v/v, and between about 1% polyethylene glycol w/v and about 50% w/v polyethylene glycol. In some embodiments, the mixture is between about 5% glycerol v/v and about 50% glycerol v/v, and between about 5% polyethylene glycol w/v and about 50% w/v polyethylene glycol. In some embodiments, the mixture is between about 5% glycerol v/v and about 30% glycerol v/v, and between about 5% polyethylene glycol w/v and about 40% w/v polyethylene glycol. In some embodiments, the mixture is between about 10% glycerol v/v and about 35% glycerol v/v, and between about 10% polyethylene glycol w/v and about 35% w/v polyethylene glycol.

In some embodiments, compositions disclosed herein comprise a solution, wherein the solution comprises glycerol and water. In some embodiments, the solution consists essentially of glycerol and water. In some embodiments, methods comprise storing synthetic bacteria in a solution, wherein the solution comprises glycerol and a saline solution. In some embodiments, the solution consists essentially of glycerol and a saline solution. In some embodiments, the solution comprises glycerol and a buffered saline solution. In some embodiments, the solution consists essentially of glycerol and a buffered saline solution. In some embodiments, the solution comprises glycerol and a buffered solution. In some embodiments, the buffered solution comprises sodium bicarbonate, citric acid or triethanolamine. In some embodiments, the solution comprises glycerol and a phosphate buffered saline solution. In some embodiments, the solution consists essentially of glycerol and a phosphate buffered saline solution.

In some embodiments, the solution is about 5% glycerol v/v in solution. In some embodiments, the solution is about 10% glycerol v/v in solution. In some embodiments, the solution is about 15% glycerol v/v in solution. In some embodiments, the solution is about 20% glycerol v/v in solution. In some embodiments, the solution is about 25% glycerol v/v in solution. In some embodiments, the solution is about 30% glycerol v/v in solution. In some embodiments, the solution is about 35% glycerol v/v in solution. In some embodiments, the solution is about 40% glycerol v/v in solution. In some embodiments, the solution is about 45% glycerol v/v in solution. In some embodiments, the solution is about 50% glycerol v/v in solution. In some embodiments, the solution is about 55% glycerol v/v in solution. In some embodiments, the solution is about 60% glycerol v/v in solution. In some embodiments, the solution is about 70% glycerol v/v in solution.

In some embodiments, compositions disclosed herein comprise a solution, wherein the solution comprises glycerol and water. In some embodiments, the solution consists essentially of glycerol and water. In some embodiments, methods comprise storing Propionibacterium in a solution, wherein the solution comprises glycerol and a saline solution. In some embodiments, the solution consists essentially of glycerol and a saline solution. In some embodiments, the solution comprises glycerol and a buffered saline solution. In some embodiments, the solution consists essentially of glycerol and a buffered saline solution. In some embodiments, the solution comprises glycerol and a buffered solution. In some embodiments, the buffered solution comprises sodium bicarbonate, citric acid or triethanolamine. In some embodiments, the solution comprises glycerol and a phosphate buffered saline solution. In some embodiments, the solution consists essentially of glycerol and a phosphate buffered saline solution.

In some embodiments, compositions disclosed herein comprise a solution, wherein the solution has a pH of between about 3.5 and about 7. In some embodiments, the solution has a pH of between about 4 and about 6.5. In some embodiments, the solution has a pH of between about 4 and about 6. In some embodiments, the solution has a pH of between about 4 and about 5.5. In some embodiments, the solution has a pH of between about 4.5 and about 5.5. In some embodiments, the solution has a pH of between about 4.8 and about 5. In some embodiments, the solution has a pH of about 4. In some embodiments, the solution has a pH of about 4.2. In some embodiments, the solution has a pH of about 4.4. In some embodiments, the solution has a pH of about 4.6. In some embodiments, the solution has a pH of about 4.8. In some embodiments, the solution has a pH of about 5. In some embodiments, the solution has a pH of about 5.2. In some embodiments, the solution has a pH of about 5.4. In some embodiments, the solution has a pH of about 5.6. In some embodiments, the solution has a pH of about 5.8. In some embodiments, the solution has a pH of about 6.

In some embodiments, compositions disclosed herein comprise a solution, wherein the solution comprises a salt or ion thereof. In some embodiments, the solution comprises an ion selected from potassium, calcium, magnesium, sodium, and boron. In some embodiments, the solution comprises potassium. In some embodiments, the solution comprises potassium. In some embodiments, the concentration of the salt or ion thereof is between about 0.001 mM and about 1 mM. In some embodiments, the concentration of the salt or ion thereof is between about 0.001 mM and about 0.1 mM. In some embodiments, the concentration of the salt or ion thereof is between about 0.01 mM and about 0.1 mM. In some embodiments, the concentration of the salt or ion thereof is between about 0.05 mM and about 0.1 mM. In some embodiments, the concentration of the salt or ion thereof is between about 0.01 mM and about 1 mM. In some embodiments, the concentration of the salt or ion thereof is between about 0.1 mM and about 1 mM. In some embodiments, the concentration of the salt or ion thereof is between about 100 mM and about 250 mM. In some embodiments, the concentration of the salt or ion thereof is between about 125 mM and about 225 mM. In some embodiments, the concentration of the salt or ion thereof is between about 150 mM and about 200 mM. In some embodiments, the concentration of potassium is between about 100 mM and about 250 mM. In some embodiments, the concentration of potassium is between about 125 mM and about 225 mM. In some embodiments, the concentration of potassium is between about 150 mM and about 200 mM. In some embodiments, the solution comprises calcium at a concentration of about 0.001 mM to about 1 mM. In some embodiments, the solution comprises calcium at a concentration of about 0.01 mM to about 0.5 mM. In some embodiments, the solution comprises calcium at a concentration of about 0.05 mM to about 0.1 mM.

In some embodiments, compositions disclosed herein comprise a solution, wherein the solution comprises at least one stabilizing agent. In some embodiments, the stabilizing agent is selected from inulin, sucrose, trehalose, cornstarch, maltodextrin, guar guy, locust bean gum, and xanathan gum. In some embodiments, trehalose or sucrose stabilizes bacteria for cold-chain free stability. In some embodiments, the stabilizing agent is inulin. In some embodiments, the stabilizing agent is present in the solution at a concentration of about 0.01% v/v to about 1% v/v. In some embodiments, the stabilizing agent is present in the solution at a concentration of about 0.01% v/v to about 0.5% v/v. In some embodiments, the stabilizing agent is present in the solution at a concentration of about 0.05% v/v to about 0.2% v/v. In some embodiments, the solution comprises inulin at a concentration of about 0.01% v/v to about 1% v/v. In some embodiments, the solution comprises inulin at a concentration of about 0.01% v/v to about 0.5% v/v. In some embodiments, the solution comprises inulin at a concentration of about 0.05% v/v to about 0.2% v/v.

In some embodiments, compositions disclosed herein comprise a solution, wherein the solution comprises an anti-acne agent, wherein the anti-acne agent is an agent that prevents, reduces or abolishes acne. In some embodiments, the anti-acne agent is selected from a retinoid, a vitamin, an antioxidant, a peroxide, an acid, an oil, an alcohol, an extract, and analogs thereof. In some embodiments, the retinoid is selected from tretinoin, tazarotene, adapalene, and retinol. In some embodiments, the vitamin or analog thereof is selected from Vitamin D, Vitamin C, Vitamin E, and calciptotriene. In some embodiments, the antioxidant is selected from Vitamin C and Vitamin E. peroxide is benzoyl peroxide. In some embodiments, the acid is selected from salicylic acid, azaelic acid, trichloracetic acid, and glycolic acid. In some embodiments, the alcohol is selected from retinol and resveratrol. In some embodiments, the oil is tea tree oil. In some embodiments, the extract is a green tea extract.

In some embodiments, compositions disclosed herein comprise a solution, wherein the solution is incorporated in a biologic stability platform. In some embodiments, the biologic stability platform eliminates a need for temperature control, e.g., cold chain storage. In some embodiments, the biologic storage platform comprises foam drying or foam formation of the solution or glycerol stock solution. In some embodiments, the biologic stability platform comprises at least one of a glyconanoparticle, a liposome, a nanoparticle, trehalose, sucrose, stachyose, hydroxyethyl starch, and a combination of glycine and mannitol.

In some embodiments, compositions disclosed herein have a temperature of about −80° C. to about 10° C. In some embodiments, the composition is at a temperature of about −80° C. to about 4° C. In some embodiments, the composition is at a temperature of about −40° C. to about 10° C. In some embodiments, the composition is at a temperature of about −25° C. to about 10° C. In some embodiments, the composition is at a temperature of about −20° C. to about 4° C. In some embodiments, the composition is at a temperature of about −90° C. to about −70° C. In some embodiments, the composition is at a temperature of about −30° C. to about −10° C. In some embodiments, the composition is at a temperature of about −80° C. In some embodiments, the composition is at a temperature of about −20° C. In some embodiments, the composition is at a temperature of about 4° C.

In some embodiments, compositions disclosed herein comprise a synthetic bacteria glycerol stock, wherein at least about 60% to at least about 90% of the synthetic bacteria sample is viable after the Propionibacterium glycerol stock is brought to ambient temperature. In some embodiments, the at least about 70% to at least about 90% of the synthetic bacteria sample is viable after the synthetic bacteria glycerol stock is brought to ambient temperature. In some embodiments, the at least about 80% to at least about 90% of the viable after the synthetic bacteria glycerol stock is brought to ambient temperature. In some embodiments, at least about 60% of the synthetic bacteria sample is viable after the synthetic bacteria glycerol stock is brought to ambient temperature. In some embodiments, at least about 70% of the synthetic bacteria sample is viable after the synthetic bacteria glycerol stock is brought to ambient temperature. In some embodiments, at least about 80% of the synthetic bacteria sample is viable after the synthetic bacteria glycerol stock is brought to ambient temperature. In some embodiments, at least about 90% of the synthetic bacteria sample is viable after the synthetic bacteria glycerol stock is brought to ambient temperature. Ambient temperature is considered an acceptable room temperature. In some embodiments, the ambient temperature is between about 25° C. and about 35° C. In some embodiments, the ambient temperature is between about 20° C. and about 30° C. In some embodiments, the ambient temperature is between about 22° C. and about 28° C. In some embodiments, the ambient temperature is about 25° C.

In some embodiments, methods comprise storing the synthetic bacteria, wherein at least about 1% of the synthetic bacteria is viable when the synthetic bacteria in the glycerol solution is brought to ambient temperature. In some embodiments, methods comprise storing the synthetic bacteria, wherein at least about 5% of the synthetic bacteria is viable when the synthetic bacteria in the glycerol solution is brought to ambient temperature. In some embodiments, methods comprise storing the synthetic bacteria, wherein at least about 10% of the synthetic bacteria is viable when the synthetic bacteria in the glycerol solution is brought to ambient temperature. In some embodiments, methods comprise storing the synthetic bacteria, wherein at least about 15% of the synthetic bacteria is viable when the synthetic bacteria in the glycerol solution is brought to ambient temperature. In some embodiments, methods comprise storing the synthetic bacteria, wherein at least about 20% of the synthetic bacteria is viable when the synthetic bacteria in the glycerol solution is brought to ambient temperature. In some embodiments, methods comprise storing the synthetic bacteria, wherein at least about 30% of the synthetic bacteria is viable when the synthetic bacteria in the glycerol solution is brought to ambient temperature. In some embodiments, methods comprise storing the synthetic bacteria, wherein at least about 40% of the synthetic bacteria is viable when the synthetic bacteria in the glycerol solution is brought to ambient temperature. In some embodiments, compositions disclosed herein comprise a synthetic bacteria glycerol stock, wherein at least about 50% of the synthetic bacteria sample is viable after at least about 30 days of storing. In some embodiments, at least about 60% of the synthetic bacteria sample is viable after at least about 30 days of storing. In some embodiments, at least about 70% of the synthetic bacteria sample is viable after at least about 20 days of storing. In some embodiments, at least about 80% of the synthetic bacteria sample is viable after at least about 30 days of storing. In some embodiments, at least about 90% of the synthetic bacteria sample is viable after at least about 30 days of storing. In some embodiments, at least about 95% of the synthetic bacteria sample is viable after at least about 30 days of storing. In some embodiments, at least about 50% of the synthetic bacteria sample is viable after at least about 60 days of storing. In some embodiments, at least about 60% of the synthetic bacteria sample is viable after at least about 60 days of storing. In some embodiments, at least about 70% of the synthetic bacteria sample is viable after at least about 60 days of storing. In some embodiments, at least about 80% of the synthetic bacteria sample is viable after at least about 60 days of storing. In some embodiments, at least about 90% of the synthetic bacteria sample is viable after at least about 60 days of storing. In some embodiments, at least about 95% of the synthetic bacteria sample is viable after at least about 60 days of storing. In some embodiments, at least about 50% of the synthetic bacteria sample is viable after at least about 90 days of storing. In some embodiments, at least about 60% of the synthetic bacteria sample is viable after at least about 90 days of storing. In some embodiments, at least about 70% of the synthetic bacteria sample is viable after at least about 90 days of storing. In some embodiments, at least about 80% of the synthetic bacteria sample is viable after at least about 90 days of storing. In some embodiments, at least about 90% of the synthetic bacteria sample is viable after at least about 90 days of storing. In some embodiments, at least about 95% of the synthetic bacteria sample is viable after at least about 90 days of storing. In some embodiments, at least about 50% of the synthetic bacteria sample is viable after at least about 120 days of storing. In some embodiments, at least about 60% of the synthetic bacteria sample is viable after at least about 120 days of storing. In some embodiments, at least about 70% of the synthetic bacteria sample is viable after at least about 120 days of storing. In some embodiments, at least about 80% of the synthetic bacteria sample is viable after at least about 120 days of storing. In some embodiments, at least about 90% of the synthetic bacteria sample is viable after at least about 120 days of storing. In some embodiments, at least about 95% of the synthetic bacteria sample is viable after at least about 120 days of storing. In some embodiments, at least about 50% of the synthetic bacteria sample is viable after at least about 180 days of storing. In some embodiments, at least about 60% of the synthetic bacteria sample is viable after at least about 180 days of storing. In some embodiments, at least about 70% of the sample is viable after at least about 180 days of storing. In some embodiments, at least about 80% of the synthetic bacteria sample is viable after at least about 180 days of storing. In some embodiments, at least about 90% of the synthetic bacteria sample is viable after at least about 180 days of storing. In some embodiments, at least about 95% of the synthetic bacteria sample is viable after at least about 180 days of storing. In some embodiments, at least about 50% of the synthetic bacteria sample is viable after at least about a year of storing. In some embodiments, at least about 60% of the synthetic bacteria sample is viable after at least about a year of storing. In some embodiments, at least about 70% of the synthetic bacteria sample is viable after at least about a year of storing. In some embodiments, at least about 80% of the synthetic bacteria sample is viable after at least about a year of storing. In some embodiments, at least about 90% of the synthetic bacteria sample is viable after at least about a year of storing. In some embodiments, at least about 95% of the synthetic bacteria sample is viable after at least about a year of storing.

In some embodiments, compositions disclosed herein have a storage life of at least about thirty days to at least about ninety days. In some embodiments, the compositions disclosed herein have a storage life of at least about 30 days to at least about 120 days. In some embodiments, the compositions disclosed herein have a storage life of at least about 30 days to at least about 180 days. In some embodiments, the compositions disclosed herein have a storage life of at least about thirty days to about ninety days. In some embodiments, the compositions disclosed herein have a storage life of at least about 30 days to about 120 days. In some embodiments, the compositions disclosed herein have a storage life of at least about 30 days to about 180 days. In some embodiments, the compositions disclosed herein have a storage life of at least about thirty days. In some embodiments, the compositions disclosed herein have a storage life of at least about sixty days. In some embodiments, the compositions disclosed herein have a storage life of at least about ninety days. In some embodiments, the compositions disclosed herein have a storage life of at least about 120 days. In some embodiments, the compositions disclosed herein have a storage life of at least about 180 days. In some embodiments, the compositions disclosed herein have a storage life of at least about 240 days. In some embodiments, the compositions disclosed herein have a storage life of at least about one year. In some embodiments, the compositions disclosed herein have a storage life of up to about one year.

In some embodiments, the compositions disclosed herein are capable of being thawed and subsequently applied to a subject in need thereof. In some embodiments, the compositions disclosed herein are capable of being warmed and subsequently applied to a subject in need thereof. In some embodiments, the compositions disclosed herein are capable of being refrigerated and subsequently applied to a subject in need thereof. In some embodiments, subsequently applied to the subject comprises applying the composition directly to the skin of the subject. In some embodiments, subsequently applied to the subject comprises applying the composition to an application composition before being applied to the skin. The application composition may be selected from a liquid, gel, lotion, emollient, paste, mask, and virtually any solution that can be applied to the skin of a subject. In some embodiments, the application composition is free of any anti-acne agent. In some embodiments, the application composition comprises an anti-acne agent. In some embodiments, the compositions disclosed herein are capable of being applied directly from a frozen stock to skin of a subject without thawing or warming.

### EXAMPLES

The following examples are set forth to illustrate more clearly the principle and practice of embodiments disclosed herein to those skilled in the art and are not to be construed as limiting the scope of any claimed embodiments.

### Example 1: Synthetic Bacteria

A synthetic bacteria is engineered from a P. acnes bacteria. One or more metabolic pathways are modified from the P. acnes bacteria such that the synthetic bacteria produce less than 4 micromolar levels of porphyrins. The synthetic bacteria produce lower levels of lipases and have lower glucose metabolism than an acne-associated P. acnes bacteria. The synthetic bacteria are further engineered to prevent activation of an immune response in a human host by lacking biomolecules that bind to host inflammatory receptors, such as TLR2, TLR4 and protease activated receptors. Some bacteria are produced with modified extracellular antigens, such as pili, to circumvent a host immune response.

### Example 2: Clinical Trial

Purpose:

The purpose of this study is to assess effectiveness of a synthetic P. acnes on acne lesions in subjects having acne.

Intervention:

A composition comprising synthetic P. acnes bacteria from Example 1 are applied to acne affected areas of subjects twice daily over a treatment period of 8-12 weeks.

Detailed Description:

Subjects are assessed prior to administration for the presence of acne lesions in an affected area. Subjects apply the composition comprising different test amounts of synthetic bacteria derived from P. acnes twice daily. After the treatment period, an assessment is performed to evaluate the number of acne lesions in the affected area.

Eligibility and Inclusion Criteria:

Male and female subjects that are 18 to 40 years old and are diagnosed as having acne lesions.

Exclusion Criteria:

Patients with a psychiatric disorder that might cause difficulty in obtaining informed consent or in conducting the trial.

Primary Outcome Measures:

Determine percentage change is number of acne lesions before, during and after treatment. Monitor long-term effects of acne lesions after treatment, such as evaluating the number of acne lesions in the affected area days, weeks, and/or months after treatment.

Secondary Outcome Measures:

Determine adverse effects.

### Example 3: Identification of Health-Associated Strains

Characteristics that may predispose a particular microbe to be a health-associated microbe can be determined using samples from healthy and disease afflicted individuals, culturing the microbes from each, and performing a comparative genomic analysis. In the present example, samples were collected from individuals afflicted with acne vulgaris in order to determine health-associated P. acnes strains.

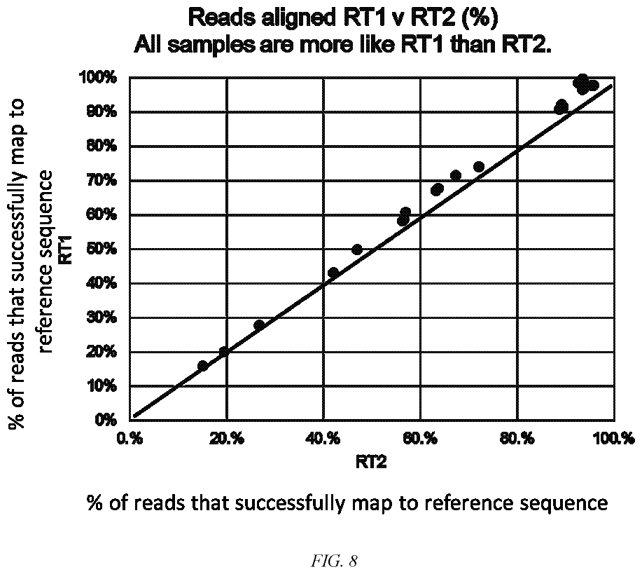

Microcomedone or swab samples were collected from consented adult subjects. Clonal samples were isolated by limiting dilution on plates, and then grown in 200 μL of liquid culture. Microbial DNA was isolated from 96 individual cultures. DNA was isolated using QIAgen's DNeasy Blood & Tissue kit, following the manufacturer's instructions. QIAgen's DNeasy Blood & Tissue kit, following the manufacturer's instructions. Paired-end DNA sequencing (2×300 bp) was done on an Illumina MiSeq using reagent kit v3, following the manufacturer's instructions, yielding 200,000 to 600,000 reads for each of the 96 samples. Initial analysis was performed in Illumina's Basespace Sequence Hub, all reads from each sample are aligned with a BWA Aligner to:
a. deoR;
b. Propionibacterium acnes ATCC 11828 (accession CP003084); or
c. pIMPLE and other reference genomes.
   Alignments were interrogated with the Broad Institute's Integrative Genomics Viewer and confirmed using Biomatter's Geneious version 9.1. All 96 clones were analyzed for the presence or absence of the deoR sequence, type I lipase or type II lipase sequence, and presence or absence of pIMPLE plasmid. Sequence alignments were performed between sequences of P. acnes from healthy volunteers and the deoR gene. Analysis revealed that approximately half of all healthy clones were positive for deoR (greater than 0.4% of reads mapping to deoR locus). Sequence alignments were also performed between P. acnes of healthy volunteers and the lipase gene locus P. acnes were positive for type I Lipase and for type II Lipase. With regard to the pIMPLE plasmid sequence alignments of reads from healthy volunteers performed against pIMPLE-HL096PA1 (GenBank: CP003294.1), revealed P. acnes from healthy volunteers are free of pIMPLE plasmid. Reads from healthy volunteers map P. acnes to ribotype RT1. FIG. 8 corroborates this by showing that more RT1 strains are deoR positive and type II lipase positive when compare to RT2. Some results are summarized in Table 8.

TABLE 8
summary of sequencing data for the P. acnes
isolated from healthy volunteers

| | | RT1; | RT1; | | | | |
|---|---|---|---|---|---|---|---|
| | RT1; | deoR+; | deoR+; | | | | |
| | deoR− | LP1 | LP2 | RT2 | Staph. | Other | sum |
| reads | 112 | 160 | 42 | 1 | 7 | 48 | 370 |
| % of total | 30.3% | 43.2% | 11.4% | 0.3% | 1.9% | 13.0% | |

RT1 = ribotype 1;
RT2 = ribotype 2;
deoR− = no deoR;
deoR+ = deoR;
LP1 = type I; Lipase;
PL2 = type II lipase;
Staph = Staphylococcus; other P. avidum, P. acidipropionici, or Staphylococcus

### Example 4: Identification of Health-Associated Strains with Hyaluronidase Genes

Health-associated P. acnes clones that were RT1 or RT2 positive were further examined for presence of a gene encoding hyaluronidase. Unexpectedly most health-associated strains that were positive for Type II lipase also possessed a hyaluronidase gene. See Table 9.

TABLE 9
Hyaluronidase presence in health-associated P. acnes strains
also positive for type II lipase

| Clone | genotype | Hyaluronidase |
|---|---|---|
| 1 | RT1; deoR+; L2 | Yes |
| 2 | RT1; deoR+; L2 | Yes |
| 3 | RT1; deoR+; L2 | Yes |
| 4 | RT1; deoR+; L2 | Yes |
| 5 | RT1; deoR+; L2 | Yes |
| 6 | RT1; deoR+; L1 | No |
| 7 | RT1; deoR+; L2 | Yes |
| 8 | RT1; deoR+; L2 | Yes |
| 9 | RT1; deoR+; L2 | Yes |
| 10 | RT1; deoR+; L2 | Yes |
| 11 | RT1; deoR+; L2 | Yes |
| 12 | RT2 | Yes |
| 13 | RT2 | Yes |
| 14 | RT2 | Yes |
| 15 | RT2 | Yes |

RT1 = Ribotype 1;
RT2 = Ribotype 2;
L1 −Lipase type I;
L2 = Lipase type 2;
Deor+ = DeoRepressor positive

### Example 5: P. acnes Viability Assay

Viability of P. acnes was assessed each week over two months of storage as shown in Table 10. At least three samples were tested at each time point.

TABLE 10
Assessed P. acnes storage conditions

| Solution | Temperature |
|---|---|
| 25% glycerol in water | 4° C. |
| 50% glycerol in water | 4° C. |
| 25% glycerol in 75% PBS | 4° C. |
| 25% glycerol in water | −20° C. |
| 50% glycerol in water | −20° C. |
| 25% glycerol in water | −80° C. |

Samples were prepared according to the following:

1. P. acnes of ribotypes RT1(HP3A11) and RT2 (HP5G4) were started at 0.066 OD600 and grown to ˜1.0 OD600 in exponential phase in reinforced clostridial medium (RCM).

2. A day later, cultures displayed a dense turbidity, and they were split 1:2 with RCM to produce four liquid culture (LC) samples of each ribotype: 4 RT2 LC and 4 RT1 LC.

3. Two days later, resulting pellets and media were separated. The media of the LC was split between two tubes (˜3 ml), and tubes were filled with 9 ml fresh media and vortexed. Pellets remained in original test tubes and were resuspended by pipetting with 8 ml fresh RCM. All LC (the 8 pellet LCs (4 RT1 and 4RT2), and 16 media-derived LCs) were placed into ajar with two sachets given a large quantity of oxygen filled the jar.

4. LCs were vortexed, split and fed fresh media as they became very turbid and large pellets formed.

5. A day before the experiment, cultures were vortexed, split, spun down at 4,300 g for 5 minutes, and media replaced.

6. On the day of initiating storage: LCs were split into sterile 50 ml conical tube (e.g., 50 ml aliquots of RT1 or RT2), avoiding the pelleted cells. Conical tubes were vortexed lightly and OD600 measured. Optionally, LCs may be diluted if OD600 is greater than 1.0.

7. LCs were split into aliquots and spun down at 4,000 rcf for 5 minutes. Media was discarded and pellets washed with 5 ml 25% v/v glycerol/water to wash the cells. Cells were centrifuged once more, and wash solution discarded.

8. Cells were added to 8.75 ml 25% glycerol in water, 6 ml 50% glycerol in water or 3.25 ml 25% glycerol in PBS to produce live bacteria solutions.

9. 250 microliters of live bacteria solutions were added to 1.5 ml eppendorf tubes, and placed at 4° C., −20° C. or −80° C.

Cell viability was assessed according to the following:

1. At each time point, Eppendorf tubes were selected from each treatment, and allowed to come to room temperature. Tubes were inverted six times.

2. 20 microliters of the thawed stocks were serially diluted in 96 well plates with RCM.

3. Thawed stocks were also spotted on Brucella plates at various dilutions.

4. Plates were imaged with a digital camera, and cells counted with 95% Confidence Interval.

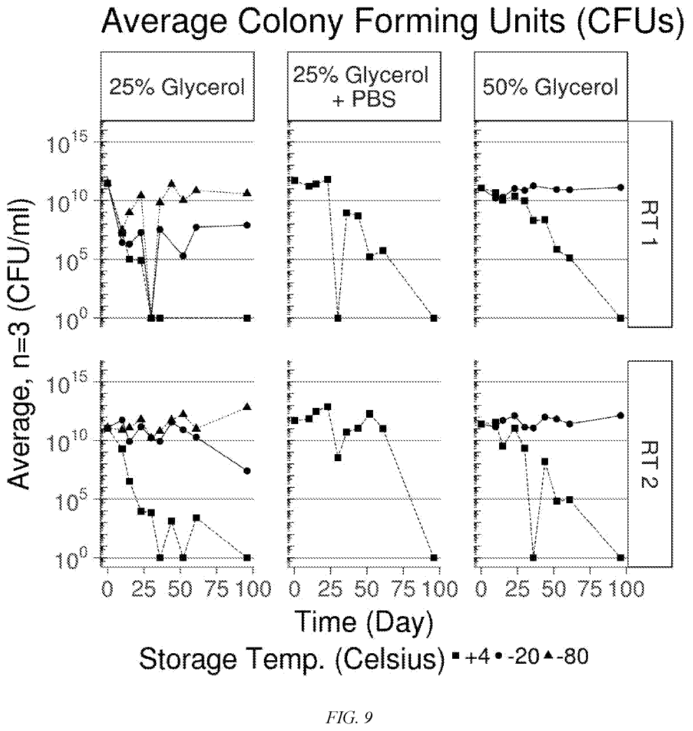

FIG. 9 shows the viability of a variety of Ribotype 1 and Ribotype 2 P. acnes preparations after 30 days, 60 days and 90 days of preservation. Heat shock of a sample, simulating direct application to skin, demonstrated that these samples would retain reported viability if used for acne treatment.

### Example 6. Identification of P. acnes RT6

In an effort to isolate and purify health-associated strains of P. acnes, (e.g., strains associated with acne) it may be useful to identify undesirable strains of P. acnes in a sample (e.g., strains found on skin of subjects with acne). For instance, in some cases, P. acnes of ribotype RT6 is undesirable. To this end, genes can be identified that are specific to strains of interest. The following example demonstrates how this can be performed.

Identities of genes that distinguish P. acnes of ribotype RT6 from healthy strains were confirmed. Genes encoding DNA binding response regulator and phosphoglycerate kinase were identified in P. acnes of ribotype RT6, but not RT1, RT2, RT3, RT4 and RT5. In addition a gene encoding ABC transporter is absent in RT6, but present in RT1, RT2, RT3, RT4 and RT5. Sequences for these genes are provided as SEQ ID NOS: 109 (ABC transporter), 110 (DNA binding response regulator), and 112 (phosphoglycerate kinase)

The presence or absence of these genes was confirmed by sequence alignment using BLAST, Megablast, (a registered trademark of the National Library of Medicine) either the whole complete genome or all of the scaffolds of a completed genome against each of these three gene sequences; the results are shown in Table 11. “Y” is a perfect match for the entire sequence OR >60 bp continuous perfect sequence alignment. “N” means there is <60 bp perfect alignment. The best match of a “N” was 26 bp.

TABLE 11
Genotypes of P. acnes strains

| | | | | DNA | |
|---|---|---|---|---|---|
| | | | | binding | Phospho- |
| | | recA | ABC | response | glycerate |
| Strain Name | Ribotype | type | transporter | regulator | kinase |
| HL002PA2 | 1 | IA | Y | N | N |
| HL025PA1 | 1 | IB | Y | N | N |
| HL030PA1 | 1 | IB | Y | N | N |
| HL050PA2 | 1 | II | Y | N | N |
| HL096PA3 | 1 | IA | Y | N | N |
| HP3A11 | 1 | IB | Y | N | N |
| HP3B4 | 1 | | Y | N | N |
| KPA171202 | 1 | IB | Y | N | N |
| ATCC 11828 | 2 | II | Y | N | N |
| HL001PA1 | 2 | II | Y | N | N |
| HL103PA1 | 2 | II | Y | N | N |
| HP4G1 | 2 | II | Y | N | N |
| HP5G4 | 2 | II | Y | N | N |
| HL002PA1 | 3 | IB | Y | N | N |
| HL005PA1 | 4 | IA | Y | N | N |
| HL007PA1 | 4 | IA | Y | N | N |
| HL038PA1 | 4 | IA | Y | N | N |
| HL045PA1 | 4 | IA | Y | N | N |
| HL053PA1 | 4 | IA | Y | N | N |
| HL056PA1 | 4 | IA | Y | N | N |
| HL074PA1 | 4 | IA | Y | N | N |
| HL099PA1 | 4 | IA | Y | N | N |
| HL043PA1 | 5 | IA | Y | N | N |
| HL043PA2 | 5 | IA | Y | N | N |
| HL072PA1 | 5 | IA | Y | N | N |
| HL072PA2 | 5 | IA | Y | N | N |
| HL096PA1 | 5 | IA | Y | N | N |
| HL096PA2 | 5 | IA | Y | N | N |
| HL097PA1 | 5 | IC | Y | N | N |
| PRP-38 | 5 | IC | Y | N | N |
| HL110PA3 | 6 | II | N | Y | Y |
| HL110PA4 | 6 | II | N | Y | Y |

### Example 7. Pan Bacterial Assay to Characterize Skin Microbiome

Robust pan-sampling of the skin microbiome is demonstrated in the following example. This can be performed with or without the use of preservatives. This method is compatible with qPCR analysis and does not require DNA purification. TaqMan qPCR assays were used to quantitate most bacteria collected from the face. Performance was confirmed with two different bacterial phyla, all Propionibacterium and Staphylococcus. This method required the assessment of only a single locus to recognize most bacteria commonly found on the face (P. acnes strains and Staphylococcus), whereas current methods in the field use multiple primer pairs to achieve similar coverage. The majority of the bacteria on the skin of a subject's face is described in the following Table 12.

TABLE 12
Bacteria on Human Facial Skin

| | P. acnes | P. avidum | S. epidermidis | S. aureus |
|---|---|---|---|---|
| Kingdom | Bacteria | Bacteria | Bacteria | Bacteria |
| Phylum | Actinobacteria | Actinobacteria | Firmicutes | Firmicutes |
| | | | Bacilli | Bacilli |
| Order | Actinomycetales | Actinomycetales | Bacillales | Bacillales |
| Family | Propionibacteriaceae | Propionibacteriaceae | Staphylococcaceae | Staphylococcaceae |
| Genus | Propionibacterium | Propionibacterium | Staphylococcus | Staphylococcus |
| Species | P. acnes | P. avidum | S. epidermidis | S. aureus |

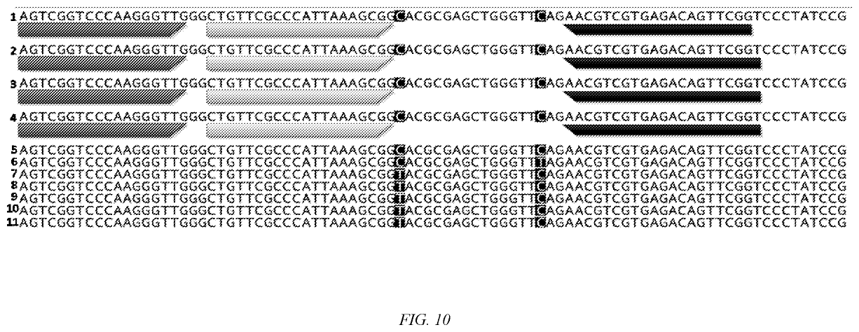

A portion of a 23 S sequence from bacteria commonly found on the human face was aligned with known sequences, see FIG. 10, and SEQ ID NOs: 114 to 124. Despite two Single Nucleotide Polymorphisms at this loci (denoted by bold and underlined letters), careful placement of primers (gray and black) and TaqMan reporter (white) enable quantification of widely diverse bacteria from both Actinobacteria and Firmicutes.

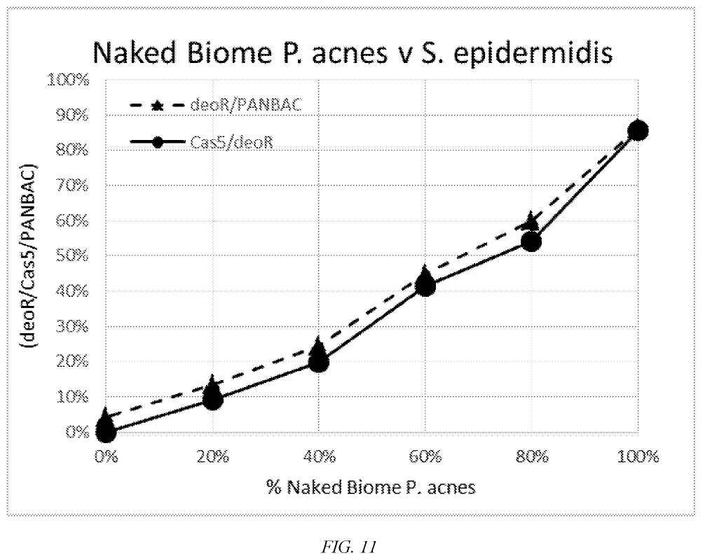

A standard curve for all assays was generated with P. acnes. Percentages of health-associated P. acnes were computed using a dilution series with S. epidermidis or pathogenic P. acnes which were used to quantitate a percentage of health-associated P. acnes in a collected sample. These percentages were determined by measuring deoR+ or Cas5+ bacteria in the overall sample of bacteria (PANBAC), see, e.g., FIG. 11.

### Example 8. Determination of Percentage of pIMPLE Plasmid

The percentage of pIMPLE plasmid was determined from biological samples.

Biological samples were collected and grown in 200 μL of liquid culture. DNA was isolated using QIAgen's DNeasy Blood & Tissue kit, following the manufacturer's instructions. Paired-end DNA sequencing (2×300 bp) was done on an Illumina MiSeq using reagent kit v3, following the manufacturer's instructions, yielding 200,000 to 600,000 reads for each sample. Initial analysis was performed in Illumina's Basespace Sequence Hub, all reads from each sample are aligned with a BWA Aligner to pIMPLE. Alignments were interrogated with the Broad Institute's Integrative Genomics Viewer and confirmed using Biomatter's Geneious version 9.1.

The percentage of pIMPLE was determined by the percentage of total sequencing reads that aligned to pIMPLE plasmid from HL096PA1. The percentage of pIMPLE was also calculated as the coverage*copy number. Using these methods, the percentage of pIMPLE in the different ribotypes was determined as seen in Table 13.

TABLE 13
Presence of pIMPLE plasmid in different P. acnes strains.

| Ribotype | Strain | % pIMPLE |
|---|---|---|
| 1 | HP3A11 | 0.23% |
| 1 | HP3A11 | 0.24% |
| 2 | HP5G4 | 0.26% |
| 2 | HP5G4 | 0.24% |
| 2 | HP4G1 | 0.26% |
| 2 | HP4G1 | 0.25% |
| 4 | HL045PA1 | 3.62% |
| 4 | HL045PA1 | 3.22% |
| 5 | HL043PA1 | 4.32% |
| 5 | HL043PA1 | 3.75% |
| 6 | HL110PA3 | 12.94% |
| 6 | HL110PA3 | 12.59% |
| 6 | HL110PA4 | 13.19% |
| 6 | HL110PA4 | 14.06% |

### Example 9. Genetic Modification of P. acnes

In order to improve healthy P. acnes clones, the expression of a gene in the porphyrin synthetic pathway was knocked out. This was accomplished by inserting stop codons in the middle of the open reading frame of the gene HemY (protoporphyrinogen oxidase, EC:1.3.3.4 1.3.3.15) in the P. acnes genome. Briefly, the RNA-guided DNA endonuclease Cas9 (CRISPR associated protein 9) was targeted to HemY with specific CRISPR RNA (crRNA), and trans-activating RNA (tracrRNA) cleaving a double stranded break at the desired location in the HemY gene. A specific sequence was inserted at the site of the cleavage with a Homology Directed Repair cassette (HDR).

The Cas9, crRNA, tracrRNA, and HDR donor template were introduced into P. acnes using electroporation to transform the cells. Cells must be electrocompetent before undergoing electroporation. Electrocompetent P. acnes were prepared by growing them to stationary phase and washing them in a buffer of sucrose, magnesium chloride, and monosodium phosphate.

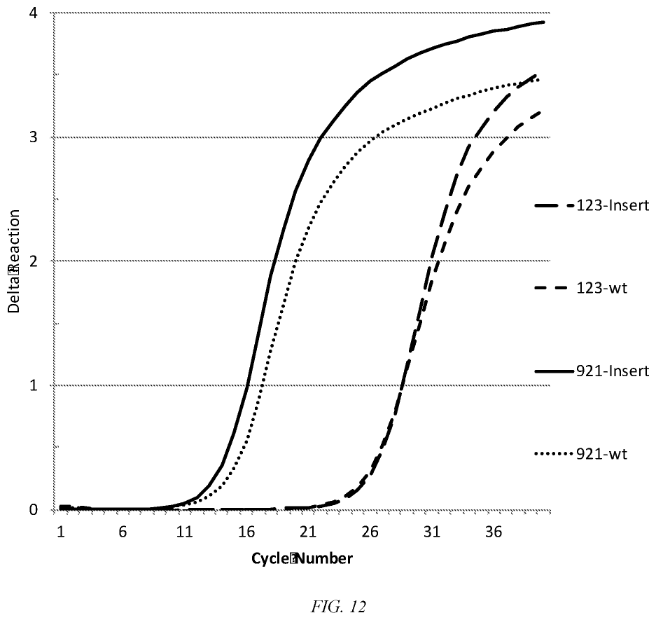

The tracrRNA and crRNA were duplexed using IDT's duplex-forming buffer. Then the tracrRNA:crRNA duplex was incubated in a solution of Cas9 and phosphate-buffered saline, forming the ribonucleoprotein (RNP) complex. The RNPs, HDR, and electrocompetent P. acnes were combined, incubated on ice (transformation culture) and transferred to a pre-chilled BioRAD electroporation cuvette. The transformation culture was electroporated using a BioRAD Micropulser. Rich clostridium medium was immediately added to the transformation culture and transferred to separate container for a 24 hour, room temperature incubation. The transformation culture was evaluated with qPCR (see FIG. 12) and spread out over multiple Brucella plates for a final 72 hour anaerobic incubation at 37° C.

FIG. 12 compares a qPCR result from cells transformed with a 921 bp (921) or a 123 bp HDR. Each sample was evaluated with primers that recognized either the inserted sequence (Insert) or the untransformed or wild-type (wt) genomic sequence. Note, using the longer, 921 bp, HDR transformed a greater percentage of the cells. The ‘921’ sample had more cells resulting the leftward shift of both Insert and wt lines.

### Example 10. Packaging Compositions of Synthetic Bacteria as Swabs for Topical Application

A packaging system was created to store and deliver therapeutically effective doses of pharmaceutical probiotic compositions disclosed herein to the human face. These devices need to safely store and deliver approximately 4 milliliters of P. acnes in a pharmaceutically acceptable excipient, anaerobically. Furthermore, these systems were amenable to storage at temperatures as low as −80° C. The packaging system prevented contamination of both the probiotic (by the environment) and the environment (by the probiotic), minimize exposure to any air, and enable easy application.

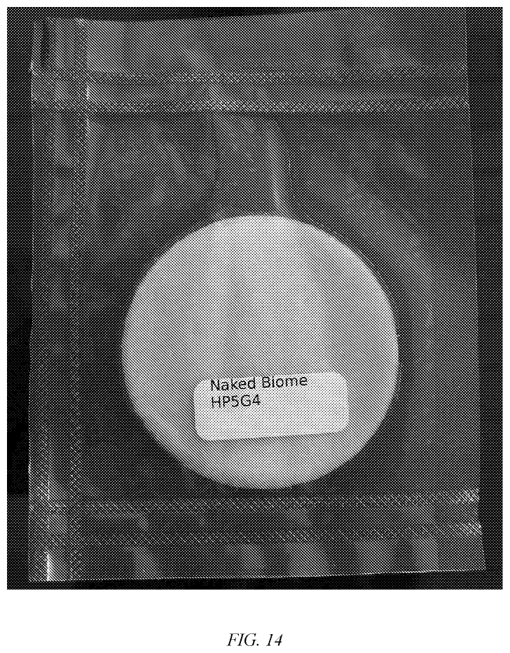

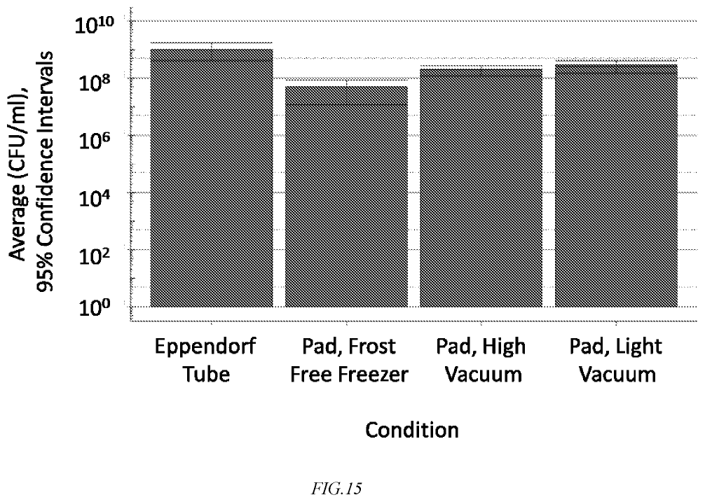

An example of an aforementioned package is shown in FIG. 14. Briefly an approximately 2 inch diameter circular cotton pad was placed in a laminated polypropylene bag. Three to five milliliters of P. acnes solution, at ˜10⁹ microbes per milliliter, was aseptically applied to the cotton pad. Almost all of the air was evacuated, and the bag was thermally sealed in a chamber vacuum sealer (Vacmaster VP215). These packages were easily opened and the pad removed for application of the probiotic or measurement of recovery and viability. Near quantitative aseptic recovery was achieved by centrifugation of the pad in a 15 milliliter conical tube. To confirm that the cotton pad, polypropylene bags and high vacuum do not compromise viability of P. acnes, or retain P. acnes, samples were collected from four different conditions and determined CFUs/milliliter by plating and counting colonies. FIG. 15 compares recovery from P. acnes samples stored for one week at ˜20° C. in either an Eppendorf tube (control), or in our packaging in a residential frost free freezer, a laboratory freezer or in the lab freezer with only a light vacuum before sealing. Large numbers of viable P. acnes were recovered from all conditions. However, as shown in FIG. 15, vacuum sealing resulted in an approximately 10-fold increase in viability as compared to standard storage conditions in a frost free freezer.

Alternative types of pads and bags could be employed for such packaging. For example, if a polyester pad is used, heat sealing could affix the pad to one side of the bag providing a shield and handle to enable application of the liquid therapeutic without mess and exposure to the hand. Similarly if a peel-open pouch is used, scissors would not be necessary for clean easy application

TABLE 13
Exemplary Sequences (Additional SEQ IDs provided in sequence listing filed herewith).
Bold characters highlight differences between Type I lipase and Type II lipase.

| SEQ | | |
|---|---|---|
| ID | | |
| NO: | Description | Sequence |
| 114 | | agtcggtccc aagggttggg ctgttcgccc attaaagcgg cacgcgagct gggttcagaa |
| | | cgtcgtgaga cagttcggtc cctatccg |
| 115 | | agtcggtccc aagggttggg ctgttcgccc attaaagcgg cacgcgagct gggttcagaa |
| | | cgtcgtgaga cagttcggtc cctatccg |
| 116 | | agtcggtccc aagggttggg ctgttcgccc attaaagcgg cacgcgagct gggttcagaa |
| | | cgtcgtgaga cagttcggtc cctatccg |
| 117 | | agtcggtccc aagggttggg ctgttcgccc attaaagcgg cacgcgagct gggttcagaa |
| | | cgtcgtgaga cagttcggtc cctatccg |
| 118 | | agtcggtccc aagggttggg ctgttcgccc attaaagcgg cacgcgagct gggttcagaa |
| | | cgtcgtgaga cagttcggtc cctatccg |
| 119 | | agtcggtccc aagggttggg ctgttcgccc attaaagcgg cacgcgagct gggtttagaa |
| | | cgtcgtgaga cagttcggtc cctatccg |
| 120 | | agtcggtccc aagggttggg ctgttcgccc attaaagcgg tacgcgagct gggttcagaa |
| | | cgtcgtgaga cagttcggtc cctatccg |
| 121 | | agtcggtcccaagggttggg ctgttcgccc attaaagcgg tacgcgagct gggttcagaa |
| | | cgtcgtgaga cagttcggtc cctatccg |
| 122 | | agtcggtccc aagggttggg ctgttcgccc attaaagcgg tacgcgagct gggttcagaa |
| | | cgtcgtgaga cagttcggtc cctatccg |
| 123 | | agtcggtccc aagggttggg ctgttcgccc attaaagcgg tacgcgagct gggttcagaa |
| | | cgtcgtgaga cagttcggtc cctatccg |
| 124 | | agtcggtccc aagggttggg ctgttcgccc attaaagcgg tacgcgagct gggttcagaa |
| | | cgtcgtgagacagttcggtc cctatccg |
| 125 | | caaccgtaga tacagataca tctgaggaga tc |
| 126 | | catgaagaaa aa |
| 127 | | ccgcgcc |
| 128 | | tcaggttcgc aatgaaga |
| 129 | | atgacagaca ggtcctatcc ggcgatgatc cggcttcggc gcaacgcctg gaccgagttc |
| | | gtcccgttcc tggattacga cgtcgagatc cgcaagatcc tctgctcgac gaacgcgatc |
| | | aagtcgttga acacccgctt ccgcacggtc atgcgggcgc agggtcattt cccgacgcgc |
| | | tga |
| 130 | Type I | gtagatacagatacatctgaggagatccatgaagaaaaactggttactcacaaccctccttgccaca |
| | lipase | atgatgatcgccatgggcacgacgaccaccgccttcgccagcccgcctaccgacatcactcccgaa |
| | | catccaggcggggttaccgcgcctcacagccccgacggaatcccctcgaatattgaggggccaagt |
| | | atgcccagctggacctctgcaatcaggttcgcaatgaagaaccccggcacgaaagtcccgggcacc |
| | | aacgacttcacctgcaaaccgaggaaaggcacccatcccgtcgtgctcatcccgggcacatccgag |
| | | gacgccttcatcacgtggtcgtactacggtccccgccaggattctgcgcctacacgttcaactacaac |
| | | ccggaaacacatccgcttgtggaagccgctgagaccagcggcaacatctactccacggcagctttc |
| | | atggcccacttcgttgacagagtgctcaaggcaaccggtgctcagaaggtcaacctcgtcggccatt |
| | | ctcagggcggcggccccctgccgcgcgcgtacatcaaatattacggggcgccaagaaagtcctcat |
| | | ctcgtcggtttggttccttccaacaggggaacacgcatgctcggcctggagaagttcctcaatgccag |
| | | cggaaacccgctcagcactatcttcaatgctgcagcacagtttcgaaagctggaatccctgccccaac |
| | | agttgcaagactccacatttctcagggaactcaacgcggatggaatgaccgtccccggcatcacata |
| | | caccgtcatcgccacccagttcgacaaccgagtatttccgtggactaataccttcatcaatgagcccg |
| | | gggtcaagaacatcgtcatccaagacgtctgtcccttggaccacagcgcccacacggatatccctag |
| | | gacccgatgacccttcagattgtcatcaacgccttggaccccgagcgggccgccccggtcacctgc |
| | | accattcgcccattcaggcccagttag |
| 131 | Type II | gcagatgcatctgagaagatccatgaagaaaaactggttactcacaaccctccttgccacaatgatga |
| | lipase | tcgccatgggcacgacgaccaccgccttcgccagcccgcctaccgacatcactcccgaacatccag |
| | | gcggggttacccgcctcacagccccgacggaatcccctcgaatattgaggggccaagtatgcccag |
| | | ctggacctctgcaatcaggttcgcaatgaagaaccccggcacgaaagtcccgggcaccaacgactt |
| | | cacctgcaaaccgaggaaaggcacccatcccgtcgtgctcatcccgggcacatccgaggacgcctt |
| | | catcacgtggtcgtactacggtccccgccaggattctgcgcctacacgttcaactacaacccggaaa |
| | | cacatccgcttgtggaagccgctgagaccagcggcaacatctactccacggcagctttcatggccca |
| | | cttcgttgacagagtgctcaaggcaaccggtgctcagaaggtcaacctcgtcggccattctcagggc |
| | | ggcggccccctgccgcgcgcgtacatcaaatattacggggcgccaagaaagtcctcatctcgtcgg |
| | | tttggttccttccaacaggggaacacgcatgctcggcctggagaagttcctcaatgccagcggaaac |
| | | ccgctcagcactatcttcaatgctgcagcacagtttcgaaagctggaatccctgccccaacagttgca |
| | | agactccacatttctcagggaactcaacgcggatggaatgaccgtccccggcatcacatacaccgtc |
| | | atcgccacccagttcgacaaccgagtatttccgtggactaataccttcatcaatgagcccggggtcaa |
| | | gaacatcgtcatccaagacgtctgtcccttggaccacagcgcccacacggatatccctaggacccga |
| | | tgacccttcagattgtcatcaacgccttggaccccgagcgggccgccccggtcacctgcaccattcg |
| | | cccattcaggcccagttag |

While preferred embodiments of the present invention have been shown and described herein, it will be obvious to those skilled in the art that such embodiments are provided by way of example only. Numerous variations, changes, and substitutions will now occur to those skilled in the art without departing from the invention. It should be understood that various alternatives to the embodiments of the invention described herein may be employed in practicing the invention.

1.-29. (canceled)

30. A synthetic bacteria derived from a non-pathogenic bacteria, the synthetic bacteria comprising:
a) a porphyrin pathway comprising a biomolecule introduced, removed, or modified relative to the porphyrin pathway of the non-pathogenic bacteria; provided that porphyrin is not produced by the synthetic bacteria or is produced at a level less than about 4 micromolar of porphyrin in the synthetic bacteria;
b) a modified lipase compared to a native lipase;
c) a biomolecule introduced, wherein the biomolecule is adapted to produce an enzyme at a tunable level to effect a second metabolic pathway of the synthetic bacteria;
d) a vitamin B12 metabolic pathway comprising a biomolecule introduced, removed, or modified relative to the vitamin B12 metabolic pathway of the non-pathogenic bacteria; provided that the synthetic bacteria produces a high levels of intracellular vitamin B12 as compared to the non-pathogenic bacteria;
e) a hyaluronidase from a heterologous species as the non-pathogenic bacteria;
f) a nitrous oxide pathway comprising a biomolecule introduced, removed, or modified relative to the nitrous oxide pathway of the non-pathogenic bacteria; provided that the synthetic bacteria produces nitrous oxide;
g) a fatty acid synthesis pathway comprising a biomolecule introduced, removed, or modified relative to the fatty acid synthesis pathway of the non-pathogenic bacteria; provided that the synthetic bacteria produces fatty acids;
h) a biomolecule introduced, removed, or modified relative to the citric acid pathway of the non-pathogenic bacteria; provided that the synthetic bacteria produces glycine;
i) a biomolecule introduced, wherein the biomolecule is adapted to secrete a TNF-alpha inhibitor, an interleukin-8 inhibitor, a tumor neutrophil chemotaxis inhibitor, an interleukin-6 inhibitor, an NFkB inhibitor, human beta defensing-1 inhibitor, human beta defensing-2 inhibitor, or a human beta defensing-3 inhibitor;
j) a biomolecule introduced, wherein the biomolecule is adapted to secrete a corticotropin releasing hormone (CRH), a corticotropin releasing hormone receptor (CRHR), a corticotropin releasing hormone binding protein (CRHBP), or a combination thereof; or
k) any combination of (a), (b), (c), (d), (e), (f), (g), (h), (i) and (j).

31. The synthetic bacteria of claim 30, wherein the non-pathogenic bacteria comprises Propionibacterium acnes.

32. The synthetic bacteria of claim 31, wherein the Propionibacterium acnes bacteria comprises Ribotype 2 and/or Ribotype 1.

33. The synthetic bacteria of claim 30, wherein the non-pathogenic bacteria has been engineered or selected to comprise at least one gene encoding at least one of a deoxyribose operon repressor and a type II lipase.

34. The synthetic bacteria of claim 30, wherein the non-pathogenic bacteria comprises less than about 10% pIMPLE plasmid.

35. The synthetic bacteria of claim 30, wherein the non-pathogenic bacteria comprises a strain selected from at least one of HP4G1, HP5G4, HP3A11, and HP3B4.

36. The synthetic bacteria of claim 30, wherein the non-pathogenic bacteria expresses an ATP binding cassette transporter.

37. The synthetic bacteria of claim 30, wherein the non-pathogenic bacteria does not express a DNA binding response regulator or a phosphoglycerate kinase.

38. The synthetic bacteria of claim 30, wherein the biomolecule comprises an enzyme encoded by one or more of the following genes: HemY, PPA2095 protoporphyrinogen oxidase (HemY homologue), HemE, HemF, HemG, HemH, COX15, cyoE, HemB, HemC, HemD, gdhA, gudB, rocG, narJ, narI, narH, narG, E.1.7.2.1, norB, cysG-cbiX, cobl-cbiL, cobM, cbiF, cobK, cbiJ, cobH, cbiC, cobB-cbiA, cobO, btuR, cobQ, cbiP, cbiB, cobD, cobS, cobV, fadD, CS, IDH1, OGDH, DLST, and fumC.

39. The synthetic bacteria of claim 30, wherein the biomolecule comprises a stop codon or truncation in any one or more of the following genes: HemY, and PPA2095 protoporphyrinogen oxidase (HemY homologue), HemE, HemF, HemG, HemH COX15, cyoE, HemB, HemC, and HemD.

40. The synthetic bacteria of claim 30, wherein the enzyme comprises at least one of ST2S, ST4S, ST6S, bmpA, PTS-Mtl-EIIABC, MFS ST1, MFS ST2, ST1P, ST3P, ST4P, ST5P, ST6P, ST7P, PTS-Mtl-EIIA, ST1P1, ST3P1, ST4P1, ST6P1, FhuD, ST7A, manA, FhuC, FhuB, FhuD, COX15, talAB, hMuV, HtaA, HmuT, GAPDH, hemH, CS, IDH1, cobA-hemD, cysG, IDH1, IDH1, TGL, OGDH, narJ, narl, narH, narG, E1.7.2.1, norB, gdhA, gudB, rocG, MDT1, MDT2, MDT3, T2SF2, T2SF1, secA, secY, secF, secD, yajC, secE, ftsY, yidC, secG, clpX, clp2, and clp1.

41. The synthetic bacteria of claim 30, wherein the modified lipase has less than about 50% of the activity of the native lipase or no lipase activity.

42. The synthetic bacteria of claim 30, wherein the modified lipase comprises a disruption to a gene selected from HMPREF0675_4856, HMPREF0675_4855, HMPREF0675_4479, HMPREF0675_4480, HMPREF0675_4481, HMPREF0675_3655/3657, HMPREF0675_4816, HMPREF0675_4817, HMPREF0675_5205, HMPREF0675_5206, HMPREF0675_5014, HMPREF0675_5101, HMPREF0675_5159, HMPREF0675_4093/4094, HMPREF0675_4163, HMPREF0675_5031, HMPREF0675_5390, HMPREF0675_3037, or a homolog thereof having greater than 90%, homology.

43. The synthetic bacteria of claim 30, wherein the hyaluronidase from a heterologous species comprises a hyaluronidase from a Group B Streptococcus.

44. A composition comprising the synthetic bacteria of claim 30 and an excipient or biological stabilizer.

45. The composition of claim 44 formulated for topical application.

46. A method of treating a skin disorder, comprising administering the composition of claim 44 to a subject in need thereof.

47. The composition of claim 46, wherein the skin disorder comprises acne, psoriasis, eczema, atopic dermatitis, or seborrheic dermatitis.

48. A method of making the synthetic bacteria of claim 30, the method comprising introducing one or more of a CRISPR RNA (crRNA), Cas9, trans-activating RNA (tracrRNA), and a homology directed repair cassette (HDR) greater than 200 base pairs in length into the non-pathogenic bacteria.
